(19) United States
(12) Patent Application Publication
(10) Pub. No.: US 2024/0067639 A1
(43) Pub. Date: Feb. 29, 2024

(54) SMALL MOLECULES THAT TARGET THE RNA THAT CAUSES ALS

(71) Applicants: EXPANSION THERAPEUTICS, INC., San Diego, CA (US); THE SCRIPPS RESEARCH INSTITUTE, La Jolla, CA (US)

(72) Inventors: Matthew Disney, Jupiter, FL (US); Timothy Allen Blizzard, Princeton, NJ (US); Ippei Usui, Jupiter, FL (US)

(73) Assignee: UNIVERSITY OF FLORIDA RESEARCH FOUNDATION, INCORPORATION, Gainesville, FL (US)

(21) Appl. No.: 18/044,260

(22) PCT Filed: Sep. 8, 2021
(86) PCT No.: PCT/US21/49357

§ 371 Date: Mar. 7, 2023

Related U.S. Application Data
(60) Provisional application No. 63/075,390, filed on Sep. 8, 2020.

(51) Int. Cl. C07D 471/04; C07D 403/12; C07D 491/048; C07D 495/04

(52) U.S. Cl. CPC C07D 471/04; C07D 403/12; C07D 491/048; C07D 495/04

(57) ABSTRACT

Disclosed herein are compounds that selectively bind an expanded transcribed repeat r(G₄C₂)^{exp}, prevent sequestration of RNA-binding proteins, and inhibit translation of repeat associated non-ATG (RAN) translation responsible for generation of toxic dipeptide repeats underlying diseases such as amyotrophic lateral sclerosis (ALS) and frontotemporal dementia (FTD). The compounds and their pharmaceutical compositions are useful in treating a disease or condition characterized by an expanded G₄C₂ repeat RNA (r(G₄C₂)^{exp}), such as ALS and FTD.

### STATEMENT OF GOVERNMENT SUPPORT

This invention was made with government support under grants Nos. DP1 NS096898-02, P01 NS099114-01, and R35NS097273, awarded by the National Institutes of Health. The U.S. government has certain rights in the invention.

### BACKGROUND

Amyotrophic lateral sclerosis (ALS), a devastating neurodegenerative disorder caused by progressive loss of motor neurons, often results in paralysis and death within three to five years of onset (Taylor et al., 2016). Current treatment options for ALS remain scarce and are only palliative (Taylor et al., 2016). With the discovery that an expanded, transcribed G₄C₂ repeat in C9ORF72 is the most common genetic cause of ALS and frontotemporal dementia (FTD) (“c9ALS/FTD”), a new therapeutic target has emerged (DeJesus-Hernandez et al., 2011; Renton et al., 2011). The transcribed repeat RNA (“r(G₄C₂)^{exp}”) plays an essential role in two putative pathological mechanisms: (i) sequestering proteins involved in RNA biogenesis to form toxic r(G₄C₂)^{exp}-protein foci (Prudencio et al., 2015); and (ii) undergoing repeat associated non-ATG (RAN) translation to produce toxic c9RAN dipeptide repeats (DPRs) in cellular (Almeida et al., 2013; Taylor et al., 2016; Zu et al., 2013), mouse (Chew et al., 2015; ORourke et al., 2015), and Drosophila (Mizielinska et al., 2014) models.

Current efforts towards developing c9ALS/FTD therapeutics include both antisense oligonucleotides (ASOs) and small molecules that target r(G₄C₂)^{exp}. For example, an ASO directed at C9ORF72 mRNA improved ALS-associated defects in mouse models (Gendron et al., 2017; Jiang et al., 2016). In addition, a small molecule 1a binds r(G₄C₂)^{exp} and inhibits RAN translation and foci formation in both cultured cells and induced neurons, (Su et al., 2014):

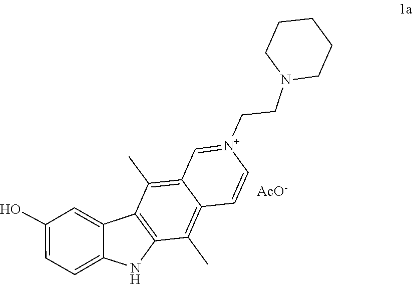 [SMILES: "AcO-" ; Cc1c2cc[n+](CCN3CCCCC3)cc2c(C)c2c1[nH]c1ccc(O)cc12]

### SUMMARY

Provided herein are compounds or pharmaceutically acceptable salts thereof, having a structure of Formula (I) and their use, e.g., in a method of treating a subject suffering from a disease or condition characterized by an expanded G₄C₂ repeat RNA (r(G₄C₂)^{exp}):

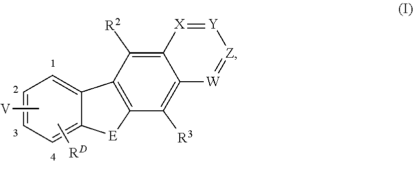

wherein one or two of W, X, Y, and Z is N and the others are CR⁴, or each of W, X, Y, and Z is CR⁴; E is NR⁶, O, or S; V is H, halo, OR¹, C(O)NR^{N}R¹, C(O)OR¹, or NR^{A}R^{B}; R¹ is H, C₁₋₆alkyl, C₁₋₆haloalkyl, C₁₋₆cyanoalkyl, C₁₋₆hydroxyalkyl, C₁₋₆alkylene-C₁₋₆alkoxy, C₂₋₆alkenyl, C₂₋₆alkynyl, C₀₋₆alkylene-C(O)C₁₋₆alkyl, C₀₋₆alkylene-CO₂R^{N}, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-aryl, C₀₋₆alkylene-(5-10 membered heteroaryl having 1-3 ring heteroatoms selected from N, O and S), C₁₋₆alkylene-C(NH)NR^{A}R^{B}, C₁₋₆alkylene-NR^{A}R^{B}, C₁₋₆alkylene-C(O)NR^{A}R^{B}, C(O)—C₃₋₁₄carbocyclyl, or C(O)-(3-14 membered heterocyclyl having 1-3 ring heteroatoms selected from N, O and S), and the carbocyclyl, aryl, heterocyclyl, or heteroaryl is optionally substituted with 1 or 2 R^{C} groups; each of R², R³, and R⁴ is independently H, halo, CN, OH, C₁₋₆alkyl, C₁₋₆haloalkyl, C₁₋₆alkoxy, C₂₋₆alkenyl, C₂₋₆alkynyl, C₀₋₆alkylene-aryl, NR^{A}R^{B}, SO₀₋₂C₁₋₆alkyl, SO₀₋₂aryl, C(O)C₁₋₆alkyl, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), C(O)C₃₋₁₄carbocyclyl, CO₂H, C(O)NR^{N}—C₁₋₆alkylene-NR^{A}R^{B}, or C₁₋₆alkylene-NR^{A}R^{B}, and the carbocyclyl, heterocyclyl, aryl, or heteroaryl is optionally substituted with 1 or 2 R^{C} groups; R⁶ is H, C₁₋₆alkyl, C₁₋₆haloalkyl, C₁₋₆alkylene-NR^{A}R^{B}, C₁₋₆alkylene-C₃₋₁₄carbocyclyl, C₁₋₆alkylene-(3-14 membered heterocyclyl having 1-3 ring heteroatoms selected from N, O and S), C₁₋₆alkylene-aryl, C₁₋₆hydroxyalkylene-aryl, or C₁₋₆alkylene-(5-10 membered heteroaryl having 1-3 ring heteroatoms selected from N, O and S), and carbocyclyl, heterocyclyl, aryl or heteroaryl is optionally substituted with 1 or 2 R^{C}; R^{A} and R^{B} are independently H, C₁₋₆alkyl, C₁₋₆haloalkyl, C₁₋₆hydroxyalkyl, C₂₋₆alkenyl, C₂₋₆alkynyl, C₁₋₆alkylene-C₁₋₆alkoxy, SO₀₋₂C₁₋₆alkyl, SO₀₋₂aryl, C(O)C₁₋₆alkyl, C₁₋₆alkylene-N(R^{N})R^{N}, C₁₋₆alkylene-C(O)NR^{N}R^{N}, C(O)C₃₋₁₄carbocyclyl, C₀₋₆alkylene-C₃₋₁₄carbocyclyl, C₀₋₆alkylene-aryl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), and the carbocyclyl, heterocyclyl, or heteroaryl is optionally substituted with 1 or 2 R^{C} groups; or R^{A} and R^{B} together with the nitrogen to which they are attached can form a 4-8 membered heterocyclyl comprising 0-2 additional ring heteroatoms selected from N, O, and S, and the heterocyclyl is optionally substituted with 1 or 2 R^{C} groups and optionally has a spiro cyclopropyl substituent; each R^{N} is independently H or C₁₋₄alkyl; each R^{C} is independently halo, OH, oxo, NR^{N}R^{N}, C₁₋₆alkyl, C₁₋₆alkoxy, C(O)C₁₋₆alkyl, CO₂C₁₋₆alkyl, or C₀₋₆alkylene-(3-14 membered heterocyclyl having 1-3 ring heteroatoms selected from N, O and S); and R^{D} is H, C₁₋₆alkyl, C₁₋₆haloalkyl, or halo; with the proviso that when R² and R³ are each Me, R^{D} is H, V is OR¹, Y is N, and each of W, X, and Z is CR⁴, then (i) X is CH and OR¹ is not OH or OMe; or (ii) E is not NH or NMe; or (iii) OR¹ is not at the 2-position. In some cases, one or two of W, X, Y, and Z is N and the others are CR⁴, or each of W, X, Y, and Z is CR⁴; E is NR⁶, O, or S; V is OR¹, C(O)NR^{N}R¹, C(O)OR¹, or NR^{A}R^{B}; R¹ is H, C₁₋₆alkyl, C₁₋₆haloalkyl, C₁₋₆cyanoalkyl, C₁₋₆hydroxyalkyl, C₁₋₆alkylene-C₁₋₆alkoxy, C₂₋₆alkenyl, C₂₋₆alkynyl, C₀₋₆alkylene-C(O)C₁₋₆alkyl, C₀₋₆alkylene-CO₂R^{N}, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-aryl, C₀₋₆alkylene-(5-10 membered heteroaryl having 1-3 ring heteroatoms selected from N, O and S), C₂₋₈alkylene-C(NH)NR^{A}R^{B}, C₁₋₆alkylene-NR^{A}R^{B}, C₁₋₆alkylene-C(O)NR^{A}R^{B}, C(O)—C₃₋₁₄carbocyclyl, or C(O)-(3-14 membered heterocyclyl having 1-3 ring heteroatoms selected from N, O and S), and the carbocyclyl, aryl, heterocyclyl, or heteroaryl is optionally substituted with 1 or 2 R^{C} groups; each of R², R³, and R⁴ is independently H, halo, CN, OH, C₁₋₆alkyl, C₁₋₆haloalkyl, C₁₋₆alkoxy, C₂₋₆alkenyl, C₂₋₆alkynyl, C₀₋₆alkylene-aryl, NR^{A}R^{B}, SO₀₋₂C₁₋₆alkyl, SO₀₋₂aryl, C(O)C₁₋₆alkyl, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), C(O)C₃₋₁₄carbocyclyl, CO₂H, C(O)NR^{N}—C₁₋₆alkylene-NR^{A}R^{B}, or C₁₋₆alkylene-NR^{A}R^{B}, and the carbocyclyl, heterocyclyl, aryl, or heteroaryl is optionally substituted with 1 or 2 R^{C} groups; R⁶ is H, C₁₋₆alkyl, C₁₋₆haloalkyl, C₁₋₆alkylene-NR^{A}R^{B}, C₁₋₆alkylene-C₃₋₁₄carbocyclyl, C₁₋₆ alkylene-(3-14 membered heterocyclyl having 1-3 ring heteroatoms selected from N, O and S), C₁₋₆alkylene-aryl, C₁₋₆hydroxyalkylene-aryl, or C₁₋₆alkylene-(5-10 membered heteroaryl having 1-3 ring heteroatoms selected from N, O and S), and carbocyclyl, heterocyclyl, aryl or heteroaryl is optionally substituted with 1 or 2 R^{C}; R^{A} and R^{B} are independently H, C₁₋₆alkyl, C₂₋₆alkenyl, C₂₋₆alkynyl, SO₀₋₂C₁₋₆alkyl, SO₀₋₂aryl, C(O)C₁₋₆alkyl, C₁₋₆alkylene-N(R^{N})R^{N}, C(O)C₃₋₁₄carbocyclyl, C₀₋₆alkylene-C₃₋₁₄carbocyclyl, C₀₋₆alkylene-aryl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), and the carbocyclyl, heterocyclyl, or heteroaryl is optionally substituted with 1 or 2 R^{C} groups; or R^{A} and R^{B} together with the nitrogen to which they are attached can form a 4-8 membered heterocyclyl comprising 0-2 additional ring heteroatoms selected from N, O, and S, and the heterocyclyl is optionally substituted with 1 or 2 R^{C} groups and optionally has a spiro cyclopropyl substituent; each R^{N} is independently H or C₁₋₄alkyl; each R^{C} is independently halo, OH, oxo, C₁₋₆alkyl, C₁₋₆alkoxy, C(O)C₁₋₆alkyl, CO₂C₁₋₆alkyl, or C₀₋₆alkylene-(3-14 membered heterocyclyl having 1-3 ring heteroatoms selected from N, O and S); and
R^{D} is H, C₁₋₆alkyl, C₁₋₆haloalkyl, or halo; with the proviso that when R² and R³ are each Me, R^{D} is H, V is OR¹, Y is N, and each of W, X, and Z is CR⁴, then (i) X is CH and OR¹ is not OH or OMe; or (ii) E is not NH or NMe; or (iii) OR¹ is not at the 2-position.

Also provided are compounds, or pharmaceutically acceptable salts thereof, of Formula (Xa):

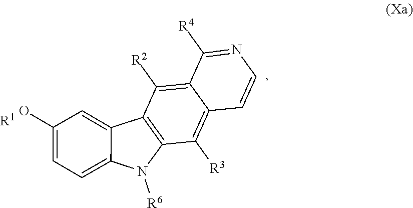

wherein R¹ is H, C₁₋₆alkyl, C₁₋₆alkylene-C(O)C₁₋₆ alkyl, C₁₋₆alkylene-NR^{A}R^{B}, C₁₋₆alkylene-(3- to 14-membered heterocyclyl), or C(O)-(3- to 14-membered heterocyclyl), wherein 1-4 heterocyclyl ring members are independently selected from N, O, and S; and the heterocyclyl is optionally substituted with 1 or 2 R^{C} groups; R^{A} and R^{B} are independently selected from the group consisting of H, C₁₋₆alkyl, C₂₋₆alkenyl, C₂₋₆alkynyl, S(O)₀₋₂—C₁₋₆alkyl, S(O)₀₋₂-aryl, C(O)C₁₋₆alkyl, C(O)C₃₋₁₄carbocyclyl, C₃₋₁₄carbocyclyl, C₂₋₈alkylene-C₃₋₁₄carbocyclyl, aryl, 3- to 14-membered heterocyclyl, C₂₋₈alkylene-(3- to 14-membered heterocyclyl), and 5- to 10-membered heteroaryl, and wherein 1-4 heteroaryl or 1-4 heterocyclyl ring atoms are independently selected from N, O, and S, and the carbocyclyl, aryl, heteroaryl, or heterocyclyl is optionally substituted with 1 or 2 R^{C} groups; each R^{C} is independently halo, OH, C₁₋₆alkoxy, C(O)C₁₋₆alkyl, C(O)OC₁₋₆alkyl, C₁₋₆alkyl, 3- to 14-membered heterocyclyl, and wherein 1-4 heterocyclyl ring members are independently selected from N, O, and S; R⁴ is H, C₁₋₆alkyl, NR^{A}R^{B}, C(O)OC₁₋₆alkyl, C(O)NR^{A}—C₁₋₆alkylene-NR^{A}R^{B}, or C(O)NR^{A}—C₁₋₆alkylene-OR^{B}; R² and R³ are each independently H or CH₃; and R⁶ is H or CH₃.

Also provided are compounds, or pharmaceutically acceptable salts thereof, of Formulae Xb, Xc, and Xd:

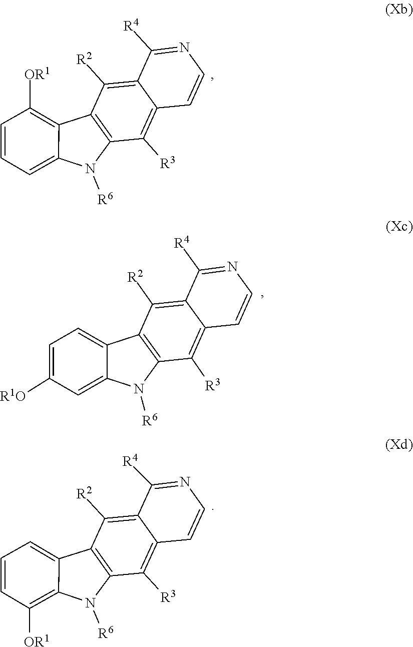

The disclosure also provides as another embodiment is a pharmaceutical composition comprising a compound described herein and a pharmaceutically acceptable carrier or excipient.

In accordance with still a further embodiment, optionally in combination with any other embodiment, the disclosure provides any compound as described herein or a pharmaceutically acceptable salt thereof, for use in a method of treating a subject suffering from a disease or condition characterized by an expanded G₄C₂ repeat RNA (r(G₄C₂)^{exp})

An additional embodiment is a method for determining whether a test compound selectively inhibits repeat associated non-ATG (RAN) translation. The method comprises the steps of (a) contacting a test cell that expresses (G₄C₂)ₙ-NoATG-[Marker1] and ATG-[Marker2] with the test compound, wherein Marker1 and Marker2 are not the same marker, and wherein n is an integer from 5 to 1000 (inclusive); (b) measuring the amounts of Marker1 and Marker2 in the test cell of (a); and (c) comparing the amounts measured in (b), wherein a lower amount of Marker1 compared to Marker2 correlates to a determination that the test compound is a selective inhibitor of (RAN) translation.

### DETAILED DESCRIPTION

Provided herein are nucleic acid-binding compounds that target a nucleotide repeat in a subject, e.g., compounds that target r(G₄C₂)^{exp}. These compounds are useful in the treatment of a variety of diseases and disorders, including but not limited to, Amyotrophic Lateral Sclerosis (ALS) or Frontotemporal Dementia, e.g., c9ALS/FTD.

### Definitions

“Alkyl” refers to straight, branched chain, or cyclic hydrocarbyl groups, e.g., “cycloalkyl,” including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH3)₂, C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(C H₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(C H₂CH₃), —CH₂CH₂CH(CH₂CH₃)₂, —CH₂CH₂C(CH₃)₃, —CH₂CH₂C(CH₂CH₃)₃, —CH(CH₃)CH₂CH(C H₃)₂, —CH(CH₃)CH(CH₃)CH(CH₃)₂, and the like. Thus, alkyl groups include primary alkyl groups, secondary alkyl groups, and tertiary alkyl groups. An “alkylene” group refers to an alkyl group that is further substituted.

Each of the terms “halogen,” “halide,” and “halo” refers to —F, —Cl, —Br, or —I. A “haloalkyl” is an alkyl group substituted with one or more halogen.

The term “alkenyl” refers to straight or branched chain groups from 2 to about 20 carbon atoms having 1-3, 1-2, or at least one carbon to carbon double bond.

“Alkynyl” refers to a straight or branched chain unsaturated hydrocarbon having the indicated number of carbon atoms and at least one triple bond. Examples of a (C₂-C₃)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne.

The term “alkoxy” refers to an —O-alkyl group having the indicated number of carbon atoms. For example, a (C₁-C₅)alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “carbocyclyl” refers to a monocyclic, bicyclic, tricyclic, or polycyclic, 3- to 14-membered ring system, which is either saturated, such as “cycloalkyl,” or unsaturated, such as “cycloalkenyl.” The carbocyclyl may be attached via any atom. Carbocyclyl, for instance, also contemplates fused rings wherein, for instance, a carbocyclyl is fused to an aryl or heteroaryl ring as defined herein. Representative examples of carbocyclyl include, but are not limited to cyclopropyl, cyclobutyl, cyclopentyl, cyclohexyl, cyclopropenyl, cyclobutenyl, cyclopentenyl, cyclohexenyl, phenyl, naphthyl, anthracyl, benzofuranyl, and benzothiophenyl. A carbocyclyl group can be unsubstituted or optionally substituted with one or more substituents as described herein below.

“Aryl” when used alone or as part of another term means a carbocyclic aromatic group whether or not fused having the number of carbon atoms designated or if no number is designated, up to 14 carbon atoms, such as a C₆-C₄-aryl. Particular aryl groups are phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). A particular aryl is phenyl. “Aryl” also includes aromatic ring systems that are optionally fused with a carbocyclyl ring, as herein defined. An aryl group can be unsubstituted or optionally substituted with one or more substituents as described herein below.

The term “heteroatom” refers to N, O, and S. Inventive compounds that contain N or S atoms can be optionally oxidized to the corresponding N-oxide, sulfoxide, or sulfone compounds.

“Heteroaryl,” alone or in combination with any other moiety described herein, refers to a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein below.

“Heterocyclyl” means a saturated or unsaturated non-aromatic monocyclic, bicyclic, tricyclic or polycyclic ring system that has from 3 to 14, such as 3 to 6, atoms in which from 1 to 3 carbon atoms in the ring are replaced by heteroatoms of O, S or N. A heterocyclyl is optionally fused with aryl or heteroaryl of 5-6 ring members, and includes oxidized S or N, such as sulfinyl, sulfonyl and N-oxide of a tertiary ring nitrogen. The point of attachment of the heterocyclyl ring is at a carbon or heteroatom such that a stable ring is retained. Examples of heterocyclyl groups include without limitation morpholino, tetrahydrofuranyl, dihydropyridinyl, piperidinyl, pyrrolidinyl, piperazinyl, dihydrobenzofuryl, dihydroimidazolyl, and dihydroindolyl. A hetercycloalkyl group can be unsubstituted or optionally substituted with one or more substituents as described herein below.

The term “nitrile” or “cyano” can be used interchangeably and refer to a —CN group. The term “cyanoalkyl” refers to an alkyl group substituted with a cyano group.

A “hydroxyl” or “hydroxy” refers to an —OH group. The term “hydroxyalkyl” refers to an alkyl group substituted with a hydroxy group.

An “oxo” refers to a ═O group.

The compounds disclosed herein include isotopically-labeled compounds wherein one or more atoms of the compounds disclosed herein are replaced by atoms having the same atomic number, but an atomic mass or mass number different from the atomic mass or mass number usually found in nature, examples of which include isotopes of hydrogen, such as ²H and ³H, isotopes of carbon, such as ¹¹C and ¹³C, and isotopes of fluorine, such as ¹⁸F. Unless otherwise specifically indicated, an “H” substituent refers to any isotope of hydrogen, ¹H, ²H, or ³H. In some cases, one or more hydrogen atoms of the compounds disclosed herein are specifically ²H (“D” or deuterium). Isotopically-labeled compounds as disclosed herein can be prepared by techniques known to those skilled in the art or by processes analogous to those described in the accompanying examples and schemes using an appropriate isotopically-labeled reagent in place of the non-labeled reagent previously employed.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound of the invention can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified, the term “compound” is inclusive in that it encompasses a compound or a pharmaceutically acceptable salt, stereoisomer, and/or tautomer thereof. Thus, for instance, a compound of Formula I includes a pharmaceutically acceptable salt of a tautomer of the compound.

In this description, a “pharmaceutically acceptable salt” is a pharmaceutically acceptable, organic or inorganic acid or base salt of a compound of the invention. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing,” and “prevention” refer to the prevention of the onset, recurrence, or spread of the disease in a patient resulting from the administration of a prophylactic or therapeutic agent.

The term “effective amount” refers to an amount of a compound of the invention or other active ingredient sufficient to provide a therapeutic or prophylactic benefit in the treatment or prevention of a disease or to delay or minimize symptoms associated with a disease. Further, a therapeutically effective amount with respect to a compound of the invention means that amount of therapeutic agent alone, or in combination with other therapies, that provides a therapeutic benefit in the treatment or prevention of a disease. Used in connection with a compound of the invention, the term can encompass an amount that improves overall therapy, reduces or avoids symptoms or causes of disease, or enhances the therapeutic efficacy of or synergies with another therapeutic agent.

A “patient” or “subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult.

### Compounds

As described generally above, the present disclosure provides compounds and pharmaceutically acceptable salts thereof, wherein the compounds conform to formula I:

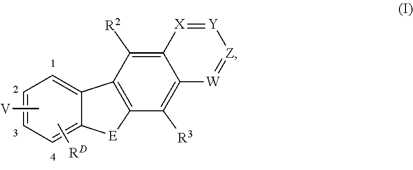

wherein
one or two of W, X, Y, and Z is N and the others are CR⁴, or each of W, X, Y, and Z is CR⁴;
E is NR⁶, O, or S;
V is H, halo, OR¹, C(O)NR^{N}R¹, C(O)OR¹, or NR^{A}R^{B};
R¹ is H, C₁₋₆alkyl, C₁₋₆haloalkyl, C₁₋₆cyanoalkyl, C₁₋₆hydroxyalkyl, C₁₋₆alkylene-C₁₋₆ alkoxy, C₂₋₆alkenyl, C₂₋₆alkynyl, C₀₋₆alkylene-C(O)C₁₋₆alkyl, C₀₋₆alkylene-CO₂R^{N}, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-aryl, C₀₋₆alkylene-(5-10 membered heteroaryl having 1-3 ring heteroatoms selected from N, O and S), C₁₋₆alkylene-C(NH)NR^{A}R^{B}, C₁₋₆alkylene-NR^{A}R^{B}, C₁₋₆alkylene-C(O)NR^{A}R^{B}, C(O)—C₃₋₁₄carbocyclyl, or C(O)-(3-14 membered heterocyclyl having 1-3 ring heteroatoms selected from N, O and S), and the carbocyclyl, aryl, heterocyclyl, or heteroaryl is optionally substituted with 1 or 2 R^{C} groups;
each of R², R³, and R⁴ is independently H, halo, CN, OH, C₁₋₆alkyl, C₁₋₆haloalkyl, C₁₋₆alkoxy, C₂₋₆alkenyl, C₂₋₆alkynyl, C₀₋₆alkylene-aryl, NR^{A}R^{B}, SO₀₋₂C₁₋₆alkyl, SO₀₋₂aryl, C(O)C₁₋₆alkyl, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), C(O)C₃₋₁₄carbocyclyl, CO₂H, C(O)NR^{N}—C₁₋₆alkylene-NR^{A}R^{B}, or C₁₋₆alkylene-NR^{A}R^{B}, and the carbocyclyl, heterocyclyl, aryl, or heteroaryl is optionally substituted with 1 or 2 R^{C} groups;
R⁶ is H, C₁₋₆alkyl, C₁₋₆haloalkyl, C₁₋₆alkylene-NR^{A}R^{B}, C₁₋₆alkylene-C₃₋₁₄carbocyclyl, C₁₋₆alkylene-(3-14 membered heterocyclyl having 1-3 ring heteroatoms selected from N, O and S), C₁₋₆alkylene-aryl, C₁₋₆hydroxyalkylene-aryl, or C₁₋₆alkylene-(5-10 membered heteroaryl having 1-3 ring heteroatoms selected from N, O and S), and carbocyclyl, heterocyclyl, aryl or heteroaryl is optionally substituted with 1 or 2 R^{C};
R^{A} and R^{B} are independently H, C₁₋₆alkyl, C₁₋₆haloalkyl, C₁₋₆hydroxyalkyl, C₂₋₆alkenyl, C₂₋₆alkynyl, C₂₋₈alkylene-C₁₋₆alkoxy, SO₀₋₂C₁₋₆alkyl, SO₀₋₂aryl, C(O)C₁₋₆alkyl, C₂₋₈alkylene-N(R^{N})R^{N}, C₁₋₆alkylene-C(O)NR^{N}R^{N}, C(O)C₃₋₁₄carbocyclyl, C₀₋₆alkylene-C₃₋₁₄carbocyclyl, C₀₋₆alkylene-aryl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), and the carbocyclyl, heterocyclyl, or heteroaryl is optionally substituted with 1 or 2 R^{C} groups; or
R^{A} and R^{B} together with the nitrogen to which they are attached can form a 4-8 membered heterocyclyl comprising 0-2 additional ring heteroatoms selected from N, O, and S, and the heterocyclyl is optionally substituted with 1 or 2 R^{C} groups and optionally has a spiro cyclopropyl substituent;
each R^{N} is independently H or C₁₋₄alkyl;
each R^{C} is independently halo, OH, oxo, NR^{N}R^{N}, C₁₋₆alkyl, C₁₋₆alkoxy, C(O)C₁₋₆alkyl, CO₂C₁₋₆alkyl, or C₀₋₆alkylene-(3-14 membered heterocyclyl having 1-3 ring heteroatoms selected from N, O and S); and
R^{D} is H, C₁₋₆alkyl, C₁₋₆haloalkyl, or halo; with the proviso that when R² and R³ are each Me, R^{D} is H, V is OR¹, Y is N, and each of W, X, and Z is CR⁴, then
(i) X is CH and OR¹ is not OH or OMe; or
(ii) E is not NH or NMe; or
(iii) OR¹ is not at the 2-position. In some cases, one or two of W, X, Y, and Z is N and the others are CR⁴, or each of W, X, Y, and Z is CR⁴; E is NR⁶, O, or S; V is OR¹, C(O)NR^{N}R¹, C(O)OR¹, or NR^{A}R^{B}; R¹ is H, C₁₋₆alkyl, C₁₋₆haloalkyl, C₁₋₆cyanoalkyl, C₁₋₆hydroxyalkyl, C₁₋₆alkylene-C₁₋₆alkoxy, C₂₋₆alkenyl, C₂₋₆alkynyl, C₀₋₆alkylene-C(O)C₁₋₆alkyl, C₀₋₆alkylene-CO₂R^{N}, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-aryl, C₀₋₆alkylene-(5-10 membered heteroaryl having 1-3 ring heteroatoms selected from N, O and S), C₁₋₆alkylene-C(NH)NR^{A}R^{B}, C₁₋₆alkylene-NR^{A}R^{B}, C₁₋₆alkylene-C(O)NR^{A}R^{B}, C(O)—C₃₋₁₄carbocyclyl, or C(O)-(3-14 membered heterocyclyl having 1-3 ring heteroatoms selected from N, O and S), and the carbocyclyl, aryl, heterocyclyl, or heteroaryl is optionally substituted with 1 or 2 R^{C} groups; each of R², R³, and R⁴ is independently H, halo, CN, OH, C₁₋₆alkyl, C₁₋₆haloalkyl, C₁₋₆alkoxy, C₂₋₆alkenyl, C₂₋₆alkynyl, C₀₋₆alkylene-aryl, NR^{A}R^{B}, SO₀₋₂C₁₋₆alkyl, SO₀₋₂aryl, C(O)C₁₋₆alkyl, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), C(O)C₃₋₁₄carbocyclyl, CO₂H, C(O)NR^{N}—C₁₋₆alkylene-NR^{A}R^{B}, or C₁₋₆alkylene-NR^{A}R^{B}, and the carbocyclyl, heterocyclyl, aryl, or heteroaryl is optionally substituted with 1 or 2 R^{C} groups; R⁶ is H, C₁₋₆alkyl, C₁₋₆haloalkyl, C₁₋₆alkylene-NR^{A}R^{B}, C₁₋₆alkylene-C₃₋₁₄carbocyclyl, C₁₋₆ alkylene-(3-14 membered heterocyclyl having 1-3 ring heteroatoms selected from N, O and S), C₁₋₆alkylene-aryl, C₁₋₆hydroxyalkylene-aryl, or C₁₋₆alkylene-(5-10 membered heteroaryl having 1-3 ring heteroatoms selected from N, O and S), and carbocyclyl, heterocyclyl, aryl or heteroaryl is optionally substituted with 1 or 2 R^{C}; R^{A} and R^{B} are independently H, C₁₋₆alkyl, C₂₋₆alkenyl, C₂₋₆alkynyl, SO₀₋₂C₁₋₆alkyl, SO₀₋₂aryl, C(O)C₁₋₆alkyl, C₁₋₆alkylene-N(R^{N})R^{N}, C(O)C₃₋₁₄carbocyclyl, C₀₋₆alkylene-C₃₋₁₄carbocyclyl, C₀₋₆alkylene-aryl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), and the carbocyclyl, heterocyclyl, or heteroaryl is optionally substituted with 1 or 2 R^{C} groups; or R^{A} and R^{B} together with the nitrogen to which they are attached can form a 4-8 membered heterocyclyl comprising 0-2 additional ring heteroatoms selected from N, O, and S, and the heterocyclyl is optionally substituted with 1 or 2 R^{C} groups and optionally has a spiro cyclopropyl substituent; each R^{N} is independently H or C₁₋₄alkyl; each R^{C} is independently halo, OH, oxo, C₁₋₆alkyl, C₁₋₆alkoxy, C(O)C₁₋₆alkyl, CO₂C₁₋₆alkyl, or C₀₋₆alkylene-(3-14 membered heterocyclyl having 1-3 ring heteroatoms selected from N, O and S); and R^{D} is H, C₁₋₆alkyl, C₁₋₆haloalkyl, or halo; with the proviso that when R² and R³ are each Me, R^{D} is H, V is OR¹, Y is N, and each of W, X, and Z is CR⁴, then (i) X is CH and OR¹ is not OH or OMe; or (ii) E is not NH or NMe; or (iii) OR¹ is not at the 2-position.

In some cases, one or two of W, X, Y, and Z is N and the others are CR⁴, or each of W, X, Y, and Z is CR⁴;
E is NR⁶, O, or S;
V is OR¹, C(O)NR^{N}R¹, C(O)OR¹, or NR^{A}R^{B};
R¹ is H, C₁₋₆alkyl, C₁₋₆haloalkyl, C₁₋₆cyanoalkyl, C₂₋₆alkenyl, C₂₋₆alkynyl, C₀₋₆alkylene-C(O)C₁₋₆alkyl, C₀₋₆alkylene-CO₂R^{N}, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-aryl, C₀₋₆alkylene-(5-10 membered heteroaryl having 1-3 ring heteroatoms selected from N, O and S), C₁₋₆alkylene-C(NH)NR^{A}R^{B}, C₁₋₆alkylene-NR^{A}R^{B}, C₁₋₆alkylene-C(O)NR^{A}R^{B}, C(O)—C₃₋₁₄carbocyclyl, or C(O)-(3-14 membered heterocyclyl having 1-3 ring heteroatoms selected from N, O and S), and the carbocyclyl, aryl, heterocyclyl, or heteroaryl is optionally substituted with 1 or 2 R^{C} groups;
each of R², R³, and R⁴ is independently H, halo, CN, OH, C₁₋₆alkyl, C₁₋₆haloalkyl, C₁₋₆alkoxy, C₂₋₆alkenyl, C₂₋₆alkynyl, C₀₋₆alkylene-aryl, NR^{A}R^{B}, SO₀₋₂C₁₋₆alkyl, SO₀₋₂aryl, C(O)C₁₋₆alkyl, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), C(O)C₃₋₁₄carbocyclyl, CO₂H, C(O)NR^{N}—C₁₋₆alkylene-NR^{A}R^{B}, or C₁₋₆alkylene-NR^{A}R^{B}, and the carbocyclyl, heterocyclyl, aryl, or heteroaryl is optionally substituted with 1 or 2 R^{C} groups;
R⁶ is H, C₁₋₆alkyl, C₁₋₆haloalkyl, C₁₋₆alkylene-NR^{A}R^{B}, C₁₋₆-alkylene-C₃₋₁₄carbocyclyl, C₁₋₆alkylene-(3-14 membered heterocyclyl having 1-3 ring heteroatoms selected from N, O and S), C₁₋₆alkylene-aryl, C₁₋₆hydroxyalkylene-aryl, or C₁₋₆alkylene-(5-10 membered heteroaryl having 1-3 ring heteroatoms selected from N, O and S), and carbocyclyl, heterocyclyl, aryl or heteroaryl is optionally substituted with 1 or 2 R^{C};
R^{A} and R^{B} are independently H, C₁₋₆alkyl, C₂₋₆alkenyl, C₂₋₆alkynyl, SO₀₋₂C₁₋₆alkyl, SO₀₋₂aryl, C(O)C₁₋₆alkyl, C₁₋₆alkylene-N(R^{N})R^{N}, C(O)C₃₋₁₄carbocyclyl, C₀₋₆alkylene-C₃₋₁₄carbocyclyl, C₀₋₆alkylene-aryl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), and the carbocyclyl, heterocyclyl, or heteroaryl is optionally substituted with 1 or 2 R^{C} groups; or
R^{A} and R^{B} together with the nitrogen to which they are attached can form a 4-8 membered heterocyclyl comprising 0-2 additional ring heteroatoms selected from N, O, and S, and the heterocyclyl is optionally substituted with 1 or 2 R^{C} groups and optionally has a spiro cyclopropyl substituent;
each R^{N} is independently H or C₁₋₄alkyl;
each R^{C} is independently halo, OH, oxo, C₁₋₆alkyl, C₁₋₆alkoxy, C(O)C₁₋₆alkyl, CO₂C₁₋₆alkyl, or C₀₋₆alkylene-(3-14 membered heterocyclyl having 1-3 ring heteroatoms selected from N, O and S); and
R^{D} is H, C₁₋₆alkyl, C₁₋₆haloalkyl, or halo; with the proviso that when R² and R³ are each Me, R^{D} is H, V is OR¹, Y is N, and each of W, X, and Z is CR⁴, then
(i) X is CH and OR¹ is not OH or OMe; or
(ii) E is not NH or NMe; or
(iii) OR¹ is not at the 2-position.

The present disclosure also provides compounds and pharmaceutically acceptable salts thereof, wherein the compounds conform to Formula (Xa):

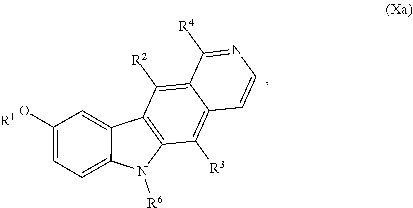

wherein
R¹ is H, C₁₋₆alkyl, C₁₋₆alkylene-C(O)C₁₋₆alkyl, C₁₋₆alkylene-NR^{A}R^{B}, C₁₋₆alkylene-(3- to 14-membered heterocyclyl), or C(O)-(3- to 14-membered heterocyclyl), wherein 1-4 heterocyclyl ring members are independently selected from N, O, and S; and the heterocyclyl is optionally substituted with 1 or 2 R^{C} groups;
R^{A} and R^{B} are independently selected from the group consisting of H, C₁₋₆alkyl, C₂₋₆alkenyl, C₂₋₆alkynyl, S(O)₀₋₂—C₁₋₆alkyl, S(O)₀₋₂-aryl, C(O)C₂₋₈alkyl, C(O)C₃₋₁₄carbocyclyl, C₃₋₁₄ carbocyclyl, C₂₋₈alkylene-C₃₋₁₄carbocyclyl, aryl, 3- to 14-membered heterocyclyl, C₁₋₆alkylene-(3- to 14-membered heterocyclyl), and 5- to 10-membered heteroaryl, and wherein 1-4 heteroaryl or 1-4 heterocyclyl ring atoms are independently selected from N, O, and S, and the carbocyclyl, aryl, heteroaryl, or heterocyclyl is optionally substituted with 1 or 2 R^{C} groups;
each R^{C} is independently halo, OH, C₁₋₆alkoxy, C(O)C₁₋₆alkyl, C(O)OC₁₋₆alkyl, C₁₋₆alkyl, 3- to 14-membered heterocyclyl, and wherein 1-4 heterocyclyl ring members are independently selected from N, O, and S;
R⁴ is H, C₁₋₆alkyl, NR^{A}R^{B}, C(O)OC₁₋₆alkyl, C(O)NR^{A}—C₁₋₆alkylene-NR^{A}R^{B}, or C(O)NR^{A}—C₁₋₆alkylene-OR^{B};
R² and R³ are each independently H or CH₃; and
R⁶ is H or CH₃.

In some cases, V is at the 1-position. In some cases, V is at the 2-position. In some cases, V is at the 3-position. In some cases, V is at the 4-position. In some cases, V is OR¹ or C(O)NR^{N}R¹. In some cases, V is OR¹. In some cases V is C(O)NR^{N}R¹ or C(O)R¹. In some cases, V is C(O)NR^{N}R¹. In some cases, V is NR^{A}NR^{B}. In some cases, V is piperazinyl optionally substituted with methyl or optionally comprising a spiro cyclopropyl substituent. In some cases, V is piperazinyl substituted with methyl or optionally comprising a spiro cyclopropyl substituent. In some cases, V is unsubstituted piperazinyl.

In some cases, R¹ is C₁₋₆alkyl, C₀₋₆alkylene-CO₂R^{N}, C₁₋₆alkylene-C(NH)NR^{A}R^{B}, C₁₋₆alkyleneNR^{A}R^{B}, C₁₋₆alkylene-C(O)NR^{A}R^{B}, or C₁₋₆alkylene(3-14 membered heterocyclyl having 1-3 ring heteroatoms selected from N, O and S). In some cases, R¹ is C₁₋₆alkyleneNR^{A}R^{B}. In some cases, R^{j} is C₁₋₆alkylene-piperazinyl, C₁₋₆alkylene-(N-methylpiperazinyl), C₁₋₆alkylene-morpholinyl, C₁₋₆alkylene-N(Me)₂, C₁₋₆alkylene-piperazinyl, C₁₋₆alkylene-N(Et)₂, C₁₋₆alkylene-pyrrolidinyl, or C₁₋₆alkylene-piperidinyl. In some cases, R¹ is C₁₋₆alkylene-C(NH)NR^{A}R^{B}. In some cases, R^{j} is C₁₋₆alkylene-C(NH)NH₂. In some cases, R¹ is C₁₋₆alkylene(3-14 membered heterocyclyl having 1-3 ring heteroatoms selected from N, O and S). In some cases, R¹ is C₁₋₆alkylene-(4,5-dihydro-2-imidazolyl). In some cases, R¹ is C₁₋₆alkylene-C(O)NR^{A}R^{B}. In some cases, R^{j} is C₁₋₆alkylene-C(O)NH₂, C₁₋₆alkylene-C(O)NMe₂, C₁₋₆alkylene-C(O)NHMe, C₁₋₆alkylene-C(O)-piperazinyl, C₁₋₆alkylene-C(O)-(methylpiperazinyl), C₁₋₆alkylene-C(O)NHC₁₋₆alkylene-morpholinyl, C₁₋₆alkylene-C(O)NHC₁₋₆alkylene-NH₂, C₁₋₆alkylene-C(O)NHC₁₋₆alkylene-NHMe, or C₁₋₆alkylene-C(O)NHC₁₋₆alkylene-NMe₂.

In some cases, E is O. In some cases, E is S. In some cases, E is NR⁶.

In some cases, R⁶ is H, C₁₋₆alkyl or C₁₋₆haloalkyl. In some cases, R⁶ is C₁₋₆alkyl or C₁₋₆haloalkyl. In some cases, R⁶ is H or Me. In some cases, R⁶ is H. In some cases, R⁶ is Me. In some specific cases, R⁶ is CD₃. In some cases, R⁶ is C₁₋₆alkylene-NR^{A}R^{B}. In some cases, R⁶ is C₁₋₆alkylene-piperazinyl, C₁₋₆alkylene-(methylpiperazinyl), C₁₋₆alkylene-morpholinyl, C₁₋₆alkylene-N(Me)₂, C₁₋₆alkylene-piperazinyl, C₁₋₆alkylene-N(Et)₂, C₁₋₆alkylene-pyrrolidinyl, or C₁₋₆alkylene-piperidinyl. In some cases, R⁶ is C₁₋₆alkylene-C₃₋₁₄carbocyclyl, C₁₋₆alkylene-(3-14 membered heterocyclyl having 1-3 ring heteroatoms selected from N, O and S), C₁₋₆alkylene-aryl, C₁₋₆hydroxyalkylene-aryl, or C₁₋₆alkylene-(5-10 membered heteroaryl having 1-3 ring heteroatoms selected from N, O and S). In some cases, R⁶ is benzyl, C₁₋₆hydroxyalkylenephenyl, C₁₋₆alkylene-pyridinyl, C₁₋₆alkylene-tetrahydrothiophenedioxide, C₁₋₆alkylene-imidazoyl, C₁₋₆alkylene-(methylimidazolyl), or C₁₋₆alkylene-(4,5-dihydro-2-imidazolyl).

In some cases, R² is H, OH, or C₁₋₆alkyl. In some cases, R² is H or C₁₋₆alkyl. In some cases, R² is H. In some cases, R² is OH. In some cases, R² is C₁₋₆alkyl. In some cases, R² is methyl. In some specific cases, R² is CD₃.

In some cases, R³ is H, halo, C₁₋₆alkyl, or C₁₋₆haloalkyl. In some cases, R³ is H or C₁₋₆alkyl. In some cases, R³ is H. In some cases, R³ is C₁₋₆alkyl. In some cases, R³ is methyl. In some specific cases, R³ is CD₃. In some cases, R³ is halo or C₁₋₆haloalkyl. In some cases, R³ is halo. In some cases, R³ is C₁₋₆haloalkyl. In some cases, R³ is F or CF₃. In some cases, R³ is F. In some cases, R³ is CF₃.

In some cases, both R² and R³ are C₁₋₆alkyl. In some cases, both R² and R³ are methyl.

In some cases, X is N. In some cases, Y is N. In some cases, Z is N. In some cases, W is N.

In some cases, each of X, Y, Z, and W is CR⁴.

In some cases, each R⁴ is independently H, CO₂H, C(O)NR^{N}—C₁-C₆-alkylene-NR^{A}R^{B}, or C₁₋₆alkylene-NR^{A}R^{B}. In some cases, each R⁴ is H. In some cases, at least one R⁴ is C(O)NHC₁₋₆alkyleneNMe₂ or CO₂H. In some cases, at least one R⁴ is C(O)NHC₁₋₆alkyleneNMe₂. In some cases, at least one R⁴ is CO₂H. In some cases, one R⁴ is CO₂H, C(O)NR^{N}—C₁-C₆-alkylene-NR^{A}R^{B}, or C₁₋₆alkylene-NR^{A}R^{B}, and all other R⁴ are H.

In some cases, R^{D} is H or halo. In some cases, R^{D} is H. In some cases, R^{D} is halo. In some cases, R^{D} is F or Cl. In some cases, R^{D} is F. In some cases, In some cases, R^{D} is Cl. In some cases, R^{D} is C₁₋₆alkyl or C₁₋₆haloalkyl. In some cases, R^{D} is C₁₋₆alkyl. In some cases, R^{D} is CH₃. In some cases, R^{D} is CD₃. In some cases, R^{D} is C₁₋₆haloalkyl. In some cases, R⁷ is CF₃.

In various embodiments, the disclosure provides specific examples of Formula I compounds, and their pharmaceutically acceptable salts, as set forth in Table A, Table B, or Table C below:

TABLE A

| ID | |
|---|---|
| No. | Structure |
| 1 | 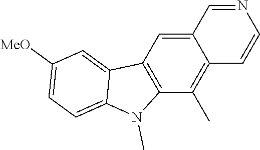 [SMILES: COc1ccc2c(c1)c1cc3cnccc3c(C)c1n2C] |
| 2 | 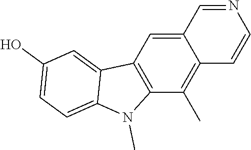 [SMILES: Cc1c2ccncc2cc2c3cc(O)ccc3n(C)c12] |
| 3 | 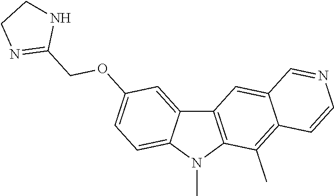 [SMILES: Cc1c2ccncc2cc2c3cc(OCC4=NCCN4)ccc3n(C)c12] |
| 4a | 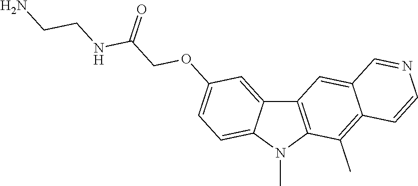 [SMILES: Cc1c2ccncc2cc2c3cc(OCC(=O)NCCN)ccc3n(C)c12] |
| 4b | 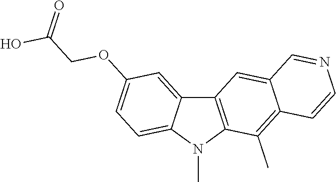 [SMILES: Cc1c2ccncc2cc2c3cc(OCC(=O)O)ccc3n(C)c12] |
| 5 | 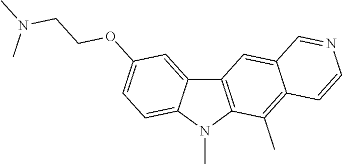 [SMILES: Cc1c2ccncc2cc2c3cc(OCCN(C)C)ccc3n(C)c12] |
| 6 | 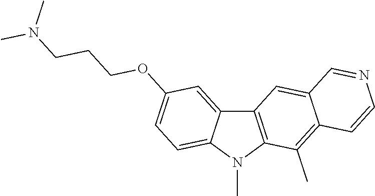 [SMILES: Cc1c2ccncc2cc2c3cc(OCCCN(C)C)ccc3n(C)c12] |
| 7 | 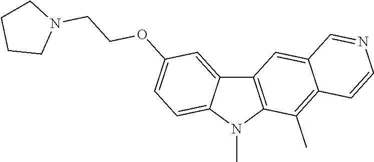 [SMILES: Cc1c2ccncc2cc2c3cc(OCCN4CCCC4)ccc3n(C)c12] |
| 8 | 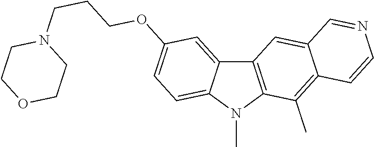 [SMILES: Cc1c2ccncc2cc2c3cc(OCCCN4CCOCC4)ccc3n(C)c12] |
| 9 | 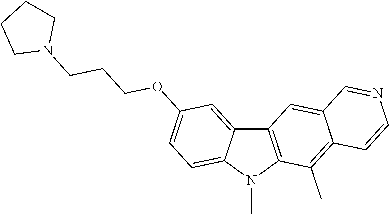 [SMILES: Cc1c2ccncc2cc2c3cc(OCCCN4CCCC4)ccc3n(C)c12] |
| 10 | 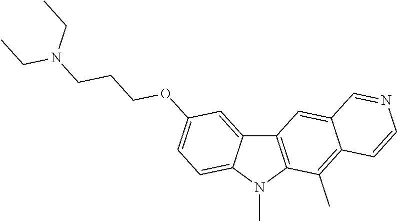 [SMILES: CCN(CC)CCCOc1ccc2c(c1)c1cc3cnccc3c(C)c1n2C] |
| 11 | 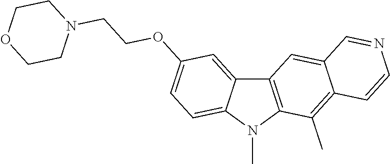 [SMILES: Cc1c2ccncc2cc2c3cc(OCCN4CCOCC4)ccc3n(C)c12] |
| 12 | 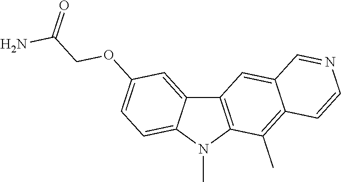 [SMILES: Cc1c2ccncc2cc2c3cc(OCC(N)=O)ccc3n(C)c12] |
| 13 | 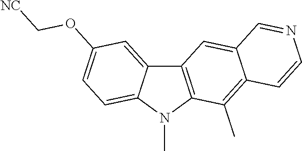 [SMILES: Cc1c2ccncc2cc2c3cc(OCC#N)ccc3n(C)c12] |
| 14 | 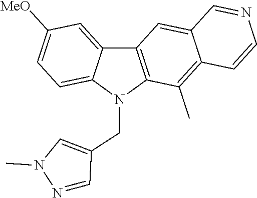 [SMILES: COc1ccc2c(c1)c1cc3cnccc3c(C)c1n2Cc1cnn(C)c1] |
| 15 | 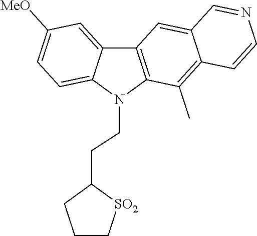 [SMILES: COc1ccc2c(c1)c1cc3cnccc3c(C)c1n2CCC1CCCS1(=O)=O] |
| 16 | 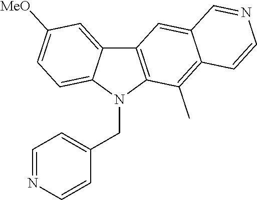 [SMILES: COc1ccc2c(c1)c1cc3cnccc3c(C)c1n2Cc1ccncc1] |
| 17 | 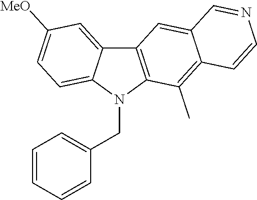 [SMILES: COc1ccc2c(c1)c1cc3cnccc3c(C)c1n2Cc1ccccc1] |
| 18 | 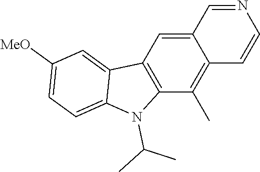 [SMILES: COc1ccc2c(c1)c1cc3cnccc3c(C)c1n2C(C)C] |
| 19 | 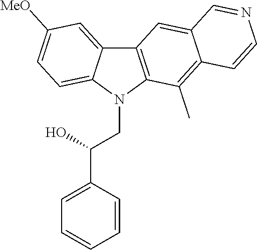 [SMILES: COc1ccc2c(c1)c1cc3cnccc3c(C)c1n2C[C@@H](O)c1ccccc1] |
| 20 | 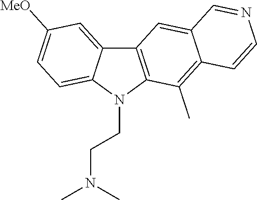 [SMILES: COc1ccc2c(c1)c1cc3cnccc3c(C)c1n2CCN(C)C] |
| 21 | 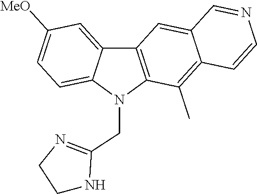 [SMILES: COc1ccc2c(c1)c1cc3cnccc3c(C)c1n2CC1=NCCN1] |
| 22 | 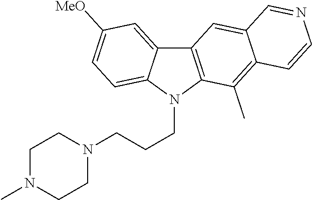 [SMILES: COc1ccc2c(c1)c1cc3cnccc3c(C)c1n2CCCN1CCN(C)CC1] |
| 23 | 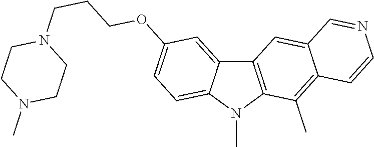 [SMILES: Cc1c2ccncc2cc2c3cc(OCCCN4CCN(C)CC4)ccc3n(C)c12] |
| 24 | 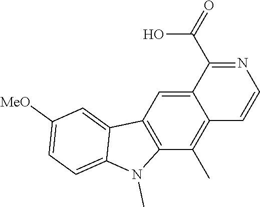 [SMILES: COc1ccc2c(c1)c1cc3c(C(=O)O)nccc3c(C)c1n2C] |
| 25 | 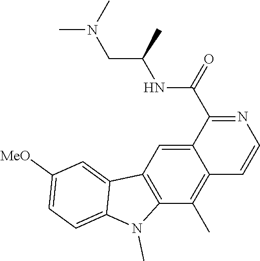 [SMILES: COc1ccc2c(c1)c1cc3c(C(=O)N[C@H](C)CN(C)C)nccc3c(C)c1n2C] |
| 26 | 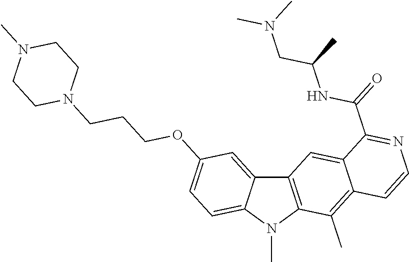 [SMILES: Cc1c2ccnc(C(=O)N[C@H](C)CN(C)C)c2cc2c3cc(OCCCN4CCN(C)CC4)ccc3n(C)c12] |
| 27 | 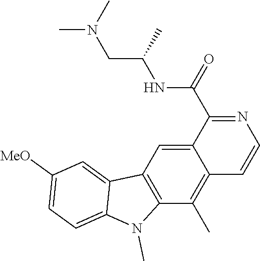 [SMILES: COc1ccc2c(c1)c1cc3c(C(=O)N[C@@H](C)CN(C)C)nccc3c(C)c1n2C] |
| 28 | 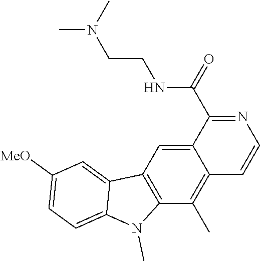 [SMILES: COc1ccc2c(c1)c1cc3c(C(=O)NCCN(C)C)nccc3c(C)c1n2C] |
| 29 | 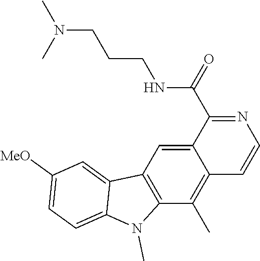 [SMILES: COc1ccc2c(c1)c1cc3c(C(=O)NCCCN(C)C)nccc3c(C)c1n2C] |
| 30 | 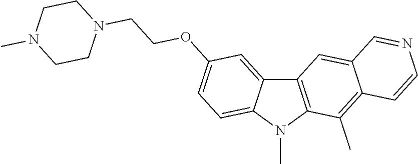 [SMILES: Cc1c2ccncc2cc2c3cc(OCCN4CCN(C)CC4)ccc3n(C)c12] |
| 31 | 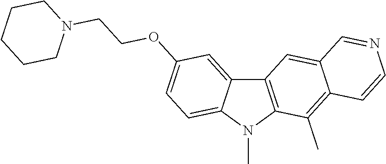 [SMILES: Cc1c2ccncc2cc2c3cc(OCCN4CCCCC4)ccc3n(C)c12] |
| 32 | 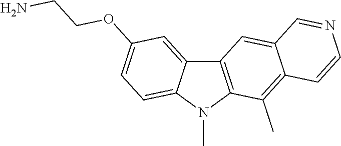 [SMILES: Cc1c2ccncc2cc2c3cc(OCCN)ccc3n(C)c12] |
| 33 | 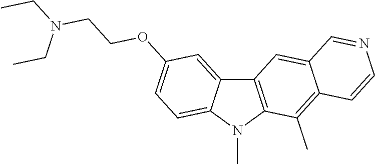 [SMILES: CCN(CC)CCOc1ccc2c(c1)c1cc3cnccc3c(C)c1n2C] |
| 34 | 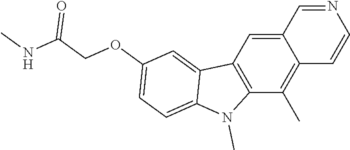 [SMILES: CNC(=O)COc1ccc2c(c1)c1cc3cnccc3c(C)c1n2C] |
| 35 | 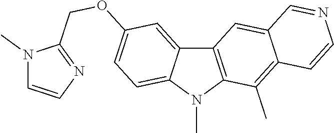 [SMILES: Cc1c2ccncc2cc2c3cc(OCc4nccn4C)ccc3n(C)c12] |
| 36 | 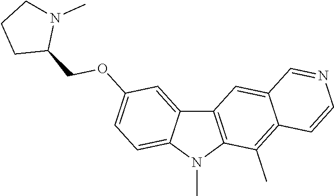 [SMILES: Cc1c2ccncc2cc2c3cc(OC[C@H]4CCCN4C)ccc3n(C)c12] |
| 37 | 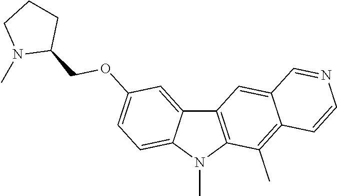 [SMILES: Cc1c2ccncc2cc2c3cc(OC[C@@H]4CCCN4C)ccc3n(C)c12] |
| 38 | 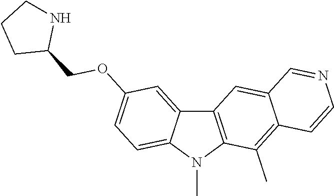 [SMILES: Cc1c2ccncc2cc2c3cc(OC[C@H]4CCCN4)ccc3n(C)c12] |
| 39 | 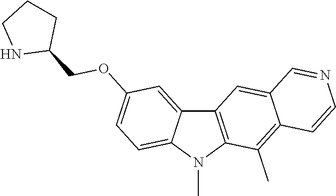 [SMILES: Cc1c2ccncc2cc2c3cc(OC[C@@H]4CCCN4)ccc3n(C)c12] |
| 40 | 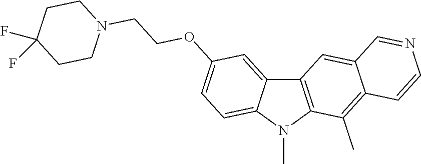 [SMILES: Cc1c2ccncc2cc2c3cc(OCCN4CCC(F)(F)CC4)ccc3n(C)c12] |
| 41 | 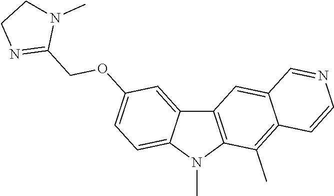 [SMILES: Cc1c2ccncc2cc2c3cc(OCC4=NCCN4C)ccc3n(C)c12] |
| 42 | 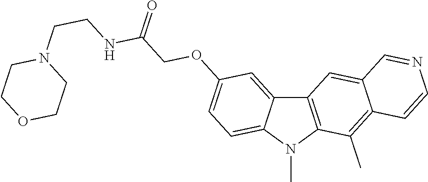 [SMILES: Cc1c2ccncc2cc2c3cc(OCC(=O)NCCN4CCOCC4)ccc3n(C)c12] |
| 43 | 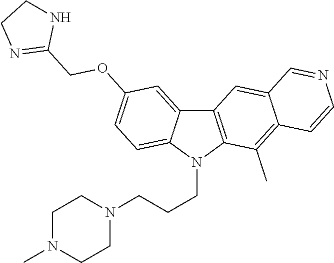 [SMILES: Cc1c2ccncc2cc2c3cc(OCC4=NCCN4)ccc3n(CCCN3CCN(C)CC3)c12] |
| 44a | 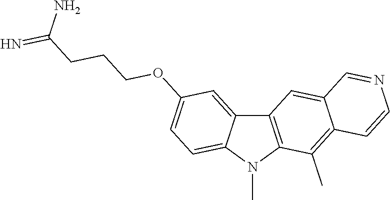 [SMILES: Cc1c2ccncc2cc2c3cc(OCCCC(=N)N)ccc3n(C)c12] |
| 44b | 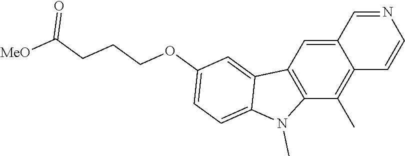 [SMILES: COC(=O)CCCOc1ccc2c(c1)c1cc3cnccc3c(C)c1n2C] |
| 45 | 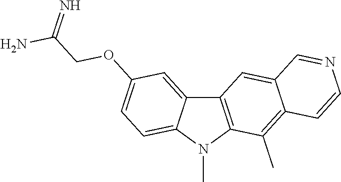 [SMILES: Cc1c2ccncc2cc2c3cc(OCC(=N)N)ccc3n(C)c12] |
| 46 | 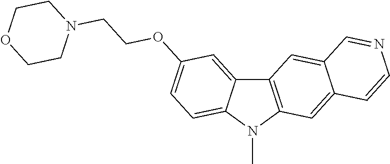 [SMILES: Cn1c2ccc(OCCN3CCOCC3)cc2c2cc3cnccc3cc21] |
| 47 | 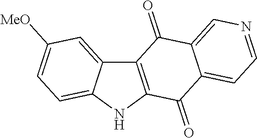 [SMILES: COc1ccc2[nH]c3c(c2c1)C(=O)c1cnccc1C3=O] |
| 48 | 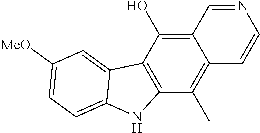 [SMILES: COc1ccc2[nH]c3c(C)c4ccncc4c(O)c3c2c1] |
| 49 | 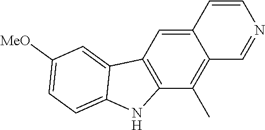 [SMILES: COc1ccc2[nH]c3c(C)c4cnccc4cc3c2c1] |
| 50 | 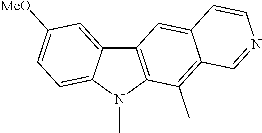 [SMILES: COc1ccc2c(c1)c1cc3ccncc3c(C)c1n2C] |
| 51 | 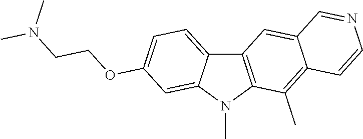 [SMILES: Cc1c2ccncc2cc2c3ccc(OCCN(C)C)cc3n(C)c12] |
| 52 | 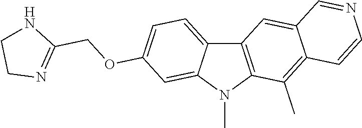 [SMILES: Cc1c2ccncc2cc2c3ccc(OCC4=NCCN4)cc3n(C)c12] |
| 53 | 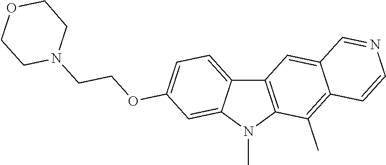 [SMILES: Cc1c2ccncc2cc2c3ccc(OCCN4CCOCC4)cc3n(C)c12] |
| 54 | 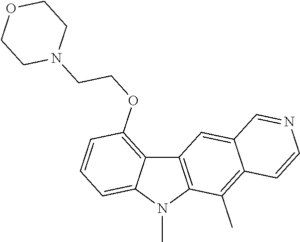 [SMILES: Cc1c2ccncc2cc2c3c(OCCN4CCOCC4)cccc3n(C)c12] |
| 55 | 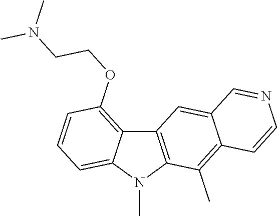 [SMILES: Cc1c2ccncc2cc2c3c(OCCN(C)C)cccc3n(C)c12] |
| 56 | 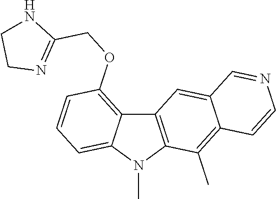 [SMILES: Cc1c2ccncc2cc2c3c(OCC4=NCCN4)cccc3n(C)c12] |
| 57 | 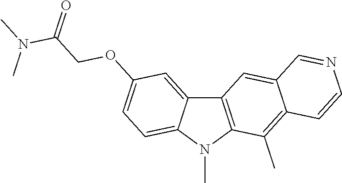 [SMILES: Cc1c2ccncc2cc2c3cc(OCC(=O)N(C)C)ccc3n(C)c12] |
| 58 | 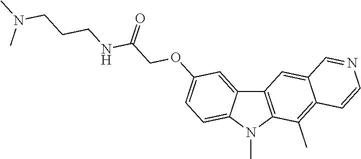 [SMILES: Cc1c2ccncc2cc2c3cc(OCC(=O)NCCCN(C)C)ccc3n(C)c12] |
| 59 | 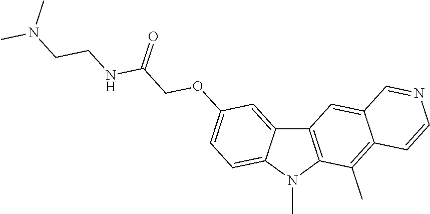 [SMILES: Cc1c2ccncc2cc2c3cc(OCC(=O)NCCN(C)C)ccc3n(C)c12] |
| 60 | 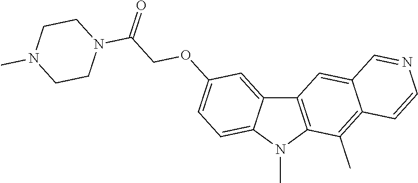 [SMILES: Cc1c2ccncc2cc2c3cc(OCC(=O)N4CCN(C)CC4)ccc3n(C)c12] |
| 61 | 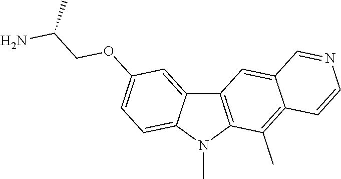 [SMILES: Cc1c2ccncc2cc2c3cc(OC[C@@H](C)N)ccc3n(C)c12] |
| 62 | 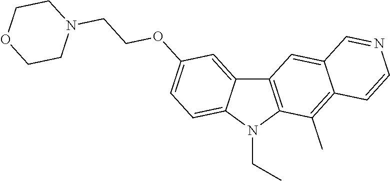 [SMILES: CCn1c2ccc(OCCN3CCOCC3)cc2c2cc3cnccc3c(C)c21] |
| 63 | 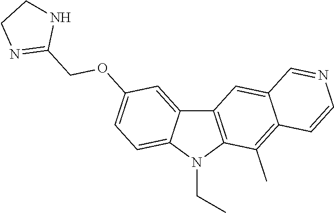 [SMILES: CCn1c2ccc(OCC3=NCCN3)cc2c2cc3cnccc3c(C)c21] |
| 64 | 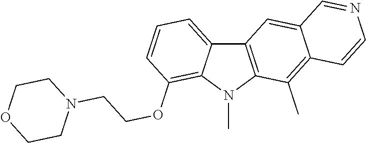 [SMILES: Cc1c2ccncc2cc2c3cccc(OCCN4CCOCC4)c3n(C)c12] |
| 65 | 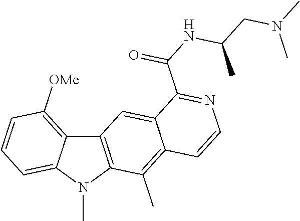 [SMILES: COc1cccc2c1c1cc3c(C(=O)N[C@H](C)CN(C)C)nccc3c(C)c1n2C] |
| 66 | 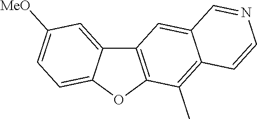 [SMILES: COc1ccc2oc3c(C)c4ccncc4cc3c2c1] |
| 67 | 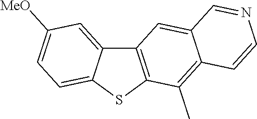 [SMILES: COc1ccc2sc3c(C)c4ccncc4cc3c2c1] |
| 68 | 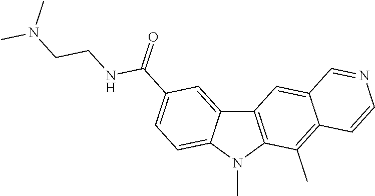 [SMILES: Cc1c2ccncc2cc2c3cc(C(=O)NCCN(C)C)ccc3n(C)c12] |
| 69 | 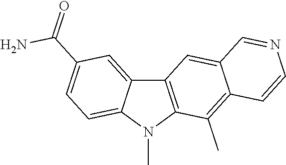 [SMILES: Cc1c2ccncc2cc2c3cc(C(N)=O)ccc3n(C)c12] |
| 70 | 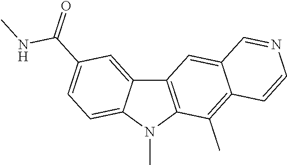 [SMILES: CNC(=O)c1ccc2c(c1)c1cc3cnccc3c(C)c1n2C] |
| 71 | 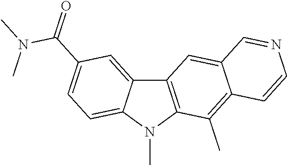 [SMILES: Cc1c2ccncc2cc2c3cc(C(=O)N(C)C)ccc3n(C)c12] |
| 72 | 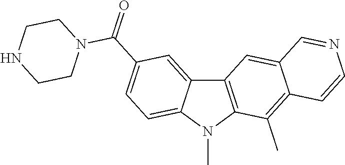 [SMILES: Cc1c2ccncc2cc2c3cc(C(=O)N4CCNCC4)ccc3n(C)c12] |
| 73 | 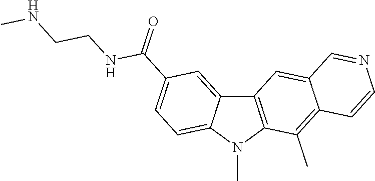 [SMILES: CNCCNC(=O)c1ccc2c(c1)c1cc3cnccc3c(C)c1n2C] |
| 74 | 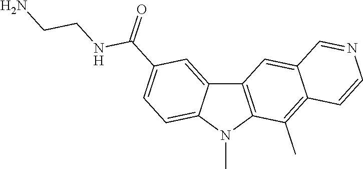 [SMILES: Cc1c2ccncc2cc2c3cc(C(=O)NCCN)ccc3n(C)c12] |
| 75 | 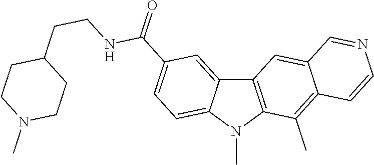 [SMILES: Cc1c2ccncc2cc2c3cc(C(=O)NCCC4CCN(C)CC4)ccc3n(C)c12] |
| 76 | 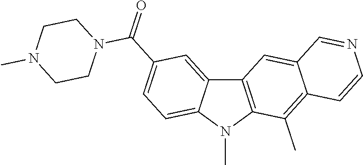 [SMILES: Cc1c2ccncc2cc2c3cc(C(=O)N4CCN(C)CC4)ccc3n(C)c12] |
| 77 | 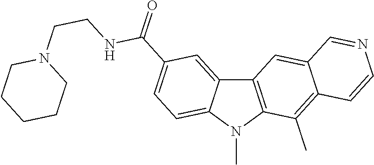 [SMILES: Cc1c2ccncc2cc2c3cc(C(=O)NCCN4CCCCC4)ccc3n(C)c12] |
| 78 | 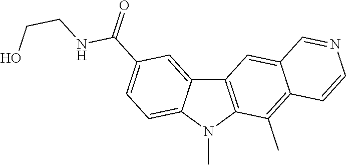 [SMILES: Cc1c2ccncc2cc2c3cc(C(=O)NCCO)ccc3n(C)c12] |
| 79 | 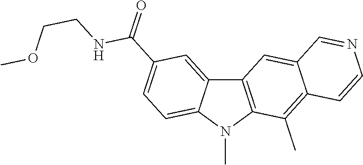 [SMILES: COCCNC(=O)c1ccc2c(c1)c1cc3cnccc3c(C)c1n2C] |
| 80a | 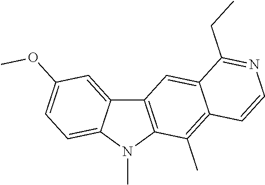 [SMILES: CCc1nccc2c(C)c3c(cc12)c1cc(OC)ccc1n3C] |
| 80b | 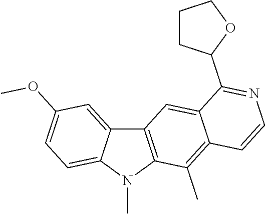 [SMILES: COc1ccc2c(c1)c1cc3c(C4CCCO4)nccc3c(C)c1n2C] |
| 81 | 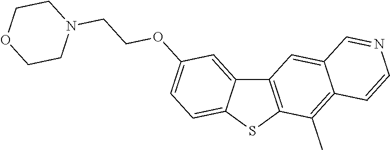 [SMILES: Cc1c2ccncc2cc2c1sc1ccc(OCCN3CCOCC3)cc12] |
| 82 | 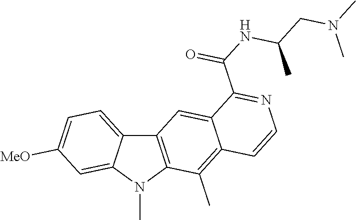 [SMILES: COc1ccc2c3cc4c(C(=O)N[C@H](C)CN(C)C)nccc4c(C)c3n(C)c2c1] |
| 83 | 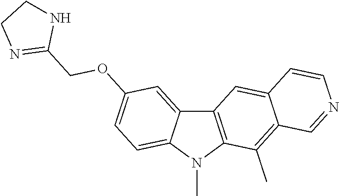 [SMILES: Cc1c2cnccc2cc2c3cc(OCC4=NCCN4)ccc3n(C)c12] |
| 84 | 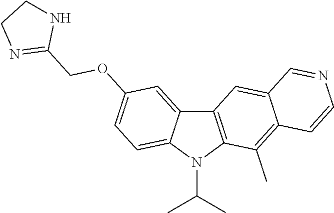 [SMILES: Cc1c2ccncc2cc2c3cc(OCC4=NCCN4)ccc3n(C(C)C)c12] |
| 85 | 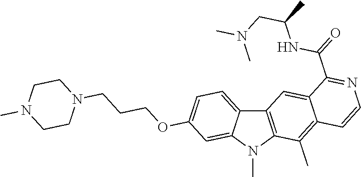 [SMILES: Cc1c2ccnc(C(=O)N[C@H](C)CN(C)C)c2cc2c3ccc(OCCCN4CCN(C)CC4)cc3n(C)c12] |
| 86 | 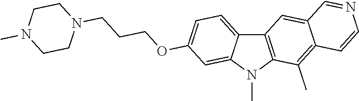 [SMILES: Cc1c2ccncc2cc2c3ccc(OCCCN4CCN(C)CC4)cc3n(C)c12] |
| 87 | 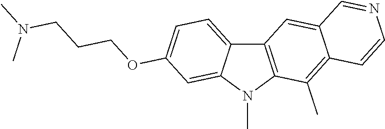 [SMILES: Cc1c2ccncc2cc2c3ccc(OCCCN(C)C)cc3n(C)c12] |
| 88 | 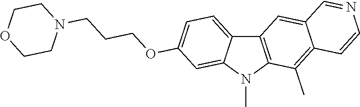 [SMILES: Cc1c2ccncc2cc2c3ccc(OCCCN4CCOCC4)cc3n(C)c12] |
| 89 | 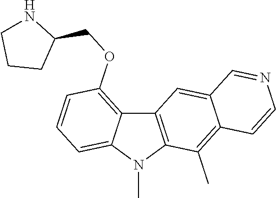 [SMILES: Cc1c2ccncc2cc2c3c(OC[C@H]4CCCN4)cccc3n(C)c12] |
| 90 | 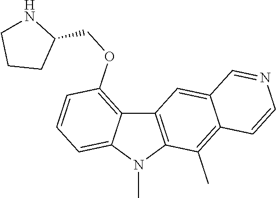 [SMILES: Cc1c2ccncc2cc2c3c(OC[C@@H]4CCCN4)cccc3n(C)c12] |
| 91 | 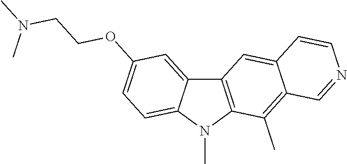 [SMILES: Cc1c2cnccc2cc2c3cc(OCCN(C)C)ccc3n(C)c12] |
| 92 | 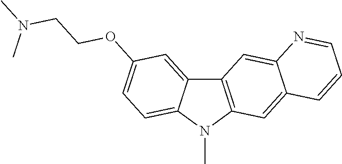 [SMILES: CN(C)CCOc1ccc2c(c1)c1cc3ncccc3cc1n2C] |
| 93 | 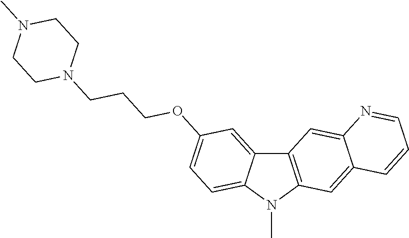 [SMILES: CN1CCN(CCCOc2ccc3c(c2)c2cc4ncccc4cc2n3C)CC1] |
| 94 | 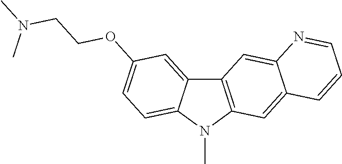 [SMILES: CN(C)CCOc1ccc2c(c1)c1cc3ncccc3cc1n2C] |
| 95 | 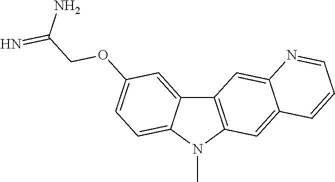 [SMILES: Cn1c2ccc(OCC(=N)N)cc2c2cc3ncccc3cc21] |
| 96 | 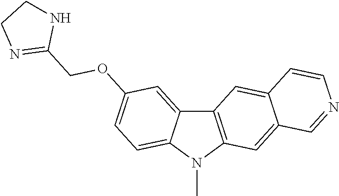 [SMILES: Cn1c2ccc(OCC3=NCCN3)cc2c2cc3ccncc3cc21] |
| 97 | 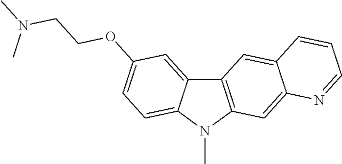 [SMILES: CN(C)CCOc1ccc2c(c1)c1cc3cccnc3cc1n2C] |
| 98 | 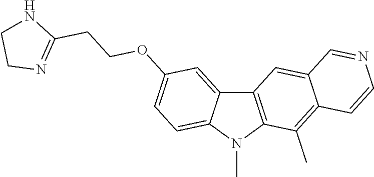 [SMILES: Cc1c2ccncc2cc2c3cc(OCCC4=NCCN4)ccc3n(C)c12] |
| 99 | 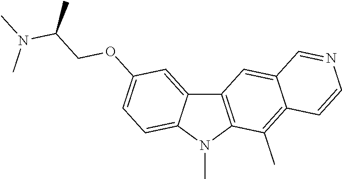 [SMILES: Cc1c2ccncc2cc2c3cc(OC[C@H](C)N(C)C)ccc3n(C)c12] |
| 100 | 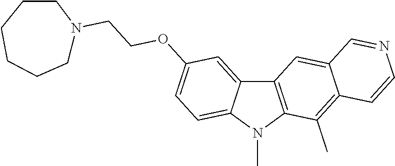 [SMILES: Cc1c2ccncc2cc2c3cc(OCCN4CCCCCC4)ccc3n(C)c12] |
| 101 | 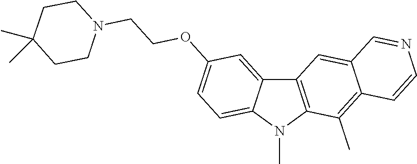 [SMILES: Cc1c2ccncc2cc2c3cc(OCCN4CCC(C)(C)CC4)ccc3n(C)c12] |
| 102 | 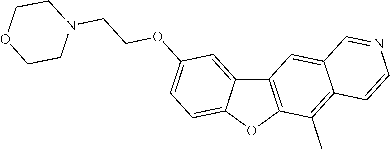 [SMILES: Cc1c2ccncc2cc2c1oc1ccc(OCCN3CCOCC3)cc12] |
| 103 | 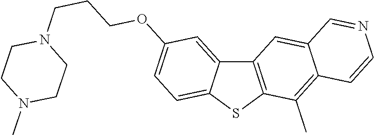 [SMILES: Cc1c2ccncc2cc2c1sc1ccc(OCCCN3CCN(C)CC3)cc12] |
| 104 | 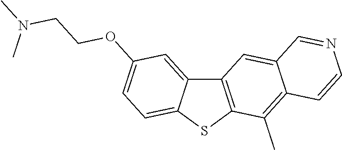 [SMILES: Cc1c2ccncc2cc2c1sc1ccc(OCCN(C)C)cc12] |
| 105 | 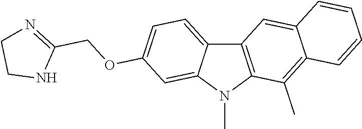 [SMILES: Cc1c2ccccc2cc2c3ccc(OCC4=NCCN4)cc3n(C)c12] |
| 106 | 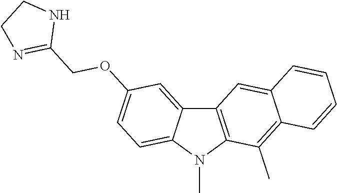 [SMILES: Cc1c2ccccc2cc2c3cc(OCC4=NCCN4)ccc3n(C)c12] |
| 107 | 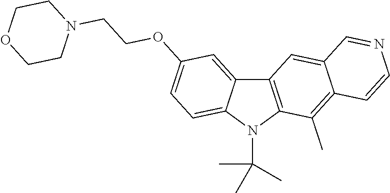 [SMILES: Cc1c2ccncc2cc2c3cc(OCCN4CCOCC4)ccc3n(C(C)(C)C)c12] |
| 108 | 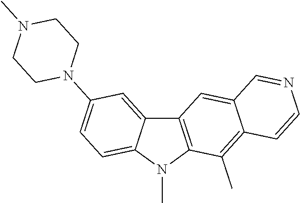 [SMILES: Cc1c2ccncc2cc2c3cc(N4CCN(C)CC4)ccc3n(C)c12] |
| 109 | 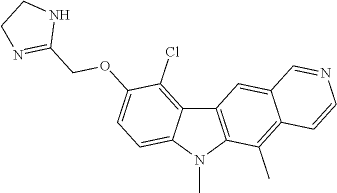 [SMILES: Cc1c2ccncc2cc2c3c(Cl)c(OCC4=NCCN4)ccc3n(C)c12] |
| 110 | 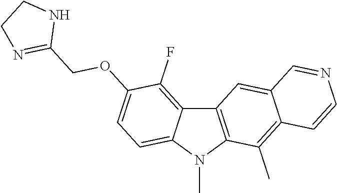 [SMILES: Cc1c2ccncc2cc2c3c(F)c(OCC4=NCCN4)ccc3n(C)c12] |
| 111 | 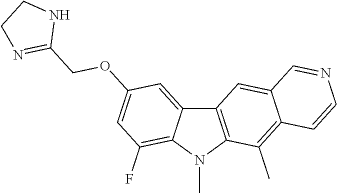 [SMILES: Cc1c2ccncc2cc2c3cc(OCC4=NCCN4)cc(F)c3n(C)c12] |
| 112 | 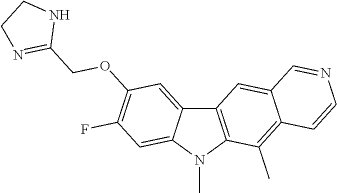 [SMILES: Cc1c2ccncc2cc2c3cc(OCC4=NCCN4)c(F)cc3n(C)c12] |
| 113 | 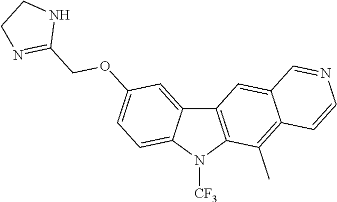 [SMILES: Cc1c2ccncc2cc2c3cc(OCC4=NCCN4)ccc3n(C(F)(F)F)c12] |
| 114 | 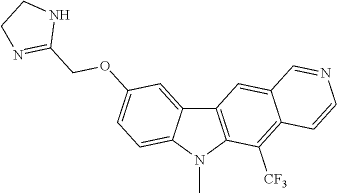 [SMILES: Cn1c2ccc(OCC3=NCCN3)cc2c2cc3cnccc3c(C(F)(F)F)c21] |
| 115 | 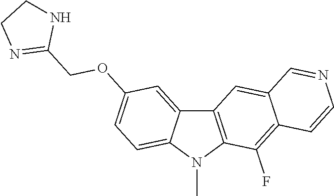 [SMILES: Cn1c2ccc(OCC3=NCCN3)cc2c2cc3cnccc3c(F)c21] |
| 116 | 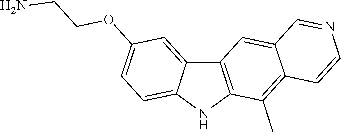 [SMILES: Cc1c2ccncc2cc2c1[nH]c1ccc(OCCN)cc12] |
| 117 | 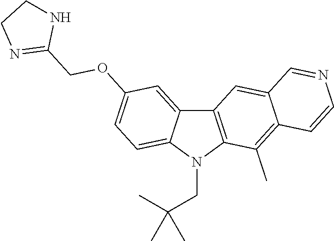 [SMILES: Cc1c2ccncc2cc2c3cc(OCC4=NCCN4)ccc3n(CC(C)(C)C)c12] |
| 118 | 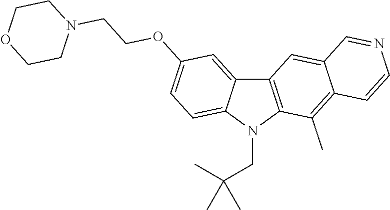 [SMILES: Cc1c2ccncc2cc2c3cc(OCCN4CCOCC4)ccc3n(CC(C)(C)C)c12] |
| 119 | 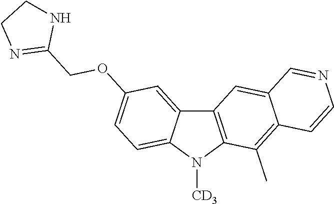 [SMILES: [2H]C([2H])([2H])n1c2ccc(OCC3=NCCN3)cc2c2cc3cnccc3c(C)c21] |
| 120 | 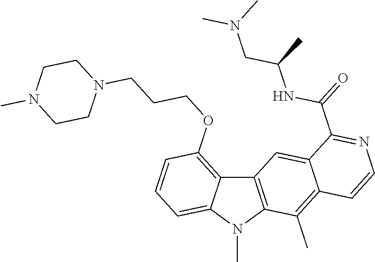 [SMILES: Cc1c2ccnc(C(=O)N[C@H](C)CN(C)C)c2cc2c3c(OCCCN4CCN(C)CC4)cccc3n(C)c12] |
| 121 | 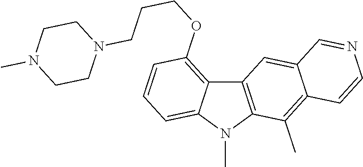 [SMILES: Cc1c2ccncc2cc2c3c(OCCCN4CCN(C)CC4)cccc3n(C)c12] |
| 122 | 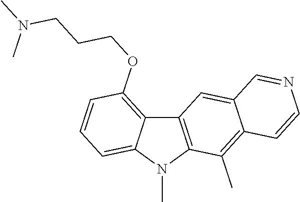 [SMILES: Cc1c2ccncc2cc2c3c(OCCCN(C)C)cccc3n(C)c12] |
| 123 | 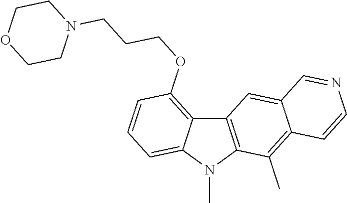 [SMILES: Cc1c2ccncc2cc2c3c(OCCCN4CCOCC4)cccc3n(C)c12] |
| 124 | 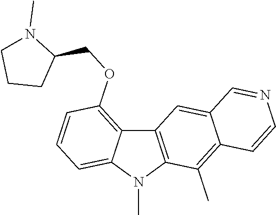 [SMILES: Cc1c2ccncc2cc2c3c(OC[C@H]4CCCN4C)cccc3n(C)c12] |
| 125 | 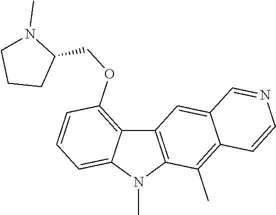 [SMILES: Cc1c2ccncc2cc2c3c(OC[C@@H]4CCCN4C)cccc3n(C)c12] |
| 126 | 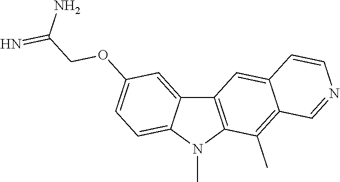 [SMILES: Cc1c2cnccc2cc2c3cc(OCC(=N)N)ccc3n(C)c12] |
| 127 | 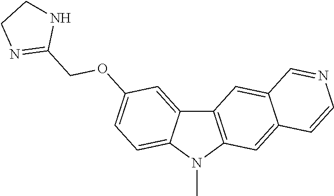 [SMILES: Cn1c2ccc(OCC3=NCCN3)cc2c2cc3cnccc3cc21] |
| 128 | 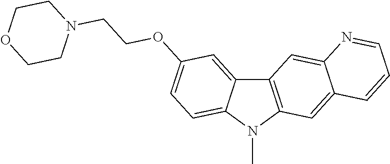 [SMILES: Cn1c2ccc(OCCN3CCOCC3)cc2c2cc3ncccc3cc21] |
| 129 | 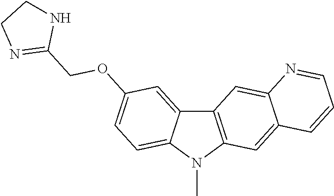 [SMILES: Cn1c2ccc(OCC3=NCCN3)cc2c2cc3ncccc3cc21] |
| 130 | 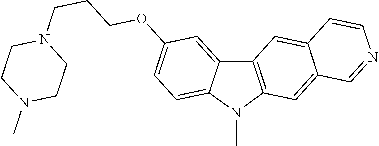 [SMILES: CN1CCN(CCCOc2ccc3c(c2)c2cc4ccncc4cc2n3C)CC1] |
| 131 | 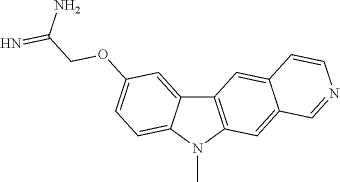 [SMILES: Cn1c2ccc(OCC(=N)N)cc2c2cc3ccncc3cc21] |
| 132 | 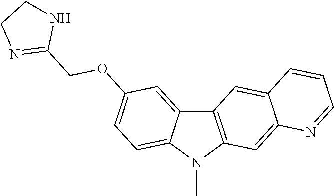 [SMILES: Cn1c2ccc(OCC3=NCCN3)cc2c2cc3cccnc3cc21] |
| 133 | 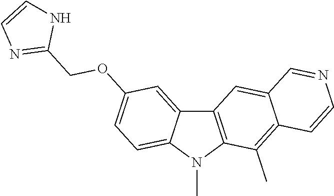 [SMILES: Cc1c2ccncc2cc2c3cc(OCc4ncc[nH]4)ccc3n(C)c12] |
| 134 | 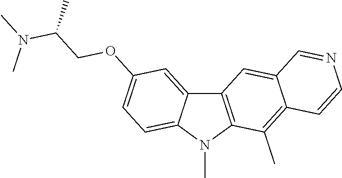 [SMILES: Cc1c2ccncc2cc2c3cc(OC[C@@H](C)N(C)C)ccc3n(C)c12] |
| 135 | 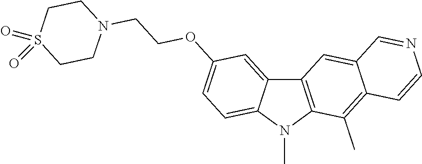 [SMILES: Cc1c2ccncc2cc2c3cc(OCCN4CCS(=O)(=O)CC4)ccc3n(C)c12] |
| 136 | 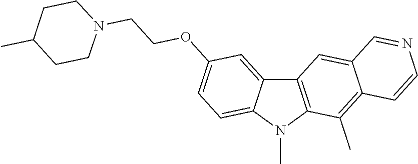 [SMILES: Cc1c2ccncc2cc2c3cc(OCCN4CCC(C)CC4)ccc3n(C)c12] |
| 137 | 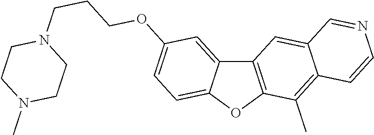 [SMILES: Cc1c2ccncc2cc2c1oc1ccc(OCCCN3CCN(C)CC3)cc12] |
| 138 | 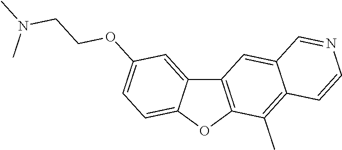 [SMILES: Cc1c2ccncc2cc2c1oc1ccc(OCCN(C)C)cc12] |
| 139 | 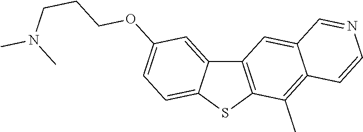 [SMILES: Cc1c2ccncc2cc2c1sc1ccc(OCCCN(C)C)cc12] |
| 140 | 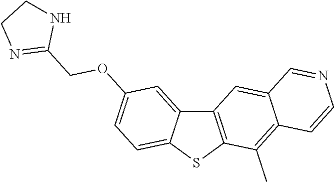 [SMILES: Cc1c2ccncc2cc2c1sc1ccc(OCC3=NCCN3)cc12] |
| 141 | 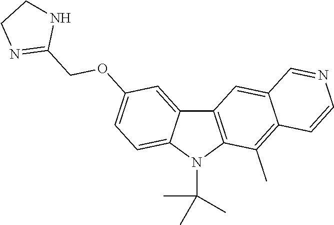 [SMILES: Cc1c2ccncc2cc2c3cc(OCC4=NCCN4)ccc3n(C(C)(C)C)c12] |
| 142 | 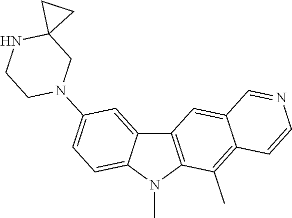 [SMILES: Cc1c2ccncc2cc2c3cc(N4CCNC5(CC5)C4)ccc3n(C)c12] |
| 143 | 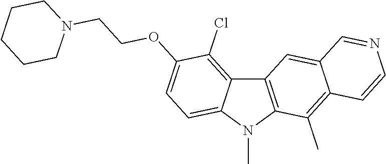 [SMILES: Cc1c2ccncc2cc2c3c(Cl)c(OCCN4CCCCC4)ccc3n(C)c12] |
| 144 | 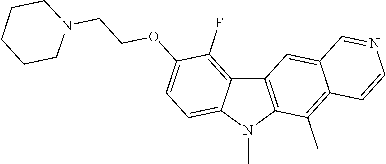 [SMILES: Cc1c2ccncc2cc2c3c(F)c(OCCN4CCCCC4)ccc3n(C)c12] |
| 145 | 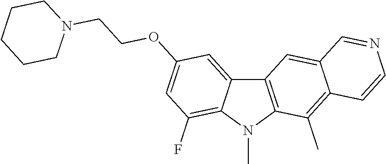 [SMILES: Cc1c2ccncc2cc2c3cc(OCCN4CCCCC4)cc(F)c3n(C)c12] |
| 146 | 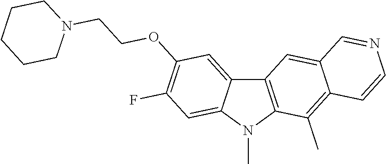 [SMILES: Cc1c2ccncc2cc2c3cc(OCCN4CCCCC4)c(F)cc3n(C)c12] |
| 147 | 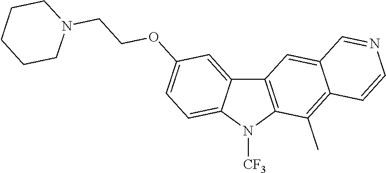 [SMILES: Cc1c2ccncc2cc2c3cc(OCCN4CCCCC4)ccc3n(C(F)(F)F)c12] |
| 148 | 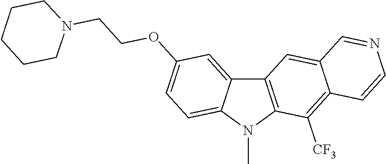 [SMILES: Cn1c2ccc(OCCN3CCCCC3)cc2c2cc3cnccc3c(C(F)(F)F)c21] |
| 149 | 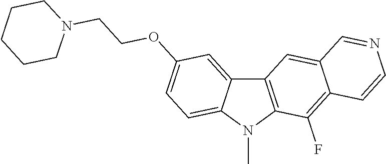 [SMILES: Cn1c2ccc(OCCN3CCCCC3)cc2c2cc3cnccc3c(F)c21] |
| 150 | 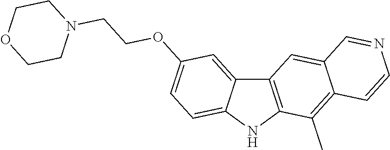 [SMILES: Cc1c2ccncc2cc2c1[nH]c1ccc(OCCN3CCOCC3)cc12] |
| 151 | 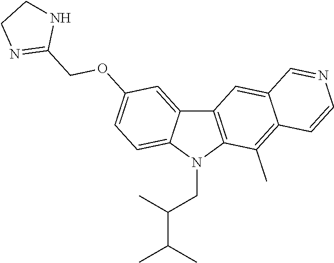 [SMILES: Cc1c2ccncc2cc2c3cc(OCC4=NCCN4)ccc3n(CC(C)C(C)C)c12] |
| 152 | 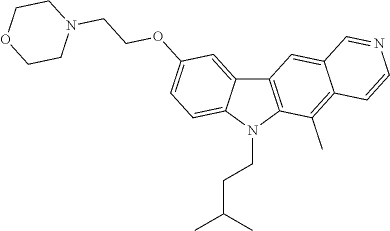 [SMILES: Cc1c2ccncc2cc2c3cc(OCCN4CCOCC4)ccc3n(CCC(C)C)c12] |
| 153 | 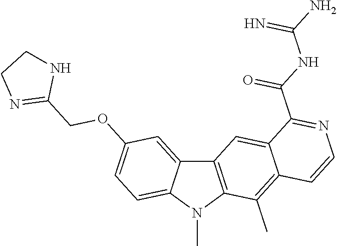 [SMILES: Cc1c2ccnc(C(=O)NC(=N)N)c2cc2c3cc(OCC4=NCCN4)ccc3n(C)c12] |
| 154 | 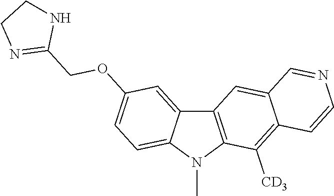 [SMILES: [2H]C([2H])([2H])c1c2ccncc2cc2c3cc(OCC4=NCCN4)ccc3n(C)c12] |
| 155 | 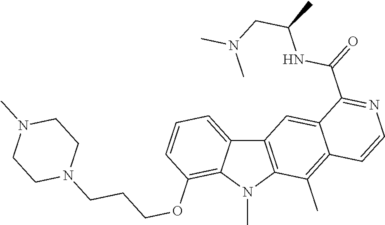 [SMILES: Cc1c2ccnc(C(=O)N[C@H](C)CN(C)C)c2cc2c3cccc(OCCCN4CCN(C)CC4)c3n(C)c12] |
| 156 | 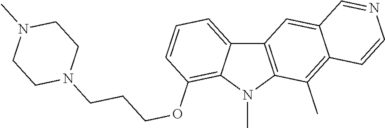 [SMILES: Cc1c2ccncc2cc2c3cccc(OCCCN4CCN(C)CC4)c3n(C)c12] |
| 157 | 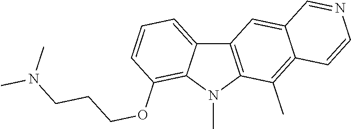 [SMILES: Cc1c2ccncc2cc2c3cccc(OCCCN(C)C)c3n(C)c12] |
| 158 | 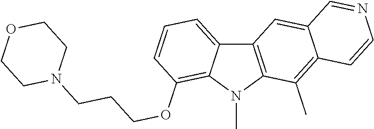 [SMILES: Cc1c2ccncc2cc2c3cccc(OCCCN4CCOCC4)c3n(C)c12] |
| 159 | 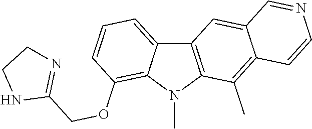 [SMILES: Cc1c2ccncc2cc2c3cccc(OCC4=NCCN4)c3n(C)c12] |
| 160 | 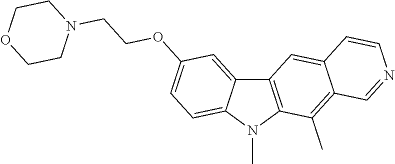 [SMILES: Cc1c2cnccc2cc2c3cc(OCCN4CCOCC4)ccc3n(C)c12] |
| 161 | 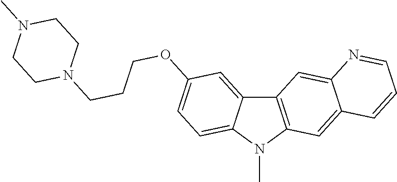 [SMILES: CN1CCN(CCCOc2ccc3c(c2)c2cc4ncccc4cc2n3C)CC1] |
| 162 | 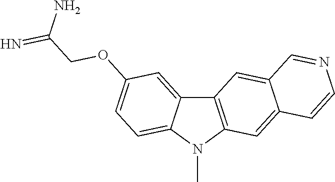 [SMILES: Cn1c2ccc(OCC(=N)N)cc2c2cc3cnccc3cc21] |
| 163 | 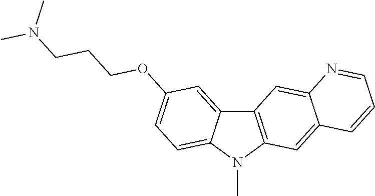 [SMILES: CN(C)CCCOc1ccc2c(c1)c1cc3ncccc3cc1n2C] |
| 164 | 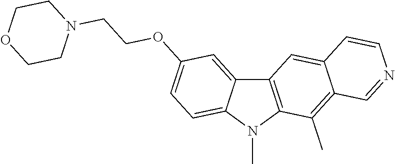 [SMILES: Cc1c2cnccc2cc2c3cc(OCCN4CCOCC4)ccc3n(C)c12] |
| 165 | 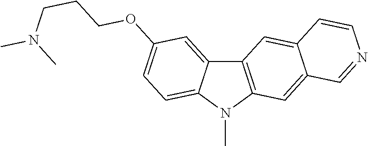 [SMILES: CN(C)CCCOc1ccc2c(c1)c1cc3ccncc3cc1n2C] |
| 166 | 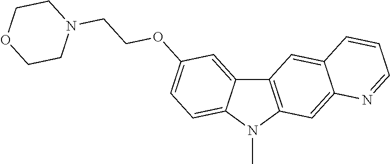 [SMILES: Cn1c2ccc(OCCN3CCOCC3)cc2c2cc3cccnc3cc21] |
| 167 | 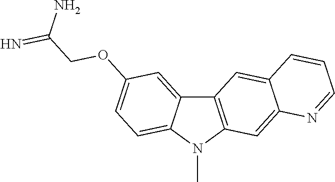 [SMILES: Cn1c2ccc(OCC(=N)N)cc2c2cc3cccnc3cc21] |
| 168 | 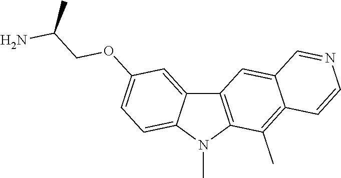 [SMILES: Cc1c2ccncc2cc2c3cc(OC[C@H](C)N)ccc3n(C)c12] |
| 169 | 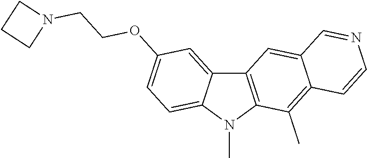 [SMILES: Cc1c2ccncc2cc2c3cc(OCCN4CCC4)ccc3n(C)c12] |
| 170 | 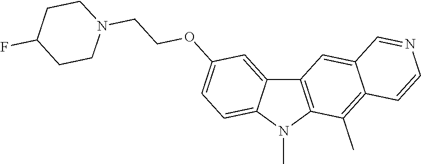 [SMILES: Cc1c2ccncc2cc2c3cc(OCCN4CCC(F)CC4)ccc3n(C)c12] |
| 171 | 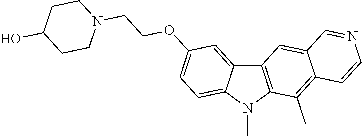 [SMILES: Cc1c2ccncc2cc2c3cc(OCCN4CCC(O)CC4)ccc3n(C)c12] |
| 172 | 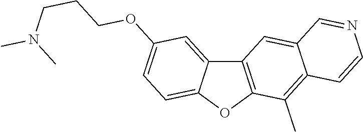 [SMILES: Cc1c2ccncc2cc2c1oc1ccc(OCCCN(C)C)cc12] |
| 173 | 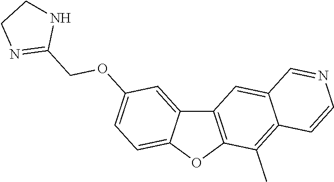 [SMILES: Cc1c2ccncc2cc2c1oc1ccc(OCC3=NCCN3)cc12] |
| 174 | 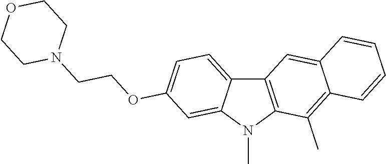 [SMILES: Cc1c2ccccc2cc2c3ccc(OCCN4CCOCC4)cc3n(C)c12] |
| 175 | 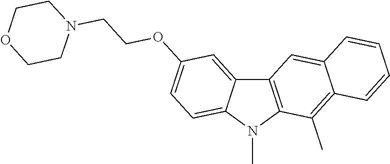 [SMILES: Cc1c2ccccc2cc2c3cc(OCCN4CCOCC4)ccc3n(C)c12] |
| 176 | 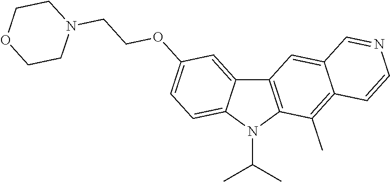 [SMILES: Cc1c2ccncc2cc2c3cc(OCCN4CCOCC4)ccc3n(C(C)C)c12] |
| 177 | 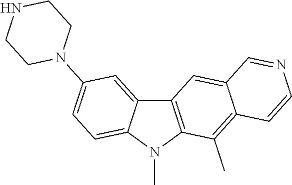 [SMILES: Cc1c2ccncc2cc2c3cc(N4CCNCC4)ccc3n(C)c12] |
| 178 | 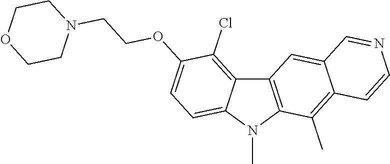 [SMILES: Cc1c2ccncc2cc2c3c(Cl)c(OCCN4CCOCC4)ccc3n(C)c12] |
| 179 | 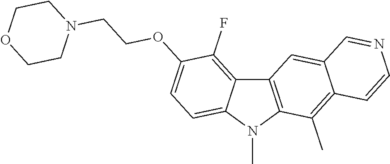 [SMILES: Cc1c2ccncc2cc2c3c(F)c(OCCN4CCOCC4)ccc3n(C)c12] |
| 180 | 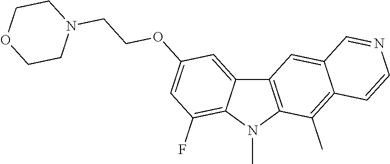 [SMILES: Cc1c2ccncc2cc2c3cc(OCCN4CCOCC4)cc(F)c3n(C)c12] |
| 181 | 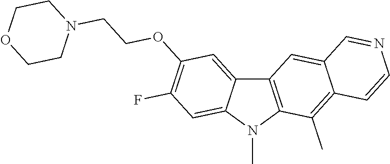 [SMILES: Cc1c2ccncc2cc2c3cc(OCCN4CCOCC4)c(F)cc3n(C)c12] |
| 182 | 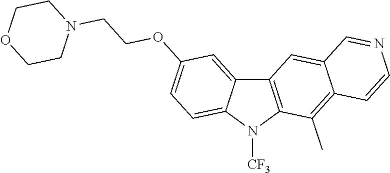 [SMILES: Cc1c2ccncc2cc2c3cc(OCCN4CCOCC4)ccc3n(C(F)(F)F)c12] |
| 183 | 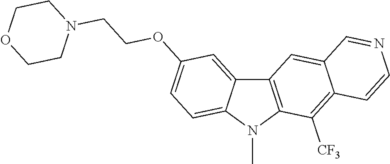 [SMILES: Cn1c2ccc(OCCN3CCOCC3)cc2c2cc3cnccc3c(C(F)(F)F)c21] |
| 184 | 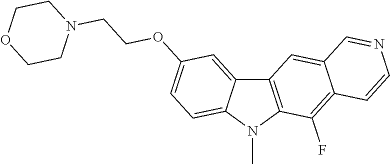 [SMILES: Cn1c2ccc(OCCN3CCOCC3)cc2c2cc3cnccc3c(F)c21] |
| 185 | 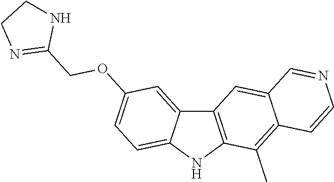 [SMILES: Cc1c2ccncc2cc2c1[nH]c1ccc(OCC3=NCCN3)cc12] |
| 186 | 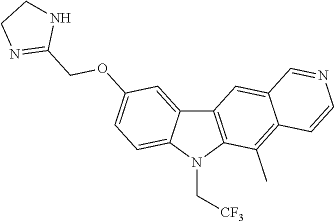 [SMILES: Cc1c2ccncc2cc2c3cc(OCC4=NCCN4)ccc3n(CC(F)(F)F)c12] |
| 187 | 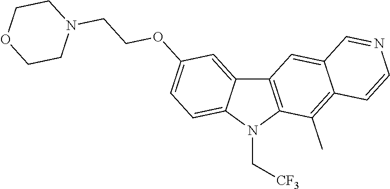 [SMILES: Cc1c2ccncc2cc2c3cc(OCCN4CCOCC4)ccc3n(CC(F)(F)F)c12] |
| 188 | 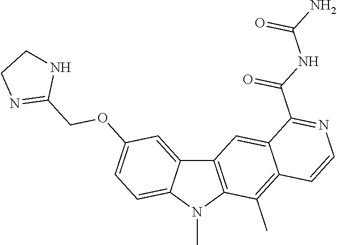 [SMILES: Cc1c2ccnc(C(=O)NC(N)=O)c2cc2c3cc(OCC4=NCCN4)ccc3n(C)c12] |
| 189 | 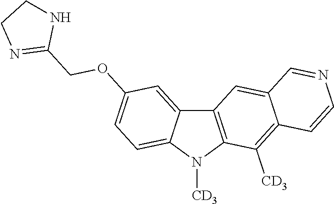 [SMILES: [2H]C([2H])([2H])c1c2ccncc2cc2c3cc(OCC4=NCCN4)ccc3n(C([2H])([2H])[2H])c12] |

TABLE B

| ID | |
|---|---|
| No | Structure |
| 190 | 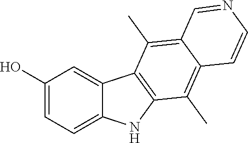 [SMILES: Cc1c2ccncc2c(C)c2c1[nH]c1ccc(O)cc12] |
| 191 | 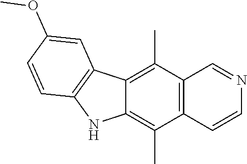 [SMILES: COc1ccc2[nH]c3c(C)c4ccncc4c(C)c3c2c1] |
| 192 | 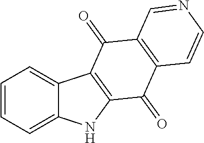 [SMILES: O=C1c2ccncc2C(=O)c2c1[nH]c1ccccc21] |
| 193 | 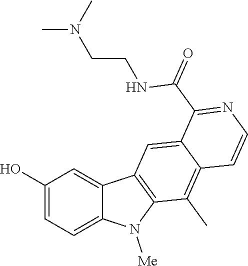 [SMILES: Cc1c2ccnc(C(=O)NCCN(C)C)c2cc2c3cc(O)ccc3n(C)c12] |
| 194 | 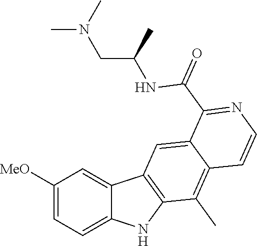 [SMILES: COc1ccc2[nH]c3c(C)c4ccnc(C(=O)N[C@H](C)CN(C)C)c4cc3c2c1] |
| 195 | 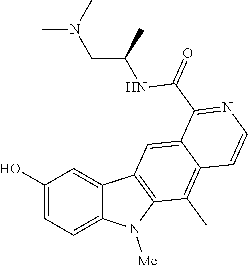 [SMILES: Cc1c2ccnc(C(=O)N[C@H](C)CN(C)C)c2cc2c3cc(O)ccc3n(C)c12] |
| 196 | 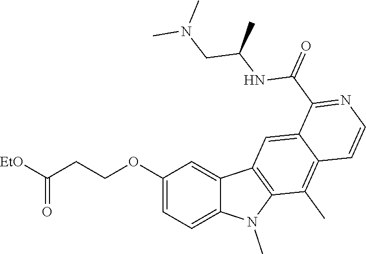 [SMILES: CCOC(=O)CCOc1ccc2c(c1)c1cc3c(C(=O)N[C@H](C)CN(C)C)nccc3c(C)c1n2C] |
| 197 | 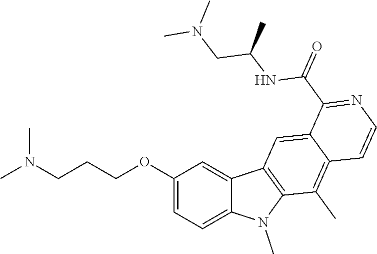 [SMILES: Cc1c2ccnc(C(=O)N[C@H](C)CN(C)C)c2cc2c3cc(OCCCN(C)C)ccc3n(C)c12] |
| 198 | 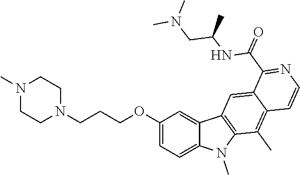 [SMILES: Cc1c2ccnc(C(=O)N[C@H](C)CN(C)C)c2cc2c3cc(OCCCN4CCN(C)CC4)ccc3n(C)c12] |
| 199 | 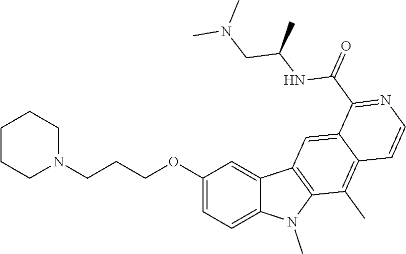 [SMILES: Cc1c2ccnc(C(=O)N[C@H](C)CN(C)C)c2cc2c3cc(OCCCN4CCCCC4)ccc3n(C)c12] |
| 200 | 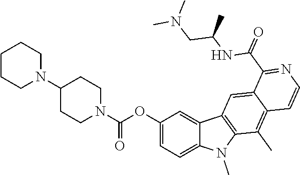 [SMILES: Cc1c2ccnc(C(=O)N[C@H](C)CN(C)C)c2cc2c3cc(OC(=O)N4CCC(N5CCCCC5)CC4)ccc3n(C)c12] |
| 201 | 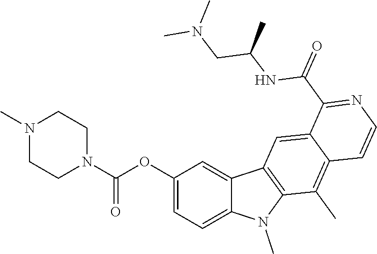 [SMILES: Cc1c2ccnc(C(=O)N[C@H](C)CN(C)C)c2cc2c3cc(OC(=O)N4CCN(C)CC4)ccc3n(C)c12] |
| 202 | 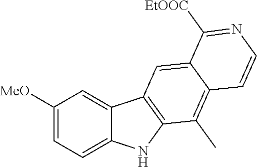 [SMILES: CCOC(=O)c1nccc2c(C)c3[nH]c4ccc(OC)cc4c3cc12] |
| 203 | 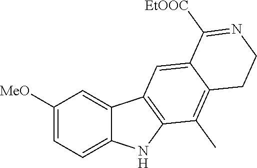 [SMILES: CCOC(=O)C1=NCCc2c1cc1c([nH]c3ccc(OC)cc31)c2C] |
| 204 | 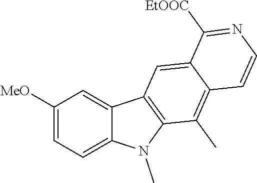 [SMILES: CCOC(=O)c1nccc2c(C)c3c(cc12)c1cc(OC)ccc1n3C] |
| 205 | 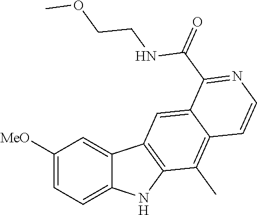 [SMILES: COCCNC(=O)c1nccc2c(C)c3[nH]c4ccc(OC)cc4c3cc12] |
| 206 | 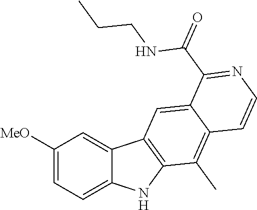 [SMILES: CCCNC(=O)c1nccc2c(C)c3[nH]c4ccc(OC)cc4c3cc12] |
| 207 | 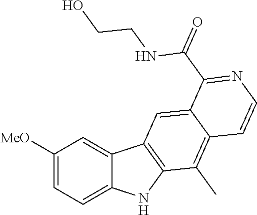 [SMILES: COc1ccc2[nH]c3c(C)c4ccnc(C(=O)NCCO)c4cc3c2c1] |
| 208 | 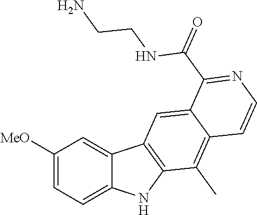 [SMILES: COc1ccc2[nH]c3c(C)c4ccnc(C(=O)NCCN)c4cc3c2c1] |
| 209 | 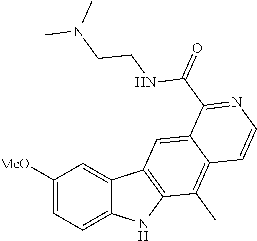 [SMILES: COc1ccc2[nH]c3c(C)c4ccnc(C(=O)NCCN(C)C)c4cc3c2c1] |
| 210 | 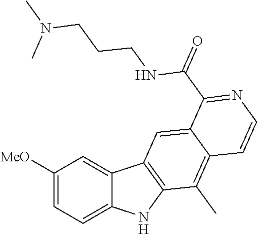 [SMILES: COc1ccc2[nH]c3c(C)c4ccnc(C(=O)NCCCN(C)C)c4cc3c2c1] |
| 211 | 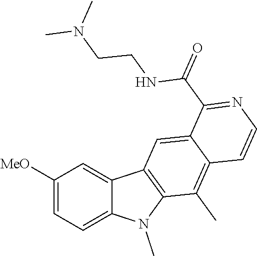 [SMILES: COc1ccc2c(c1)c1cc3c(C(=O)NCCN(C)C)nccc3c(C)c1n2C] |
| 212 | 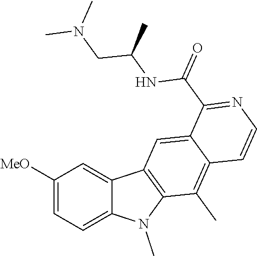 [SMILES: COc1ccc2c(c1)c1cc3c(C(=O)N[C@H](C)CN(C)C)nccc3c(C)c1n2C] |

TABLE C

| ID | |
|---|---|
| No | Structure |
| 213 | 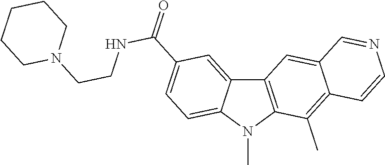 [SMILES: Cc1c2ccncc2cc2c3cc(C(=O)NCCN4CCCCC4)ccc3n(C)c12] |
| 214 | 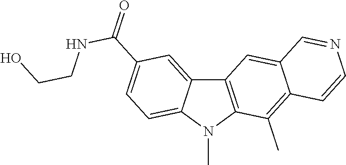 [SMILES: Cc1c2ccncc2cc2c3cc(C(=O)NCCO)ccc3n(C)c12] |
| 215 | 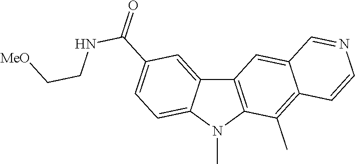 [SMILES: COCCNC(=O)c1ccc2c(c1)c1cc3cnccc3c(C)c1n2C] |
| 216 | 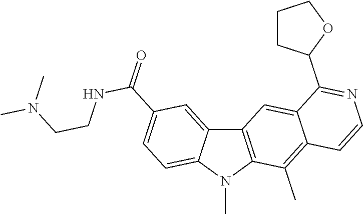 [SMILES: Cc1c2ccnc(C3CCCO3)c2cc2c3cc(C(=O)NCCN(C)C)ccc3n(C)c12] |
| 217 | 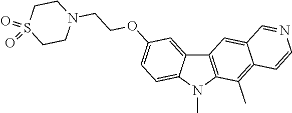 [SMILES: Cc1c2ccncc2cc2c3cc(OCCN4CCS(=O)(=O)CC4)ccc3n(C)c12] |
| 218 | 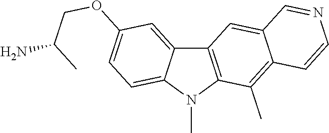 [SMILES: Cc1c2ccncc2cc2c3cc(OC[C@H](C)N)ccc3n(C)c12] |
| 219 | 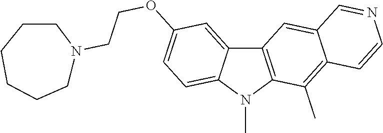 [SMILES: Cc1c2ccncc2cc2c3cc(OCCN4CCCCCC4)ccc3n(C)c12] |
| 220 | 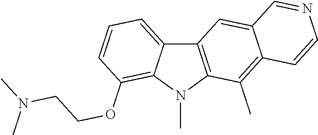 [SMILES: Cc1c2ccncc2cc2c3cccc(OCCN(C)C)c3n(C)c12] |
| 221 | 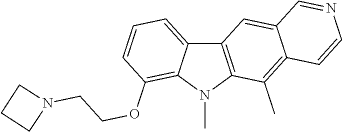 [SMILES: Cc1c2ccncc2cc2c3cccc(OCCN4CCC4)c3n(C)c12] |
| 222 | 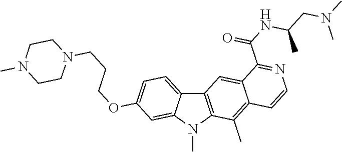 |
| 223 | 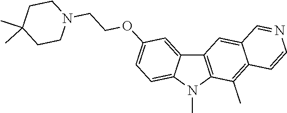 [SMILES: Cc1c2ccncc2cc2c3cc(OCCN4CCC(C)(C)CC4)ccc3n(C)c12] |
| 224 | 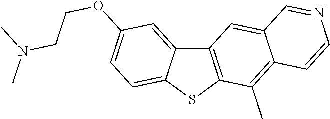 [SMILES: Cc1c2ccncc2cc2c1sc1ccc(OCCN(C)C)cc12] |
| 225 | 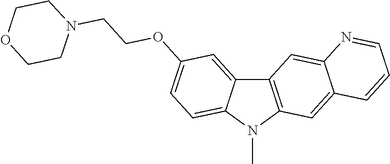 [SMILES: Cn1c2ccc(OCCN3CCOCC3)cc2c2cc3ncccc3cc21] |
| 226 | 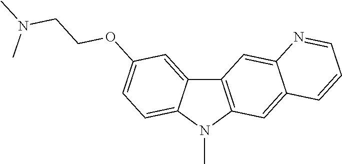 [SMILES: CN(C)CCOc1ccc2c(c1)c1cc3ncccc3cc1n2C] |
| 227 | 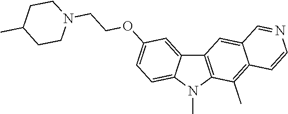 [SMILES: Cc1c2ccncc2cc2c3cc(OCCN4CCC(C)CC4)ccc3n(C)c12] |
| 228 | 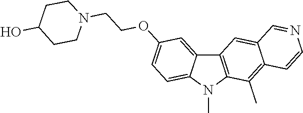 [SMILES: Cc1c2ccncc2cc2c3cc(OCCN4CCC(O)CC4)ccc3n(C)c12] |
| 229 | 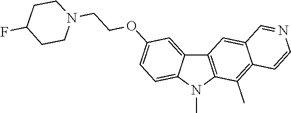 [SMILES: Cc1c2ccncc2cc2c3cc(OCCN4CCC(F)CC4)ccc3n(C)c12] |
| 230 | 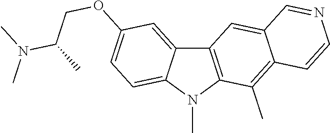 [SMILES: Cc1c2ccncc2cc2c3cc(OC[C@H](C)N(C)C)ccc3n(C)c12] |
| 231 | 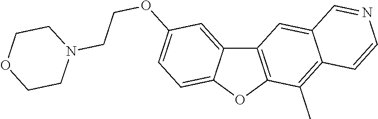 [SMILES: Cc1c2ccncc2cc2c1oc1ccc(OCCN3CCOCC3)cc12] |
| 232 | 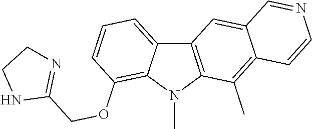 [SMILES: Cc1c2ccncc2cc2c3cccc(OCC4=NCCN4)c3n(C)c12] |
| 233 | 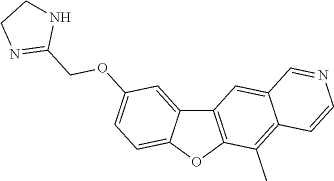 [SMILES: Cc1c2ccncc2cc2c1oc1ccc(OCC3=NCCN3)cc12] |
| 234 | 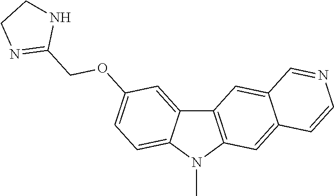 [SMILES: Cn1c2ccc(OCC3=NCCN3)cc2c2cc3cnccc3cc21] |
| 235 | 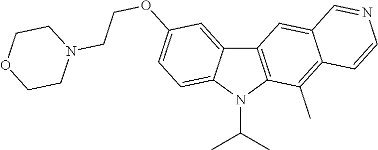 [SMILES: Cc1c2ccncc2cc2c3cc(OCCN4CCOCC4)ccc3n(C(C)C)c12] |
| 236 | 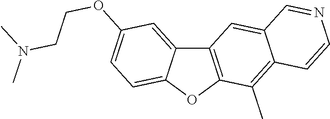 [SMILES: Cc1c2ccncc2cc2c1oc1ccc(OCCN(C)C)cc12] |
| 237 | 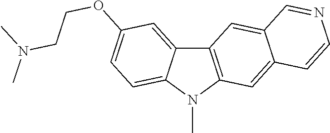 [SMILES: CN(C)CCOc1ccc2c(c1)c1cc3cnccc3cc1n2C] |
| 238 | 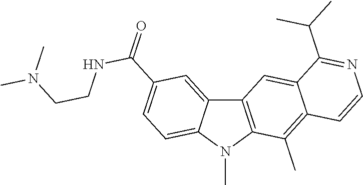 [SMILES: Cc1c2ccnc(C(C)C)c2cc2c3cc(C(=O)NCCN(C)C)ccc3n(C)c12] |
| 239 | 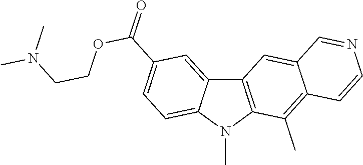 [SMILES: Cc1c2ccncc2cc2c3cc(C(=O)OCCN(C)C)ccc3n(C)c12] |
| 240 | 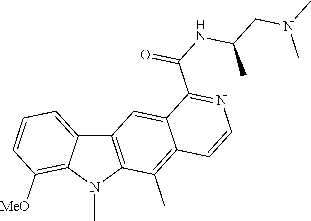 [SMILES: COc1cccc2c3cc4c(C(=O)N[C@H](C)CN(C)C)nccc4c(C)c3n(C)c12] |
| 241 | 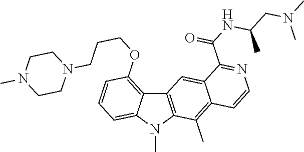 |
| 242 | 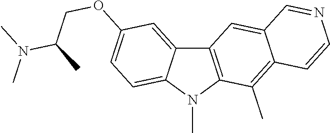 [SMILES: Cc1c2ccncc2cc2c3cc(OC[C@@H](C)N(C)C)ccc3n(C)c12] |
| 243 | 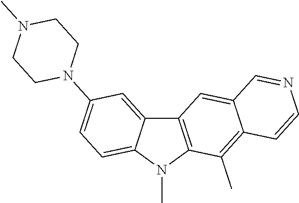 [SMILES: Cc1c2ccncc2cc2c3cc(N4CCN(C)CC4)ccc3n(C)c12] |
| 244 | 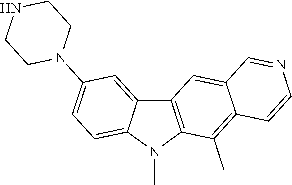 [SMILES: Cc1c2ccncc2cc2c3cc(N4CCNCC4)ccc3n(C)c12] |
| 245 | 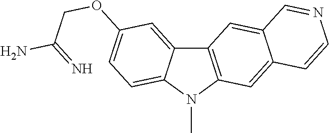 [SMILES: Cn1c2ccc(OCC(=N)N)cc2c2cc3cnccc3cc21] |
| 246 | 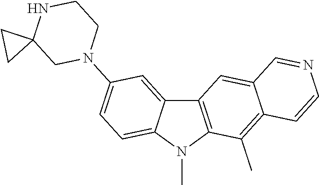 [SMILES: Cc1c2ccncc2cc2c3cc(N4CCNC5(CC5)C4)ccc3n(C)c12] |
| 247 | 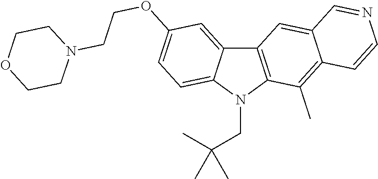 [SMILES: Cc1c2ccncc2cc2c3cc(OCCN4CCOCC4)ccc3n(CC(C)(C)C)c12] |
| 248 | 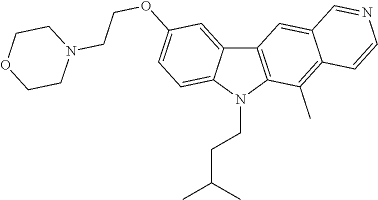 [SMILES: Cc1c2ccncc2cc2c3cc(OCCN4CCOCC4)ccc3n(CCC(C)C)c12] |
| 249 | 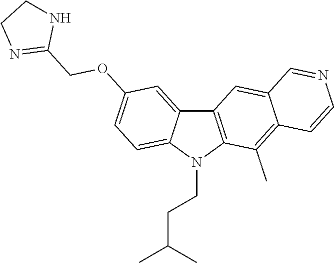 [SMILES: Cc1c2ccncc2cc2c3cc(OCC4=NCCN4)ccc3n(CCC(C)C)c12] |
| 250 | 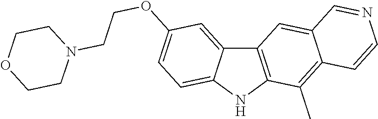 [SMILES: Cc1c2ccncc2cc2c1[nH]c1ccc(OCCN3CCOCC3)cc12] |
| 251 | 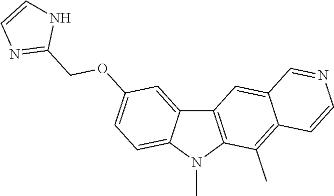 [SMILES: Cc1c2ccncc2cc2c3cc(OCc4ncc[nH]4)ccc3n(C)c12] |
| 252 | 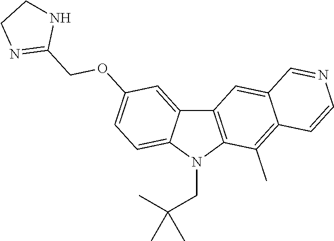 [SMILES: Cc1c2ccncc2cc2c3cc(OCC4=NCCN4)ccc3n(CC(C)(C)C)c12] |
| 253 | 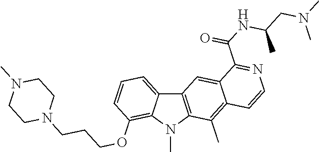 |
| 254 | 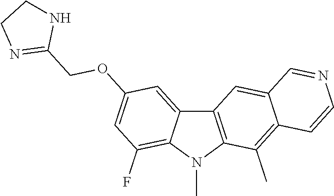 [SMILES: Cc1c2ccncc2cc2c3cc(OCC4=NCCN4)cc(F)c3n(C)c12] |
| 255 | 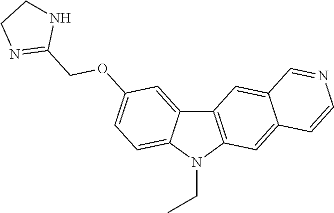 [SMILES: CCn1c2ccc(OCC3=NCCN3)cc2c2cc3cnccc3cc21] |
| 256 | 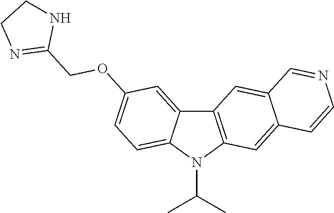 [SMILES: CC(C)n1c2ccc(OCC3=NCCN3)cc2c2cc3cnccc3cc21] |
| 257 | 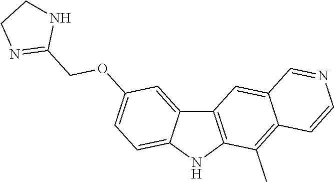 [SMILES: Cc1c2ccncc2cc2c1[nH]c1ccc(OCC3=NCCN3)cc12] |
| 258 | 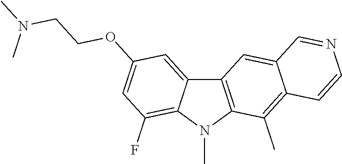 [SMILES: Cc1c2ccncc2cc2c3cc(OCCN(C)C)cc(F)c3n(C)c12] |
| 259 | 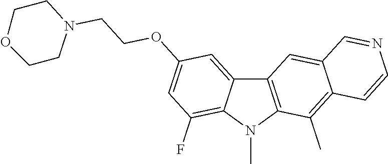 [SMILES: Cc1c2ccncc2cc2c3cc(OCCN4CCOCC4)cc(F)c3n(C)c12] |
| 260 | 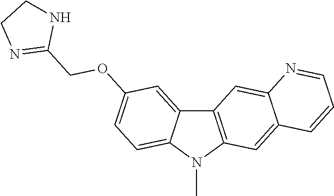 [SMILES: Cn1c2ccc(OCC3=NCCN3)cc2c2cc3ncccc3cc21] |
| 261 | 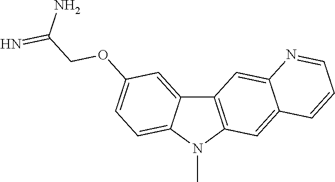 [SMILES: Cn1c2ccc(OCC(=N)N)cc2c2cc3ncccc3cc21] |
| 262 | 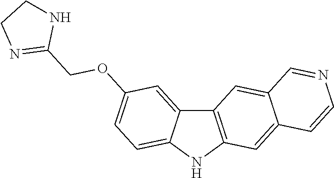 [SMILES: c1cc2cc3[nH]c4ccc(OCC5=NCCN5)cc4c3cc2cn1] |
| 263 | 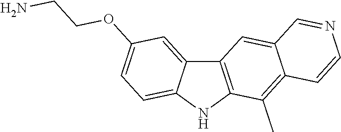 [SMILES: Cc1c2ccncc2cc2c1[nH]c1ccc(OCCN)cc12] |
| 264 | 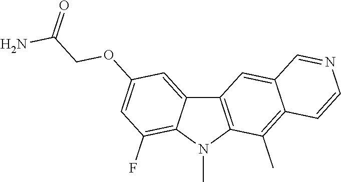 [SMILES: Cc1c2ccncc2cc2c3cc(OCC(N)=O)cc(F)c3n(C)c12] |
| 265 | 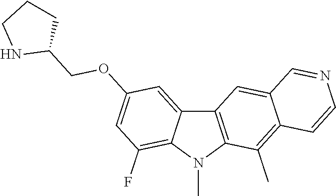 [SMILES: Cc1c2ccncc2cc2c3cc(OC[C@H]4CCCN4)cc(F)c3n(C)c12] |
| 266 | 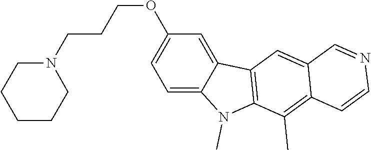 [SMILES: Cc1c2ccncc2cc2c3cc(OCCCN4CCCCC4)ccc3n(C)c12] |
| 267 | 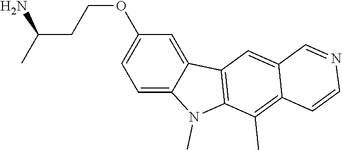 [SMILES: Cc1c2ccncc2cc2c3cc(OCC[C@@H](C)N)ccc3n(C)c12] |
| 268 | 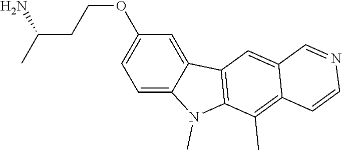 [SMILES: Cc1c2ccncc2cc2c3cc(OCC[C@H](C)N)ccc3n(C)c12] |
| 269 | 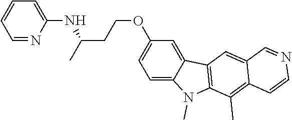 [SMILES: Cc1c2ccncc2cc2c3cc(OCC[C@H](C)Nc4ccccn4)ccc3n(C)c12] |
| 270 | 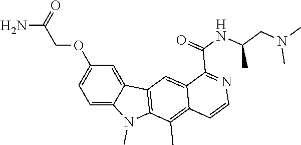 [SMILES: Cc1c2ccnc(C(=O)N[C@H](C)CN(C)C)c2cc2c3cc(OCC(N)=O)ccc3n(C)c12] |
| 271 | 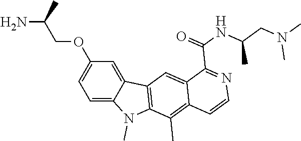 [SMILES: Cc1c2ccnc(C(=O)N[C@H](C)CN(C)C)c2cc2c3cc(OC[C@H](C)N)ccc3n(C)c12] |
| 272 | 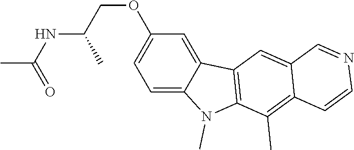 [SMILES: CC(=O)N[C@@H](C)COc1ccc2c(c1)c1cc3cnccc3c(C)c1n2C] |
| 273 | 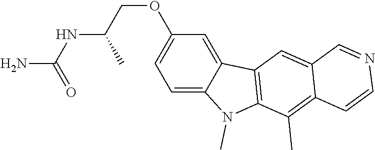 [SMILES: Cc1c2ccncc2cc2c3cc(OC[C@H](C)NC(N)=O)ccc3n(C)c12] |
| 274 | 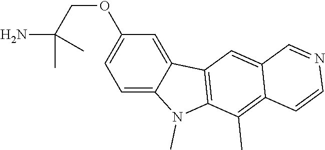 [SMILES: Cc1c2ccncc2cc2c3cc(OCC(C)(C)N)ccc3n(C)c12] |
| 275 | 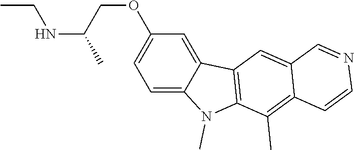 [SMILES: CCN[C@@H](C)COc1ccc2c(c1)c1cc3cnccc3c(C)c1n2C] |
| 276 | 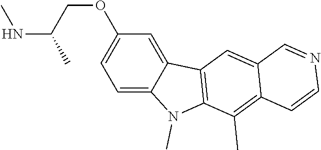 [SMILES: CN[C@@H](C)COc1ccc2c(c1)c1cc3cnccc3c(C)c1n2C] |
| 277 | 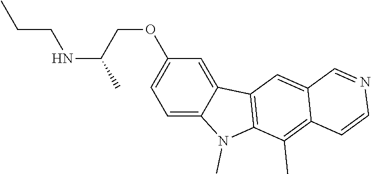 [SMILES: CCCN[C@@H](C)COc1ccc2c(c1)c1cc3cnccc3c(C)c1n2C] |
| 278 | 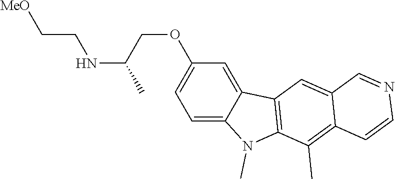 [SMILES: COCCN[C@@H](C)COc1ccc2c(c1)c1cc3cnccc3c(C)c1n2C] |
| 279 | 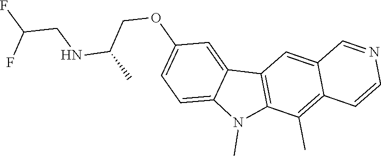 [SMILES: Cc1c2ccncc2cc2c3cc(OC[C@H](C)NCC(F)F)ccc3n(C)c12] |
| 280 | 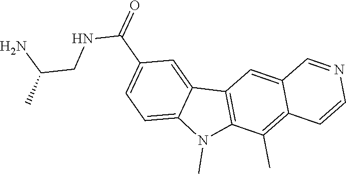 [SMILES: Cc1c2ccncc2cc2c3cc(C(=O)NC[C@H](C)N)ccc3n(C)c12] |
| 281 | 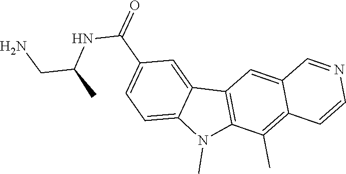 [SMILES: Cc1c2ccncc2cc2c3cc(C(=O)N[C@@H](C)CN)ccc3n(C)c12] |
| 282 | 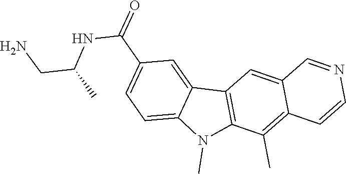 [SMILES: Cc1c2ccncc2cc2c3cc(C(=O)N[C@H](C)CN)ccc3n(C)c12] |
| 283 | 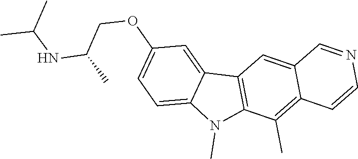 [SMILES: Cc1c2ccncc2cc2c3cc(OC[C@H](C)NC(C)C)ccc3n(C)c12] |
| 284 | 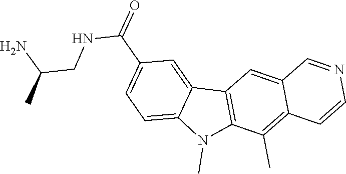 [SMILES: Cc1c2ccncc2cc2c3cc(C(=O)NC[C@@H](C)N)ccc3n(C)c12] |
| 285 | 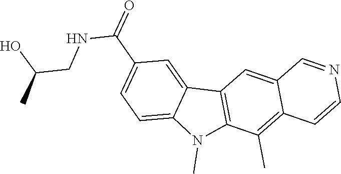 [SMILES: Cc1c2ccncc2cc2c3cc(C(=O)NC[C@@H](C)O)ccc3n(C)c12] |
| 286 | 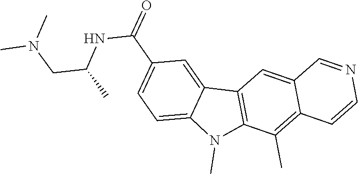 [SMILES: Cc1c2ccncc2cc2c3cc(C(=O)N[C@H](C)CN(C)C)ccc3n(C)c12] |
| 287 | 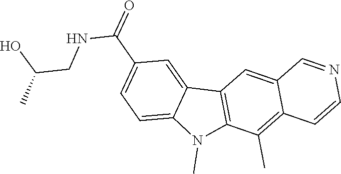 [SMILES: Cc1c2ccncc2cc2c3cc(C(=O)NC[C@H](C)O)ccc3n(C)c12] |
| 288 | 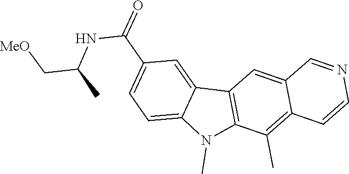 [SMILES: COC[C@H](C)NC(=O)c1ccc2c(c1)c1cc3cnccc3c(C)c1n2C] |
| 289 | 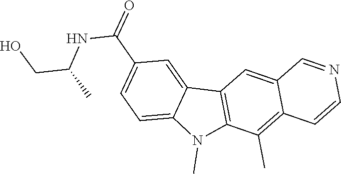 [SMILES: Cc1c2ccncc2cc2c3cc(C(=O)N[C@H](C)CO)ccc3n(C)c12] |
| 290 | 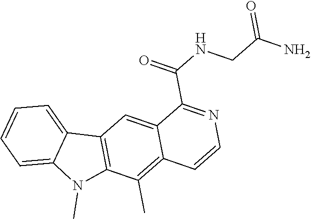 [SMILES: Cc1c2ccnc(C(=O)NCC(N)=O)c2cc2c3ccccc3n(C)c12] |
| 291 | 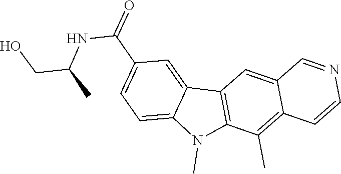 [SMILES: Cc1c2ccncc2cc2c3cc(C(=O)N[C@@H](C)CO)ccc3n(C)c12] |
| 292 | 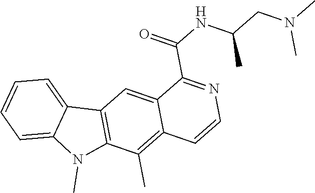 [SMILES: Cc1c2ccnc(C(=O)N[C@H](C)CN(C)C)c2cc2c3ccccc3n(C)c12] |
| 293 | 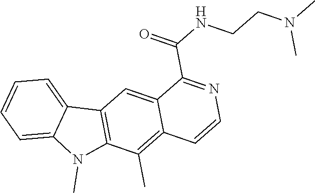 [SMILES: Cc1c2ccnc(C(=O)NCCN(C)C)c2cc2c3ccccc3n(C)c12] |
| 294 | 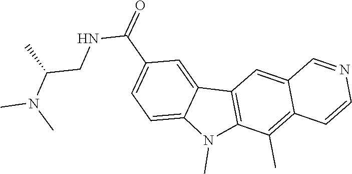 [SMILES: Cc1c2ccncc2cc2c3cc(C(=O)NC[C@@H](C)N(C)C)ccc3n(C)c12] |
| 295 | 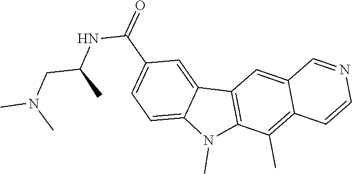 [SMILES: Cc1c2ccncc2cc2c3cc(C(=O)N[C@@H](C)CN(C)C)ccc3n(C)c12] |
| 296 | 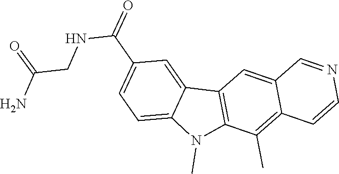 [SMILES: Cc1c2ccncc2cc2c3cc(C(=O)NCC(N)=O)ccc3n(C)c12] |
| 297 | 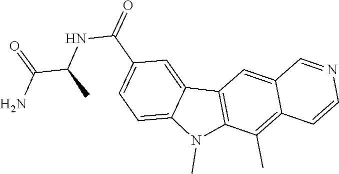 [SMILES: Cc1c2ccncc2cc2c3cc(C(=O)N[C@@H](C)C(N)=O)ccc3n(C)c12] |
| 298 | 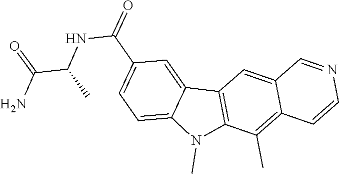 [SMILES: Cc1c2ccncc2cc2c3cc(C(=O)N[C@H](C)C(N)=O)ccc3n(C)c12] |
| 299 | 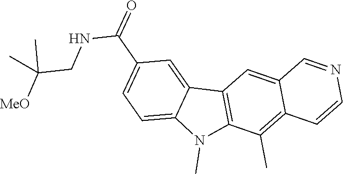 [SMILES: COC(C)(C)CNC(=O)c1ccc2c(c1)c1cc3cnccc3c(C)c1n2C] |
| 300 | 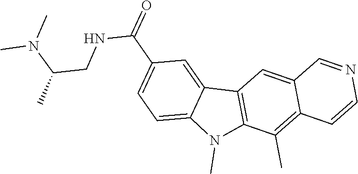 [SMILES: Cc1c2ccncc2cc2c3cc(C(=O)NC[C@H](C)N(C)C)ccc3n(C)c12] |
| 301 | 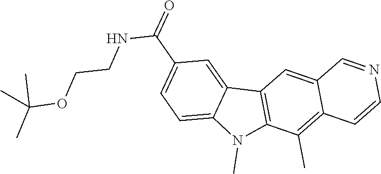 [SMILES: Cc1c2ccncc2cc2c3cc(C(=O)NCCOC(C)(C)C)ccc3n(C)c12] |
| 302 | 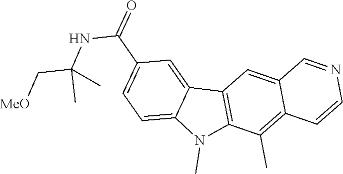 [SMILES: COCC(C)(C)NC(=O)c1ccc2c(c1)c1cc3cnccc3c(C)c1n2C] |
| 303 | 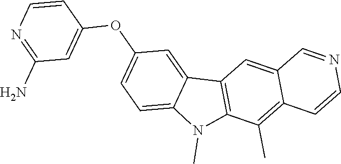 [SMILES: Cc1c2ccncc2cc2c3cc(Oc4ccnc(N)c4)ccc3n(C)c12] |
| 304 | 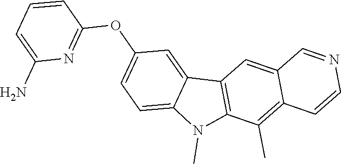 [SMILES: Cc1c2ccncc2cc2c3cc(Oc4cccc(N)n4)ccc3n(C)c12] |
| 305 | 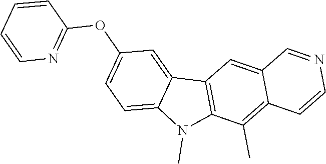 [SMILES: Cc1c2ccncc2cc2c3cc(Oc4ccccn4)ccc3n(C)c12] |
| 306 | 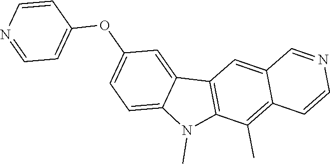 [SMILES: Cc1c2ccncc2cc2c3cc(Oc4ccncc4)ccc3n(C)c12] |
| 307 | 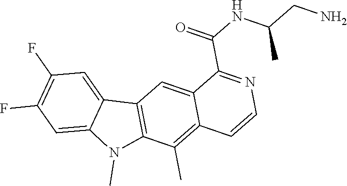 [SMILES: Cc1c2ccnc(C(=O)N[C@H](C)CN)c2cc2c3cc(F)c(F)cc3n(C)c12] |
| 308 | 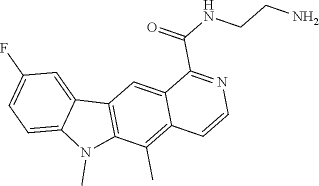 [SMILES: Cc1c2ccnc(C(=O)NCCN)c2cc2c3cc(F)ccc3n(C)c12] |
| 309 | 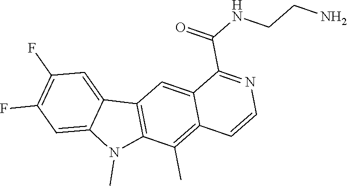 [SMILES: Cc1c2ccnc(C(=O)NCCN)c2cc2c3cc(F)c(F)cc3n(C)c12] |
| 310 | 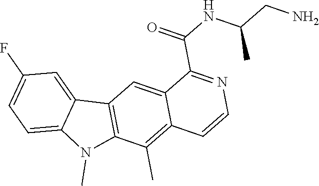 [SMILES: Cc1c2ccnc(C(=O)N[C@H](C)CN)c2cc2c3cc(F)ccc3n(C)c12] |
| 311 | 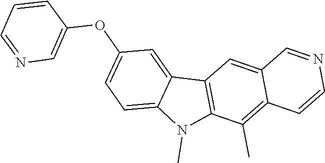 [SMILES: Cc1c2ccncc2cc2c3cc(Oc4cccnc4)ccc3n(C)c12] |
| 312 | 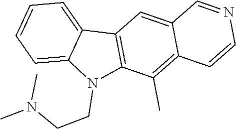 [SMILES: Cc1c2ccncc2cc2c3ccccc3n(CCN(C)C)c12] |
| 313 | 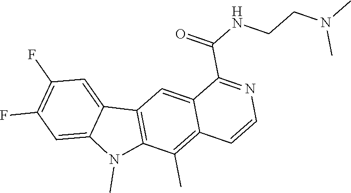 [SMILES: Cc1c2ccnc(C(=O)NCCN(C)C)c2cc2c3cc(F)c(F)cc3n(C)c12] |
| 314 | 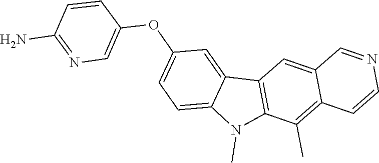 [SMILES: Cc1c2ccncc2cc2c3cc(Oc4ccc(N)nc4)ccc3n(C)c12] |
| 315 | 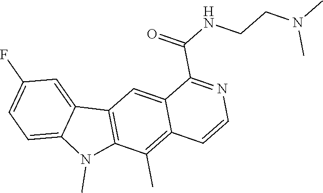 [SMILES: Cc1c2ccnc(C(=O)NCCN(C)C)c2cc2c3cc(F)ccc3n(C)c12] |
| 316 | 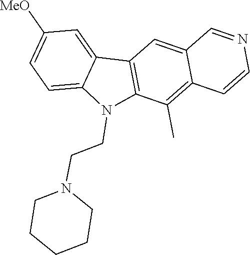 [SMILES: COc1ccc2c(c1)c1cc3cnccc3c(C)c1n2CCN1CCCCC1] |
| 317 | 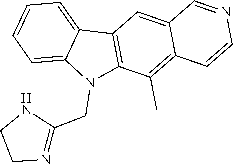 [SMILES: Cc1c2ccncc2cc2c3ccccc3n(CC3=NCCN3)c12] |
| 318 | 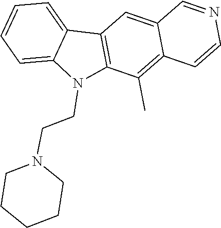 [SMILES: Cc1c2ccncc2cc2c3ccccc3n(CCN3CCCCC3)c12] |
| 319 | 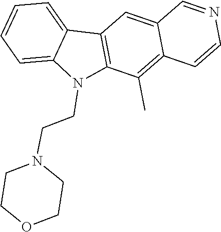 [SMILES: Cc1c2ccncc2cc2c3ccccc3n(CCN3CCOCC3)c12] |
| 320 | 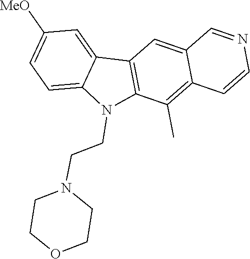 [SMILES: COc1ccc2c(c1)c1cc3cnccc3c(C)c1n2CCN1CCOCC1] |
| 321 | 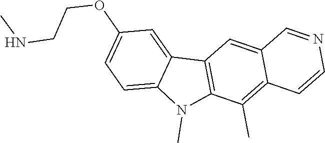 [SMILES: CNCCOc1ccc2c(c1)c1cc3cnccc3c(C)c1n2C] |

### Pharmaceutical Composition

The disclosure also provides a pharmaceutical composition comprising a therapeutically effective amount of one or more compounds as disclosed herein or a pharmaceutically acceptable salt, stereoisomer, or tautomer, in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In some embodiments, the pharmaceutical composition comprises a compound selected from those illustrated in Table A or Table B or a pharmaceutically acceptable salt, stereoisomer, or tautomer thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound (or a pharmaceutically acceptable salt, stereoisomer, or tautomer thereof) that is administered is governed by such considerations, and is the minimum amount necessary to inhibit expanded G4C2 repeat RNA (r(G4C2)exp). Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 1 mg to about 1000 mg of a compound (or a pharmaceutically acceptable salt, stereoisomer, or tautomer thereof) of the present disclosure. In some embodiments, such dosage forms contain from about 50 mg to about 500 mg of a compound (or a pharmaceutically acceptable salt, stereoisomer, or tautomer thereof) of the present disclosure. In various embodiments, such dosage forms contain from about 25 mg to about 200 mg of a compound (or a pharmaceutically acceptable salt, stereoisomer, or tautomer thereof) of the present disclosure. In some embodiments, such dosage forms contain from about 10 mg to about 100 mg of a compound (or a pharmaceutically acceptable salt, stereoisomer, or tautomer thereof) of the present disclosure. In some embodiments, such dosage forms contain from about 5 mg to about 50 mg of a compound (or a pharmaceutically acceptable salt, stereoisomer, or tautomer thereof) of the present disclosure.

The disclosed compositions can be administered orally, topically, parenterally, by inhalation or spray or rectally in dosage unit formulations. The term parenteral as used herein includes subcutaneous injections, intravenous, intramuscular, intrasternal, and intrathecal injection or infusion techniques.

Suitable oral compositions in accordance with the invention include without limitation tablets, troches, lozenges, aqueous or oily suspensions, dispersible powders or granules, emulsion, hard or soft capsules, syrups or elixirs.

Also provided herein are pharmaceutical compositions suitable for single unit dosages that comprise a compound of the disclosure or its pharmaceutically acceptable stereoisomer, salt, or tautomer and a pharmaceutically acceptable carrier.

Compositions suitable for oral use may be prepared according to any method known to the art for the manufacture of pharmaceutical compositions. For instance, liquid formulations of the inventive compounds contain one or more agents selected from the group consisting of sweetening agents, flavoring agents, coloring agents and preserving agents.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensaturatedion products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds disclosed herein may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Methods of Use

The present disclosure provides methods of treating a subject suffering from a disease or condition characterized by an expanded G₄C₂ repeat RNA (r(G₄C₂)^{exp}). The disclosed methods comprise administering to the subject a therapeutically effective amount of any compound as described herein, such as a compound of Formula I or Formulae (Xa), (Xb), (Xc), (Xd), or a compound shown in Table A, Table B, or Table C.

Transcription of r(G₄C₂)^{exp} leads to accumulation of nuclear foci that sequester various RNA binding proteins (RBPs) such as HNRNPA3, PURA, ADARB2, HNRNPH1 among many others (Jazurek, M., Ciesiolka, A., Starega-Roslan, J., Bilinska, K., and Krzyzosiak, W. J. (2016) Identifying proteins that bind to specific RNAs—focus on simple repeat expansion diseases, Nucleic Acids Research 44, 9050-9070; Kumar, V., Hasan, G. M., and Hassan, M. I. (2017) Unraveling the Role of RNA Mediated Toxicity of C9orf72 Repeats in C9-FTD/ALS, Frontiers in Neuroscience 11). Consequently, their ability to properly regulate RNA targets is impaired, ultimately leading to global mis-splicing events, defects in RNA metabolism and nucleocytoplasmatic transport. The r(G₄C₂)^{exp} transcripts undergo repeat associated non-ATG (RAN) translation. (Green, K. M., Linsalata, A. E., and Todd, P. K. (2016) RAN translation-What makes it run?, Brain Research 1647, 30-42; Cleary, J. D., and Ranum, L. P. W. (2017) New developments in RAN translation: insights from multiple diseases, Current Opinion in Genetics & Development 44, 125-134). This non-canonical translation occurs across expanded repeats despite the absence of an ATG initiating codon and results in the production of toxic dipeptide repeats (DPRs) among which poly(GP) was identified as a pharmacodynamic marker in c9ALS patients (Gendron, T. F., et al. (2017) Poly(GP) proteins are a useful pharmacodynamic marker for C9ORF72-associated amyotrophic lateral sclerosis, Science Translational Medicine 9). Toxic dipeptide repeats are associated with the production of dipeptide repeat proteins. These dipeptide repeat proteins are toxic and prone to aggregation, and are associated with various diseases and disorders, such as Amyotrophic lateral sclerosis (ALS) and frontotemporal dementia (FTD).

Accordingly, in some aspects, the disclosure provides methods of treating physiological states associated with diseases and disorders such as ALS and FTD. In some cases, the disclosure provides methods of inhibiting RAN translation which produces toxic dipeptide repeat proteins. In some cases, the disclosure provides methods of inhibiting nuclear foci. In some cases, the disclosure provides methods of rescuing nuclear pore dysfunction. In some cases, the disclosure provides methods of rescuing protein localization.

Amyotrophic lateral sclerosis (ALS) and frontotemporal dementia (FTD) are fatal neurodegenerative disorders that share common clinical pathologies and genetic causes (Taylor, J. P., Brown Jr, R. H., and Cleveland, D. W. (2016) Decoding ALS: from genes to mechanism, Nature 539, 197-206). Both disorders are highly heterogeneous with respect to underlying causal genetics, site and age onset (Swinnen, B., and Robberecht, W. (2014) The phenotypic variability of amyotrophic lateral sclerosis, Nat Rev Neurol 10, 661-670), partially explaining the lack of approved drugs despite more than 20 years of ALS research (Petrov, D., Mansfield, C., Moussy, A., and Hermine, O. (2017) ALS Clinical Trials Review: 20 Years of Failure. Are We Any Closer to Registering a New Treatment?, Frontiers in Aging Neuroscience 9). The discovery of G4C2 hexanucleotide repeat expansions (r(G₄C₂)^{exp}) in the non-coding region of C9ORF72 gene is the most common cause of ALS/FTD, designated c9ALS/FTD (DeJesus-Hernandez, M. et al. (2011) Expanded GGGGCC Hexanucleotide Repeat in Noncoding Region of C9ORF72 Causes Chromosome 9p-Linked FTD and ALS, Neuron 72, 245-256; Renton, Alan E et al. (2011) A Hexanucleotide Repeat Expansion in C9ORF72 Is the Cause of Chromosome 9p21-Linked ALS-FTD, Neuron 72, 257-268). Healthy individuals carry up to 30 G₄C₂ sequences, whereas c9ALS/FTD disease phenotypes are triggered by hundreds to thousand such repeats (Beck, J. et al. (2013) Large C9orf72 Hexanucleotide Repeat Expansions Are Seen in Multiple Neurodegenerative Syndromes and Are More Frequent Than Expected in the UK Population, The American Journal of Human Genetics 92, 345-353; van Blitterswijk, M. et al. Association between repeat sizes and clinical and pathological characteristics in carriers of C9ORF72 repeat expansions (Xpansize-72): a cross-sectional cohort study, The Lancet Neurology 12, 978-988).

The present disclosure is predicated, in some embodiments, upon the discovery of small molecule compounds described herein that selectively inhibit repeat associated non-ATG (RAN) translation. Accordingly, the compounds are useful in treating diseases that are characterized by an expanded G₄C₂ repeat RNA (r(G₄C₂)^{exp}). Thus, in some embodiments, the disease or condition is amyotrophic lateral sclerosis (ALS) or frontotemporal dementia (FTD).

### EXAMPLES

TR-FRET hnRNP H Binding Assay. The TR-FRET assays were performed as previously described (Disney et al., 2012) except the final concentrations of biotinylated r(G₄C₂)₈ (SEQ ID NO: 2) RNA and hnRNP H1-His₆ were 80 nM and 75 nM, respectively. The RNA was folded by heating to 95° C. for 5 min in 1× Folding Buffer followed by slowly cooling to room temperature. The buffer was adjusted to 1×TR-FRET Assay Buffer followed by addition of compound. The mixture was incubated for 15 min at room temperature and then hnRNP H1-His₆ was added. After an additional 15 min incubation, streptavidin-XL665 (HTRF, Cisbio Bioassays) and anti-His₆-Tb (HTRF, Cisbio Bioassays) were added to a final concentration of 40 nM and 0.44 ng/μL, respectively. After incubating for 1 h, TR-FRET was measured as previously described. The IC₅₀s were either calculated by fitting the equation (1) in Graphpad Prism.

$$y = B + \frac{A - B}{1 + {( \frac{IC 50}{x} )}^{hillslope}} \qquad ( eq . 1 )$$

Cell culture. The HEK293T cells were cultured in 1×DMEM containing 4.5 g/L glucose, 10% FBS, 2 mM L-glutamine, and 1×apenicillin/streptomycin (growth medium) at 37° C. in 5% C02.

Cell-based assay to assess RAN translation and Poly(GP) response. The HEK293T cells (80% confluent) were batch-transfected in growth medium for 4 h with (G₄C₂)₆₆-No ATG-GFP (SEQ ID NO: 11) and a plasmid encoding mCherry (for normalization) using JetPrime transfection reagent (Polyplus) according to the manufacturer's instructions. The cells were then seeded into a 384-well plate and incubated for 2 h before addition of compound using Biomek NXP Laboratory Automation Workstation that was equipped with a 100 nL 384-pin head. After treatment for 24 h, the medium was removed, and the cells were lysed in 100 mM potassium phosphate lysis buffer, pH 7.8, and 0.2% Triton X-100. Fluorescence was measured using a BioTek FLx800 with 530/25 nm (excitation) and 590/35 nm (emission) filters for mCherry and 485/20 nm (excitation) and 528/20 nm (emission) filters for GFP. Background was determined by measuring the corresponding fluorescence intensities in untransfected cells. The background-corrected ratio of GFP to mCherry was used to determine the effect of compound on RAN translation. Poly (GP) measurement was followed by previous protocol. (Su et al., 2014) The HEK293T cells were batch-transfected with (G₄C₂)₆₆ (SEQ ID NO: 11) or (TG₄C2)₆₂ plasmid and were treated different concentration of compound. Data from this assay is presented in Table C.

Effect of compound treatment on (G₄C₂)₆₆ (SEQ ID NO: 11)-No ATG-GFP transcript levels. Approximately 2×10⁶ HEK293T cells were plated in 60 mm cell culture dishes and transfected with 3.5 μg (G₄C₂)₆₆-No ATG-GFP (SEQ ID NO: 11)plasmid using JetPrime transfect agent per manufacturer's protocol. After 4 h, cells were trypsinized and plated into 48-well plates in growth medium. After an additional 2 h, the compound of interest or vehicle were added in growth medium. After overnight incubation, the compound-containing medium was removed, and cells were washed with 1× Dulbecco® phosphate-buffered saline (DPBS). Total RNA was harvested with a Quick RNA™ Mini-Prep Kit (Zymo Research) per the manufacturer's recommended protocol.

The cDNA was generated from 100 ng RNA using a QScript cDNA Synthesis Kit (Quanta Biosciences) according to manufacturer's recommended procedure; qPCR was completed with Power SYBR Green PCR Master Mix (Life Technologies) and 1 μL of 10-fold diluted cDNA on an Applied Biosystems 7900HT Fast Real Time PCR System. The levels of (G₄C₂)₆₆-No ATG-GFP (SEQ ID NO: 11) were measured with primers specific for GFP and normalized to 18S or p-actin values. Table S3 lists the sequences of all PCR primers.

CD spectroscopy. The CD spectroscopic experiments were conducted using a spectropolarimeter (J-815 Jasco) with a bandwidth of 2 nm at a scan speed of 50 nm/min and a step resolution of 0.2 nm over the spectral range of 210-450 nm. The RNA sample concentration was 10 to 20 μM in 10 mM Na₂HPO₄ buffer with 50 mM LiCl. Thermal melting curves were recorded by a peltier thermal coupler chamber (Jasco), was monitored at 26 5 nm between 20 and 95° C. with a temperature ramping rate of 1° C./min rate. The observed signals were baseline subtracted, and the first derivative zero points were defined as the melting temperature. Shape analysis of the melting curves yielded Van′t Hoff enthalpy (ΔH_{vH}) using standard procedures (Marky and Breslauer, 1987). The cooperativity of binding of a compound to r(G₄C₂) repeats was measured by plotting the normalized signal intensity at 300 nm or 330 nm in CD spectra as a function of the molar ratio of ([compound]/[RNA]) and fitted to the following equation:

y=x₀+(xⁿ^{_{Hill}})/(xⁿ^{_{Hill}}+Kⁿ^{_{Hill}}) (eq 2)

where y is the normalized signal intensity, x₀ is initial molar ratio of ([compound]/[RNA]); x is the molar ratio of ([compound]/[RNA]), n_{Hill} is Hill coefficient, and K is the Hill binding constant.

In some embodiments, RNA (5 μM final concentration) was annealed at 95° C. for 4 min in 100 mM LiCl and 10 mM sodium phosphate buffer (pH=7) and let to cool down to room temperature. CD spectra were recorded upon dose dependent addition of compound (from 10 mM stock solution in 100% DMSO) on a JASCO J-815 spectrometer using a 10 mm cuvette and PMT CD detector. Data was collected at 25° C. by acquiring the CD signal within 210-500 nm range at a scanning speed of 50 nm/min using standard sensitivity, band width of 1 nm and D.I.T. of 1 s.

Gel shift assay. Gel shift assays were performed using self-cast 15% native PAGE gel. Briefly, RNAs (80 μM final concentration) were annealed at 95° C. for 4 min in 10 mM sodium phosphate buffer (pH=7) and let to cool down to room temperature. Samples were loaded on gel and ran at 125 V for 1 hour at room temperature using 1×TBE buffer. Bromophenol blue and xylene cyanol were used as size markers. Bands were visualized using UV lamp.

RNA FISH and immunostaining: nuclear foci. The RNA FISH and hnRNP H immunostaining were completed as previously described. (Lee et al., 2013) Briefly, HEK293T cells (80% confluent) were batch-transfected as described above. The cells were then seeded into 12-well plates with coverslips and incubated for 2 h before addition of compound. After incubation with compound for 24 h, the cells were fixed in 4% paraformaldehyde for 15 min and washed 5 times with 1×DPBS. After washing with 1×DPBS for 15 min at room temperature, the cells were washed with 0.1% Triton X-100 in 1×DPBS for 5 min at room temperature followed by washing with 40% formamide in 2×SSC buffer for 15 min at room temperature. The RNA FISH probe was then added (5 ng/μL of 5′-cy3-d(G₂C4)₈-3′) in 40% formamide in 2×SSC containing 8 μg/mL BSA, 66 μg/mL yeast tRNA, and 2 mM vanadyl complex. The samples were incubated at 48° C. and 5% CO₂ overnight. Following overnight hybridization, the cells were washed three times with 2×SSC and then three times with 1×DPBS at room temperature for 15 min each. To immunostain hnRNP H, cells were fixed with 2% (v/v) formaldehyde in 1×DPBS, permeabilized with 0.1% (v/v) Triton X-100 in 1×DPBS, blocked with 5% goat serum for 40 min, and then incubated with a 1:250 dilution of anti-hnRNP H (Novus Bio) in 1×DPBS for 1 h at room temperature. After washing with 1×DPBS twice, the cells were incubated with a 1:250 dilution anti-rabbit IgG DyLight 650 in 1×DPBS at room temperature for 1 h. After washing (1×DPBS three times at room temperature for 15 min each), cells were stained with 1 μg/mL DAPI in 1×DPBS for 10 min at room temperature and then washed again with 1×DPBS immediately before imaging. Images were collected using an Olympus Fluoview 1000 confocal microscope.

Foci Imaging by Compound and BG4, a G-quadruplex antibody. Briefly, HEK293T cells (80% confluent) were batch-transfected as described above. The cells were then seeded into 12-well plates with coverslips and incubated for 2 h before addition of compound. After incubation with compound for 24 h, the cells were fixed in 2% paraformaldehyde for 15 min and washed five times with 1×DPBS. The cells were then washed with 0.1% Triton X-100 in 1×DPBS for 5 min and three times with 1×DPBS. Compound (0.5 μM) was added to the cells for 2 h at 37° C.

For experiments employing compound or RNase, the cells were incubated compound (5 μM for 24 h) or RNase, the cells were incubated an RNase solution (25 mg/ml in 1×DPBS) at 37° C. for 2 h and washed 2 times with 1×DPBS before treatment with compound. For BG4 studies, (Moye et al., 2015) the cells were fixed with 2% (v/v) formaldehyde in 1×DPBS, permeabilized with 0.1% (v/v) Triton X-100 in PBS, and blocked with 2% (w/v) milk in 1×DPBS. After washing with 1×DPBS twice, the slides were incubated with BG4 primary antibody (600 nM) for 1 h at 37° C., and washed three times for 5 min with 0.01% Tween-20 in 1×DPBS and then overlaid with secondary antibody (Rabbit anti-flag Tag antibody, Cell Signaling; 1:800 dilution) for 1 h at 37° C. After washing three times with 0.01% Tween-20 in 1×DPBS, the cells were incubated with anti-rabbit IgG DyLight 650. The cells were stained with 1 μg/mL DAPI in 1×DPBS for 10 min at room temperature and then washed with 1×DPBS. Images were collected using an Olympus Fluoview 1000 confocal microscope.

Polysome profiling. Polysome profiling studies were completed similarly to previously described methods (Yang et al., 2016; Yang et al., 2015). Briefly, 100 mm dishes were plated with 4.5×10⁶ HEK293T cells and batch-transfected as described above. Approximately 2 h after plating, cells were treated with vehicle or compound (5 μM) and incubated for 16-18 h. Cells were then lysed with 250 μL of ice-cold Cell Lysis Buffer [10 mM NaCl, 10 mM MgCl₂, 10 mM Tris, pH 7.5, 1% Triton X-100, 1% sodium deoxycholate, 1 mM DTT supplemented with 0.1 mg/L cycloheximide and 0.2 U/μL RNAsin (Promega)]. The lysate was transferred to an Eppendorf tube, gently vortexed and centrifuged for 5 min at 13,200 rpm and 4° C. The supernatant was transferred to a new tube and stored at −80° C. until further use.

Cellular lysates were separated using a 10-50% sucrose gradient in 500 μL fractions. A 100 μL aliquot from each fraction was removed and the RNA isolated by using a Quick RNA™ Mini-Prep (Zymo Research) according to manufacturer's protocol. cDNA was generated from 250 ng of RNA using a QScript cDNA Synthesis Kit according to manufacturer's recommended procedure. The levels of (G₄C₂)₆₆-No ATG-GFP (SEQ ID NO: 11) mRNA in each fraction were measured by qPCR as described above with primers specific for GFP. Data from vehicle- and compound-treated samples were normalized as follows: triplicate Cₜ values were averaged and ΔCₜ values were calculated by comparison to β-actin; ΔΔCₜ values were afforded by comparing treated to untreated ΔCₜ values. Data were then normalized to the fraction with the lowest abundance of GFP.

In vitro translation assay. The RNA template for translational assays was transcribed from the (G₄C₂)₆₆-No ATG-NanoLuc (SEQ ID NO: 11) plasmid using an in vitro transcription system (RiboMax, Promega). In vitro translation reactions (50 μL) using the Flexi Rabbit Reticulocyte Lysate System (Promega) were programmed with 4 μg (G₄C₂)₆₆-No ATG-NanoLuc (SEQ ID NO: 11) RNA and 1 μg Firefly Luciferase RNA (Promega) as the template. The RNA was folded into the G-quadruplex form in buffer containing KCl (384 mM) by heating at 95° C. for 10 min followed by slow cooling to 25° C. in a total volume of 10 μL. The RNA was folded into the hairpin form in buffer by placing on ice. (NMR spectroscopy shows that only the hairpin is formed at low temperatures.) After folding, the RNA was added to the reticulocyte lysate, amino acid mixtures, and RNasin ribonuclease inhibitor (Promega) in a total volume of 50 μL (manufacturer's protocol). Translation proceed for 90 min at 30° C. for 90 min and then luciferase activity was measured. The reactions were diluted in Glo Lysis Buffer followed by addition of Nano-Glo substrate (Promega).

### Example 1

### 9-methoxy-5,6-dimethyl-6H-pyrido[4,3-b]carbazole

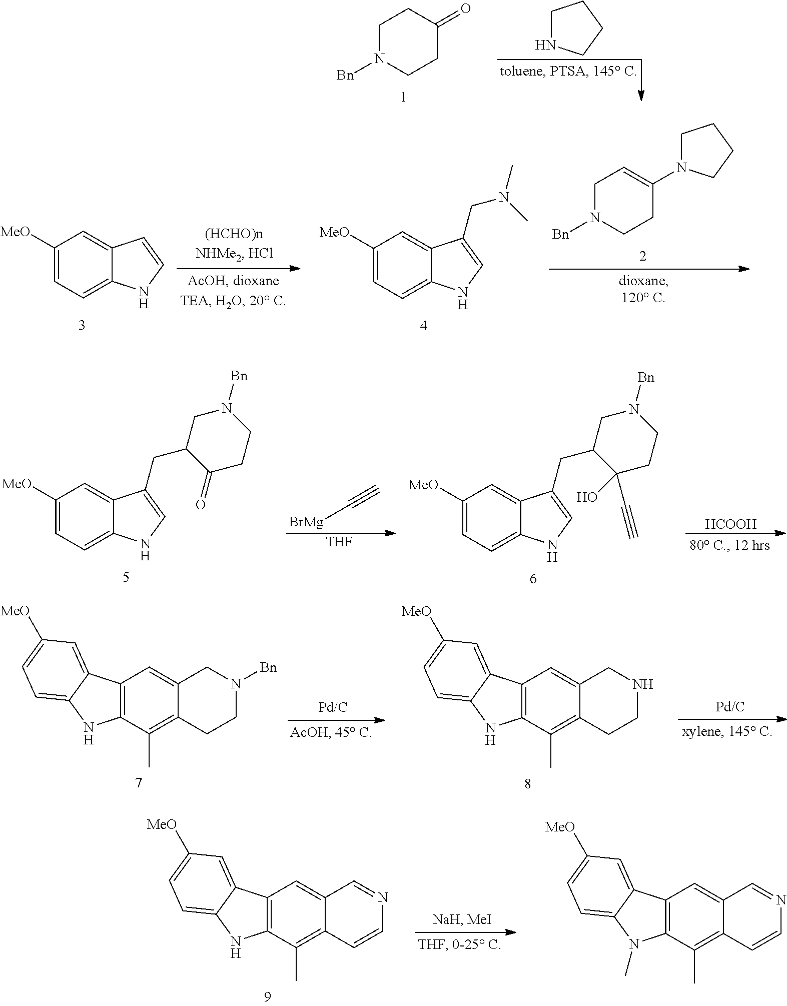

### Step 1: benzyl-4-(pyrrolidin-1-yl)-1,2,3,6-tetrahydropyridine

To a solution of 1-benzylpiperidin-4-one (350 g, 1.8 mol, 343.1 mL, 1 eq), pyrrolidine (197.1 g, 2.7 mol, 231.4 mL, 1.5 eq) in toluene (1000 mL) was added PTSA (968.3 mg, 5.6 mmol, 3 eq). The mixture was stirred at 140° C. for 12 hr. TLC indicated one major new spot with lower polarity was detected. The reaction mixture was concentrated under reduced pressure to remove solvent and to give 480 g of crude benzyl-4-(pyrrolidin-1-yl)-1,2,3,6-tetrahydropyridine.

### Step 2: 1-(5-methoxy-1H-indol-3-yl)-N,N-dimethylmethanamine

A mixture of 5-methoxy-1H-indole (50 g, 339.7 mmol, 1 eq, 2 batch), formaldehyde (30.3 g, 373.7 mmol, 27.8 mL, 37% purity, 1.1 eq), N-methylmethanamine; hydrochloride (30.4 g, 373.7 mmol, 1.1 eq), TEA (37.8 g, 373.7 mmol, 52.0 mL, 1.1 eq), AcOH (30.6 g, 509.6 mmol, 29.1 mL, 1.5 eq) in dioxane (300 mL) and H₂O (300 mL) was stirred at 20° C. for 12 hr. LC-MS showed 89.8% of desired compound was detected. The reaction mixture was concentrated under reduced pressure to remove dioxane. The aqueous was added to NaOH·aq (100 mL, 2M) at 25° C. and then the mixture was filtered and the filter cake was concentrated under reduced pressure to give 1-(5-methoxy-1H-indol-3-yl)-N,N-dimethylmethanamine.

### Step 3: 1-benzyl-3-((5-methoxy-1H-indol-3-yl)methyl)piperidin-4-one

A solution of 1-(5-methoxy-1H-indol-3-yl)-N,N-dimethylmethanamine (50 g, 244.7 mmol, 1 eq, 2 batch) in dioxane (400 mL) was added 1-benzyl-4-(pyrrolidin-1-yl)-1,2,3,6-tetrahydropyridine (148.3 g, 611.9 mmol, 2.5 eq), then the mixture was stirred at 120° C. for 12 hr. LC-MS showed 1-(5-methoxy-1H-indol-3-yl)-N,N-dimethylmethanamine was consumed completely and one main peak with desired m/z was detected. The reaction mixture was concentrated under reduced pressure to remove solvent. The residue was diluted with H₂O (200 mL) and extracted with EtOAc (100 mL×5). The combined organic layers were washed with brine (20 mL×3), dried over Na₂SO₄, filtered and concentrated under reduced pressure to give a residue. The residue was purified by flash silica gel chromatography (Eluent of 0˜60% Ethyl acetate/Petroleum ether) to give 1-benzyl-3-((5-methoxy-1H-indol-3-yl)methyl) piperidin-4-one.

### Step 4: 1-benzyl-4-ethynyl-3-((5-methoxy-1H-indol-3-yl)methyl)piperidin-4-ol

A mixture of 1-benzyl-3-((5-methoxy-1H-indol-3-yl)methyl)piperidin-4-one (40 g, 114.8 mmol, 1 eq) in THF (500 mL) was added bromo(ethynyl)magnesium (0.5 M, 918.3 mL, 4 eq) at 0° C. and then the mixture was stirred at 0° C. for 2 hr under N₂ atmosphere. LC-MS showed 1-benzyl-3-((5-methoxy-1H-indol-3-yl)methyl)piperidin-4-one was consumed completely and one main peak with desired m/z was detected. The reaction mixture was quenched by addition aq. NH₄Cl (150 mL) at 20° C., and extracted with EtOAc (100 ml×5). The combined organic layers were washed with brine (20 mL×3), dried over Na₂SO₄, filtered and concentrated under reduced pressure to give a crude 1-benzyl-4-ethynyl-3-((5-methoxy-1H-indol-3-yl)methyl)piperidin-4-ol (42 g, 112.1 mmol).

### Step 5: 2-benzyl-9-methoxy-5-methyl-2,3,4,6-tetrahydro-1H-pyrido[4,3-b]carbazole

A solution of 1-benzyl-4-ethynyl-3-((5-methoxy-1H-indol-3-yl)methyl)piperidin-4-ol (42 g, 112.1 mmol, 1 eq) in HCOOH (500 mL) was stirred at 80° C. for 12 hr. TLC indicated 1-benzyl-4-ethynyl-3-((5-methoxy-1H-indol-3-yl)methyl)piperidin-4-ol was consumed completely and many new spots formed. The reaction mixture was concentrated under reduced pressure to remove solvent. The residue was diluted with H₂O (50 mL), adjusted pH to 8 with sat NaHCO₃ and extracted with EtOAc (100 mL×5). The combined organic layers were washed with brine (30 mL×2), dried over Na₂SO₄, filtered and concentrated under reduced pressure to give a residue. The residue was purified by flash silica gel chromatography (ISCO®; 120 g SepaFlash® Silica Flash Column, Eluent of 0˜100% Ethyl acetate/Petroleum ether gradient @ 120 mL/min) to afford 2-benzyl-9-methoxy-5-methyl-2,3,4,6-tetrahydro-1H-pyrido[4,3-b]carbazole.

### Step 6: 9-methoxy-5-methyl-2,3,4,6-tetrahydro-1H-pyrido[4,3-b]carbazole

A solution of 2-benzyl-9-methoxy-5-methyl-2,3,4,6-tetrahydro-1H-pyrido[4,3-b]carbazole (5.3 g, 14.8 mmol, 1 eq) in AcOH (60 mL) was added Pd/C (3 g, 14.87 mmol, 10% purity). The mixture was stirred at 45° C. for 2 hr under H₂ (15 psi). LCMS showed the reaction was completed and main peak with desired product. The mixture was filtered and concentrated under reduced pressure to give 9-methoxy-5-methyl-2,3,4,6-tetrahydro-1H-pyrido [4,3-b]carbazole (as AcOH salt).

### Step 7: 9-methoxy-5-methyl-6H-pyrido[4,3-b]carbazole

To a solution of 9-methoxy-5-methyl-2,3,4,6-tetrahydro-1H-pyrido [4,3-b]carbazole (3.5 g, 10.7 mmol, 1 eq, HOAC) in xylene (100 mL) was added Pd/C (2 g, 10.72 mmol, 10% purity, 1 eq). The mixture was stirred at 145° C. for 12 hr. LCMS showed the reaction was completed and desired product was detected. The mixture was filtered and concentrated under reduced pressure to give 9-methoxy-5-methyl-6H-pyrido[4,3-b]carbazole.

### Step 8: 9-methoxy-5,6-dimethyl-6H-pyrido[4,3-b]carbazole

To a solution of 9-methoxy-5-methyl-6H-pyrido[4,3-b]carbazole (2.2 g, 8.3 mmol, 1 eq) in THF (40 mL) was added NaH (1.0 g, 25.1 mmol, 60% purity, 3 eq) and Mel (1.1 g, 8.3 mmol, 522.1 uL, 1 eq) at 0° C. Then the mixture was stirred at 25° C. for 2 hr. LCMS showed the reaction was completed and desired product was detected. The mixture was poured into NH₄Cl (100 mL) and extracted with EtOAc (50 mL×3). The combined organic layers were washed with brine (50 mL×3), dried over Na₂SO₄, filtered and concentrated under reduced pressure to give 9-methoxy-5,6-dimethyl-6H-pyrido[4,3-b] carbazole.

### Example 2

### 5,6-dimethyl-6H-pyrido[4,3-b]carbazol-9-ol

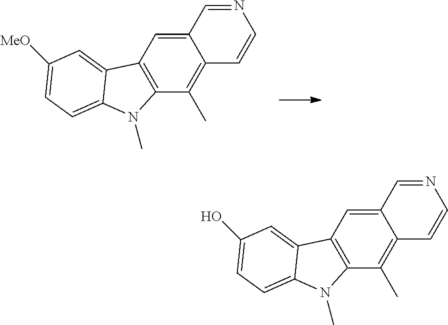 [SMILES: COc1ccc2c(c1)c1cc3cnccc3c(C)c1n2C ; Cc1c2ccncc2cc2c3cc(O)ccc3n(C)c12]

To a solution of E-1780 (500 mg, 1.81 mmol) in DCM (20 ml) was added BBr3 (1.7 ml, 18 mmol) at 0° C. and warmed to rt and stirred overnight. The mixture was added NH4OH aq (10%) and added DCM. The combined org phase was washed with brine, dried over Na2SO4 and concentrated in vacuo. The part of residue was purified with HPLC and the rest was used in further reaction as crude.

### Preparatory Reverse-Phase HPLC Method

Reverse-phase high pressure liquid chromatography (HPLC) purification was performed using a Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm.

### Example 3

### 9-((4,5-dihydro-1H-imidazol-2-yl)methoxy)-5,6-dimethyl-6H-pyrido[4,3-b] carbazole

To a solution of 5,6-dimethyl-6H-pyrido[4,3-b]carbazol-9-ol (400 mg, 1.5 mmol, 1 eq) in DMF (10 mL) was added 2-(chloromethyl)-4,5-dihydro-1H-imidazole; hydrochloride (283.6 mg, 1.8 mmol, 1.2 eq), LiOH·H₂O (255.9 mg, 6.1 mmol, 4 eq) and NaI (228.5 mg, 1.5 mmol, 1 eq). The mixture was stirred at 80° C. for 12 hr. LCMS showed the reaction was completely. The reaction was concentrated to give a residue. The residue was purified by prep-HPLC (TFA condition, column: Phenomenex luna C18 250*50 mm*10 um; mobile phase: [water(0.1% TFA)-ACN]; B %: 5%-35%, 10 min) to afford 9-((4,5-dihydro-1H-imidazol-2-yl)methoxy)-5,6-dimethyl-6H-pyrido[4,3-b] carbazole.

MS (ESI): m/z=345.0 [M+H]₊

### Example 4

### N-(2-aminoethyl)-2-((5,6-dimethyl-6H-pyrido[4,3-b]carbazol-9-yl)oxy)acetamide (4a) and 2-((5,6-dimethyl-6H-pyrido[4,3-b]carbazol-9-yl)oxy)acetic acid (4b)

To a solution of 9-((4,5-dihydro-1H-imidazol-2-yl)methoxy)-5,6-dimethyl-6H-pyrido[4,3-b] (17 mg, 0.05 mmol) in DMF was added LiOH—H2O (4 mg, 0.1 mmol) and the mixture was heated at 70° C. for 6 h and then purified with HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm.) to afford 4a and 4b.
4a: MS (ESI): 363.2 [M+H]⁺
4b: MS (ESI): 321.1 [M+H]⁺

### Examples 5-13

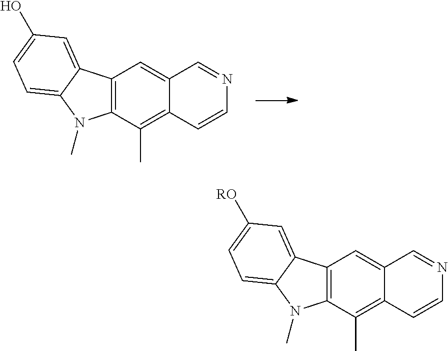

### General Procedure for Preparation of Compounds 5-13

To a solution of the product of Example 2 (52 mg, 0.20 mmol) in DMF (1 ml) was added LiOH·H2O (21 mg, 0.50 mmol) and NaI (2 mg, 0.013 mmol) and alkyl chloride (R—Cl, 0.25 mmol) and the mixture was heated at 70° C. for 4 h. The mixture was purified by HPLC (Reverse-phase high pressure liquid chromatography (HPLC) purification was performed using a Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm) to afford the product shown in table below:

| Example | | | |
|---|---|---|---|
| # | Compound Name | MS (ESI) | Structure |
| 5 | (2-((5,6-dimethyl-6H- pyrido[4,3-b]carbazol-9- yl)oxy)-N,N-dimethylethan-1- amine | 334.5 [M + H]+ | 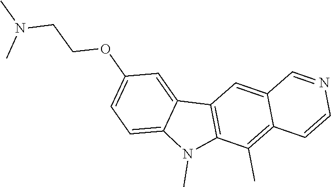 [SMILES: Cc1c2ccncc2cc2c3cc(OCCN(C)C)ccc3n(C)c12] |
| 6 | 3-((5,6-dimethyl-6H- pyrido[4,3-b]carbazol-9- yl)oxy)-N,N-dimethylpropan- 1-amine | 348.5 [M + H]+ | 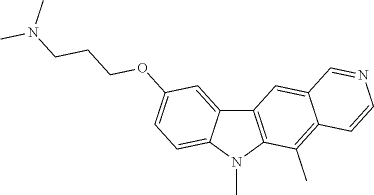 [SMILES: Cc1c2ccncc2cc2c3cc(OCCCN(C)C)ccc3n(C)c12] |
| 7 | 5,6-dimethyl-9-(2-(pyrrolidin- 1-yl)ethoxy)-6H-pyrido[4,3- b]carbazole | 457.6 [M + H]+ | 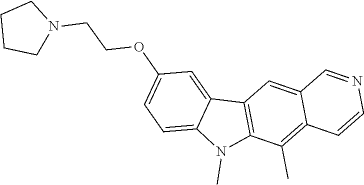 [SMILES: Cc1c2ccncc2cc2c3cc(OCCN4CCCC4)ccc3n(C)c12] |
| 8 | 4-(3-((5,6-dimethyl-6H- pyrido[4,3-b]carbazol-9- yl)oxy)propyl)morpholine | 390.5 [M + H]+ | 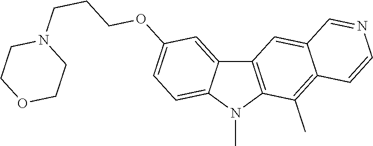 [SMILES: Cc1c2ccncc2cc2c3cc(OCCCN4CCOCC4)ccc3n(C)c12] |
| 9 | 5,6-dimethyl-9-(3-(pyrrolidin- 1-yl)propoxy)-6H-pyrido[4,3- b]carbazole | 374.5 [M + H]+ | 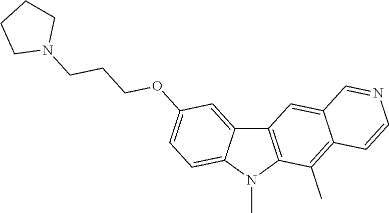 [SMILES: Cc1c2ccncc2cc2c3cc(OCCCN4CCCC4)ccc3n(C)c12] |
| 10 | 3-((5,6-dimethyl-6H- pyrido[4,3-b]carbazol-9- yl)oxy)-N,N-diethylpropan-1- amine | 376.5 [M + H]+ | 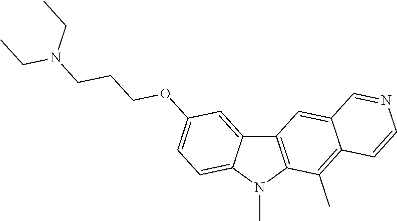 [SMILES: CCN(CC)CCCOc1ccc2c(c1)c1cc3cnccc3c(C)c1n2C] |
| 11 | 4-(2-((5,6-dimethyl-6H- pyrido[4,3-b]carbazol-9- yl)oxy)ethyl)morpholine | 376.5 [M + H]+ | 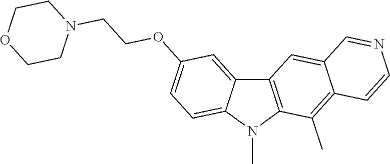 [SMILES: Cc1c2ccncc2cc2c3cc(OCCN4CCOCC4)ccc3n(C)c12] |
| 12 | 2-((5,6-dimethyl-6H- pyrido[4,3-b]carbazol-9- yl)oxy)acetamide | 320.1 [M + H]+ | 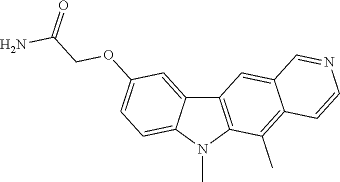 [SMILES: Cc1c2ccncc2cc2c3cc(OCC(N)=O)ccc3n(C)c12] |
| 13 | 2-((5,6-dimethyl-6H- pyrido[4,3-b]carbazol-9- yl)oxy)acetonitrile | 302.4 [M + H]+ | 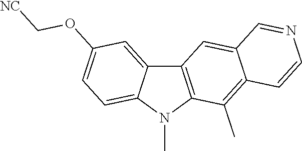 [SMILES: Cc1c2ccncc2cc2c3cc(OCC#N)ccc3n(C)c12] |

### Examples 14-19

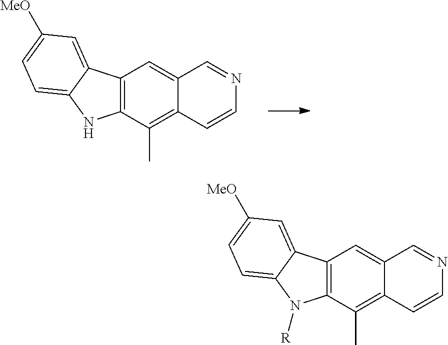

### General Procedure for Preparation of Compounds 14-19

To a solution of 9-methoxy-5-methyl-6H-pyrido[4,3-b]carbazole (26 mg, 0.1 mmol; Example 1, step 7) in THF (1 ml) was added KOtBu (22 mg, 0.2 mmol), NaI (15 mg, 0.1 mmol) and electrophile (0.2 mmol) and the mixture was stirred at rt overnight, then purified by prep HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm) to afford the product as shown in the Table below.

| Example # | Compound Name | Structure |
|---|---|---|
| 14 | 9-methoxy-5-methyl-6-((1- methyl-1H-pyrazol-4-yl)methyl)- 6H-pyrido[4,3-b]carbazol | 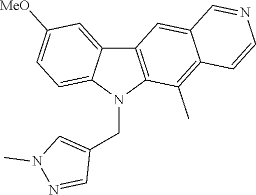 [SMILES: COc1ccc2c(c1)c1cc3cnccc3c(C)c1n2Cc1cnn(C)c1] |
| 15 | 3-(2-(9-methoxy-5-methyl-6H- pyrido[4,3-b]carbazol-6- yl)ethyl)tetrahydrothiophene 1,1- dioxide | 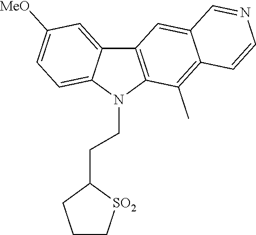 [SMILES: COc1ccc2c(c1)c1cc3cnccc3c(C)c1n2CCC1CCCS1(=O)=O] |
| 16 | 9-methoxy-5-methyl-6-(pyridin-4- ylmethyl)-6H-pyrido[4,3- b]carbazole | 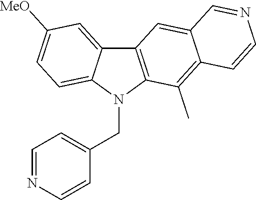 [SMILES: COc1ccc2c(c1)c1cc3cnccc3c(C)c1n2Cc1ccncc1] |
| 17 | 6-benzyl-9-methoxy-5-methyl- 6H-pyrido[4,3-b]carbazole | 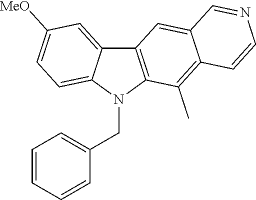 [SMILES: COc1ccc2c(c1)c1cc3cnccc3c(C)c1n2Cc1ccccc1] |
| 18 | 6-isopropyl-9-methoxy-5-methyl- 6H-pyrido[4,3-b]carbazole | 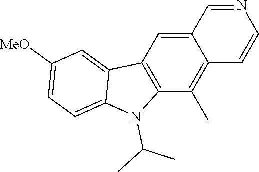 [SMILES: COc1ccc2c(c1)c1cc3cnccc3c(C)c1n2C(C)C] |
| 19 | (S)-2-(9-methoxy-5-methyl-6H- pyrido[4,3-b]carbazol-6-yl)-1- phenylethan-1-ol | 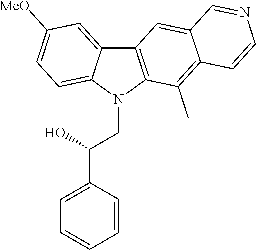 [SMILES: COc1ccc2c(c1)c1cc3cnccc3c(C)c1n2C[C@@H](O)c1ccccc1] |

### Examples 20-22

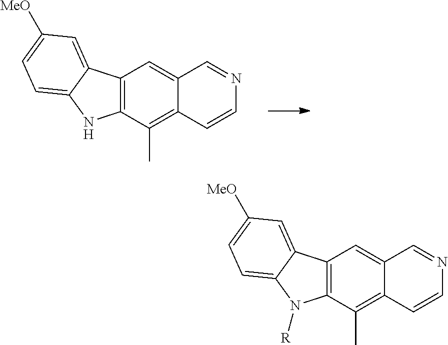

### General Procedure for Preparation of Compounds 20-22

To a solution of 9-methoxy-5-methyl-6H-pyrido[4,3-b]carbazole (26 mg, 0.1 mmol; Example 1, step 7) in THF was added NaH (8 mg, 0.20 mmol) and NaI (1.5 mg, 0.01 mmol), alkyl chloride (0.2 mmol) and the mixture was stirred at rt overnight and then purified with HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm) to afford the product as shown in the table below.

| Example # | Compound Name | Structure |
|---|---|---|
| 20 | 2-(9-methoxy-5-methyl-6H- pyrido[4,3-b]carbazol-6-yl)-N,N- dimethylethan-1-amine | 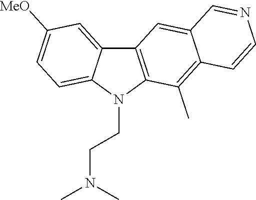 [SMILES: COc1ccc2c(c1)c1cc3cnccc3c(C)c1n2CCN(C)C] |
| 21 | 6-((4,5-dihydro-1H-imidazol-2- yl)methyl)-9-methoxy-5-methyl- 6H-pyrido[4,3-b]carbazole | 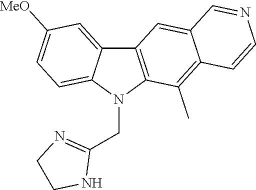 [SMILES: COc1ccc2c(c1)c1cc3cnccc3c(C)c1n2CC1=NCCN1] |
| 22 | 9-methoxy-5-methyl-6-(3-(4- methylpiperazin-1-yl)propyl)-6H- pyrido[4,3-b]carbazole | 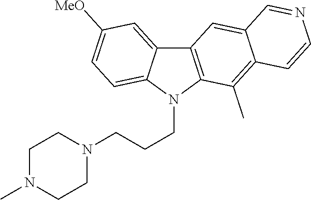 [SMILES: COc1ccc2c(c1)c1cc3cnccc3c(C)c1n2CCCN1CCN(C)CC1] |

### Example 23

### 5,6-dimethyl-9-(3-(4-methylpiperazin-1-yl)propoxy)-6H-pyrido[4,3-b]carbazole

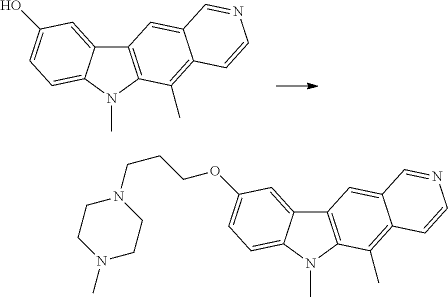

### 5,6-dimethyl-9-(3-(4-methylpiperazin-1-yl)propoxy)-6H-pyrido[4,3-b]carbazole

To a solution of 5,6-dimethyl-6H-pyrido[4,3-b]carbazol-9-ol (26 mg, 0.10 mmol) in DMF (1 ml) was added LiOH—H2O (21 mg, 0.50 mmol) and NaI (2 mg, 0.013 mmol) and 1-(3-chloropropyl)-4-methylpiperazine (44 mg, 0.25 mmol) and the mixture was heated at 70° C. for 4 h. The mixture was purified with HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm) to afford the product.

### Example 24

### 9-methoxy-5,6-dimethyl-6H-pyrido[4,3-b]carbazole-1-carboxylic acid

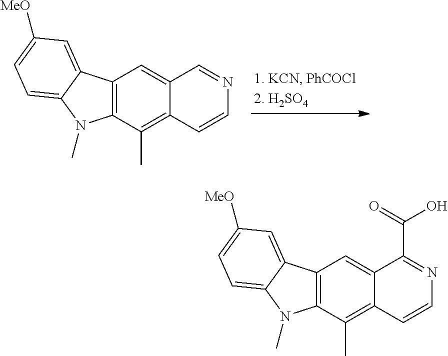 [SMILES: COc1ccc2c(c1)c1cc3cnccc3c(C)c1n2C ; COc1ccc2c(c1)c1cc3c(C(=O)O)nccc3c(C)c1n2C]

### 9-methoxy-5,6-dimethyl-6H-pyrido[4,3-b]carbazole-1-carboxylic acid

To a solution of 9-methoxy-5,6-dimethyl-6H-pyrido[4,3-b]carbazole (11 mg, FW: 320.21, 0.039 mmol) and KCN (7 mg, 0.1 mmol) in water (0.5 ml) and DCM (0.5 ml) was added benzoyl chloride (6 uL, 0.05 mmol) at 0° C., then the mixture was warmed to RT, stirred for 2 h, and then added water and DCM. The org phase was dried over Na₂SO₄ and concentrated in vacuo. The residue was added H₂SO₄/H₂O (1:1.1 ml) and heated at 110° C. overnight. The mixture was purified with RP-HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H₂O and B=MeOH or ACN; Flow rate: 5 mL/min, Injection volume: 4 mL, Runtime: 60 min, gradient: variable concentrations of B in A; UV detection at 220 & 345 nm.) to afford the desired product.

MS (ESI): 321.4

### Example 25

### (R)—N-(1-(dimethylamino)propan-2-yl)-9-methoxy-5,6-dimethyl-6H-pyrido[4,3-b]carbazole-1-carboxamide

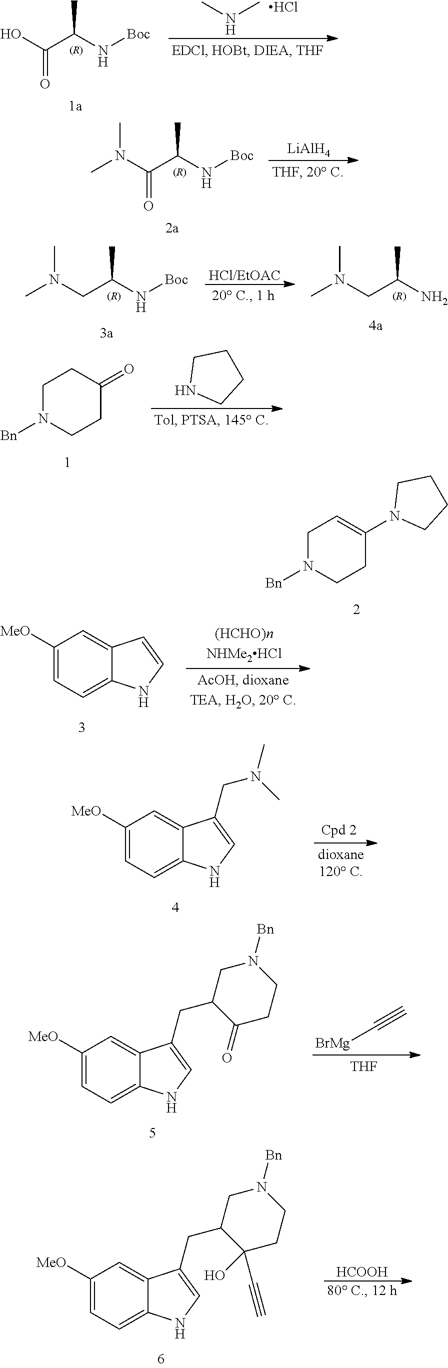

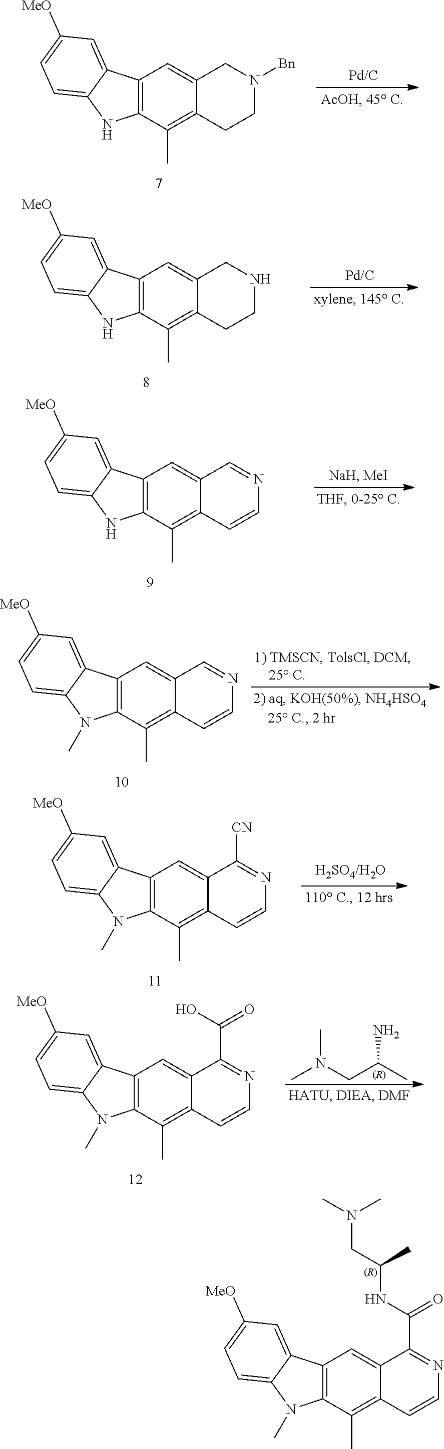

### Step 1: (R)-tert-butyl (1-(dimethylamino)-1-oxopropan-2-yl)carbamate

To a solution of (2R)-2-(tert-butoxycarbonylamino)propanoic acid (20 g, 105.7 mmol, 1 eq) in THF (400 mL) was added DIEA (54.6 g, 422.8 mmol, 73.6 mL, 4 eq), EDCI (30.4 g, 158.5 mmol, 1.5 eq), HOBt (21.4 g, 158.5 mmol, 1.5 eq) and N-methylmethanamine (8.6 g, 105.7 mmol, 9.6 mL, 1 eq, HCl). The mixture was stirred at 20° C. for 12 hr. TLC indicated one major new spot was detected. The reaction mixture was partitioned between H₂O (500 mL) and EtOAc (500 mL). The aqueous phase was washed with EtOAc (300 mL×3). The organic phase was separated and washed with brine (100 mL×3), dried over Na₂SO₄, filtered and concentrated under reduced pressure to give a residue. The residue was purified by flash silica gel chromatography (ISCO®; 80 g SepaFlash® Silica Flash Column, Eluent of 0˜20% Ethyl acetate/Petroleum ether gradient @50 mL/min) to give (R)-tert-butyl (1-(dimethylamino)-1-oxopropan-2-yl)carbamate 2b.

### Step 2: (R)-tert-butyl (1-(dimethylamino)propan-2-yl)carbamate

To a solution of tert-butyl N-[(1R)-2-(dimethylamino)-1-methyl-2-oxoethyl]carbamate (20.3 g, 93.8 mmol, 1 eq) in THF (400 mL) was added LiAlH₄ (7.1 g, 187.7 mmol, 2 eq) at 0° C. The mixture was stirred at 20° C. for 2 hr. TLC indicated one major new spot was detected. The reaction was quenched with saturated MgSO₄ (7.1 mL) and MgSO₄ (20 g) solid was added. The mixture was filtered and the cake was washed with ethyl acetate (200 mL×3). The combined filtrate was concentrated to give the crude product. The residue was purified by flash silica gel chromatography (ISCO®; 40 g SepaFlash® Silica Flash Column, Eluent of 0˜10% Ethyl acetate/Petroleum ether gradient @ 100 mL/min) to give (R)-tert-butyl (1-(dimethylamino)propan-2-yl)carbamate 3a.

### Step 3: (R)—N1,N1-dimethylpropane-1,2-diamine

A solution of tert-butyl N-[(1R)-2-(dimethylamino)-1-methyl-ethyl]carbamate (3.5 g, 17.3 mmol, 1 eq) in HCl/EtOAc (30 mL, 4 mol/L) was stirred at 20° C. for 1 hr. TLC indicated one major new spot was detected. The reaction mixture was concentrated under reduced pressure to give (R)—N1,N1-dimethylpropane-1,2-diamine 4a.

### Step 4: benzyl-4-(pyrrolidin-1-yl)-1,2,3,6-tetrahydropyridine

To a solution of 1-benzylpiperidin-4-one (350 g, 1.8 mol, 343.1 mL, 1 eq), pyrrolidine (197.1 g, 2.7 mol, 231.4 mL, 1.5 eq) in toluene (1000 mL) was added PTSA (968.3 mg, 5.6 mmol, 3 eq). The mixture was stirred at 140° C. for 12 hr. TLC indicated one major new spot with lower polarity was detected. The reaction mixture was concentrated under reduced pressure to remove solvent and to give 480 g of crude benzyl-4-(pyrrolidin-1-yl)-1,2,3,6-tetrahydropyridine 2.

### Step 5: 1-(5-methoxy-1H-indol-3-yl)-N,N-dimethylmethanamine

A mixture of 5-methoxy-1H-indole (50 g, 339.7 mmol, 1 eq, 2 batch), formaldehyde (30.3 g, 373.7 mmol, 27.8 mL, 37% purity, 1.1 eq), N-methylmethanamine; hydrochloride (30.4 g, 373.7 mmol, 1.1 eq), TEA (37.8 g, 373.7 mmol, 52.0 mL, 1.1 eq), AcOH (30.6 g, 509.6 mmol, 29.1 mL, 1.5 eq) in dioxane (300 mL) and H₂O (300 mL) was stirred at 20° C. for 12 hr. LC-MS showed 89.8% of desired compound was detected. The reaction mixture was concentrated under reduced pressure to remove dioxane. The aqueous was added to NaOH·aq (100 mL, 2M) at 25° C. and then the mixture was filtered and the filter cake was concentrated under reduced pressure to give 1-(5-methoxy-1H-indol-3-yl)-N,N-dimethylmethanamine 4.

### Step 6: 1-benzyl-3-((5-methoxy-1H-indol-3-yl)methyl)piperidin-4-one

A solution of 1-(5-methoxy-1H-indol-3-yl)-N,N-dimethylmethanamine (50 g, 244.7 mmol, 1 eq, 2 batch) in dioxane (400 mL) was added 1-benzyl-4-(pyrrolidin-1-yl)-1,2,3,6-tetrahydropyridine (148.3 g, 611.9 mmol, 2.5 eq), then the mixture was stirred at 120° C. for 12 hr. LC-MS showed 1-(5-methoxy-1H-indol-3-yl)-N,N-dimethylmethanamine was consumed completely and one main peak with desired m/z was detected. The reaction mixture was concentrated under reduced pressure to remove solvent. The residue was diluted with H₂O (200 mL) and extracted with EtOAc (100 mL×5). The combined organic layers were washed with brine (20 mL×3), dried over Na₂SO₄, filtered and concentrated under reduced pressure to give a residue. The residue was purified by flash silica gel chromatography (Eluent of 0˜60% Ethyl acetate/Petroleum ether) to give 1-benzyl-3-((5-methoxy-1H-indol-3-yl)methyl) piperidin-4-one 5

### Step 7: 1-benzyl-4-ethynyl-3-((5-methoxy-1H-indol-3-yl)methyl)piperidin-4-ol

A mixture of 1-benzyl-3-((5-methoxy-1H-indol-3-yl)methyl)piperidin-4-one (40 g, 114.8 mmol, 1 eq) in THF (500 mL) was added bromo(ethynyl)magnesium (0.5 M, 918.3 mL, 4 eq) at 0° C. and then the mixture was stirred at 0° C. for 2 hr under N₂ atmosphere. LC-MS showed 1-benzyl-3-((5-methoxy-1H-indol-3-yl)methyl)piperidin-4-one was consumed completely and one main peak with desired m/z was detected. The reaction mixture was quenched by addition aq. NH₄Cl (150 mL) at 20° C., and extracted with EtOAc (100 ml×5). The combined organic layers were washed with brine (20 mL×3), dried over Na₂SO₄, filtered and concentrated under reduced pressure to give a crude 1-benzyl-4-ethynyl-3-((5-methoxy-1H-indol-3-yl)methyl)piperidin-4-ol 6.

### Step 8: 2-benzyl-9-methoxy-5-methyl-2,3,4,6-tetrahydro-1H-pyrido[4,3-b]carbazole

A solution of 1-benzyl-4-ethynyl-3-((5-methoxy-1H-indol-3-yl)methyl)piperidin-4-ol (42 g, 112.1 mmol, 1 eq) in HCOOH (500 mL) was stirred at 80° C. for 12 hr. TLC indicated 1-benzyl-4-ethynyl-3-((5-methoxy-1H-indol-3-yl)methyl)piperidin-4-ol was consumed completely and many new spots formed. The reaction mixture was concentrated under reduced pressure to remove solvent. The residue was diluted with H₂O (50 mL), adjusted pH to 8 with sat NaHCO₃ and extracted with EtOAc (100 mL×5). The combined organic layers were washed with brine (30 mL×2), dried over Na₂SO₄, filtered and concentrated under reduced pressure to give a residue. The residue was purified by flash silica gel chromatography (ISCO®; 120 g SepaFlash® Silica Flash Column, Eluent of 0˜100% Ethyl acetate/Petroleum ether gradient @ 120 mL/min) to afford 2-benzyl-9-methoxy-5-methyl-2,3,4,6-tetrahydro-1H-pyrido[4,3-b]carbazole 7.

### Step 9: 9-methoxy-5-methyl-2,3,4,6-tetrahydro-1H-pyrido[4,3-b]carbazole

A solution of 2-benzyl-9-methoxy-5-methyl-2,3,4,6-tetrahydro-1H-pyrido[4,3-b]carbazole (5.3 g, 14.8 mmol, 1 eq) in AcOH (60 mL) was added Pd/C (3 g, 14.87 mmol, 10% purity). The mixture was stirred at 45° C. for 2 hr under H₂ (15 psi). LCMS showed the reaction was completed and main peak with desired product. The mixture was filtered and concentrated under reduced pressure to give 9-methoxy-5-methyl-2,3,4,6-tetrahydro-1H-pyrido [4,3-b]carbazole (8, as AcOH salt).

### Step 10: 9-methoxy-5-methyl-6H-pyrido[4,3-b]carbazole

To a solution of 9-methoxy-5-methyl-2,3,4,6-tetrahydro-1H-pyrido [4,3-b]carbazole (3.5 g, 10.7 mmol, 1 eq, HOAC) in xylene (100 mL) was added Pd/C (2 g, 10.72 mmol, 10% purity, 1 eq). The mixture was stirred at 145° C. for 12 hr. LCMS showed the reaction was completed and desired product was detected. The mixture was filtered and concentrated under reduced pressure to give 9-methoxy-5-methyl-6H-pyrido[4,3-b]carbazole 9

### Step 11: 9-methoxy-5,6-dimethyl-6H-pyrido[4,3-b]carbazole

To a solution of 9-methoxy-5-methyl-6H-pyrido[4,3-b]carbazole (2.2 g, 8.3 mmol, 1 eq) in THF (40 mL) was added NaH (1.0 g, 25.1 mmol, 60% purity, 3 eq) and Mel (1.1 g, 8.3 mmol, 522.1 uL, 1 eq) at 0° C. Then the mixture was stirred at 25° C. for 2 hr. LCMS showed the reaction was completed and desired product was detected. The mixture was poured into NH₄Cl (100 mL) and extracted with EtOAc (50 mL×3). The combined organic layers were washed with brine (50 mL×3), dried over Na₂SO₄, filtered and concentrated under reduced pressure to give 9-methoxy-5,6-dimethyl-6H-pyrido[4,3-b]carbazole 10.

### Step 12: 9-methoxy-5,6-dimethyl-6H-pyrido[4,3-b]carbazole-1-carbonitrile

To a solution of 9-methoxy-5,6-dimethyl-6H-pyrido[4,3-b] carbazole (2 g, 7.24 mmol, 1 eq) in DCM (40 mL) was added TMSCN (1.8 g, 18.8 mmol, 2.3 mL, 2.6 eq). To this mixture a solution of TosCl (2.6 g, 13.7 mmol, 1.9 eq) in DCM (20 mL) was added at 25° C. Then the mixture was stirred at 30° C. for 12 hr. LCMS showed the intermediate was detected. Then KOH (10 mL, 50% W/W) and NH₄HSO₄ (15 mg) were added. The mixture was stirred at 25° C. for 2 hr. The mixture was extracted with DCM (100 mL×3). The combined organic layers were washed with brine (100 mL×3), dried over Na₂SO₄, filtered and concentrated under reduced pressure to give 9-methoxy-5,6-dimethyl-6H-pyrido[4,3-b]carbazole-1-carbonitrile 11.

### Step 13: 9-methoxy-5,6-dimethyl-6H-pyrido[4,3-b]carbazole-1-carboxylic acid

A solution of 9-methoxy-5,6-dimethyl-6H-pyrido[4,3-b]carbazole-1-carbonitrile (2.1 g, 6.9 mmol, 1 eq) in H₂SO₄ (10 mL, 98% purity) and H₂O (10 mL) was stirred at 110° C. for 12 hr.

LCMS showed the reaction was completed and mainly peak with desired product. The mixture was poured into NaHCO₃ (500 ml) and the red solid was separate out. Then the mixture was filtered and collected the solid to afford 9-methoxy-5,6-dimethyl-6H-pyrido[4,3-b]carbazole-1-carboxylic acid 12.

### Step 14: (R)—N-(1-(dimethylamino)propan-2-yl)-9-methoxy-5,6-dimethyl-6H-pyrido[4,3-b]carbazole-1-carboxamide

To a solution of 9-methoxy-5,6-dimethyl-pyrido[4,3-b]carbazole-1-carboxylic acid (1.5 g, 4.6 mmol, 1 eq) in DMF (30 mL) was added (2R)—N1,N1-dimethylpropane-1,2-diamine (2.4 g, 14.0 mmol, 3 eq, 2 HCl), DIEA (3.0 g, 23.4 mmol, 4.0 mL, 5 eq). Then HATU (3.5 g, 9.3 mmol, 2 eq) was added to the mixture at 0° C. Then the mixture was warmed to 25° C. for 1 hrs. LCMS showed the reaction was completed and desired product was detected. The mixture was concentrated to get crude residue. The residue was purified by prep-HPLC (TFA condition, column: Phenomenex luna C18 (250×70 mm, 15 μm); mobile phase: [water(0.1% TFA)-ACN]; B %: 20%-50%, 20 min) to give (R)—N-(1-(dimethylamino)propan-2-yl)-9-methoxy-5,6-dimethyl-6H-pyrido[4,3-b]carbazole-1-carboxamide) or as TFA salt crude product.

MS (ESI): m/z=405.2 [M+H]⁺

### Example 26

### (R)—N-(1-(dimethylamino)propan-2-yl)-5,6-dimethyl-9-(3-(4-methylpiperazin-1-yl)propoxy)-6H-pyrido[4,3-b]carbazole-1-carboxamide

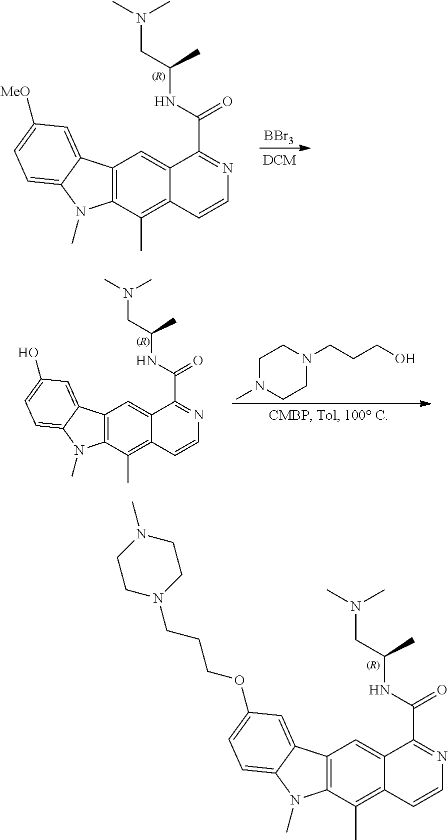

### Step 1: (R)—N-(1-(dimethylamino)propan-2-yl)-9-hydroxy-5,6-dimethyl-6H-pyrido[4,3-b]carbazole-1-carboxamide

To a solution of (R)—N-(1-(dimethylamino)propan-2-yl)-9-methoxy-5,6-dimethyl-6H-pyrido [4,3-b]carbazole-1-carboxamide (0.7 g, 1.3 mmol, 1 eq, TFA) in DCM (100 mL) was added BBr₃ (1.6 g, 6.7 mmol, 650.3 uL, 5 eq) at 0° C. The mixture was stirred at 45° C. for 1 hr. LCMS showed the reaction was completed and main peak with desired product. The mixture was poured into NH₄OH (30 mL) and extracted with DCM (100 mL×3) at 0° C. The combined organic layers were washed with brine (30 mL×3), dried over Na₂SO₄, filtered and concentrated under reduced pressure to give (R)—N-(1-(dimethylamino)propan-2-yl)-9-hydroxy-5,6-dimethyl-6H-pyrido[4,3-b]carbazole-1-carboxamide.

### Step 2: (R)—N-(1-(dimethylamino)propan-2-yl)-5,6-dimethyl-9-(3-(4-methylpiperazin-1-yl)propoxy)-6H-pyrido[4,3-b]carbazole-1-carboxamide

To a solution of N-[(1R)-2-(dimethylamino)-1-methyl-ethyl]-9-hydroxy-5,6-dimethyl-pyrido [4,3-b]carbazole-1-carboxamide (340 mg, 870.7 umol, 1 eq) in toluene (10 mL) was added 3-(4-methylpiperazin-1-yl)propan-1-ol (151.5 mg, 957.8 umol, 1.1 eq) and 2-(tributyl-λ5-phosphanylidene)acetonitrile (420.3 mg, 1.7 mmol, 2 eq). The mixture was stirred at 100° C. for 12 hr. LCMS showed the reaction was completed and desired product was detected. The mixture was concentrated to get crude residue. The residue was purified by prep-HPLC (TFA condition, column: Phenomenex luna C18 100*40 mm*5 um; mobile phase: [water(0.1% TFA)-ACN]; B %: 10%-30%, 8 min) to give (R)—N-(1-(dimethylamino)propan-2-yl)-5,6-dimethyl-9-(3-(4-methylpiperazin-1-yl)propoxy)-6H-pyrido[4,3-b]carbazole-1-carboxamide (538 mg, contained other batch amount 200 mg, the product solid was dissolved with DMSO and further lyophilization).

MS (ESI): m/z=531.3 [M+H]⁺

### Example 27

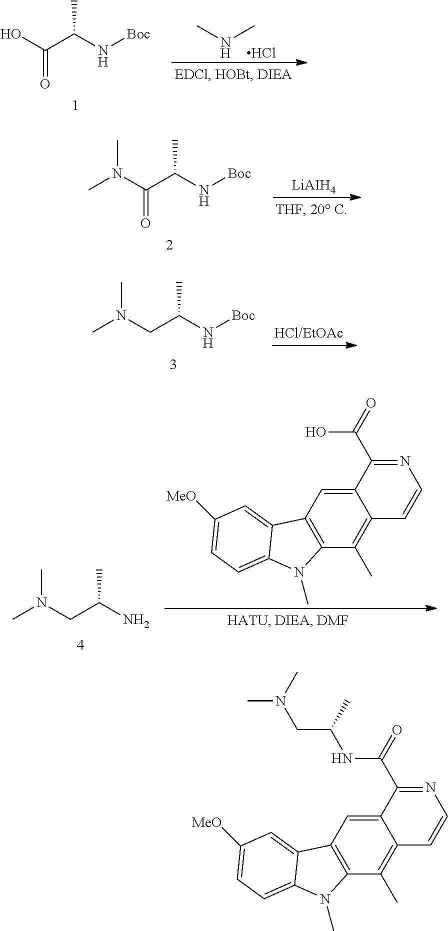

### Step 1: (S)-tert-butyl (1-(dimethylamino)-1-oxopropan-2-yl)carbamate

A mixture of (2S)-2-(tert-butoxycarbonylamino)propanoic acid (5 g, 26.4 mmol, 1 eq), N-methylmethanamine (2.1 g, 26.4 mmol, 2.4 mL, 1 eq, HCl), HOBt (5.3 g, 39.6 mmol, 1.5 eq), EDCI (7.6 g, 39.6 mmol, 1.5 eq) and DIEA (13.6 g, 105.7 mmol, 18.4 mL, 4 eq) in THF (50 mL) was degassed and purged with N₂ for 3 times, and then the mixture was stirred at 25° C. for 16 hr under N₂ atmosphere. TLC showed the reaction was completely. The reaction mixture was diluted with water (70 mL) and extracted with ethyl acetate (2×70 mL). The combined organic layers were washed with brine (70 mL), dried over anhydrous sodium sulfate, filtered and concentrated in vacuum. The residue was purified by flash silica gel chromatography (ISCO®; 20 g SepaFlash® Silica Flash Column, Eluent of 0˜10% Ethylacetate/Petroleum ether gradient @ 100 mL/min) to afford (S)-tert-butyl (1-(dimethylamino)-1-oxopropan-2-yl)carbamate 2.

### Step 2: (S)-tert-butyl (1-(dimethylamino)propan-2-yl)carbamate

To a solution of (S)-tert-butyl (1-(dimethylamino)-1-oxopropan-2-yl)carbamate (6.5 g, 30.3 mmol, 1 eq) in THF (200 mL) was added LiAlH₄ (2.3 g, 60.7 mmol, 2 eq) at 0° C. under N₂ atmosphere. The mixture was stirred at 20° C. for 2 hr. TLC showed the reaction was completely. The reaction was quenched with saturated MgSO₄ (2.3 mL) and 10 g MgSO4 solid was added. The mixture was filtrated and the cake was washed with ethyl acetate (50 mL×3). The combined filtrate was concentrated to give the crude product. The residue was purified by flash silica gel chromatography (ISCO®; 20 g SepaFlash® Silica Flash Column, Eluent of 0˜50% Ethyl acetate/Petroleum ether gradient @ 50 mL/min) to give (S)-tert-butyl (1-(dimethylamino)propan-2-yl)carbamate 3.

### Step 3: (S)—N1,N1-dimethylpropane-1,2-diamine

A solution of (S)-tert-butyl (1-(dimethylamino)propan-2-yl)carbamate (1.9 g, 9.3 mmol, 1 eq) in HCl/EtOAc (10 mL) (4M) was stirred at 15° C. for 2 hr. LCMS showed the reaction was completely. The reaction was concentrated to give the crude product (S)—N1,N1-dimethylpropane-1,2-diamine 4.

### Step 4: (S)—N-(1-(dimethylamino)propan-2-yl)-9-methoxy-5,6-dimethyl-6H-pyrido[4,3-b]carbazole-1-carboxamide

To a solution of 9-methoxy-5,6-dimethyl-6H-pyrido[4,3-b]carbazole-1-carboxylic acid (80 mg, 249.7 umol, 1 eq) in DMF (3 mL) was added DIEA (161.3 mg, 1.2 mmol, 217.5 uL, 5 eq) and (S)—N1,N1-dimethylpropane-1,2-diamine (131.1 mg, 749.2 umol, 3 eq, 2 HCl). The mixture was cooled to 0° C. and HATU (284.8 mg, 749.2 umol, 3 eq) was added. The mixture was stirred at 25° C. for 1 hr. LCMS showed the reaction was completely. The reaction was concentrated to give a residue. The residue was purified by prep-HPLC (FA condition, column: 3-Phenomenex Luna C18 75*30 mm*3 um; mobile phase: [water(0.2% FA)-ACN]; B %: 15%-45%, 9 min) to afford (S)—N-(1-(dimethylamino)propan-2-yl)-9-methoxy-5,6-dimethyl-6H-pyrido[4,3-b]carbazole-1-carboxamide.

MS (ESI): m/z=405.1 [M+H]⁺

### Example 28

### N-[2-(dimethylamino)ethyl]-9-methoxy-5,6-dimethyl-pyrido[4,3-b]carbazole-1-carboxamide

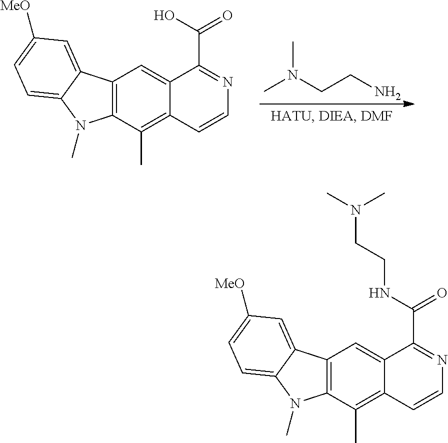

To a solution of 9-methoxy-5,6-dimethyl-pyrido[4,3-b]carbazole-1-carboxylic acid (50 mg, 156.0 umol, 1 eq) in DMF (2 mL) was added N,N-dimethylethane-1,2-diamine (43.1 mg, 488.9 umol, 53.4 uL, 3.1 eq), DIEA (100.8 mg, 780.4 umol, 135.9 uL, 5 eq). Then HATU (118.7 mg, 312.1 umol, 2 eq) was added to the mixture at 0° C. and warmed to 25° C. for 1 hr. LCMS showed the reaction was completed and desired product was detected. The mixture was concentrated to get crude residue. The residue was purified by prep-HPLC (neutral condition, column: Waters Xbridge BEH C18 100*30 mm*10 um; mobile phase: [water(10 mM NH4HCO3)-ACN]; B %: 35%-55%, 6 min) to give N-[2-(dimethylamino)ethyl]-9-methoxy-5,6-dimethyl-pyrido[4,3-b]carbazole-1-carboxamide.

MS (ESI): m/z=391.1 [M+H]⁺

### Example 29

### N-(3-(dimethylamino)propyl)-9-methoxy-5,6-dimethyl-6H-pyrido[4,3-b]carbazole-1-carboxamide

To a solution of 9-methoxy-5,6-dimethyl-6H-pyrido[4,3-b]carbazole-1-carboxylic acid (50 mg, 156.0 umol, 1 eq) in DMF (2 mL) was added DIEA (100.8 mg, 780.4 umol, 135.9 uL, 5 eq) and N,N-dimethylpropane-1,3-diamine (47.8 mg, 468.2 umol, 58.5 uL, 3 eq). The mixture was cooled to 0° C. and HATU (118.7 mg, 312.1 umol, 2 eq) was added. The mixture was stirred at 25° C. for 12 hr. LC-MS showed the desired compound was detected. The reaction mixture was concentrated under reduced pressure. The residue was purified by prep-HPLC (FA condition column: Phenomenex Luna C18 150*30 mm*5 um; mobile phase: [water(0.1% TFA)-ACN]; B %: 25%-55%, 8 min). The residue was purified by prep-HPLC (TFA condition column: Phenomenex Luna C18 150*30 mm*5 um; mobile phase: [water(0.1% TFA)-ACN]; B %: 25%-55%, 8 min) to give N-(3-(dimethylamino)propyl)-9-methoxy-5,6-dimethyl-6H-pyrido[4,3-b]carbazole-1-carboxamide.

MS (ESI): m/z=405.1 [M+H]⁺

### Example 30

### 5,6-dimethyl-9-(2-(4-methylpiperazin-1-yl)ethoxy)-6H-pyrido[4,3-b]carbazole

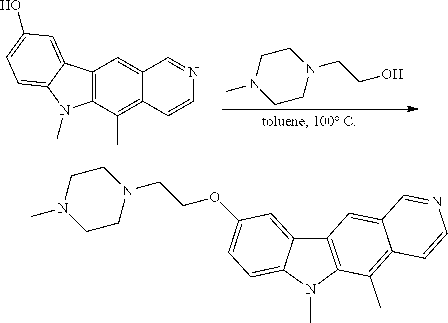

To a solution of 5,6-dimethyl-6H-pyrido[4,3-b]carbazol-9-ol (40 mg, 152.4 umol, 1 eq) in Tol. (2 mL) was added 2-(4-methylpiperazin-1-yl)ethanol (24.1 mg, 167.7 umol, 1.1 eq) and 2-(tributyl-λ⁵-phosphanylidene)acetonitrile (110.4 mg, 457.4 umol, 3 eq). The mixture was stirred at 100° C. for 2 hr under N₂ atmosphere. LC-MS showed the desired compound was detected. The reaction mixture was concentrated under reduced pressure to give a residue. The residue was purified by prep-HPLC (FA condition, column: 3-Phenomenex Luna C18 75*30 mm*3 um; mobile phase: [water(0.2% FA)-ACN]; B %: 5%-40%, 9 min) to give 5,6-dimethyl-9-(2-(4-methylpiperazin-1-yl)ethoxy)-6H-pyrido[4,3-b]carbazole.

MS (ESI): m/z=389.1 [M+H]⁺

### Example 31

### 5,6-dimethyl-9-(2-(piperidin-1-yl)ethoxy)-6H-pyrido[4,3-b]carbazole

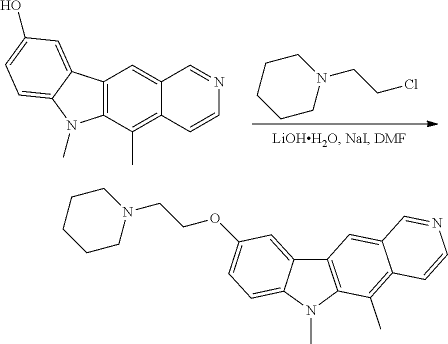

To a solution of 5,6-dimethyl-6H-pyrido[4,3-b]carbazol-9-ol (50 mg, 190.6 umol, 1 eq) in DMF (2 mL) was added LiOH·H₂O (8.0 mg, 190.6 umol, 1 eq), NaI (2.8 mg, 19.0 umol, 0.1 eq) and 1-(2-chloroethyl)piperidine; hydrochloride (35.0 mg, 190.6 umol, 1 eq). The mixture was stirred at 80° C. for 12 hr. LCMS showed 5,6-dimethyl-6H-pyrido[4,3-b]carbazol-9-ol has remained, 1-(2-chloroethyl)piperidine; hydrochloride (17.5 mg, 95.3 umol, 0.5 eq) and LiOH·H₂O (16.0 mg, 381.2 umol, 2 eq) were added to the mixture and stirred for 30 min. LC-MS showed the desired compound was detected. The reaction mixture was concentrated under reduced pressure. The residue was purified by prep-HPLC (FA condition, column: Phenomenex Luna C18 200*40 mm*10 um; mobile phase: [water(0.2% FA)-ACN]; B %: 1%-25%, 8 min) to give 5,6-dimethyl-9-(2-(piperidin-1-yl)ethoxy)-6H-pyrido[4,3-b]carbazole.

MS (ESI): m/z=374.1 [M+H]⁺

### Example 32

### 2-((5,6-dimethyl-6H-pyrido[4,3-b]carbazol-9-yl)oxy)ethanamine

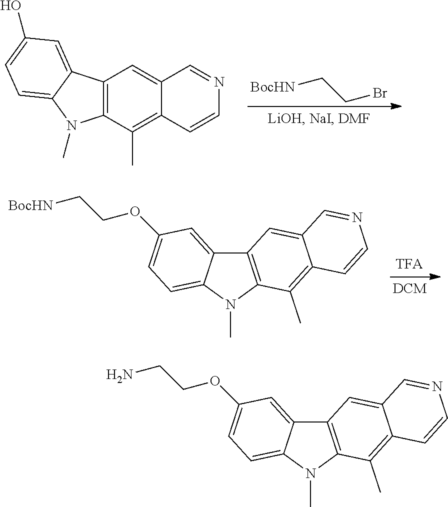

### Step 1: tert-butyl (2-((5,6-dimethyl-6H-pyrido[4,3-b]carbazol-9-yl)oxy)ethyl)carbamate

To a solution of 5,6-dimethyl-6H-pyrido[4,3-b]carbazol-9-ol (100 mg, 381.2 umol, 1 eq) in DMF (4 mL) was added LiOH·H₂O (16.0 mg, 381.2 umol, 1 eq), NaI (5.7 mg, 38.1 umol, 0.1 eq) and tert-butyl N-(2-bromoethyl)carbamate (85.4 mg, 381.2 umol, 1 eq). The mixture was stirred at 75° C. for 12 hr. LCMS showed the reaction was completely. The reaction was concentrated to give crude tert-butyl (2-((5,6-dimethyl-6H-pyrido[4,3-b]carbazol-9-yl)oxy)ethyl)carbamate.

### Step 2: 2-((5,6-dimethyl-6H-pyrido[4,3-b]carbazol-9-yl)oxy)ethanamine

To a solution of tert-butyl (2-((5,6-dimethyl-6H-pyrido[4,3-b]carbazol-9-yl)oxy)ethyl)carbamate (150 mg, 369.9 umol, 1 eq) in DCM (4 mL) was added TFA (2 mL). The mixture was stirred at 25° C. for 1 hr. LCMS showed the reaction was completely. The reaction was concentrated to give a residue. The residue was purified by prep-HPLC (TFA condition, column: Phenomenex Luna C18 150*30 mm*5 um; mobile phase: [water (0.1% TFA)-ACN]; B %: 1%-30%, 8 min). The residue was purified by prep-HPLC (TFA condition, column: Phenomenex Gemini-NX 150*30 mm*5 um; mobile phase: [water(0.1% TFA)-ACN]; B %: 20%) to afford 2-((5,6-dimethyl-6H-pyrido[4,3-b]carbazol-9-yl)oxy)ethanamine.

MS (ESI): m/z=306.0 [M+H]⁺

### Example 33

### 2-((5,6-dimethyl-6H-pyrido[4,3-b]carbazol-9-yl)oxy)-N,N-diethylethanamine

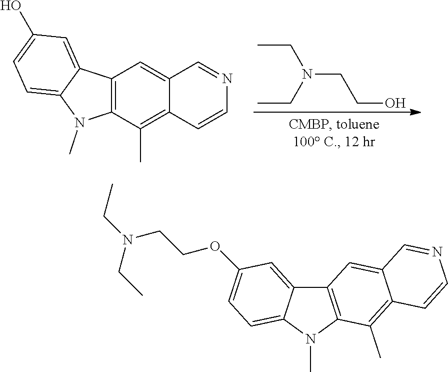

To a solution of 5,6-dimethylpyrido[4,3-b]carbazol-9-ol (50 mg, 190.6 umol, 1 eq), 2-(diethylamino)ethanol (26.8 mg, 228.7 umol, 30.3 uL, 1.2 eq) in toluene (3 mL) was added 2-(tributyl-λ5-phosphanylidene) acetonitrile (69 mg, 285.9 umol, 1.5 eq) under N₂. The mixture was stirred at 100° C. for 12 hrs under N₂. LC-MS showed was consumed and desired mass was detected. The reaction mixture was concentrated under reduced pressure to remove solvent. The residue was purified by prep-HPLC (FA condition; column: 3-Phenomenex Luna C18 75*30 mm*3 um; mobile phase: [water(0.2% FA)-ACN]; B %: 5%-40%, 9 min) to give 2-(5,6-dimethylpyrido[4,3-b]carbazol-9-yl)oxy-N,N-diethyl-ethanamine.

MS (ESI): m/z=362.1 [M+H]⁺

### Example 34

### 2-((5,6-dimethyl-6H-pyrido[4,3-b]carbazol-9-yl)oxy)-N-methylacetamide

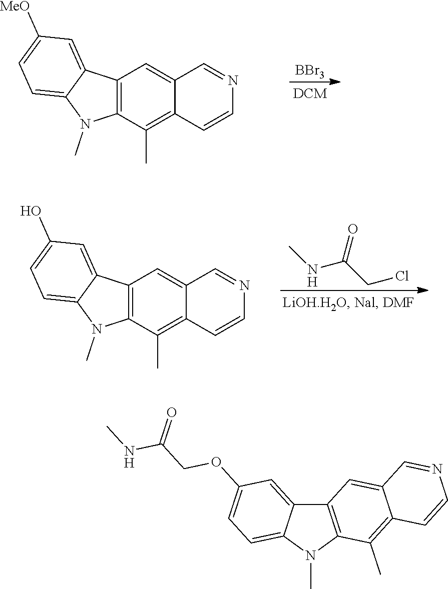

### Step 1: 5,6-dimethyl-6H-pyrido[4,3-b]carbazol-9-ol

To a solution of 9-methoxy-5,6-dimethyl-6H-pyrido[4,3-b]carbazole (50 mg, 180.9 umol, 1 eq) in DCM (2 mL) was added BBr₃ (226.6 mg, 904.7 umol, 87.1 uL, 5 eq) at 0° C. The mixture was stirred at 45° C. for 12 hrs. LCMS showed the reaction was completely. H₂O (2 mL) and NH₃·H₂O (2 mL) was added to the mixture. The mixture was extracted with DCM (2 mL×3). The combined organic phase was concentrated to give crude 5,6-dimethyl-6H-pyrido[4,3-b]carbazol-9-ol.

### Step 2: 2-((5,6-dimethyl-6H-pyrido[4,3-b]carbazol-9-yl)oxy)-N-methylacetamide

To a solution of 5,6-dimethylpyrido[4,3-b]carbazol-9-ol (44 mg, 167.7 umol, 1 eq) in DMF (2 mL) was added LiOH·H₂O (7.0 mg, 167.7 umol), NaI (2.5 mg, 16.7 umol, 0.1 eq) and 2-chloro-N-methyl-acetamide (18.0 mg, 167.7 umol, 1 eq). The mixture was stirred at 75° C. for 1 hr. LCMS showed the reaction was completely. The reaction was concentrated to give a residue. The residue was purified by prep-HPLC (FA condition, column: 3-Phenomenex Luna C18 75*30 mm*3 um; mobile phase: [water(0.2% FA)-ACN]; B %: 20%-50%, 9 min) to afford 2-((5,6-dimethyl-6H-pyrido[4,3-b]carbazol-9-yl)oxy)-N-methylacetamide.

MS (ESI): m/z=334.0 [M+H]⁺

### Example 35

### 5,6-dimethyl-9-((1-methyl-1H-imidazol-2-yl)methoxy)-6H-pyrido[4,3-b]carbazole

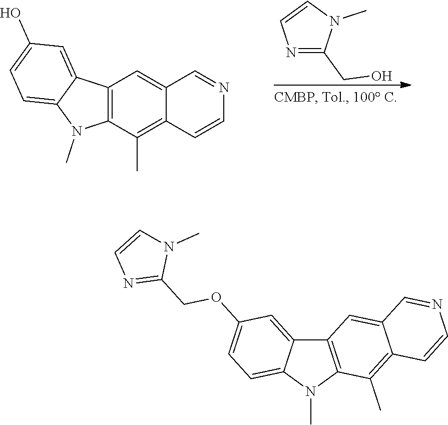

To a solution of 5,6-dimethyl-6H-pyrido[4,3-b]carbazol-9-ol (100 mg, 381.2 umol, 1 eq) in Tol. (2 mL) was added (1-methylimidazol-2-yl)methanol (42.7 mg, 381.2 umol, 1 eq) and 2-(tributyl-λ⁵-phosphanylidene)acetonitrile (184.0 mg, 762.4 umol, 2 eq). The mixture was stirred at 100° C. for 12 hr. LCMS showed 5,6-dimethyl-6H-pyrido[4,3-b]carbazol-9-ol was remained, so 2-(tributyl-λ⁵-phosphanylidene)acetonitrile (138.0 mg, 571.8 umol, 1.5 eq) was added to the mixture and stirred at 100° C. for 12 hr. LC-MS showed the desired compound was detected. The reaction mixture was concentrated under reduced pressure to give a residue. The residue was purified by prep-HPLC (TFA condition, column: Phenomenex Gemini-NX 150*30 mm*5 um; mobile phase: [water(0.1% TFA)-ACN]; B %: 20%-50%, 9 min) to give 5,6-dimethyl-9-((1-methyl-1H-imidazol-2-yl)methoxy)-6H-pyrido[4,3-b]carbazole.

MS (ESI): m/z=357.1 [M+H]⁺

### Example 36

### (R)-5,6-dimethyl-9-((1-methylpyrrolidin-2-yl)methoxy)-6H-pyrido[4,3-b]carbazole

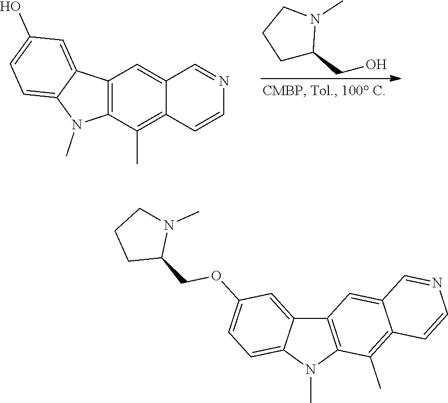

To a solution of 5,6-dimethyl-6H-pyrido[4,3-b]carbazol-9-ol (50 mg, 190.6 umol, 1 eq) in Tol. (2 mL) was added [(2R)-1-methylpyrrolidin-2-yl]methanol (21.9 mg, 190.6 umol, 22.6 uL, 1 eq) and 2-(tributyl-λ⁵-phosphanylidene)acetonitrile (92.0 mg, 381.2 umol, 2 eq). The mixture was stirred at 100° C. for 12 hr. LC-MS showed the desired compound was detected. The reaction mixture was concentrated under reduced pressure to give a residue. The residue was purified by prep-HPLC (TFA condition, column: Phenomenex Gemini-NX 150*30 mm*5 um; mobile phase: [water(0.1% TFA)-ACN]; B %: 20%-50%, 9 min) to give (R)-5,6-dimethyl-9-((1-methylpyrrolidin-2-yl)methoxy)-6H-pyrido[4,3-b]carbazole.

MS (ESI): m/z=360.1 [M+H]⁺

### Example 37

### (S)-5,6-dimethyl-9-((1-methylpyrrolidin-2-yl)methoxy)-6H-pyrido[4,3-b]carbazole

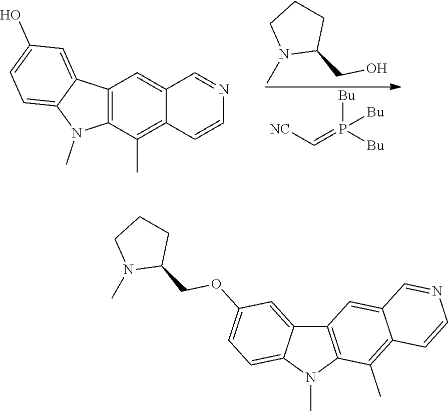

To a solution of 5,6-dimethyl-6H-pyrido[4,3-b]carbazol-9-ol (50 mg, 190.6 umol, 1 eq) in toluene (2 mL) was added (S)-(1-methylpyrrolidin-2-yl)methanol (21.9 mg, 190.6 umol, 22.6 uL, 1 eq) and 2-(tributylphosphoranylidene)acetonitrile (92.0 mg, 381.2 umol, 2 eq). The mixture was stirred at 100° C. for 12 hr. LCMS showed the reaction was completely. The reaction was concentrated to give a residue. The residue was purified by prep-HPLC (TFA condition, column: Phenomenex Synergi C18 150*25*10 um; mobile phase: [water(0.1% TFA)-ACN]; B %: 10%-35%, 8 min) to afford (S)-5,6-dimethyl-9-((1-methylpyrrolidin-2-yl)methoxy)-6H-pyrido[4,3-b]carbazole.

MS (ESI): m/z=360.1 [M+H]⁺

### Example 38

### (R)-5,6-dimethyl-9-(pyrrolidin-2-ylmethoxy)-6H-pyrido[4,3-b]carbazole

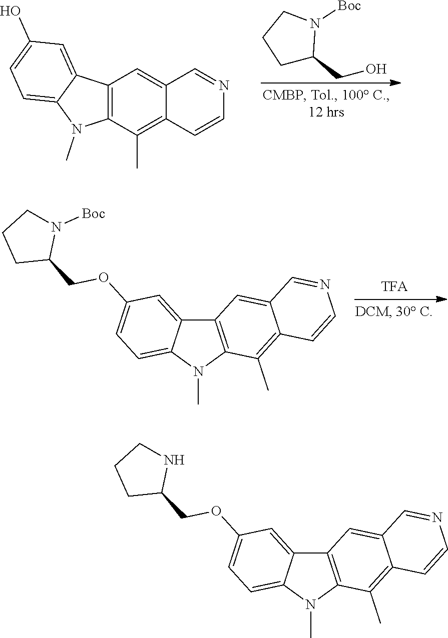 [SMILES: Cc1c2ccncc2cc2c3cc(O)ccc3n(C)c12 ; CC(C)(C)OC(=O)N1CCC[C@@H]1CO ; Cc1c2ccncc2cc2c3cc(OC[C@H]4CCCN4C(=O)OC(C)(C)C)ccc3n(C)c12 ; Cc1c2ccncc2cc2c3cc(OC[C@H]4CCCN4)ccc3n(C)c12]

### Step 1: (R)-tert-butyl 2-(((5,6-dimethyl-6H-pyrido[4,3-b]carbazol-9-yl)oxy)methyl)-pyrrolidine-1-carboxylate

To a solution of 5,6-dimethyl-6H-pyrido[4,3-b]carbazol-9-ol (100 mg, 381.2 umol, 1 eq) in Tol. (7 mL) was added tert-butyl (2R)-2-(hydroxymethyl)pyrrolidine-1-carboxylate (76.7 mg, 381.2 umol, 1 eq) and 2-(tributyl-λ5-phosphanylidene)acetonitrile (184.0 mg, 762.4 umol, 2 eq). The mixture was degassed and purged with N₂ for 3 times, and then the mixture was stirred at 100° C. for 12 hrs under N₂ atmosphere. LC-MS showed the desired compound was detected. The reaction mixture was concentrated under reduced pressure to give a residue. The residue was purified by prep-TLC (SiO₂, Petroleum ether/Ethyl acetate=0/1) to give (R)-tert-butyl 2-(((5,6-dimethyl-6H-pyrido[4,3-b]carbazol-9-yl)oxy)methyl)pyrrolidine-1-carboxylate.

### Step 2: (R)-5,6-dimethyl-9-(pyrrolidin-2-ylmethoxy)-6H-pyrido[4,3-b]carbazole

To a solution of (R)-tert-butyl 2-(((5,6-dimethyl-6H-pyrido[4,3-b]carbazol-9-yl)oxy)methyl)pyrrolidine-1-carboxylate (150 mg, 336.6 umol, 1 eq) in DCM (3 mL) was added TFA (767.7 mg, 6.7 mmol, 498.5 uL, 20 eq). The mixture was stirred at 30° C. 1 hr. LC-MS showed most desired compound was detected. The reaction mixture was concentrated under reduced pressure to give a residue. The residue was purified by prep-HPLC (TFA condition; column: Phenomenex luna C18 100*40 mm*5 um; mobile phase: [water(0.1% TFA)-ACN]; B %: 1%-30%, 8 min) to give (R)-5,6-dimethyl-9-(pyrrolidin-2-ylmethoxy)-6H-pyrido[4,3-b]carbazole.

MS (ESI): m/z=346.1 [M+H]⁺

### Example 39

### (S)-5,6-dimethyl-9-(pyrrolidin-2-ylmethoxy)-6H-pyrido[4,3-b]carbazole

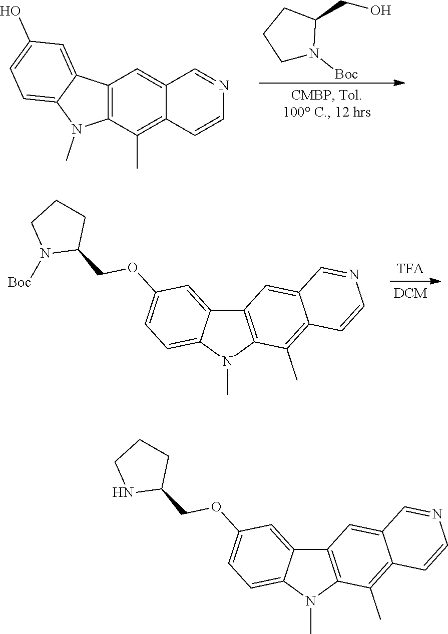

### Step 1: (S)-tert-butyl 2-(((5,6-dimethyl-6H-pyrido[4,3-b]carbazol-9-yl)oxy)methyl)-pyrrolidine-1-carboxylate

To a solution of 5,6-dimethylpyrido[4,3-b]carbazol-9-ol (100 mg, 380.8 umol, 1 eq) in Tol. (0.5 mL) was added tert-butyl (2S)-2-(hydroxymethyl)pyrrolidine-1-carboxylate (84.3 mg, 418.9 umol, 1.1 eq) and 2-(tributyl-λ5-phosphanylidene)acetonitrile (110.3 mg, 457.0 umol, 1.2 eq). The mixture was stirred at 100° C. for 12 hr. LCMS showed the reaction was completed and desired product was detected. The reaction mixture was concentrated under reduced pressure to remove solvent. The residue was purified by prep-TLC (SiO2, DCM:MeOH=10:1) to give (S)-tert-butyl 2-(((5,6-dimethyl-6H-pyrido[4,3-b]carbazol-9-yl)oxy)methyl)pyrrolidine-1-carboxylate.

### Step 2: (S)-5,6-dimethyl-9-(pyrrolidin-2-ylmethoxy)-6H-pyrido[4,3-b]carbazole

To a solution of tert-butyl (2S)-2-[(5,6-dimethylpyrido[4,3-b]carbazol-9-yl)oxymethyl]pyrrolidine-1-carboxylate (190 mg, 426.4 umol, 1 eq) in DCM (2 mL) was added TFA (770.0 mg, 6.7 mmol, 0.5 mL, 15.8 eq). The mixture was stirred at 20° C. for 2 hr. LCMS showed the reaction was completed and desired product was detected. The reaction mixture was concentrated under reduced pressure to remove solvent. The residue was purified by prep-HPLC (TFA condition, column: Phenomenex Gemini-NX C18 75*30 mm*3 um; mobile phase: [water(0.1% TFA)-ACN]; B %: 5%-45%, 8 min) to give (S)-5,6-dimethyl-9-(pyrrolidin-2-ylmethoxy)-6H-pyrido[4,3-b]carbazole.

MS (ESI): m/z=346.1 [M+H]⁺

### Example 40

### 9-(2-(4,4-difluoropiperidin-1-yl)ethoxy)-5,6-dimethyl-6H-pyrido[4,3-b]carbazole

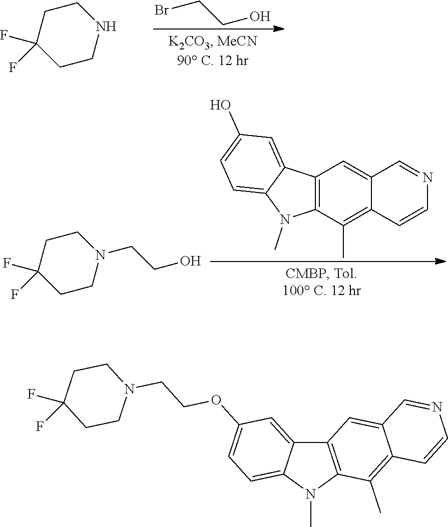

### Step 1: 2-(4,4-difluoropiperidin-1-yl)ethanol

To a solution of 4,4-difluoropiperidine (500 mg, 4.1 mmol, 1 eq), 2-bromoethanol (491.2 mg, 3.9 mmol, 279.1 uL, 1 eq) and K₂CO₃ (461.8 mg, 3.3 mmol, 0.8 eq) in MeCN (20 mL) was heated at 90° C. for 12 hrs. The reaction mixture was concentrated under reduced pressure to remove solvent. The residue was taken in DCM 30 mL, filtered off, and the filtrate concentrated to give 2-(4,4-difluoro-1-piperidyl)ethanol.

### Step 2: 9-(2-(4,4-difluoropiperidin-1-yl)ethoxy)-5,6-dimethyl-6H-pyrido[4,3-b]carbazole

A mixture of 5,6-dimethylpyrido[4,3-b]carbazol-9-ol (100 mg, 381.2 umol, 1 eq), 2-(4,4-difluoro-1-piperidyl)ethanol (75.5 mg, 457.4 umol, 1.2 eq) and 2-(tributyl-λ5-phosphanylidene) acetonitrile (184 mg, 762.4 umol, 2 eq) in toluene (5 mL) was degassed and purged with N₂ for 3 times, and then the mixture was stirred at 100° C. for 12 hrs under N₂ atmosphere. LC-MS showed desired compound was detected. The reaction mixture was concentrated under reduced pressure to remove solvent. The residue was purified by prep-HPLC (FA condition; 3-Phenomenex Luna C18 75*30 mm*3 um; mobile phase: [water(0.2% FA)-ACN]; B %: 5%-50%, 9 min) to give 9-[2-(4,4-difluoro-1-piperidyl)ethoxy]-5,6-dimethyl-pyrido[4,3-b]carbazole.

MS (ESI): m/z=410.1 [M+H]⁺

### Example 41

### 5,6-dimethyl-9-((1-methyl-4,5-dihydro-1H-imidazol-2-yl)methoxy)-6H-pyrido[4,3-b]carbazole

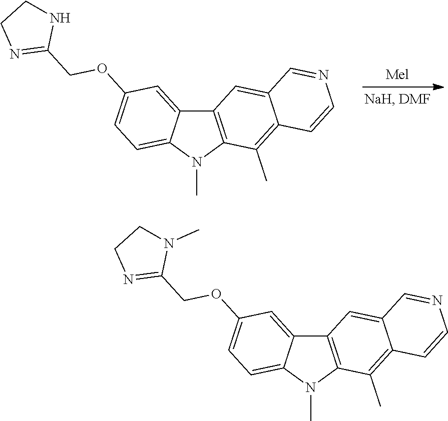

To a solution of 9-(4,5-dihydro-1H-imidazol-2-ylmethoxy)-5,6-dimethyl-pyrido[4,3-b] carbazole (50 mg, 109.0 umol, 1 eq, TFA) in DMF (2 mL) was added NaH (13.0 mg, 327.2 umol, 60% purity, 3 eq) at 0° C. The mixture was stirred at 0° C. for 0.5 hr. A solution of Mel (15.4 mg, 109.0 umol, 6.7 uL, 1 eq) in DMF (0.5 mL) was added. The mixture was stirred at 25° C. for 12 hr. LCMS showed the reaction was completely. The reaction was concentrated to give residue. The residue was purified by prep-HPLC (TFA condition, column: Phenomenex luna C18 100*40 mm*5 um; mobile phase: [water(0.1% TFA)-ACN]; B %: 18%-38%, 8 min) to afford 5,6-dimethyl-9-((1-methyl-4,5-dihydro-1H-imidazol-2-yl)methoxy)-6H-pyrido[4,3-b]carbazole.

MS (ESI): m/z=359.1 [M+H]⁺

### Example 42

### 2-((5,6-dimethyl-6H-pyrido[4,3-b]carbazol-9-yl)oxy)-N-(2-morpholinoethyl)acetamide

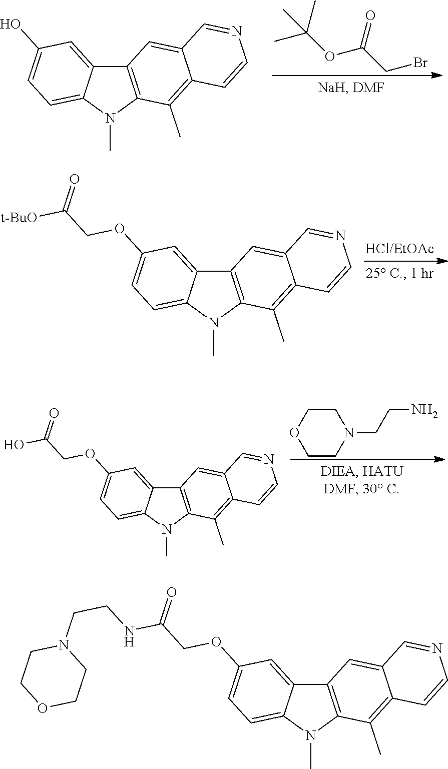

### Step 1: tert-butyl 2-((5,6-dimethyl-6H-pyrido[4,3-b]carbazol-9-yl)oxy)acetate

To a solution of 5,6-dimethyl-6H-pyrido[4,3-b]carbazol-9-ol (200 mg, 762.4 umol, 1 eq) in THF (5 mL) was added NaH (45.7 mg, 1.1 mmol, 60% purity, 1.5 eq) at 0° C. The mixture was stirred for 0.5 hr. Tert-butyl 2-bromoacetate (178.4 mg, 914.9 umol, 135.2 uL, 1.2 eq) was added and the mixture was stirred at 25° C. for 12 hr. LCMS showed some desired product was formed. The reaction was quenched with saturated NH₄Cl (10 mL) and extracted with ethyl acetate (10 mL×3). The combined organic phase was washed with brine (10 mL×3), dried over anhydrous Na₂SO₄, filtered and concentrated to give a residue. The residue was purified by prep-TLC (SiO₂, Petroleum ether/Ethyl acetate=0/1) to give tert-butyl 2-((5,6-dimethyl-6H-pyrido[4,3-b]carbazol-9-yl)oxy)acetate.

### Step 2: 2-((5,6-dimethyl-6H-pyrido[4,3-b]carbazol-9-yl)oxy)acetic acid

A solution of tert-butyl 2-((5,6-dimethyl-6H-pyrido[4,3-b]carbazol-9-yl)oxy)acetate (75 mg, 199.2 umol, 1 eq) in HCl/EtOAc (2 mL) (4M) was stirred at 25° C. for 1 hr. LCMS showed the reaction was completely. The reaction was concentrated to give the crude product of 2-((5,6-dimethyl-6H-pyrido[4,3-b]carbazol-9-yl)oxy)acetic acid.

### Step 3: 2-((5,6-dimethyl-6H-pyrido[4,3-b]carbazol-9-yl)oxy)-N-(2-morpholinoethyl)-acetamide

To a solution of 5,6-dimethyl-6H-pyrido[4,3-b]carbazol-9-ol (30 mg, 84.0 umol, 1 eq, HCl) in DMF (2 mL) was added DIEA (27.1 mg, 210.2 umol, 36.6 uL, 2.5 eq), HATU (47.9 mg, 126.1 umol, 1.5 eq) and 2-morpholinoethanamine (12.0 mg, 92.4 umol, 12.1 uL, 1.1 eq). The mixture was stirred at 30° C. for 12 hr. LCMS showed some desired product was formed. The reaction was concentrated to give a residue. The residue was purified by prep-HPLC (TFA condition, column: Phenomenex Gemini-NX C18 75*30 mm*3 um; mobile phase: [water(0.1% TFA)-ACN]; B %: 10%-45%, 8 min) to afford 2-((5,6-dimethyl-6H-pyrido[4,3-b]carbazol-9-yl)oxy)-N-(2-morpholinoethyl)acetamide.

MS (ESI): m/z=433.1 [M+H]⁺

### Example 43

### 9-((4,5-dihydro-1H-imidazol-2-yl)methoxy)-5-methyl-6-(3-(4-methylpiperazin-1-yl)propyl)-6H-pyrido[4,3-b]carbazole

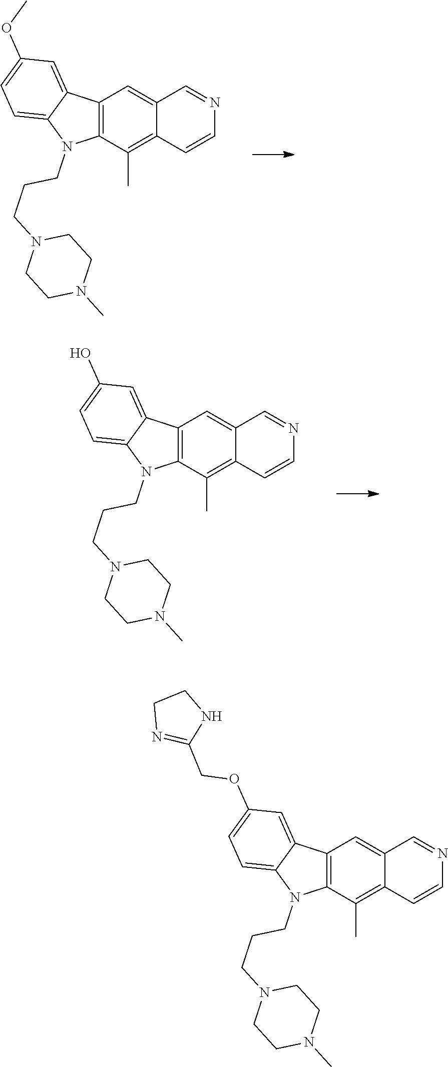

### Step 1: 9-hydroxy-5-methyl-6-(3-(4-methylpiperazin-1-yl)propyl)-6H-pyrido[4,3-b]carbazole

To a solution of 9-methoxy-5-methyl-6-(3-(4-methylpiperazin-1-yl)propyl)-6H-pyrido[4,3-b]carbazole (20 mg, 0.05 mmol) in DCM (0.5 ml) was added BBr₃ (23 uL, 0.25 mmol) and the mixture was stirred at rt overnight, then added NH₄OH aq and DCM/MeOH (10:1). The org phase was washed with brine, dried over Na₂SO₄ and concentrated in vacuo. The residue was purified with HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm) to afford the title compound.

### Step 2: 9-((4,5-dihydro-1H-imidazol-2-yl)methoxy)-5-methyl-6-(3-(4-methylpiperazin-1-yl)propyl)-6H-pyrido[4,3-b]carbazole

To a solution of the product of step 1 (14 mg, 0.036 mmol) in THF (1 ml) was added NaI (5 mg, 0.036 mmol), KOt-Bu (8 mg, 0.072 mmol) and alkyl chloride (10 mg, 0.072 mmol) and the mixture was heated at 70° C. for 6 h, then purified with HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=01% TFA in H2O and B=MeOH or ACN: Flow rate: 5 mL/min, Injection volume: 4 mL, Runtime: 60 min, gradient: variable concentrations of B in A; UV detection at 220 & 345 nm) to afford the title compound.

MS (ESI): 471.3 [M+H]⁺

### Example 44

### 4-((5,6-dimethyl-6H-pyrido[4,3-b]carbazol-9-yl)oxy)butanimidamide (44a) & methyl 4-((5,6-dimethyl-6H-pyrido[4,3-b]carbazol-9-yl)oxy)butanoate (44b)

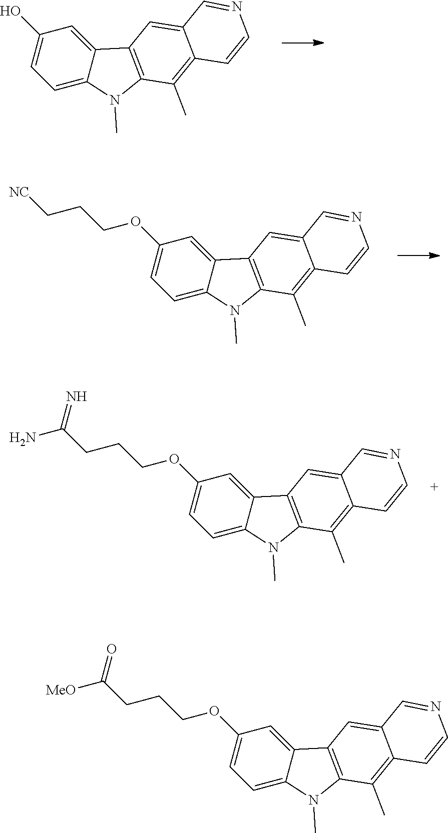 [SMILES: Cc1c2ccncc2cc2c3cc(O)ccc3n(C)c12 ; Cc1c2ccncc2cc2c3cc(OCCCC#N)ccc3n(C)c12 ; Cc1c2ccncc2cc2c3cc(OCCCC(=N)N)ccc3n(C)c12 ; COC(=O)CCCOc1ccc2c(c1)c1cc3cnccc3c(C)c1n2C]

### Step 1: 4-((5,6-dimethyl-6H-pyrido[4,3-b]carbazol-9-yl)oxy)butyronitrile

To a solution of 5,6-dimethyl-6H-pyrido[4,3-b]carbazol-9-ol (26 mg, 0.10 mmol) in THF was added NaI (15 mg, 0.1 mmol), KOtBu (17 mg, 0.15 mmol) and chlorobutyronitrile (31 mg, 0.3 mmol) and the mixture was stirred at 70° C. overnight and then purified with HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm) to afford the title compound.

### Step 2: 4-((5,6-dimethyl-6H-pyrido[4,3-b]carbazol-9-yl)oxy)butanimidamide (44a) & methyl 4-((5,6-dimethyl-6H-pyrido[4,3-b]carbazol-9-yl)oxy)butanoate (44b)

To a solution of the product of Step 1 (21 mg, 0.64 mmol) in MeOH was added AcCl (0.2 ml) and the mixture was stirred for 6 h at rt, then the volatile was removed and then NH4OH aq (1 ml) was added and stirred at rt overnight. The resulting mixture was purified with HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm) to afford compound 44a and compound 44b.

44a: MS (ESI): 347.2 [M+H]⁺; 44b: MS (ESI): 363.2 [M+H]⁺

### Example 45

### 2-((5,6-dimethyl-6H-pyrido[4,3-b]carbazol-9-yl)oxy)acetimidamide

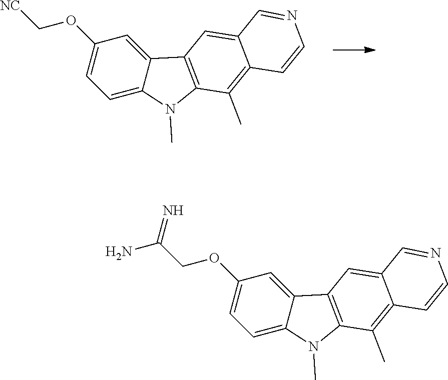 [SMILES: Cc1c2ccncc2cc2c3cc(OCC#N)ccc3n(C)c12 ; Cc1c2ccncc2cc2c3cc(OCC(=N)N)ccc3n(C)c12]

To a solution of 2-((5,6-dimethyl-6H-pyrido[4,3-b]carbazol-9-yl)oxy)acetonitrile (13 mg, 0.043 mmol) in MeOH was added AcCl (0.2 ml) at rt overnight, then the volatile was removed. The residue was added NH4OH aq (1 ml) and the mixture was stirred for 3 h at rt and purified with HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm) to afford the title compound.

MS (ESI): 319.2 [M+H]⁺

### Example 46

### 4-[2-(6-methylpyrido[4,3-b]carbazol-9-yl)oxyethyl]morpholine

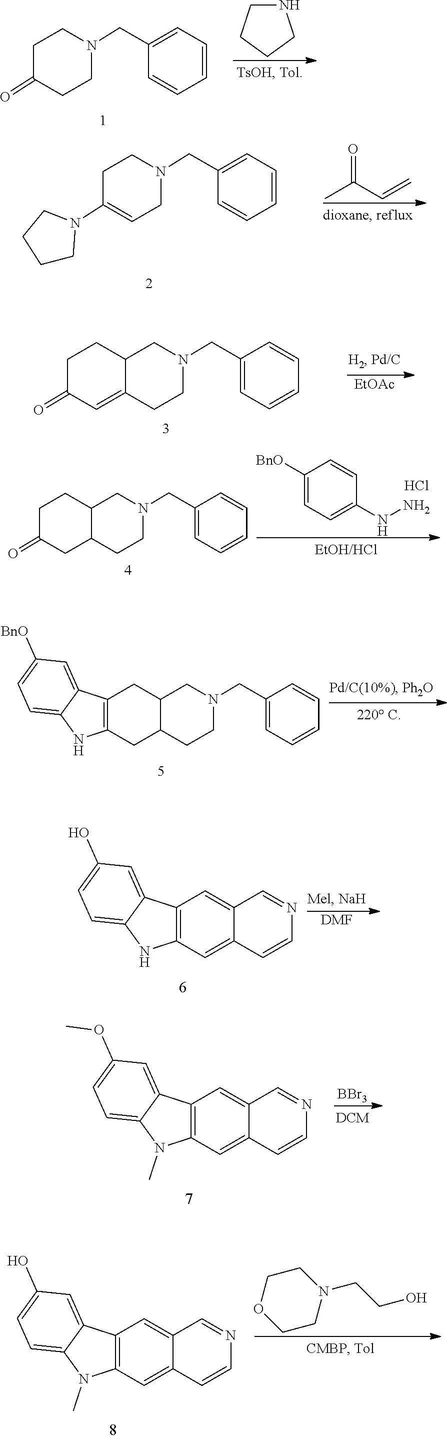

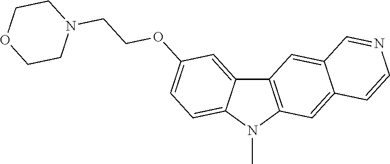 [SMILES: Cn1c2ccc(OCCN3CCOCC3)cc2c2cc3cnccc3cc21]

### Step 1: 1-benzyl-4-pyrrolidin-1-yl-3,6-dihydro-2H-pyridine

A mixture of 1-benzylpiperidin-4-one (50 g, 264.20 mmol, 49.02 mL, 1 eq), pyrrolidine (56.3 g, 792.5 mmol, 66.1 mL, 3 eq), 4-methylbenzenesulfonic acid (4.5 g, 26.4 mmol, 0.1 eq), in 100 mL of toluene was stirred at 110° C. for 1 hr. TLC showed the reaction was completed. The reaction mixture was filtered and concentrated under reduced pressure to give 1-benzyl-4-pyrrolidin-1-yl-3,6-dihydro-2H-pyridine.

### Step 2: 2-benzyl-1,3,4,7,8,8a-hexahydroisoquinolin-6(2H)-one

To a solution of 1-benzyl-4-pyrrolidin-1-yl-3,6-dihydro-2H-pyridine (40 g, 165.04 mmol, 1 eq) in dioxane (300.0 mL) was added but-3-en-2-one (12.7 g, 181.5 mmol, 15.1 mL, 1.1 eq). The mixture was stirred at 100° C. for 12 hr. LCMS showed the reaction was completed. The reaction mixture was quenched by addition 60 mL HCl (10%), and then diluted with 500 mL of H₂O and extracted with 1000 mL of ethyl acetate. The combined organic layers were washed with 500 mL of brine, dried over Na₂SO₄, filtered and concentrated under reduced pressure to give a residue. The residue was purified by column chromatography (SiO₂, Petroleum ether/Ethyl acetate=1/0 to 0/1) to give 2-benzyl-1,3,4,7,8,8a-hexahydroisoquinolin-6(2H)-one.

### Step 3: 2-benzyl-1,3,4,4a, 5,7,8,8a-octahydroisoquinolin-6-one

To a solution of 2-benzyl-1,3,4,7,8,8a-hexahydroisoquinolin-6-one (9 g, 37.2 mmol, 1 eq) in 50 mL of ethyl acetate was added Pd/C (1 g, 10% purity) under N₂ atmosphere. The suspension was degassed and purged with H₂ for 3 times. The mixture was stirred under H₂ (15 Psi) at 20° C. for 0.5 hr. LCMS showed the 2-benzyl-1,3,4,7,8,8a-hexahydroisoquinolin-6-one remained, and then the mixture was stirred at 20° C. for another 2 hr. LCMS showed the reaction was completed. The reaction mixture was filtered and concentrated under reduced pressure to give 2-benzyl-1,3,4,4a, 5,7,8,8a-octahydroisoquinolin-6-one.

### Step 4: 2-benzyl-9-benzyloxy-1,3,4,4a, 5,6,11,11a-octahydropyrido[4,3-b]carbazole

A mixture of 2-benzyl-1,3,4,4a, 5,7,8,8a-octahydroisoquinolin-6-one (18 g, 73.9 mmol, 1 eq), (4-benzyloxyphenyl)hydrazine; hydrochloride (21.2 g, 73.9 mmol, 1 eq, HCl), HCl (12 M, 4.00 mL, 6.49e-1 eq), in 200 mL of EtOH was degassed and purged with N₂ for 3 times, and then the mixture was stirred at 20° C. for 12 hr under N₂ atmosphere. LCMS showed the reaction was completed. The reaction mixture was quenched by addition 500 mL of sat·NaHCO₃, and extracted with 1000 mL of ethyl acetate. The combined organic layers were washed with 500 mL of brine, dried over Na₂SO₄, filtered and concentrated under reduced pressure to give a residue. The residue was purified by prep-HPLC (basic condition, column: Agela DuraShell C18 250*70 mm*10 um; mobile phase: [water(0.05% NH₃H₂O+10mMNH₄HCO₃)-ACN]; B %: 70%-95%, 20 min) to give 2-bezyl-9-benzyloxy-1,3,4,4a, 5,6,11,11a-octahydropyrido[4,3-b]carbazole.

### Step 5: 6H-pyrido[4,3-b]carbazol-9-ol

A mixture of 2-benzyl-9-benzyloxy-1,3,4,4a, 5,6,11,11a-octahydropyrido[4,3-b]carbazole (600 mg, 1.4 mmol, 1 eq) and Pd/C (300 mg, 10% purity) in 10 mL of diphenyl ether, and then the mixture was stirred at 250° C. for 30 min. LCMS showed the reaction was completed. The reaction mixture was filtered and concentrated under reduced pressure to give 6H-pyrido[4,3-b]carbazol-9-ol.

### Step 6: 9-methoxy-6-methyl-pyrido[4,3-b]carbazole

To a solution of 6H-pyrido[4,3-b]carbazol-9-ol (80 mg, 341.51 μmol, 1 eq) in 1 mL of DMF was added NaH (27.32 mg, 683.02 μmol, 60% purity, 2 eq) at 0° C. The mixture was stirred a 0° C. for 0.5 hr, then Mel (96.9 mg, 683.0 μmol, 2 eq) in 0.1 mL of DMF was added to above solution at 0° C., then the mixture was stirred 20° C. for 0.5 hr. LCMS showed the reaction was completed. The reaction mixture was quenched by addition 30 mL of water at 0° C., extracted with 200 mL of ethyl acetate. The combined organic layers were washed with 50 mL of brine, dried over Na₂SO₄, filtered and concentrated under reduced pressure to give a residue. The residue was purified by prep-TLC (SiO₂, eluting with Ethyl acetate:Methanol=10:1) to give 9-methoxy-6-methyl-pyrido[4,3-b]carbazole.

### Step 7: 6-methylpyrido[4,3-b]carbazol-9-ol

To a solution of 9-methoxy-6-methyl-pyrido[4,3-b]carbazole (70 mg, 266.86 μmol, 1 eq) in 3 mL of DCM was added BBr₃ (133.71 mg, 533.73 μmol, 2 eq) at 0° C. The mixture was stirred at 0° C. for 1 hr. LCMS showed the reaction was completed. The reaction mixture was poured into 10 mL of sat·NaHCO₃ at 0° C., and then diluted with 30 mL of H₂O and extracted with 100 mL of ethyl acetate. The combined organic layers were washed with 200 mL of brine, dried over Na₂SO₄, filtered and concentrated under reduced pressure to give 6-methylpyrido[4,3-b]carbazol-9-ol.

### Step 8: 4-[2-(6-methylpyrido[4,3-b]carbazol-9-yl)oxyethyl]morpholine

A mixture of 2-morpholinoethanol (39.6 mg, 302.0 μmol, 1.5 eq), 6-methylpyrido [4,3-b]carbazol-9-ol (50 mg, 201.3 μmol, 1 eq), 2-(tributyl-λ⁵-phosphanylidene)acetonitrile (72.9 mg, 302.0 μmol, 1.5 eq), in 3 mL of toluene and then the mixture was stirred at 100° C. for 12 hr. LCMS showed the reaction was completed. The reaction mixture was filtered and concentrated under reduced pressure to give a residue. The residue was purified by prep-HPLC (FA condition, column: Phenomenex Luna C18 200*40 mm*10 um; mobile phase: [water(0.2% FA)-ACN]; B %: 1%-45%, 8 min) to give 4-[2-(6-methylpyrido[4,3-b carbazol-9-yl)oxyethyl]morpholine.

MS (ESI): m/z=362.1 [M+H]⁺

### Example 47

### 9-methoxy-5H-pyrido[4,3-b]carbazole-5,11(6H)-dione

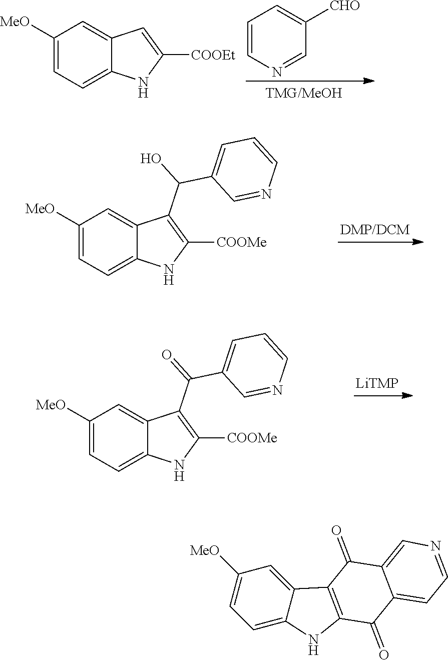 [SMILES: CCOC(=O)c1cc2cc(OC)ccc2[nH]1 ; O=Cc1cccnc1 ; COC(=O)c1[nH]c2ccc(OC)cc2c1C(O)c1cccnc1 ; COC(=O)c1[nH]c2ccc(OC)cc2c1C(=O)c1cccnc1 ; COc1ccc2[nH]c3c(c2c1)C(=O)c1cnccc1C3=O]

### Step 1: methyl 3-(hydroxy(pyridin-3-yl)methyl)-5-methoxy-1H-indole-2-carboxylate

To a solution of ethyl 5-methoxyindolecarboxylate (2.2 g, 10.0 mmol) in DCM (50 ml) was added AICI3 (2.66 g, 20 mmol) and stirred at rt for 30 min and cooled to 0° C., added 3-pyridinecarbaldehyde (2.1 g, 20 mmol). The mixture was slowly warmed to rt and stirred overnight at rt (observed a big chunk). The mixture was quenched with water and the aq phase was extracted with EtOAc (2 times). The combined org phase was washed with brine and dried over Na2SO4 and concentrated in vacuo. The residue was purified with column chromatography (Hexanes: EtOAc=0 to 100%) to afford the title compound methyl 3-(hydroxy(pyridin-3-yl)methyl)-5-methoxy-1H-indole-2-carboxylate which was used in next step.

### Step 2: methyl 5-methoxy-3-nicotinoyl-1H-indole-2-carboxylate

To a solution of methyl 3-(hydroxy(pyridin-3-yl)methyl)-5-methoxy-1H-indole-2-carboxylate in DCM (67 ml) was added DMP (2.8 g, 6.7 mmol) at 0° C. and warmed to rt. The mixture was stirred for 1 h and added NaHCO₃ aq and Na2S2O5, EtOAc. The org phase was washed with brine, dried over Na2SO4 and concentrated in vacuo. The residue was purified with column chromatography (Hexanes: EtOAc=0 to 100%) to afford the desired product.

### Step 3: 9-methoxy-5H-pyrido[4,3-b]carbazole-5,11(6H)-dione

To a solution of tetramethylpiperidine (7.2 ml, 45.0 mmol) in THF (15 ml) was added n-BuLi (2.5M, 18 ml, 40 mmol) at 0° C. and stirred for 15 min. The mixture was cooled to −78° C. and added 5-methoxy-3-nicotinoyl-1H-indole-2-carboxylate (3.0 g, 9.2 mmol) in 15 ml of THF and stirred for 3 h, then quenched with water and added 200 ml of hexanes and 200 ml of sat NaHCO₃ aq. The suspension was filtered over disposable 10 Micron Polyethylene Frit (required 8 of them since it get clogged) and washed with water and EtOAc to remove impurity. The residue was dried under high vac overnight to provide the title compound

### Example 48

### 9-methoxy-5-methyl-6H-pyrido[4,3-b]carbazol-11-ol

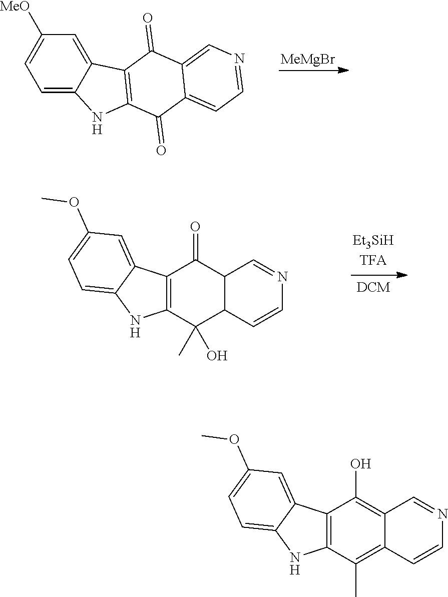 [SMILES: "MeMgBr" ; COc1ccc2[nH]c3c(c2c1)C(=O)c1cnccc1C3=O ; COc1ccc2[nH]c3c(c2c1)C(=O)C1C=NC=CC1C3(C)O ; COc1ccc2[nH]c3c(C)c4ccncc4c(O)c3c2c1]

To a solution of 9-methoxy-5H-pyrido[4,3-b]carbazole-5,11(6H)-dione (120 mg, 0.43 mmol) in THF (5 ml) was added MeMgBr (1 ml, 3.0 mmol) at 0° C., and stirred at rt and stirred for 1 h, then added with NH4Cl aq and EtOAc. The org phase was washed with NaHCO₃ aq and brine, dried over Na2SO4 and concentrated in vacuo.

The residue was dissolved in DCM (1 ml) then TFA (0.1 ml) and triethyl silane (0.1 ml) were added and the mixture was stirred overnight at rt. The volatiles were removed and the residue purified with RP-HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm.) to afford the title compound.

MS (ESI): 279.2 [M+H]⁺

### Example 49

### 7-methoxy-11-methyl-10H-pyrido[3,4-b]carbazole

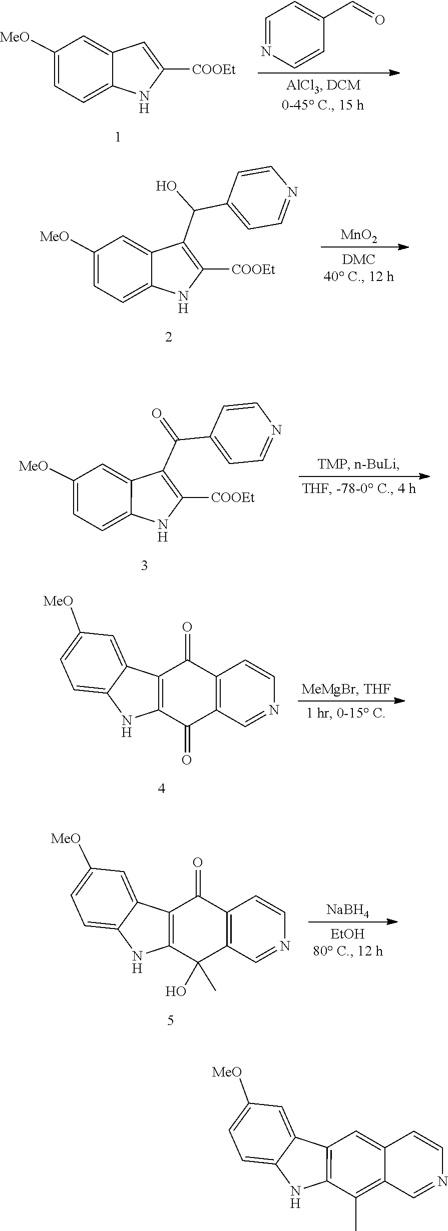

### Step 1: ethyl 3-(hydroxy(pyridin-4-yl)methyl)-5-methoxy-1H-indole-2-carboxylate

To a solution of pyridine-4-carbaldehyde (1.9 g, 18.2 mmol, 1.7 mL, 2 eq) in DCM (30 mL) was added AlCl₃ (2.4 g, 18.2 mmol, 997.0 uL, 2 eq), and the mixture was stirred at 20° C. for 30 min under N₂. then ethyl 5-methoxy-1H-indole-2-carboxylate (2.0 g, 9.1 mmol, 1 eq) was added to the mixture at 0° C., which was brought back to 20° C. and stirred for 3 hrs. LC-MS showed Reactant 1 was not consumed completely then the reaction was stirred at 45° C. for 12 hrs. LC-MS showed Reactant 1 was consumed and desired mass was detected. The mixture was then quenched with ice cold water (100 mL) and extracted with DCM (3×50 mL). The organic layer was dried over anhydrous Na₂SO₄ and concentrated to give solid material. The residue was purified by flash silica gel chromatography (ISCO®; 20 g SepaFlash® Silica Flash Column, Eluent of 0˜100% Ethyl acetate/Petroleum ether gradient @ 70 mL/min) to give ethyl 3-[hydroxy(4-pyridyl)methyl]-5-methoxy-1H-indole-2-carboxylate.

### Step 2: ethyl 3-isonicotinoyl-5-methoxy-1H-indole-2-carboxylate

To a solution of ethyl 3-[hydroxy(4-pyridyl)methyl]-5-methoxy-1H-indole-2-carboxylate (0.9 g, 2.7 mmol, 1 eq) in DCM (30 mL) was added MnO₂ (4.8 g, 55.1 mmol, 20 eq). The mixture was stirred at 40° C. for 12 hrs. LC-MS showed ethyl 3-[hydroxy(4-pyridyl)methyl]-5-methoxy-1H-indole-2-carboxylate was consumed and desired mass was detected. The reaction mixture was filtered and concentrated under reduced pressure to give ethyl 5-methoxy-3-(pyridine-4-carbonyl)-1H-indole-2-carboxylate.

### Step 3: 7-methoxy-10H-pyrido[3,4-b] carbazole-5,11-dione

To a solution of TMP (979.9 mg, 6.9 mmol, 1.1 mL, 5 eq) in THF (15 mL) was added n-BuLi (2.5 M, 2.4 mL, 4.3 eq) at 0° C. and stirred for 1 hr. The mixture was cooled to −78° C. and added ethyl 5-methoxy-3-(pyridine-4-carbonyl)-1H-indole-2-carboxylate (0.4 g, 1.3 mmol, 1 eq) in THF (5 mL) and stirred for 3 hrs at −78° C. LC-MS showed ethyl 5-methoxy-3-(pyridine-4-carbonyl)-1H-indole-2-carboxylate was consumed and desired mass was detected. The reaction mixture was quenched by addition NH₄Cl 50 mL, and extracted with ethyl acetate (30 mL×3). The combined organic layers were washed with brine (50 mL×2), dried over Na₂SO₄, filtered and concentrated under reduced pressure to give a residue. The residue was purified by flash silica gel chromatography (ISCO®; 4 g SepaFlash® Silica Flash Column, Eluent of 0˜30% Ethyl acetate/Petroleum ether gradient @ 70 mL/min) to give 7-methoxy-10H-pyrido[3,4-b] carbazole-5,11-dione.

### Step 4: 11-hydroxy-7-methoxy-11-methyl-10H-pyrido[3,4-b]carbazol-5-one

To a solution of 7-methoxy-10H-pyrido[3,4-b]carbazole-5,11-dione (120 mg, 431.2 umol, 1 eq) in THF (4 mL) was added bromo(methyl)magnesium (3 M, 316.2 uL, 2.2 eq) at 0° C. The mixture was stirred at 15° C. for 1 hr. TLC indicated 7-methoxy-10H-pyrido[3,4-b] carbazole-5,11-dione was consumed completely and one new spot formed. The reaction was added to saturated aqueous NH₄Cl (10 mL) and extracted with ethyl acetate (10 mL×3). The combined organic phase was washed with brine (10 mL×3), dried over anhydrous Na₂SO₄, filtered and concentrated to give 11-hydroxy-7-methoxy-11-methyl-10H-pyrido[3,4-b]carbazol-5-one.

### Step 5: 7-methoxy-11-methyl-10H-pyrido[3,4-b]carbazole

To a solution of 11-hydroxy-7-methoxy-11-methyl-10H-pyrido[3,4-b]carbazol-5-one (120 mg, 407.7 umol, 1 eq) in EtOH (3 mL) was added NaBH₄ (107.9 mg, 2.8 mmol, 7 eq). The mixture was stirred at 80° C. for 12 hrs. LC-MS showed 11-hydroxy-7-methoxy-11-methyl-10H-pyrido[3,4-b]carbazol-5-one was consumed and desired mass was detected. The reaction was poured into saturated aqueous NaHCO₃ (10 mL) and extracted with ethyl acetate (5 mL×3). The combined organic phase was washed with brine (10 mL×2), dried over anhydrous Na₂SO₄, filtered and concentrated to give a residue. The residue was purified by prep-TLC (SiO₂, Petroleum ether/Ethyl acetate=0:1) and then the residue was purified by prep-HPLC (FA condition; column: 3-Phenomenex Luna C18 75*30 mm*3 um; mobile phase: [water(0.2% FA)-ACN]; B %: 30%-60%, 9 min) to afford 7-methoxy-11-methyl-10H-pyrido[3,4-b]carbazole

MS (ESI): m/z=263.0 [M+H]⁺

### Example 50

### 7-methoxy-10,11-dimethyl-10H-pyrido[3,4-b]carbazole

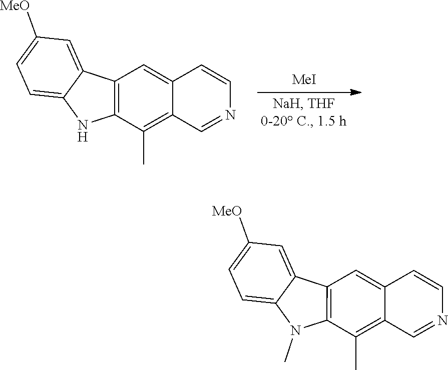 [SMILES: "MeI" ; COc1ccc2[nH]c3c(C)c4cnccc4cc3c2c1 ; COc1ccc2c(c1)c1cc3ccncc3c(C)c1n2C]

To a solution of 7-methoxy-11-methyl-10H-pyrido[3,4-b]carbazole (40 mg, 106.2 umol, 1 eq, TFA) in THF (4 mL) was added NaH (12.7 mg, 318.8 umol, 60% purity, 3 eq) was stirred at 0° C., then the reaction was stirred at 20° C. for 30 min, then Mel (18.1 mg, 127.5 umol, 7.9 uL, 1.2 eq) was added at 20° C. The mixture was stirred at 20° C. for 1 hr. LC-MS showed 7-methoxy-11-methyl-10H-pyrido[3,4-b]carbazole was consumed and desired mass was detected. The reaction mixture was quenched by addition NH₄Cl 5 mL, and then diluted with water 5 mL and extracted with Ethyl acetate (5 mL×3). The combined organic layers were washed with brine (5 mL×3), dried over Na₂SO₄, filtered and concentrated under reduced pressure to give a residue. The residue was purified by prep-HPLC (FA condition; column: 3-Phenomenex Luna C18 75*30 mm*3 um; mobile phase: [water(0.2% FA)-ACN]; B %: 15%-45%, 10 min) to give 7-methoxy-10,11-dimethyl-pyrido[3,4-b]carbazole.

MS (ESI): m/z=277.1 [M+H]⁺

### Example 51

### 2-((5,6-dimethyl-6H-pyrido[4,3-b]carbazol-8-yl)oxy)-N,N-dimethylethanamine

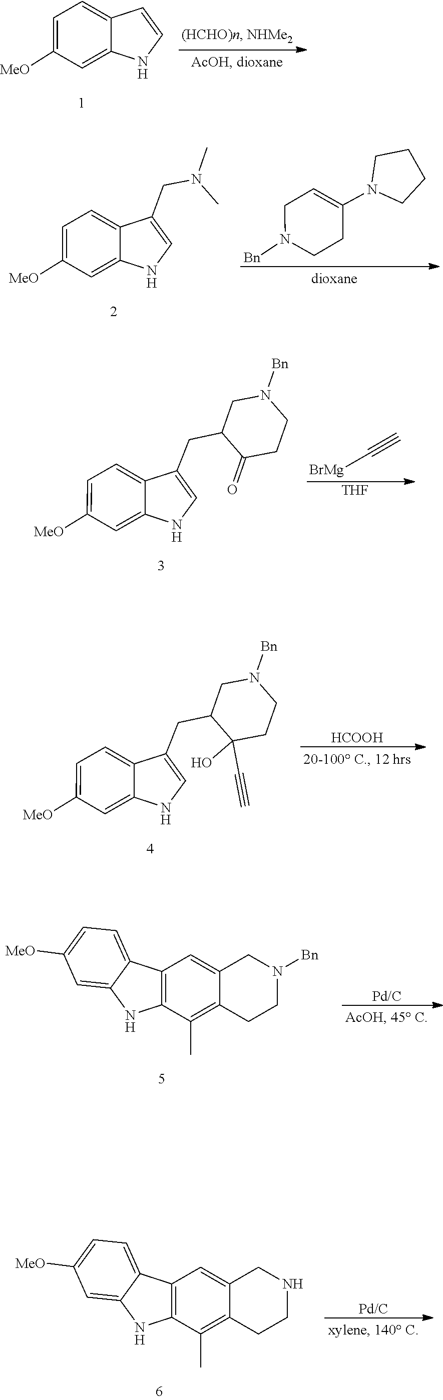

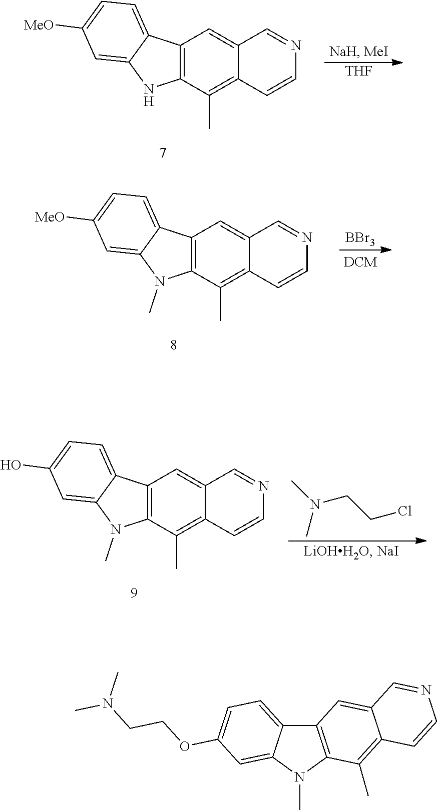

### Step 1: 1-6-methoxy-1H-indol-3-yl)-N,N-dimethylmethanamine

To a stirred solution of 6-methoxy-1H-indole (5 g, 33.9 mmol, 1 eq) and N-methylmethanamine; hydrochloride (3.0 g, 37.3 mmol, 1.1 eq) in dioxane (30 mL) and H₂O (30 mL) was added formaldehyde (3.0), 37.3 mmol, 2.7 mL, 37% purity, 1.1 eq), TEA (3.7 g, 37.3 mmol, 5.2 mL, 1.1 eq) and AcOH (3.0 g, 50.9 mmol, 2.9 mL, 1.5 eq) at 20° C. The reaction mixture was stirred at 20° C. for 12 hr. LC-MS showed 6-methoxy-1H-indole was consumed completely and about 68.9% of desired compound was detected. The reaction mixture was concentrated to give a residue. The residue was adjusted to pH 12 with 2M NaOH solution and filtered. The cake was dried to afford the crude product of 1-(6-methoxy-1H-indol-3-yl)-N,N-dimethylmethanamine.

### Step 2: 1-benzyl-3-((6-methoxy-1H-indol-3-yl)methyl)piperidin-4-one

To a stirred solution of 1-(6-methoxy-1H-indol-3-yl)-N,N-dimethylmethanamine (3 g, 14.6 mmol, 1 eq) in dioxane (60 mL) was added 1-benzyl-4-(pyrrolidin-1-yl)-1,2,3,6-tetrahydropyridine (8.9 g, 36.7 mmol, 2.5 eq) at 20° C., the reaction mixture was stirred at 120° C. for 12 hr. TLC (Petroleum ether/Ethyl acetate=1/1, Rf=0.35) and LCMS indicated 1-(6-methoxy-1H-indol-3-yl)-N,N-dimethylmethanamine was consumed completely and one new spot formed. After cooling to 20° C., the reaction mixture was concentrated. The residue was diluted with water (50 mL) and extracted with ethyl acetate (50 mL×3). The combined organic phase was washed with brine (50 mL×1), dried over anhydrous Na₂SO₄, filtered and concentrated in vacuum. The residue was purified by column chromatography plate 1 (SiO₂, Petroleum ether/Ethyl acetate=I/O to 1/1) (Petroleum ether/Ethyl acetate=1/1, Rf=0.35) to give 1-benzyl-3-((6-methoxy-1H-indol-3-yl)methyl)piperidin-4-one.

### Step 3: 1-benzyl-4-ethynyl-3-((6-methoxy-1H-indol-3-yl)methyl)piperidin-4-ol

To a stirred solution of 1-benzyl-3-((6-methoxy-1H-indol-3-yl)methyl)piperidin-4-one (4.4 g, 12.6 mmol, 1 eq) in THF (50 mL) was added bromo(ethynyl)magnesium (0.5 M, 75.7 mL, 3 eq) dropwise at 0° C., the reaction mixture was stirred at 0° C. for 3 hr under N₂. TLC (Petroleum ether/Ethyl acetate=0/1, Rf=0.43) indicated -benzyl-3-((6-methoxy-1H-indol-3-yl)methyl)piperidin-4-one was consumed completely and one new spot formed. The reaction mixture was poured into ice saturated NH₄Cl solution (50 mL) and the aqueous phase was extracted with ethyl acetate (50 mL×3). The combined organic phase was washed with brine (50 mL×1), dried over anhydrous Na₂SO₄, filtered and concentrated in vacuum to afford 1-benzyl-4-ethynyl-3-((6-methoxy-1H-indol-3-yl)methyl)piperidin-4-ol.

### Step 4: 2-benzyl-8-methoxy-5-methyl-2,3,4,6-tetrahydro-1H-pyrido[4,3-b]carbazole

1-benzyl-4-ethynyl-3-((6-methoxy-1H-indol-3-yl)methyl)piperidin-4-ol (5 g, 13.3 mmol, 1 eq) was dissolved into formic acid (30 mL) at 20° C. and the reaction mixture was stirred at 100° C. for 12 hr. LC-MS showed 1-benzyl-4-ethynyl-3-((6-methoxy-1H-indol-3-yl)methyl)piperidin-4-ol was consumed completely and about 55% of desired compound was detected. After cooling to 20° C., the reaction mixture was concentrated. The residue was diluted with ethyl acetate (50 mL) and adjusted to pH 8 with saturated NaHCO₃ solution. The aqueous phase was extracted with ethyl acetate (50 mL×3). The combined organic phase was washed with brine (50 mL×1), dried over anhydrous Na₂SO₄, filtered and concentrated in vacuum. The residue was purified by column chromatography (SiO₂, Petroleum ether/(Ethyl acetate/Ethanol=3/1)=I/O to 1/1) (Petroleum ether/(Ethyl acetate/Ethanol=3/1)=1/1, Rf=0.70) to afford 2-benzyl-8-methoxy-5-methyl-2,3,4,6-tetrahydro-1H-pyrido[4,3-b]carbazole.

### Step 5: 8-methoxy-5-methyl-2,3,4,6-tetrahydro-1H-pyrido[4,3-b]carbazole

To a solution of 2-benzyl-8-methoxy-5-methyl-2,3,4,6-tetrahydro-1H-pyrido[4,3-b]carbazole (1 g, 2.8 mmol, 1 eq) in AcOH (5 mL) was added Pd/C (500 mg, 10% purity) at 20° C. under N₂. The suspension was degassed under vacuum and purged with H₂ for 3 times. The mixture was stirred under H₂ (15 psi) at 45° C. for 2 hr. LC-MS showed 2-benzyl-8-methoxy-5-methyl-2,3,4,6-tetrahydro-1H-pyrido[4,3-b]carbazole was consumed completely and about 45.5% of desired compound was detected. After cooling to 20° C., the reaction mixture was filtered and the filtrate was concentrated. The residue was triturated with ethyl acetate (5 mL) at 20° C. for 15 min to afford 8-methoxy-5-methyl-2,3,4,6-tetrahydro-1H-pyrido[4,3-b]carbazole.

### Step 6: 8-methoxy-5-methyl-6H-pyrido[4,3-b]carbazole

To a stirred solution of Pd/C (500 mg, 10% purity) in xylene (10 mL) was added 8-methoxy-5-methyl-2,3,4,6-tetrahydro-1H-pyrido[4,3-b]carbazole (500 mg, 1.8 mmol, 1 eq) at 20° C., the reaction mixture was stirred at 140° C. for 12 hr. LCMS showed 8-methoxy-5-methyl-2,3,4,6-tetrahydro-1H-pyrido[4,3-b]carbazole was consumed completely and about 94.5% of desired compound was detected. After cooling to 20° C., the reaction mixture was diluted with methanol (20 mL) and filtered through Celite. The filtrate was concentrated to give 8-methoxy-5-methyl-6H-pyrido[4,3-b]carbazole Step 7: 8-methoxy-5,6-dimethyl-6H-pyrido[4,3-b]carbazole

To a stirred solution of 8-methoxy-5-methyl-6H-pyrido[4,3-b]carbazole (320 mg, 1.2 mmol, 1 eq) in THF (5 mL) was added NaH (146.3 mg, 3.6 mmol, 60% purity, 3 eq) at 0° C., the reaction mixture was stirred at 0° C. for 1 hr. Then Mel (155.8 mg, 1.1 mmol, 68.3 uL, 0.9 eq) was added and the reaction mixture was warmed to 20° C. and stirred for 2 hr. LCMS showed 8-methoxy-5-methyl-6H-pyrido[4,3-b]carbazole was consumed completely and about 79% of desired compound was detected. The reaction mixture was poured into ice saturated NH₄Cl solution (3 mL). The aqueous phase was extracted with ethyl acetate (3 mL×4). The combined organic phase was washed with brine (3 mL×1), dried over anhydrous Na₂SO₄, filtered and concentrated in vacuum to afford 8-methoxy-5,6-dimethyl-6H-pyrido[4,3-b]carbazole.

### Step 8: 5,6-dimethyl-6H-pyrido[4,3-b]carbazol-8-ol

To a stirred solution of 8-methoxy-5,6-dimethyl-6H-pyrido[4,3-b]carbazole (430 mg, 1.5 mmol, 1 eq) in DCM (10 mL) was added BBr₃ (1.9 g, 7.7 mmol, 749.6 uL, 5 eq) at 0° C., the reaction mixture was stirred at 45° C. for 1 hr under N₂. LCMS showed 8-methoxy-5,6-dimethyl-6H-pyrido[4,3-b]carbazole was consumed completely and about 82% of desired compound was detected. After cooling to 20° C., the reaction mixture was poured into ice saturated NaHCO₃ solution (20 mL) and adjusted to pH 8 with NaHCO₃ solid. The mixture was concentrated under reduced pressure to remove DCM and filtered. The cake was washed with water (10 mL) and dried to afford 5,6-dimethyl-6H-pyrido[4,3-b]carbazol-8-ol.

### Step 9: 2-((5,6-dimethyl-6H-pyrido[4,3-b]carbazol-8-yl)oxy)-N,N-dimethylethanamine

To a stirred solution of 5,6-dimethyl-6H-pyrido[4,3-b]carbazol-8-ol (50 mg, 190.6 umol, 1 eq) and 2-chloro-N,N-dimethyl-ethanamine (32.9 mg, 228.7 umol, 1.2 eq, HCl) in DMF (2 mL) was added LiOH·H₂O (31.9 mg, 762.4 umol, 4 eq) and NaI (28.5 mg, 190.6 umol, 1 eq) at 20° C., the reaction mixture was stirred at 80° C. for 12 hr. LCMS showed the reaction was completely. The reaction was concentrated to give a residue. The residue was purified by prep-HPLC (TFA condition, column: Phenomenex Gemini-NX 150*30 mm*5 um; mobile phase: [water(0.1% TFA)-ACN]; B %: 10%-40%, 9 min) to afford 2-((5,6-dimethyl-6H-pyrido[4,3-b]carbazol-8-yl)oxy)-N,N-dimethylethanamine.

MS (ESI): m/z=334.1 [M+H]⁺

### Example 52

### 8-((4,5-dihydro-1H-imidazol-2-yl)methoxy)-5,6-dimethyl-6H-pyrido[4,3-b]carbazole

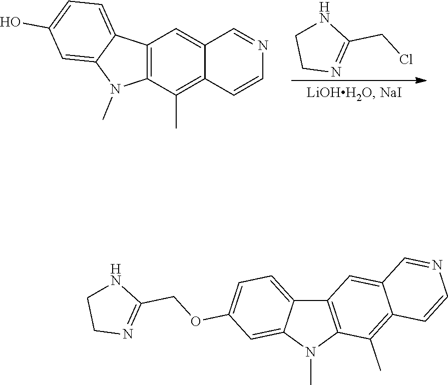

To a stirred solution of 5,6-dimethyl-6H-pyrido[4,3-b]carbazol-8-ol (50 mg, 190.6 umol, 1 eq) and 2-(chloromethyl)-4,5-dihydro-1H-imidazole (35.4 mg, 228.7 umol, 1.2 eq, HCl) in DMF (4 mL) was added LiOH·H₂O (31.9 mg, 762.4 umol, 4 eq) and NaI (28.5 mg, 190.6 umol, 1 eq) at 20° C., the reaction mixture was stirred at 80° C. for 12 hr. LCMS showed the reaction was completely. The reaction was concentrated to give a residue. The residue was purified by prep-HPLC (TFA condition, column: Phenomenex Gemini-NX 150*30 mm*5 um; mobile phase: [water(0.1% TFA)-ACN]; B %: 10%-40%, 9 min) to afford 8-((4,5-dihydro-1H-imidazol-2-yl)methoxy)-5,6-dimethyl-6H-pyrido[4,3-b]carbazole.

MS (ESI): m/z=345.1 [M+H]⁺

### Example 53

### 4-(2-((5,6-dimethyl-6H-pyrido[4,3-b]carbazol-8-yl)oxy)ethyl)morpholine

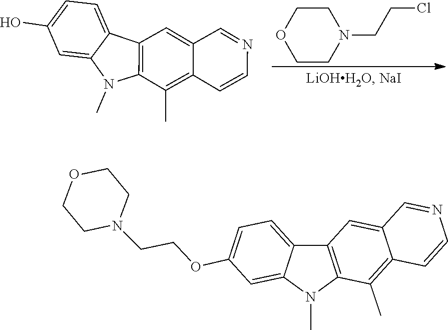

To a solution of 5,6-dimethylpyrido[4,3-b]carbazol-10-ol (50.3 mg, 192.0 umol, 1 eq) in DMF (4 mL) was added 4-(2-chloroethyl)morpholine (42.8 mg, 230.4 umol, 1.2 eq, HCl), LiOH·H₂O (32.2 mg, 768.2 umol, 4 eq) and NaI (28.7 mg, 192.0 umol, 1 eq). The mixture was stirred at 80° C. for 12 hr. LCMS showed the reaction was completely. The reaction mixture was concentrated under reduced pressure to remove solvent. The residue was purified by prep-HPLC (TFA condition, column: Phenomenex luna C18 250*50 mm*10 um; mobile phase: [water(0.1% TFA)-ACN]; B %: 10%-40%, 10 min) to give 4-(2-((5,6-dimethyl-6H-pyrido[4,3-b]carbazol-8-yl)oxy)ethyl)morpholine.

MS (ESI): m/z=376.1 [M+H]⁺

### Example 54

### 4-(2-((5,6-dimethyl-6H-pyrido[4,3-b]carbazol-10-yl)oxy)ethyl)morpholine

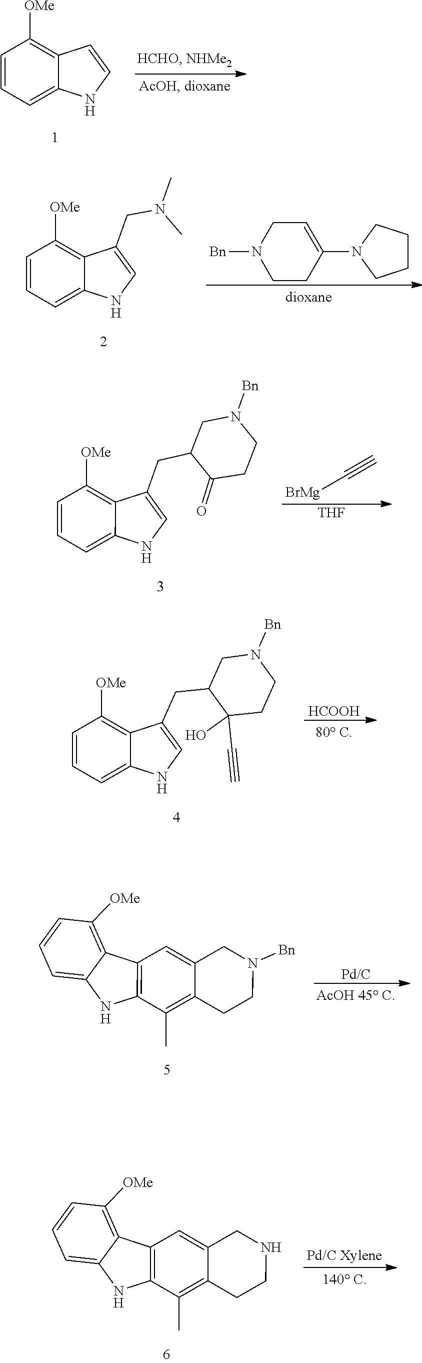

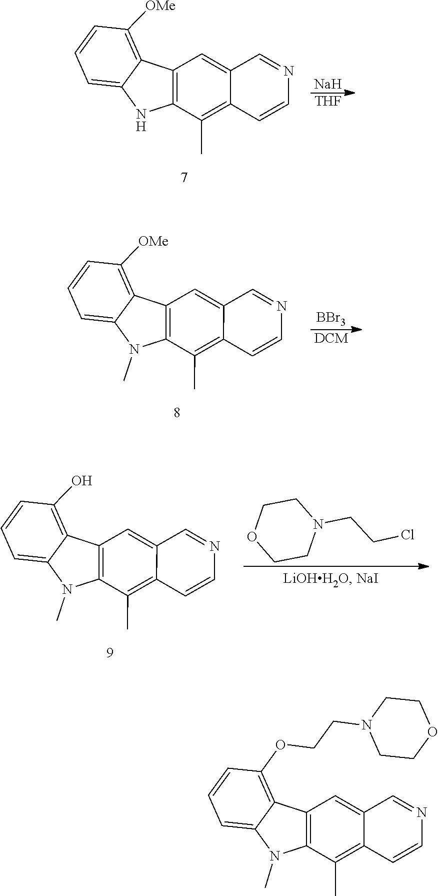

### Step 1: 1-(4-methoxy-1H-indol-3-yl)-N,N-dimethylmethanamine

To a solution of N-methylmethanamine; hydrochloride (15.2 g, 129.1 mmol, 0.7 eq, HCl) in dioxane (200 mL) and H₂O (200 mL) was added TEA (18.9 g, 186.8 mmol, 26.0 mL, 1.1 eq), AcOH (15.3 g, 254.8 mmol, 14.5 mL, 1.5 eq), 4-methoxy-1H-indole (25 g, 169.8 mmol, 1 eq) and formaldehyde (15.1 g, 186.8 mmol, 13.9 mL, 37% purity, 1.1 eq). The mixture was stirred at 20° C. for 12 hr. LC-MS showed the desired compound was detected. The mixture was concentrated to remove the dioxane. The reaction was added to 100 mL of NaOH (2M). The reaction mixture was filtered and the filter cake was washed with 300 mL of H₂O, dried in vacuum to give crude 1-(4-methoxy-1H-indol-3-yl)-N,N-dimethylmethanamine.

### Step 2: 1-benzyl-3-((4-methoxy-1H-indol-3-yl)methyl)piperidin-4-one

To a solution of 1-(4-methoxy-1H-indol-3-yl)-N,N-dimethylmethanamine (22 g, 107.7 mmol, 1 eq) in dioxane (400 mL) was added 1-benzyl-4-pyrrolidin-1-yl-3,6-dihydro-2H-pyridine (65.2 g, 269.2 mmol, 2.5 eq). The mixture was stirred at 120° C. for 12 hr. LC-MS showed the desired compound was detected. The reaction mixture was partitioned between H₂O 200 mL and EtOAc 200 mL. The aqueous phase was washed with EtOAc (200 mL×3), The organic phase was separated and washed with brine (100 mL×3), dried over Na₂SO₄, filtered and concentrated under reduced pressure to give a residue. The residue was purified by flash silica gel chromatography (ISCO®; 120 g SepaFlash® Silica Flash Column, Eluent of 0˜30% Ethyl acetate/Petroleum ether gradient @ 100 mL/min) to give 1-benzyl-3-((4-methoxy-1H-indol-3-yl)methyl)piperidin-4-one.

### Step 3: benzyl-4-ethynyl-3-((4-methoxy-1H-indol-3-yl)methyl)piperidin-4-ol

To a solution of 1-benzyl-3-((4-methoxy-1H-indol-3-yl)methyl)piperidin-4-one (25 g, 71.7 mmol, 1 eq) in THF (300 mL) was added bromo(ethynyl)magnesium (0.5 M, 430.4 mL, 3 eq). The mixture was stirred at 0° C. for 2 hr. LC-MS showed the desired compound was detected. The reaction mixture was partitioned between NH₄Cl 300 mL and EtOAc 200 mL. The aqueous phase was washed with EtOAc (500 mL×3). The organic phase was separated, washed with brine (200 mL×3), dried over Na₂SO₄, filtered and concentrated under reduced pressure to give benzyl-4-ethynyl-3-((4-methoxy-1H-indol-3-yl)methyl)piperidin-4-ol.

### Step 4: 2-benzyl-10-methoxy-5-methyl-2,3,4,6-tetrahydro-1H-pyrido[4,3-b]carbazole

HCOOH (300 mL) was added to benzyl-4-ethynyl-3-((4-methoxy-1H-indol-3-yl)methyl)piperidin-4-ol (26.8 g, 71.5 mmol, 1 eq). The mixture was stirred at 80° C. for 12 hr. LC-MS showed the desired compound was detected. The reaction mixture was concentrated under reduced pressure. The residue was diluted with NaHCO₃ 300 mL and extracted with EtOAc (300 mL×3). The combined organic layers were washed with brine (150 mL×3), dried over Na₂SO₄, filtered and concentrated under reduced pressure to give a residue. The residue was purified by flash silica gel chromatography (ISCO®; 80 g SepaFlash® Silica Flash Column, Eluent of 0˜100% Ethyl acetate/Petroleum ether gradient @ 50 mL/min) to give 2-benzyl-10-methoxy-5-methyl-2,3,4,6-tetrahydro-1H-pyrido[4,3-b]carbazole.

### Step 5: 10-methoxy-5-methyl-2,3,4,6-tetrahydro-1H-pyrido[4,3-b]carbazole

A mixture of 2-benzyl-10-methoxy-5-methyl-2,3,4,6-tetrahydro-1H-pyrido[4,3-b]carbazole (6.5 g, 18.2 mmol, 1 eq), Pd/C (3 g, 561.0 umol, 10% purity) in AcOH (100 mL) was degassed and purged with H₂ (15 psi) for 3 times, and then the mixture was stirred at 45° C. for 2 hr under H₂ (15 psi) atmosphere. LC-MS showed the desired compound was detected. The reaction mixture was filtered and the filtrate dried in vacuum to give 10-methoxy-5-methyl-2,3,4,6-tetrahydro-1H-pyrido[4,3-b]carbazole.

### Step 6: 10-methoxy-5-methyl-6H-pyrido[4,3-b]carbazole

To a solution of Pd/C (500 mg, 10% purity) in xylene (30 mL) was added 10-methoxy-5-methyl-2,3,4,6-tetrahydro-1H-pyrido[4,3-b]carbazole (1 g, 3.7 mmol, 1 eq). The mixture was stirred at 145° C. for 12 hr. LC-MS showed the desired compound was detected. The reaction mixture was filtered and the filtrate dried in vacuum to give crude 10-methoxy-5-methyl-6H-pyrido[4,3-b]carbazole.

### Step 7: 10-methoxy-5,6-dimethyl-6H-pyrido[4,3-b]carbazole

To a solution of 10-methoxy-5-methyl-6H-pyrido[4,3-b]carbazole (650 mg, 2.4 mmol, 1 eq) in THF (10 mL) was added NaH (297.3 mg, 7.4 mmol, 60% purity, 3 eq) at 0° C. and stirred for 30 min, Mel (351.7 mg, 2.4 mmol, 154.2 uL, 1 eq) was added to the mixture at 0° C. The mixture was stirred at 25° C. for 2 hr. LC-MS showed the desired compound was detected. The reaction mixture was partitioned between NH₄Cl 100 mL and EtOAc 50 mL. The aqueous phase was washed with EtOAc (50 mL×3), the organic phase was separated and washed with brine (20 mL×3), dried over Na₂SO₄, filtered and concentrated under reduced pressure to give a residue. The residue was purified by flash silica gel chromatography (ISCO®; 12 g SepaFlash® Silica Flash Column, Eluent of 0˜100% Ethyl acetate/Petroleum ether gradient @ 75 mL/min) to give 10-methoxy-5,6-dimethyl-6H-pyrido[4,3-b]carbazole.

### Step 8: 5,6-dimethyl-6H-pyrido[4,3-b]carbazol-10-ol

To a solution of 10-methoxy-5,6-dimethyl-6H-pyrido[4,3-b]carbazole (300 mg, 1.0 mmol, 1 eq) in DCM (10 mL) was added BBr₃ (1.3 g, 5.4 mmol, 523.0 uL, 5 eq) at 0° C. The mixture was stirred at 25° C. for 12 hr. LC-MS showed the desired compound was detected. The reaction mixture was washed with NaHCO₃ (30 mL) and filtered and the filter cake was washed with 20 mL of H₂O, dried in vacuum to give crude 5,6-dimethyl-6H-pyrido[4,3-b]carbazol-10-ol.

### Step 9: 4-(2-((5,6-dimethyl-6H-pyrido[4,3-b]carbazol-10-yl)oxy)ethyl)morpholine

To a solution of 5,6-dimethyl-6H-pyrido[4,3-b]carbazol-10-ol (50 mg, 190.6 umol, 1 eq) in DMF (4 mL) was added 4-(2-chloroethyl)morpholine (42.5 mg, 228.7 umol, 1.2 eq, HCl), NaI (28.5 mg, 190.6 umol, 1 eq) and LiOH·H₂O (32.0 mg, 762.4 umol, 4 eq). The mixture was stirred at 80° C. for 12 hr. LCMS showed the reaction was completely. The reaction mixture was concentrated under reduced pressure to remove solvent. The residue was purified by prep-HPLC (TFA condition, column: 3-Phenomenex Luna C18 75*30 mm*3 um; mobile phase: [water(0.2% FA)-ACN]; B %: 10%-45%, 9 min) to give 4-(2-((5,6-dimethyl-6H-pyrido[4,3-b]carbazol-10-yl)oxy)ethyl)morpholine.

MS (ESI): m/z=376.1 [M+H]⁺

### Example 55

### 2-((5,6-dimethyl-6H-pyrido[4,3-b]carbazol-10-yl)oxy)-N,N-dimethylethanamine

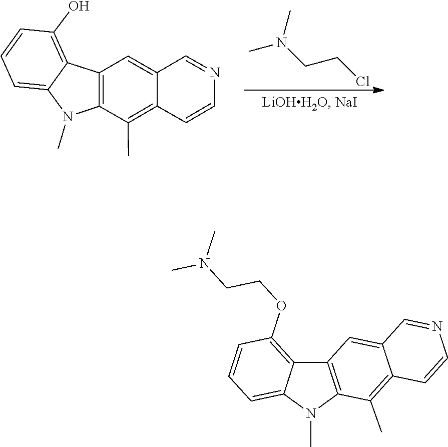

To a solution of 5,6-dimethyl-6H-pyrido[4,3-b]carbazol-10-ol (70 mg, 266.8 umol, 1 eq) in DMF (4 mL) was added 2-chloro-N,N-dimethyl-ethanamine (34.4 mg, 239.1 umol, 1.2 eq, HCl), LiOH·H₂O (44.7 mg, 1.0 mmol, 4 eq) and NaI (40.0 mg, 266.8 umol, 1 eq). The mixture was stirred at 80° C. for 12 hr. LC-MS showed the desired compound was detected. The reaction mixture was filtered and the filtrate was a crude product. The residue was purified by prep-HPLC (TFA condition, column: Phenomenex Gemini-NX 150*30 mm*5 um; mobile phase: [water(0.1% TFA)-ACN]; B %: 20%-50%, 9 min) to give 2-((5,6-dimethyl-6H-pyrido[4,3-b]carbazol-10-yl)oxy)-N,N-dimethylethanamine.

MS (ESI): m/z=334.1 [M+H]⁺

### Example 56

### 10-((4,5-dihydro-1H-imidazol-2-yl)methoxy)-5,6-dimethyl-6H-pyrido[4,3-b]carbazole

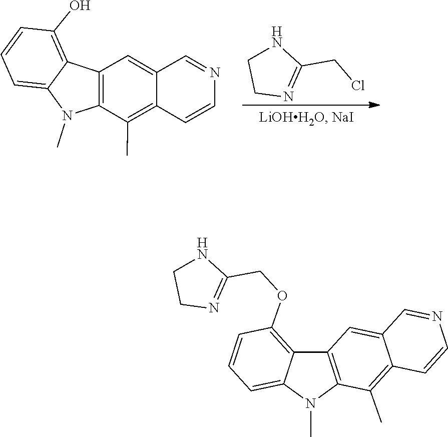

To a solution of 5,6-dimethyl-6H-pyrido[4,3-b]carbazol-10-ol (50 mg, 190.6 umol, 1 eq) in DMF (4 mL) was added 2-(chloromethyl)-4,5-dihydro-1H-imidazole (35.4 mg, 228.7 umol, 1.2 eq, HCl), LiOH·H₂O (31.9 mg, 762.4 umol, 4 eq) and NaI (28.5 mg, 190.6 umol, 1 eq). The mixture was stirred at 80° C. for 12 hr. LC-MS showed the desired compound was detected. The reaction mixture was filtered and the filtrate to give crude product. The residue was purified by prep-HPLC (TFA condition, column: Phenomenex Gemini-NX 150*30 mm*5 um; mobile phase: [water(0.1% TFA)-ACN]; B %: 20%-50%, 9 min) to give 10-((4,5-dihydro-1H-imidazol-2-yl)methoxy)-5,6-dimethyl-6H-pyrido[4,3-b]carbazole.

MS (ESI): m/z=345.1 [M+H]⁺

### Example 57

### 2-((5,6-dimethyl-6H-pyrido[4,3-b]carbazol-9-yl)oxy)-N,N-dimethylacetamide

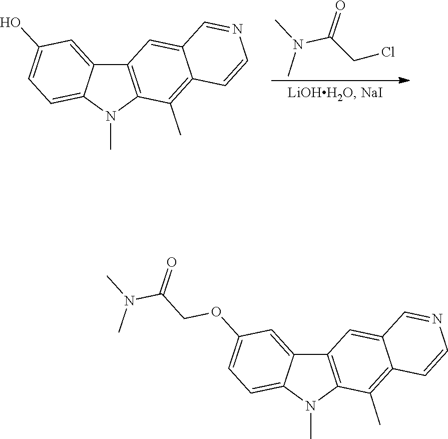

To a solution of 5,6-dimethyl-6H-pyrido[4,3-b]carbazol-9-ol (50 mg, 190.6 umol, 1 eq) in DMF (2 mL) was added 2-chloro-N,N-dimethylacetamide (27.8 mg, 228.7 umol, 23.5 uL, 1.2 eq), LiOH·H₂O (32.0 mg, 762.4 umol, 4 eq) and NaI (28.5 mg, 190.6 umol, 1 eq). The mixture was stirred at 80° C. for 12 hr. LCMS showed some desired product was formed. The reaction was concentrated to give a residue. The residue was purified by prep-HPLC (TFA condition, column: Phenomenex luna C18 100*40 mm*5 um; mobile phase: [water(0.1% TFA)-ACN]; B %: 10%-35%, 8 min) to afford 2-((5,6-dimethyl-6H-pyrido[4,3-b]carbazol-9-yl)oxy)-N,N-dimethylacet amide.

MS (ESI): m/z=348.1 [M+H]⁺

### Example 58

### 2-((5,6-dimethyl-6H-pyrido[4,3-b]carbazol-9-yl)oxy)-N-(3(dimethylamino)propyl)-acetamide

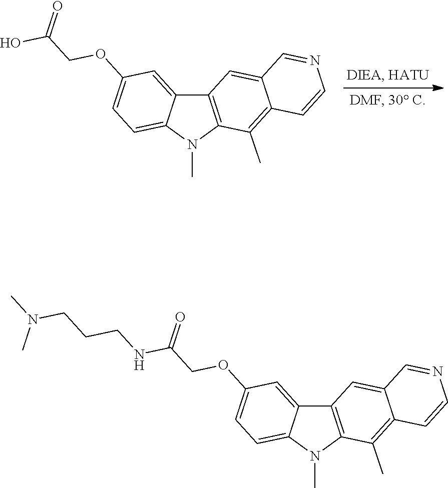 [SMILES: Cc1c2ccncc2cc2c3cc(OCC(=O)O)ccc3n(C)c12 ; Cc1c2ccncc2cc2c3cc(OCC(=O)NCCCN(C)C)ccc3n(C)c12]

To a solution of 2-((5,6-dimethyl-6H-pyrido[4,3-b]carbazol-9-yl)oxy)acetic acid (30 mg, 84.0 umol, 1 eq, HCl) in DMF (3 mL) was added DIEA (27.1 mg, 210.2 umol, 36.6 uL, 2.5 eq) and HATU (47.9 mg, 126.1 umol, 1.5 eq) and N,N-dimethylpropane-1,3-diamine (9.4 mg, 92.4 umol, 11.5 uL, 1.1 eq). The mixture was stirred at 30° C. for 12 hr. LC-MS showed the desired compound was detected. The reaction mixture was filtered and the filtrate dried in vacuum to give crude product. The residue was purified by prep-HPLC (TFA condition, column: Phenomenex Gemini-NX C18 75*30 mm*3 um; mobile phase: [water(0.1% TFA)-ACN]; B %: 5%-40%, 8 min) to give 2-((5,6-dimethyl-6H-pyrido[4,3-b]carbazol-9-yl)oxy)-N-(3-(dimethylamino)propyl)acetamide.

MS (ESI): m/z=405.1 [M+H]⁺

### Example 59

### 2-((5,6-dimethyl-6H-pyrido[4,3-b]carbazol-9-yl)oxy)-N-(2-(dimethylamino)ethyl)-acetamide

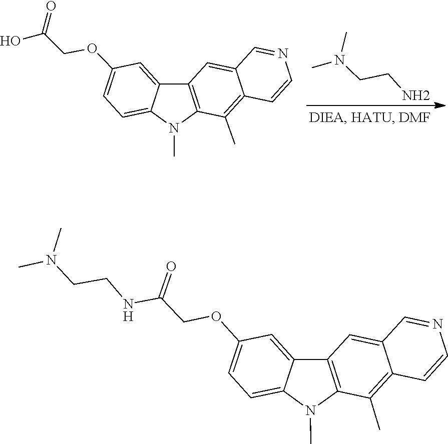

To a solution of 2-(5,6-dimethylpyrido[4,3-b]carbazol-9-yl)oxyacetic acid (30 mg, 84.0 umol, 1 eq, HCl) in DMF (2 mL) were added N,N-dimethylethane-1,2-diamine (8.1 mg, 92.4 umol, 10.1 uL, 1.1 eq), DIEA (27.1 mg, 210.2 umol, 36.6 uL, 2.5 eq) and HATU (47.9 mg, 126.12 umol, 1.5 eq). The mixture was stirred at 30° C. for 12 hr. LCMS showed the reaction was completely. The reaction mixture was concentrated under reduced pressure to remove solvent to give the residue. The residue was purified by prep-HPLC (TFA condition, column: Phenomenex Gemini-NX C18 75*30 mm*3 um; mobile phase: [water(0.1% TFA)-ACN]; B %: 5%-40%, 8 min) to afford 2-((5,6-dimethyl-6H-pyrido[4,3-b]carbazol-9-yl)oxy)-N-(2-(dimethylamino)ethyl)acetamide.

MS (ESI): m/z=391.1 [M+H]⁺

### Example 60

### 2-((5,6-dimethyl-6H-pyrido[4,3-b]carbazol-9-yl)oxy)-1-(4-methylpiperazin-1-yl)ethanone

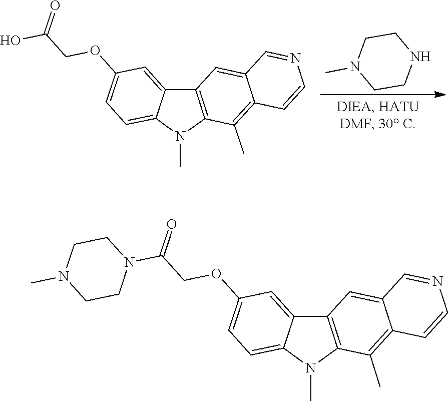

To a solution of 2-((5,6-dimethyl-6H-pyrido[4,3-b]carbazol-9-yl)oxy)acetic acid (28 mg, 78.4 umol, 1 eq, HCl) in DMF (2 mL) was added DIEA (25.3 mg, 196.1 umol, 34.1 uL, 2.5 eq), HATU (44.7 mg, 117.7 umol, 1.5 eq) and 1-methylpiperazine (8.6 mg, 86.3 umol, 9.5 uL, 1.1 eq). The mixture was stirred at 30° C. for 12 hr. LCMS showed the reaction was completely. The reaction was concentrated to give a residue. The residue was purified by prep-HPLC (TFA condition, column: Phenomenex luna C18 100*40 mm*5 um; mobile phase: [water(0.1% TFA)-ACN]; B %: 1%-30%, 8 min) to afford 2-((5,6-dimethyl-6H-pyrido[4,3-b]carbazol-9-yl)oxy)-1-(4-methylpiperazin-1-yl)ethanone.

MS (ESI): m/z=403.1 [M+H]⁺

### Example 61

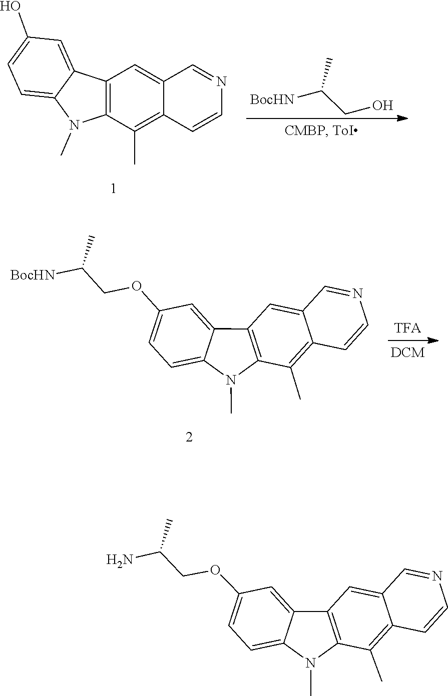 [SMILES: Cc1c2ccncc2cc2c3cc(O)ccc3n(C)c12 ; C[C@H](CO)NC(=O)OC(C)(C)C ; Cc1c2ccncc2cc2c3cc(OC[C@@H](C)NC(=O)OC(C)(C)C)ccc3n(C)c12 ; Cc1c2ccncc2cc2c3cc(OC[C@@H](C)N)ccc3n(C)c12]

### (R)-tert-butyl (1-((5,6-dimethyl-6H-pyrido[4,3-b]carbazol-9-yl)oxy)propan-2-yl)carbamate (2)

To a solution of 5,6-dimethyl-6H-pyrido[4,3-b]carbazol-9-ol (60 mg, 228.7 umol, 1 eq) in toluene (4 mL) was added tert-butyl N-[(1R)-2-hydroxy-1-methyl-ethyl]carbamate (40.0 mg, 228.7 umol, 1 eq) and 2-(tributyl-λ⁵-phosphanylidene)acetonitrile (110.4 mg, 457.4 umol, 2 eq). The mixture was stirred at 100° C. for 12 hr. LC-MS showed the desired compound was detected. The reaction mixture was concentrated under reduced pressure to give a residue. The residue was purified by prep-TLC (SiO₂, Petroleum ether/Ethyl acetate=0/1) to give 80 mg of (R)-tert-butyl(1-((5,6-dimethyl-6H-pyrido[4,3-b]carbazol-9-yl)oxy)propan-2-yl)carbamate 2.

### (R)-1-((5,6-dimethyl-6H-pyrido[4,3-b]carbazol-9-yl)oxy)propan-2-amine

To a solution of (R)-tert-butyl (1-((5,6-dimethyl-6H-pyrido[4,3-b]carbazol-9-yl)oxy)propan-2-yl)carbamate (80 mg, 190.7 umol, 1 eq) in DCM (3 mL) was added TFA (3 mL). The mixture was stirred at 25° C. for 2 hr. LC-MS showed the desired compound was detected. The reaction mixture was concentrated under reduced pressure to give a residue. The residue was purified by prep-HPLC (TFA condition column: Phenomenex Gemini-NX 150*30 mm*5 um; mobile phase: [water(0.1% TFA)-ACN]; B %: 20%-50%, 9 min) to give (R)-1-((5,6-dimethyl-6H-pyrido[4,3-b]carbazol-9-yl)oxy)propan-2-amine.

MS (ESI): m/z=320.1 [M+H]⁺

### Example 62

### 4-[2-(6-ethyl-5-methyl-pyrido[4,3-b]carbazol-9-yl)oxyethyl]morpholine

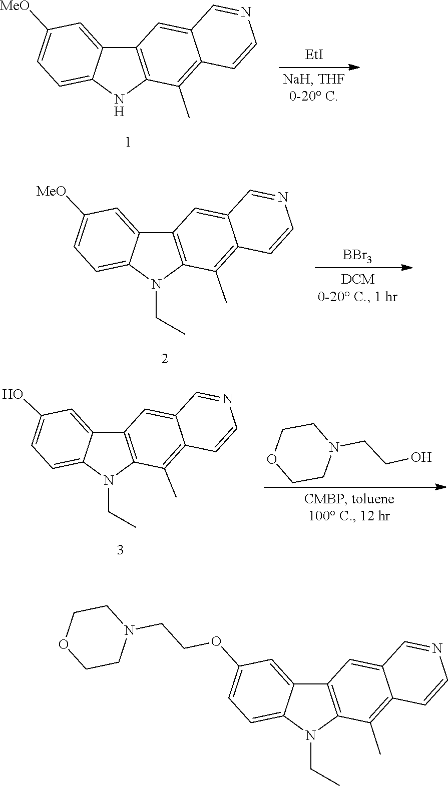

### Step 1: 6-ethyl-9-methoxy-5-methyl-6H-pyrido[4,3-b]carbazole

To a solution of 9-methoxy-5-methyl-6H-pyrido[4,3-b]carbazole (0.3 g, 1.1 mmol, 1 eq) in THF (5 mL) was added NaH (91.4 mg, 2.29 mmol, 60% purity, 2 eq) at 0° C., the mixture was stirred at 0° C. for 10 min, then iodoethane (214.0 mg, 1.3 mmol, 109.7 uL, 1.2 eq) was added. The mixture was stirred at 20° C. for 12 hrs. LC-MS showed 9-methoxy-5-methyl-6H-pyrido[4,3-b]carbazole was consumed and desired mass was detected. The reaction mixture was quenched by addition NH₄Cl (10 mL), then diluted with water (5 mL) and extracted with EtOAc (10 mL×3). The combined organic layers were washed with brine (15 mL×3), dried over Na₂SO₄, filtered and concentrated under reduced pressure to give 6-ethyl-9-methoxy-5-methyl-pyrido[4,3-b]carbazole.

### Step 2: 6-ethyl-5-methyl-6H-pyrido[4,3-b]carbazol-9-ol

To a solution of 6-ethyl-9-methoxy-5-methyl-pyrido[4,3-b]carbazole (0.2 g, 861.0 umol, 1 eq) in DCM (5 mL) was added BBr₃ (1.0 g, 4.3 mmol, 414.8 uL, 5 eq) at 0° C. The mixture was stirred at 20° C. for 1 hr. LC-MS showed 6-ethyl-9-methoxy-5-methyl-pyrido [4,3-b]carbazole was consumed and desired mass was detected. The reaction mixture was quenched by addition NaHCO₃ 10 mL, and pressure to give a residue. 100 mL (EtOAc:MeOH=10:1) was added, then filtered and concentrated under reduced pressure to give 6-ethyl-5-methyl-6H-pyrido[4,3-b]carbazol-9-ol.

### Step 3: 4-(2-((6-ethyl-5-methyl-6H-pyrido[4,3-b]carbazol-9-yl)oxy)ethyl)morpholine

To a solution of 6-ethyl-5-methyl-6H-pyrido[4,3-b]carbazol-9-ol (20 mg, 72.3 umol, 1 eq), 2-morpholinoethanol (11.3 mg, 86.8 umol, 10.6 uL, 1.2 eq) in toluene (4 mL) was added 2-(tributyl-λ5-phosphanylidene)acetonitrile (52.4 mg, 217.1 umol, 3 eq). The mixture was stirred at 100° C. for 12 hrs under N₂. LC-MS showed 6-ethyl-5-methyl-6H-pyrido [4,3-b]carbazol-9-ol was consumed and desired mass was detected. The reaction mixture was concentrated under reduced pressure to remove solvent. The residue was purified by prep-HPLC (neutral condition; column: Waters Xbridge BEH C18 100*25 mm*5 um; mobile phase: [water(10 mM NH4HCO3)-ACN]; B %: 35%-65%, 10 min) to give 4-[2-(6-ethyl-5-methyl-pyrido[4,3-b]carbazol-9-yl)oxyethyl]morpholine.

MS (ESI): m/z=390.1 [M+H]⁺

### Example 63

### 9-((4,5-dihydro-1H-imidazol-2-yl)methoxy)-6-ethyl-5-methyl-6H-pyrido[4,3-b]carbazole

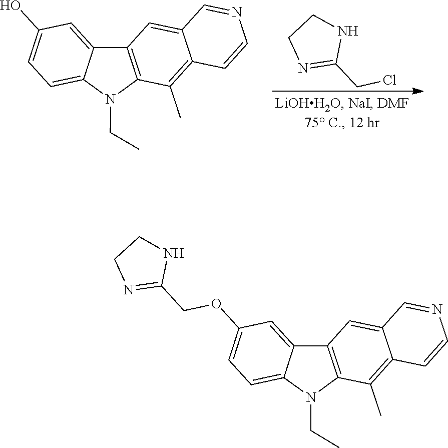

To a solution of 2-(chloromethyl)-1H-imidazole; hydrochloride (27.6 mg, 180.9 umol, 1 eq) in DMF (3 mL) was added LiOH·H₂O (30.3 mg, 723.7 umol, 4 eq) and 6-ethyl-5-methyl-pyrido[4,3-b]carbazol-9-ol (50 mg, 180.9 umol, 1 eq), NaI (27.1 mg, 180.94 umol, 1 eq). The mixture was stirred at 75° C. for 12 hrs. LC-MS showed 6-ethyl-5-methyl-pyrido[4,3-b]carbazol-9-ol was consumed and desired mass was detected. The reaction mixture was filtered and concentrated under reduced pressure to give a residue. The residue was purified by prep-HPLC (FA condition; column: 3-Phenomenex Luna C18 75*30 mm*3 um; mobile phase: [water(0.2% FA)-ACN]; B %: 5%-30%, 8 min) to give 9-((4,5-dihydro-1H-imidazol-2-yl)methoxy)-6-ethyl-5-methyl-6H-pyrido[4,3-b]carbazole.

MS (ESI): m/z=359.1 [M+H]⁺

### Example 64

### 4-(2-((5,6-dimethyl-6H-pyrido[4,3-b]carbazol-7-yl)oxy)ethyl) morpholine

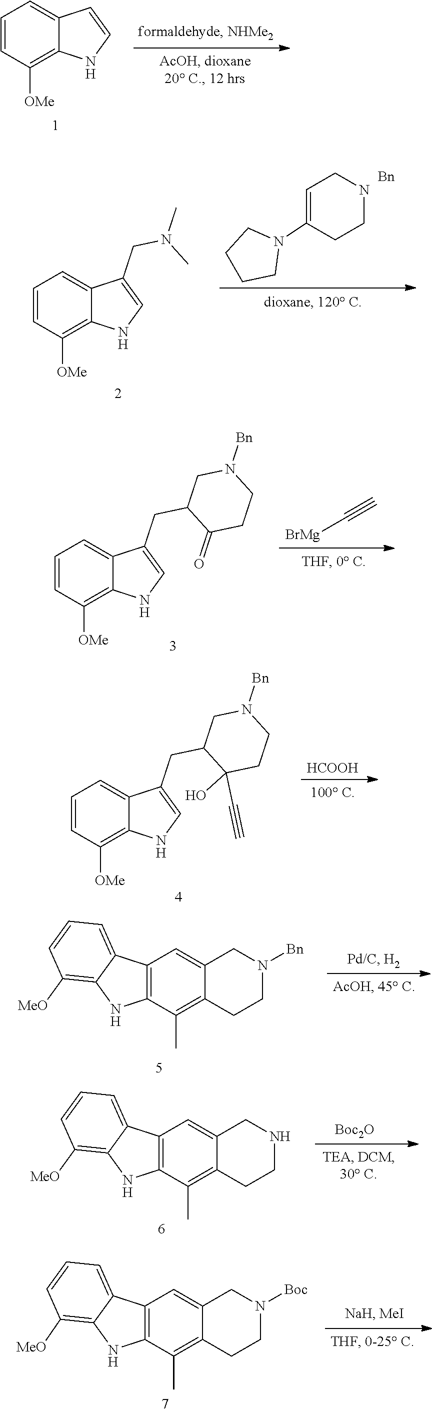

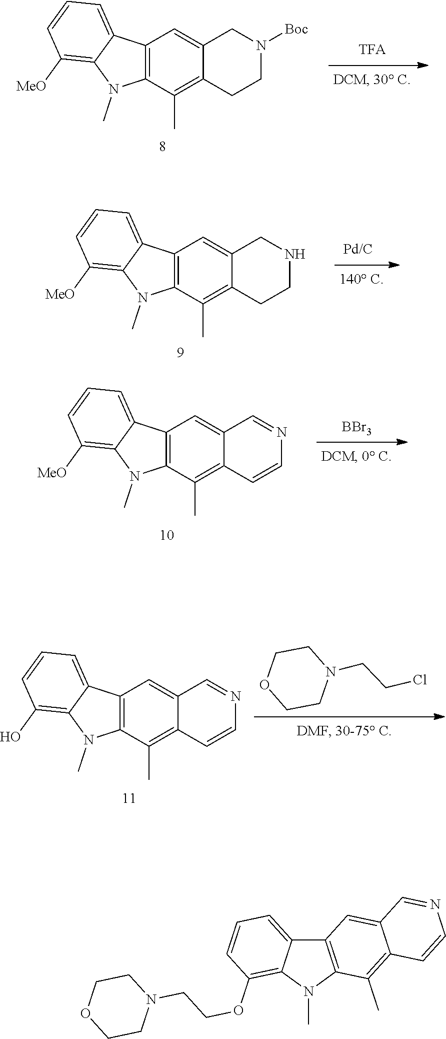

### Step 1: 1-(7-methoxy-1H-indol-3-yl)-N,N-dimethylmethanamine

To a solution of 7-methoxy-1H-indole (24 g, 163.0 mmol, 21.2 mL, 1 eq) in H₂O (100 mL) and dioxane (100 mL) was added TEA (18.1 g, 179.3 mmol, 24.9 mL, 1.1 eq), AcOH (14.69 g, 244.6 mmol, 13.9 mL, 1.5 eq), formaldehyde (14.5 g, 179.3 mmol, 13.3 mL, 37% purity, 1.1 eq) and N-methylmethanamine (25.2 g, 123.9 mmol, 28.3 mL, HCl). The mixture was stirred at 20° C. for 12 hrs. LC-MS showed the desired compound was detected. The reaction was quenched by adding to saturated aqueous NaOH (2M) (150 mL), filtrated and the cake was washed with H₂O (50 mL×3). The cake was dried under reduced pressure to give 1-(7-methoxy-1H-indol-3-yl)-N,N-dimethyl-methanamine.

### Step 2: 1-benzyl-3-((7-methoxy-1H-indol-3-yl)methyl)piperidin-4-one

To a solution of 1-(7-methoxy-1H-indol-3-yl)-N,N-dimethylmethanamine (12 g, 58.7 mmol, 1 eq) in dioxane (300 mL) was added 1-benzyl-4-pyrrolidin-1-yl-3,6-dihydro-2H-pyridine (35.5 g, 146.8 mmol, 2.5 eq). The mixture was stirred at 120° C. for 12 hrs. LC-MS showed the desired compound was detected. The reaction mixture was partitioned between H₂O (80 mL) and EtOAc (80 mL). The aqueous phase was washed with EtOAc (60 mL×3). The organic phase was separated, dried over Na₂SO₄, filtered and concentrated under reduced pressure to give a residue. The residue was purified by flash silica gel chromatography (ISCO®; 40 g SepaFlash® Silica Flash Column, Eluent of 0˜50% Ethyl acetate/Petroleum ether gradient @ 50 mL/min) to give 1-benzyl-3-((7-methoxy-1H-indol-3-yl)methyl) piperidin-4-one.

### Step 3: 1-benzyl-4-ethynyl-3-((7-methoxy-1H-indol-3-yl)methyl)piperidin-4-ol

To a solution of 1-benzyl-3-((7-methoxy-1H-indol-3-yl)methyl)piperidin-4-one (20 g, 57.4 mmol, 1 eq) in THF (200 mL) was added bromo(ethynyl)magnesium (0.5 M, 344.3 mL, 3 eq). The mixture was stirred at 0° C. for 2 hrs. LC-MS showed the desired compound was detected. The reaction mixture was partitioned between NH₄Cl (80 mL) and EtOAc (80 mL). The aqueous phase was washed with EtOAc (80 mL×2). The organic phase was separated, dried over Na₂SO₄, filtered and concentrated under reduced pressure to give 1-benzyl-4-ethynyl-3-((7-methoxy-1H-indol-3-yl)methyl)piperidin-4-ol.

### Step 4: 2-benzyl-7-methoxy-5-methyl-2,3,4,6-tetrahydro-1H-pyrido[4,3-b]carbazole

The solution of 1-benzyl-4-ethynyl-3-((7-methoxy-1H-indol-3-yl)methyl)piperidin-4-ol (18 g, 48.0 mmol, 1 eq) in HCOOH (200 mL) was stirred at 100° C. for 12 hrs. LC-MS showed the desired compound was detected. The reaction mixture was concentrated under reduced pressure to give a residue. The reaction mixture was partitioned between NaHCO₃ (60 mL) and EtOAc (60 mL). The aqueous phase was washed with EtOAc (50 mL×2). The organic phase was separated, dried over Na₂SO₄, filtered and concentrated under reduced pressure to give a residue. The residue was purified by flash silica gel chromatography (ISCO®; 40 g SepaFlash® Silica Flash Column, Eluent of 0˜50% Ethyl acetate/Petroleum ether gradient @ 50 mL/min) to give 2-benzyl-7-methoxy-5-methyl-2,3,4,6-tetrahydro-1H-pyrido[4,3-b]carbazole.

### Step 5: 7-methoxy-5-methyl-2,3,4,6-tetrahydro-1H-pyrido[4,3-b]carbazole

A mixture of 2-benzyl-7-methoxy-5-methyl-2,3,4,6-tetrahydro-1H-pyrido[4,3-b]carbazole (5 g, 14.0 mmol, 1 eq), Pd/C (2 g, 10% purity, 1 eq) in AcOH (20 mL) was degassed and purged with H₂ (15 Psi) for 3 times, and then the mixture was stirred at 45° C. for 2 hrs under H₂ (15 Psi) atmosphere. LC-MS showed the desired compound was detected. The reaction mixture was filtered and concentrated under reduced pressure to give 7-methoxy-5-methyl-2,3,4,6-tetrahydro-1H-pyrido[4,3-b]carbazole.

### Step 6: tert-butyl 7-methoxy-5-methyl-3,4-dihydro-1H-pyrido[4,3-b]carbazole-2(6H)-carboxylate

To a solution of 7-methoxy-5-methyl-2,3,4,6-tetrahydro-1H-pyrido[4,3-b]carbazole (850 mg, 3.1 mmol, 1 eq) in DCM (5 mL) was added Boc₂O (1.3 g, 6.3 mmol, 1.47 mL, 2 eq) and TEA (968.8 mg, 9.5 mmol, 1.3 mL, 3 eq). The mixture was stirred at 30° C. for 3 hrs. LC-MS showed desired compound was detected. The reaction mixture was concentrated under reduced pressure to give a residue. The residue was purified by prep-TLC (SiO₂, Petroleum ether/Ethyl acetate=3/1) to give tert-butyl 7-methoxy-5-methyl-3,4-dihydro-1H-pyrido[4,3-b]carbazole-2(6H)-carboxylate.

### Step 7: tert-butyl7-methoxy-5,6-dimethyl-3,4-dihydro-1H-pyrido[4,3-b]carbazole-2(6H)-carboxylate

To a solution of tert-butyl 7-methoxy-5-methyl-1,3,4,6-tetrahydropyrido[4,3-b]carbazole-2-carboxylate (530 mg, 1.4 mmol, 1 eq) in THF (8 mL) was added NaH (115.7 mg, 2.8 mmol, 60% purity, 2 eq) at 0° C. and stirred for 10 min, Mel (184.7 mg, 1.3 mmol, 81.0 uL, 0.9 eq) was added to the mixture at 0° C. The mixture was stirred at 25° C. for 12 hrs. LC-MS showed most desired compound was detected. The reaction mixture was quenched by addition NH₄Cl (10 mL) and extracted with Ethyl acetate (6 mL×3). The combined organic layers were washed with brine (10 mL), dried over Na₂SO₄, filtered and concentrated under reduced pressure to give a residue. The residue was purified by column chromatography (SiO₂, Petroleum ether/Ethyl acetate=1/1) to give tert-butyl 7-methoxy-5,6-dimethyl-3,4-dihydro-1H-pyrido[4,3-b]carbazole-2(6H)-carboxylate.

### Step 8: 7-methoxy-5,6-dimethyl-2,3,4,6-tetrahydro-1H-pyrido[4,3-b]carbazole

To a solution of tert-butyl 7-methoxy-5,6-dimethyl-3,4-dihydro-1H-pyrido[4,3-b]carbazole-2(6H)-carboxylate (310 mg, 814.7 umol, 1 eq) in DCM (4 mL) was added TFA (1.8 g, 16.3 mmol, 1.2 mL, 20 eq). The mixture was stirred at 30° C. for 1 hr. LC-MS showed desired compound was detected. The reaction mixture was concentrated under reduced pressure to give a residue. The reaction mixture was added MeOH (15 mL), pH was adjusted to around 7-8 by progressively adding TEA, then the reaction mixture was concentrated under reduced pressure to give 7-methoxy-5,6-dimethyl-2,3,4,6-tetrahydro-1H-pyrido[4,3-b]carbazole.

### Step 9: 7-methoxy-5,6-dimethyl-6H-pyrido[4,3-b]carbazole

To a solution of Pd/C (100 mg, 10% purity, 1 eq) in xylene (8 mL) was added 7-methoxy-5,6-dimethyl-1,2,3,4-tetrahydropyrido[4,3-b]carbazole (220 mg, 784.7 umol, 1 eq). The mixture was stirred at 140° C. for 12 hrs. LC-MS showed desired compound was detected. The reaction mixture was filtered and the filtrate dried in vacuum to give 7-methoxy-5,6-dimethyl-6H-pyrido[4,3-b]carbazole.

### Step 10: 5,6-dimethyl-6H-pyrido[4,3-b]carbazol-7-ol

To a solution of 7-methoxy-5,6-dimethyl-6H-pyrido[4,3-b]carbazole (38 mg, 137.5 umol, 1 eq) in DCM (2 mL) was added BBr₃ (206.7 mg, 825.0 umol, 79.5 uL, 6 eq) at 0° C. The mixture was stirred at 0° C. for 2 hrs. LC-MS showed most desired compound was detected. The reaction mixture was diluted with NaHCO₃ (15 mL) and extracted with DCM (10 mL×3). The combined organic layers were washed with brine (10 mL×2), dried over (Na₂SO₄), filtered and concentrated under reduced pressure to give 5,6-dimethyl-6H-pyrido[4,3-b]carbazol-7-ol.

### Step 11: 4-(2-((5,6-dimethyl-6H-pyrido[4,3-b]carbazol-7-yl)oxy)ethyl)morpholine

To a solution of 5,6-dimethyl-6H-pyrido[4,3-b]carbazol-7-ol (50 mg, 190.6 umol, 1 eq) in DMF (4 mL) was added NaI (5.71 mg, 38.1 umol, 0.2 eq) and 4-(2-chloroethyl)morpholine; hydrochloride (35.47 mg, 190.6 umol, 1 eq), LiOH·H₂O (39.9 mg, 953.0 umol, 5 eq) at 30° C. The mixture was stirred at 75° C. for 2 hrs. LC-MS showed the desired compound was detected. The reaction mixture was concentrated under reduced pressure to give a residue. The residue was purified by prep-HPLC (TFA condition; column: Phenomenex Gemini-NX C18 75*30 mm*3 um; mobile phase: [water(0.1% TFA)-ACN]; B %: 2%-25%, 8 min) to give 4-(2-((5,6-dimethyl-6H-pyrido[4,3-b]carbazol-7-yl)oxy)ethyl) morpholine.

MS (ESI): m/z=376.1 [M+H]⁺

### Example 65

### (R)—N-(1-(dimethylamino)propan-2-yl)-10-methoxy-5,6-dimethyl-6H-pyrido[4,3-b]carbazole-1-carboxamide

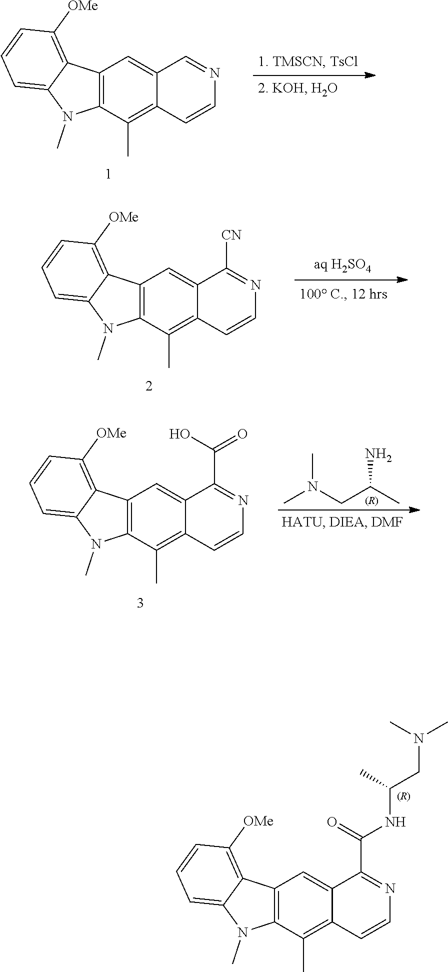

### Step 1: 10-methoxy-5,6-dimethyl-6H-pyrido[4,3-b]carbazole-1-carbonitrile

To a solution of 10-methoxy-5,6-dimethyl-6H-pyrido[4,3-b]carbazole (550 mg, 1.9 mmol, 1 eq) in DCM (5 mL) was added TMSCN (513.3 mg, 5.1 mmol, 647.4 uL, 2.6 eq). A solution of TosCl (720.9 mg, 3.7 mmol, 1.9 eq) in DCM (5 mL) was dropwsie added to the above mixture at 25° C. The mixture was stirred at 25° C. for 12 hr. LCMS showed the intermediate was detected and 5 mL KOH (50% W/W) was added. The mixture was stirred at 25° C. for 2 hr. LC-MS showed the desired compound was detected. The reaction mixture was partitioned between H₂O 50 mL and CH₂Cl₂ 50 mL. The aqueous phase was separated, washed with CH₂Cl₂ (50 mL×3), the organic phase was dried over Na₂SO₄, filtered and concentrated under reduced pressure to give 10-methoxy-5,6-dimethyl-6H-pyrido[4,3-b]carbazole-1-carbonitrile.

### Step 2: 10-methoxy-5,6-dimethyl-6H-pyrido[4,3-b]carbazole-1-carboxylic acid

To a solution of 10-methoxy-5,6-dimethyl-6H-pyrido[4,3-b]carbazole-1-carbonitrile (100 mg, 331.8 umol, 1 eq) in H₂SO₄ (2 mL, 98% purity) and H₂O (2 mL) was stirred at 110° C. for 12 hr. LC-MS showed the desired compound was detected. The mixture was poured into NaHCO₃ (200 mL) and the brown solid was separate out. Then the mixture was filtered and collect the solid to afford 10-methoxy-5,6-dimethyl-6H-pyrido[4,3-b]carbazole-1-carboxylic acid.

### Step 3: (R)—N-(1-(dimethylamino)propan-2-yl)-10-methoxy-5,6-dimethyl-6H-pyrido[4,3-b]carbazole-1-carboxamide

To a solution of 10-methoxy-5,6-dimethyl-6H-pyrido[4,3-b]carbazole-1-carboxylic acid (100 mg, 312.1 umol, 1 eq) in DMF (2 mL) was added (2R)—N1,N1-dimethylpropane-1,2-diamine (163.9 mg, 936.5 umol, 3 eq, 2 HCl), DIEA (201.7 mg, 1.5 mmol, 271.8 uL, 5 eq) and HATU (237.3 mg, 624.3 umol, 2 eq) at 0° C. The mixture was stirred at 25° C. for 1 hr. LC-MS showed the desired compound was detected. The reaction mixture was filtered and the filtrate dried in vacuum to give crude product. The residue was purified by prep-HPLC (TFA condition, column: Phenomenex luna C18 100*40 mm*5 um; mobile phase: [water(0.1% TFA)-ACN]; B %: 10%-40%, 8 min) to give (R)—N-(1-(dimethylamino)propan-2-yl)-10-methoxy-5,6-dimethyl-6H-pyrido[4,3-b]carbazole-1-carboxamide.

MS (ESI): m/z=405.1 [M+H]⁺

### Example 66

### 9-methoxy-5-methyl-benzofuro[2,3-g]isoquinoline

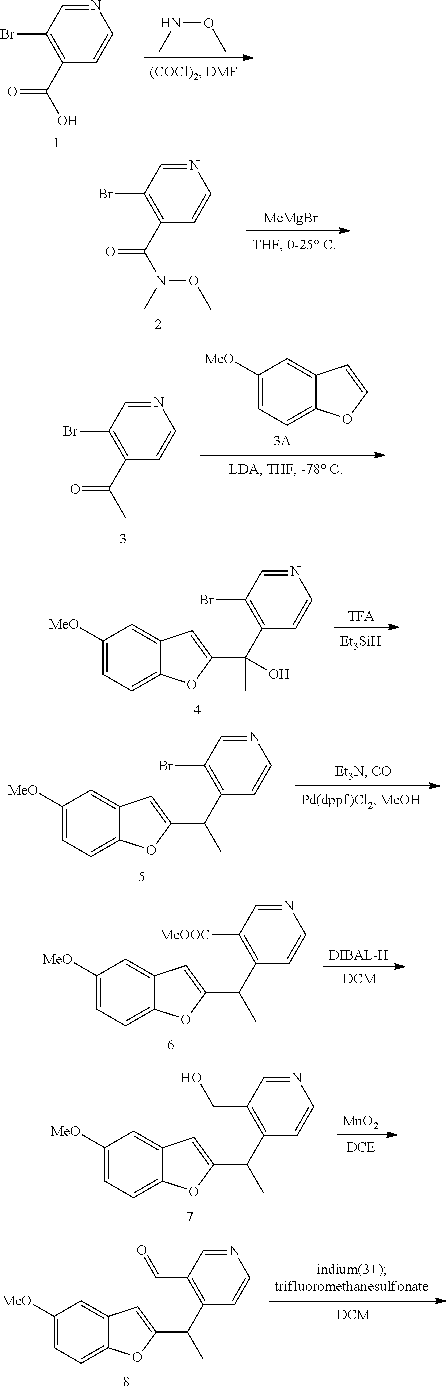

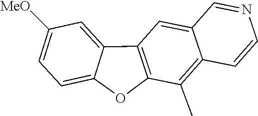 [SMILES: COc1ccc2oc3c(C)c4ccncc4cc3c2c1]

### Step 1: 3-bromo-N-methoxy-N-methyl-pyridine-4-carboxamide

To a solution of 3-bromopyridine-4-carboxylic acid (5 g, 24.7 mmol, 1 eq) and DMF (1.8 g, 24.7 mmol, 1.9 mL, 1 eq) in DCM (100 mL) was added (COCl)₂ (3.8 g, 29.7 mmol, 2.6 mL, 1.2 eq) and then the reaction mixture was stirred at 15° C. for 1 hr. Then the mixture was concentrated to give residue. The residue was dissolved in DCM (100 mL) and Py (4.3 g, 54.4 mmol, 4.4 mL, 2.2 eq), N-methoxymethanamine (2.66 g, 27.23 mmol, 1.1 eq, HCl) was added. The reaction mixture was stirred at 15° C. for 12 hr. TLC (Petroleum ether/Ethyl acetate=1/1, Rf=0.30) indicated 3-bromopyridine-4-carboxylic acid was consumed completely and one new spot formed. The reaction mixture was added to saturated NaHCO₃ (100 mL) and extracted with ethyl acetate (100 mL×3). The combined organic phase was washed with brine (100 mL×1), dried over anhydrous Na₂SO₄, filtered and concentrated in vacuum. The residue was purified by flash silica gel chromatography (ISCO®; 40 g Sepa Flash® Silica Flash Column, Eluent of 0˜30% Ethyl acetate/Petroleum ether gradient @ 100 mL/min) (Petroleum ether/Ethyl acetate=1/1, Rf=0.30) to give 3-bromo-N-methoxy-N-methyl-pyridine-4-carboxamide.

### Step 2: 1-(3-bromo-4-pyridyl)ethanone

To a solution of 3-bromo-N-methoxy-N-methyl-pyridine-4-carboxamide (2.5 g, 10.2 mmol, 1 eq) in THF (25 mL) was added bromo(methyl)magnesium (3 M, 6.8 mL, 2 eq) at 0° C. and then the reaction mixture was stirred at 20° C. for 12 hr. TLC (Petroleum ether/Ethyl acetate=0/1, Rf=0.70) indicated 3-bromo-N-methoxy-N-methyl-pyridine-4-carboxamide was consumed and one new spot formed. The reaction mixture was quenched with saturated aqueous NH₄Cl (30 mL) and extracted with ethyl acetate (50 mL×3). The combined organic phase was washed with brine (30 mL), dried with anhydrous Na₂SO₄, filtered and concentrated in vacuum. The residue was purified by flash silica gel chromatography (ISCO®; 12 g Sepa Flash® Silica Flash Column, Eluent of 0˜25% Ethyl acetate/Petroleum ether gradient @ 100 mL/min) (Petroleum ether/Ethyl acetate=0/1, Rf=0.70) to give purified 1-(3-bromo-4-pyridyl)ethanone.

### Step 3: 1-(3-bromo-4-pyridyl)-1-(5-methoxybenzofuran-2-yl)ethanol

To a stirred solution of 5-methoxybenzofuran (814.7 mg, 5.5 mmol, 1.1 eq) in THF (10 mL) was added LDA (2 M, 3.0 mL, 1.2 eq) at −78° C. under N₂ for 0.5 hr. Then a solution of 1-(3-bromo-4-pyridyl)ethanone (1 g, 5.00 mmol, 1 eq) in THF (10 mL) was added and stirred at −78° C. under N₂ for 1 hr. TLC (Petroleum ether/Ethyl acetate=0/1, Rf=0.60) indicated 1-(3-bromo-4-pyridyl)ethanone was remained and one new spot formed. The reaction mixture was quenched with saturated aqueous NH₄Cl (30 mL) and extracted with ethyl acetate (30 mL×3). The combined organic phase was dried with anhydrous Na₂SO₄, filtered and concentrated in vacuum. The residue was purified by flash silica gel chromatography (ISCO®; 12 g SepaFlash® Silica Flash Column, Eluent of 28˜50% Ethyl acetate/Petroleum ether gradient @ 100 mL/min) TLC (Petroleum ether/Ethyl acetate=0/1, Rf=0.60) to give 11-(3-bromo-4-pyridyl)-1-(5-methoxybenzofuran-2-yl)ethanol.

### Step 4: 3-bromo-4-[1-(5-methoxybenzofuran-2-yl)ethyl]pyridine

To a solution of 1-(3-bromo-4-pyridyl)-1-(5-methoxybenzofuran-2-yl)ethanol (500 mg, 1.44 mmol, 1 eq) in TFA (8 mL) was added Et₃SiH (333.9 mg, 2.9 mmol, 458.7 uL, 2 eq) at 20° C. and then the reaction mixture was stirred at 30° C. for 12 hr. LCMS showed 1-(3-bromo-4-pyridyl)-1-(5-methoxybenzofuran-2-yl)ethanol was consumed and 46% of desired compound was detected. The reaction mixture was concentrated and then adjusted to pH=8 by saturated NaHCO₃ solution. The aqueous phase was extracted with dichloromethane (20 mL×4). The combined organic phase was dried over anhydrous Na₂SO₄, filtered and concentrated in vacuum. The residue was purified by flash silica gel chromatography (ISCO®; 12 g SepaFlash® Silica Flash Column, Eluent of 0˜40% Ethyl acetate/Petroleum ether gradient @ 100 mL/min) TLC (Petroleum ether/Ethyl acetate=0/1, Rf=0.70) to give purified 3-bromo-4-[1-(5-methoxybenzofuran-2-yl)ethyl]pyridine.

### Step 5: methyl 4-[1-(5-methoxybenzofuran-2-yl)ethyl]pyridine-3-carboxylate

To a solution of 3-bromo-4-[1-(5-methoxybenzofuran-2-yl)ethyl]pyridine (500 mg, 1.51 mmol, 1 eq) in MeOH (15 mL) was added Et₃N (456.92 mg, 4.52 mmol, 628.50 uL, 3 eq) and Pd(dppf)Cl₂ (220.27 mg, 301.03 umol, 0.2 eq) under N₂ at 20° C. Then the reaction mixture was stirred at 80° C. under CO (50 psi) for 12 hr. LCMS showed 3-bromo-4-[1-(5-methoxybenzofuran-2-yl)ethyl]pyridine was consumed and 60% of desired compound was detected. The reaction mixture was filtered and the filtrate was concentrated. The residue was purified by flash silica gel chromatography (ISCO®; 4 g Sepa Flash® Silica Flash Column, Eluent of 0˜50% Ethyl acetate/Petroleum ether gradient @ 100 mL/min) TLC (Petroleum ether/Ethyl acetate=0/1, Rf=0.60) to give purified methyl 4-[1-(5-methoxybenzofuran-2-yl)ethyl]pyridine-3-carboxylate.

### Step 6: [4-[1-(5-methoxybenzofuran-2-yl)ethyl]-3-pyridyl]methanol

To a solution of methyl 4-[1-(5-methoxybenzofuran-2-yl)ethyl]pyridine-3-carboxylate (75 mg, 240.9 umol, 1 eq) in DCM (2 mL) was added DIBAL-H (1 M, 722.7 uL, 3 eq) slowly at −78° C. under N₂ for 4 hr. LCMS showed 49% of methyl 4-[1-(5-methoxybenzofuran-2-yl)ethyl]pyridine-3-carboxylate was remained and 30% of desired compound was detected. The reaction mixture was quenched with saturated aqueous potassium sodium tartrate (8 mL) and extracted with dichloromethane (8 mL×3). The combined organic phase was dried with anhydrous Na₂SO₄, filtered and concentrated in vacuum. The residue was purified by prep-TLC (SiO₂, Ethyl acetate/Ethanol=3/1) TLC (Ethyl acetate/Ethanol=3/1, Rf=0.35) to give purified [4-[1-(5-methoxybenzofuran-2-yl)ethyl]-3-pyridyl]methanol.

### Step 7: 4-[1-(5-methoxybenzofuran-2-yl)ethyl]pyridine-3-carbaldehyde

To a solution of [4-[1-(5-methoxybenzofuran-2-yl)ethyl]-3-pyridyl]methanol (26 mg, 91.77 umol, 1 eq) in DCE (2 mL) was added MnO₂ (159.56 mg, 1.84 mmol, 20 eq) at 20° C. and then the reaction mixture was stirred at 20° C. for 12 hr. LCMS showed 5% of Reactant 1 was remained and 73% of desired compound was detected. The reaction mixture was filtered and the filtrate was concentrated. The residue was purified by column chromatography by prep-TLC (SiO₂, Petroleum ether/Ethyl acetate=0/1) TLC (Petroleum ether/Ethyl acetate=0/1 Rf=0.60) to give purified 4-[1-(5-methoxybenzofuran-2-yl)ethyl]pyridine-3-carbaldehyde.

### Step 8: 9-methoxy-5-methyl-benzofuro[2,3-g]isoquinoline

To a solution of 4-[1-(5-methoxybenzofuran-2-yl)ethyl]pyridine-3-carbaldehyde (15 mg, 53.3 umol, 1 eq) in DCE (2 mL) was added indium (3+); trifluoromethanesulfonate (3.00 mg, 5.3 umol, 0.1 eq) and then the reaction mixture was stirred at 115° C. for 12 hr. LCMS showed 4% of Reactant 1 was remained and 78% of desired compound was detected. The reaction mixture was concentrated. The residue was purified by prep-HPLC (column: Waters Xbridge BEH C18 100*30 mm*10 um; mobile phase: [water(10 mM NH4HCO3)-ACN]; B %: 40%-75%, 8 min) to give purified 9-methoxy-5-methyl-benzofuro[2,3-g]isoquinoline.

MS (ESI): m/z=264.0 [M+H]⁺

### Example 67

### 9-methoxy-5-methyl-benzothiopheno[2,3-g]isoquinoline

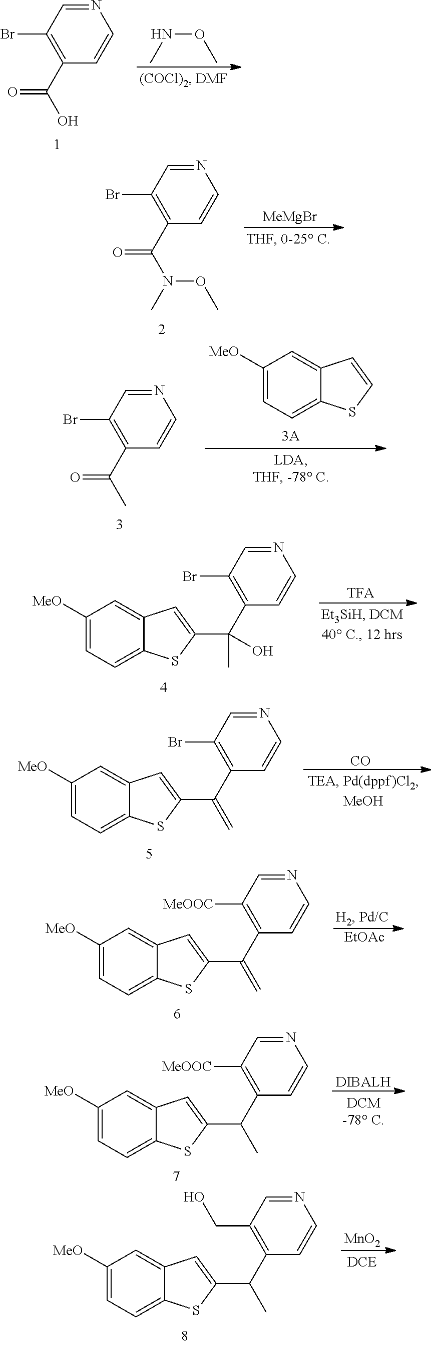

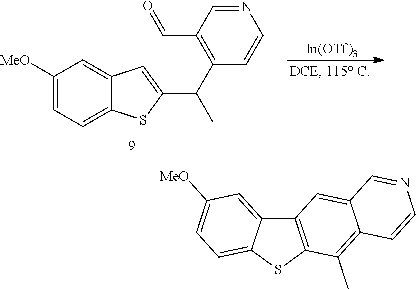 [SMILES: COc1ccc2sc(C(C)c3ccncc3C=O)cc2c1 ; COc1ccc2sc3c(C)c4ccncc4cc3c2c1 ; "In(OTf)3"]

### Step 1: 3-bromo-N-methoxy-N-methyl-pyridine-4-carboxamide

To a solution of 3-bromopyridine-4-carboxylic acid (2 g, 9.9 mmol, 1 eq) in 20 mL of DCM and DMF (72.4 mg, 990.1 μmol, 0.1 eq) was added (COCl)₂ (1.5 g, 11.9 mmol, 1.0 mL, 1.2 eq) at 20° C. The mixture was stirred at 20° C. for 1 hr to give a colorless solution, which was cooled to 0° C., N-methoxymethanamine hydrochloride (1.5 g, 14.9 mmol, 1.5 eq) and Py (1.7 g, 21.8 mmol, 1.8 mL, 2.2 eq) were added and the mixture was stirred at 20° C. for 12 hrs. LCMS showed the reaction was complete and desired product was detected. The reaction mixture was quenched by addition of 30 mL of sat. NaHCO₃, and then extracted three times with 300 mL of ethyl acetate. The combined organic layers were washed with 100 mL of brine, dried over Na₂SO₄, filtered and concentrated under reduced pressure to give a residue, which was purified by column chromatography (SiO₂, eluting with a gradient of petroleum ether/ethyl acetate=1/0 to 0/1) to give crude 3-bromo-N-methoxy-N-methyl-pyridine-4-carboxamide.

### Step 2: 1-(3-bromo-4-pyridyl)ethanone

To a solution of 3-bromo-N-methoxy-N-methyl-pyridine-4-carboxamide (1.9 g, 7.8 mmol, 1 eq) in 20 mL of THF was added MeMgBr (3M, 7.8 mL, 3 eq) at 0° C. The mixture was stirred at 0° C. for 2 hrs. LCMS showed the reaction was complete and desired product was detected. The reaction mixture was quenched by addition of 50 mL sat. NH₄Cl, extracted three times with 60 mL of ethyl acetate. The combined organic layers were washed with 20 mL of brine, dried over Na₂SO₄, filtered and concentrated under reduced pressure to give a residue, which was purified by column chromatography (SiO₂, eluting with a gradient of petroleum ether/ethyl acetate=100/1 to 20/1) to give crude 1-(3-bromo-4-pyridyl)ethanone.

### Step 3: 1-(3-bromo-4-pyridyl)-1-(5-methoxybenzothiophen-2-yl) ethanol

To a solution of 5-methoxybenzothiophene (0.7 g, 4.3 mmol, 1 eq) in 15 mL of THF was added dropwise LDA (2M, 2.6 mL, 1.2 eq) at −78° C. After addition, the mixture was stirred at this temperature for 0.5 hr, and then 1-(3-bromo-4-pyridyl)ethanone (852.6 mg, 4.3 mmol, 1 eq) in 5 mL of THF was added dropwise at −78° C. The resulting mixture was stirred at −78° C. for 0.5 hr. TLC showed the reaction was complete. The reaction mixture was quenched by addition of 100 mL of sat. NH₄Cl, and then diluted with 50 mL of water and extracted twice with 200 mL of ethyl acetate. The combined organic layers were washed with 100 mL of brine, dried over Na₂SO₄, filtered and concentrated under reduced pressure to give a residue, which was purified by column chromatography (SiO₂, eluting with a gradient of petroleum ether/ethyl acetate=1/1 to 0/1) to give crude 1-(3-bromo-4-pyridyl)-1-(5-methoxybenzothiophen-2-yl) ethanol.

### Step 4: 3-bromo-4-[1-(5-methoxybenzothiophen-2-yl)vinyl]pyridine

A mixture of 1-(3-bromo-4-pyridyl)-1-(5-methoxybenzothiophen-2-yl)ethanol (1.25 g, 3.4 mmol, 1 eq) in 5 mL of DCM was cooled to 0° C., triethylsilane (1.0 g, 8.9 mmol, 1.4 mL, 2.6 eq) and TFA (6.5 g, 56.8 mmol, 4.2 mL, 16.6 eq) were added, then the mixture was stirred at 45° C. for 12 hrs. TLC showed the reaction was complete. The reaction mixture was quenched by addition of 50 mL of sat. NaHCO₃ at 0° C., and then diluted with 20 mL of H₂O and extracted twice with 200 mL of ethyl acetate. The combined organic layers were washed with 50 mL of brine, dried over Na₂SO₄, filtered and concentrated under reduced pressure to give a residue, which was purified by column chromatography (SiO₂, eluting with a gradient of petroleum ether/ethyl acetate=1/0 to 4/1) to give crude 3-bromo-4-[1-(5-methoxybenzothiophen-2-yl)vinyl]pyridine.

### Step 5: 4-[1-(5-methoxybenzothiophen-2-yl)vinyl]pyridine-3-carboxylate

A mixture of 3-bromo-4-[1-(5-methoxybenzothiophen-2-yl)vinyl]pyridine (0.1 g, 288.8 μmol, 1 eq), TEA (146.1 mg, 1.4 mmol, 201.0 μL, 5 eq), Pd(dppf)Cl₂ (21.1 mg, 28.9 μmol, 0.1 eq) in 10 mL of MeOH was degassed and purged with CO for 3 times, and then the mixture was stirred at 75° C. for 12 hrs under CO atmosphere (50 Psi). LCMS showed the reaction was complete. The reaction mixture was filtered and concentrated under reduced pressure to give a residue. The residue was purified by prep-TLC (SiO₂, eluting with petroleum ether: ethyl acetate=1:1) to give Methyl4-[1-(5-methoxy benzothiophen-2-yl)vinyl]pyridine-3-carboxylate.

### Step 6: methyl 4-[1-(5-methoxybenzothiophen-2-yl)ethyl]pyridine-3-carboxylate

A mixture of methyl 4-[1-(5-methoxybenzothiophen-2-yl)vinyl]pyridine-3-carboxylate (50 mg, 153.7 μmol, 1 eq), Pd/C (30 mg, 10% purity) in 10 mL of ethyl acetate was degassed and purged with H₂ for 3 times, and then the mixture was stirred at 20° C. for 0.5 hr under H₂ atmosphere (15 Psi). LCMS showed the reaction was complete. The reaction mixture was filtered and concentrated under reduced pressure to give crude methyl 4-[1-(5-methoxybenzothiophen-2-yl)ethyl]pyridine-3-carboxylate.

### Step 7: [4-[1-(5-methoxybenzothiophen-2-yl)ethyl]-3-pyridyl]methanol

A mixture of methyl 4-[1-(5-methoxybenzothiophen-2-yl)ethyl]pyridine-3-carboxylate (100 mg, 305.4 μmol, 1 eq) in 2 mL of DCM was degassed and purged with N₂ for 3 times, then DIBAL-H (1M, 916.32 μL, 3 eq) was added at −78° C., and then the mixture was stirred at −78° C. for 4 hrs under N₂ atmosphere. LCMS showed the reaction was complete. The mixture was cooled to 0° C., 0.04 mL of H₂O and 0.04 mL of 15% NaOH solution was added, followed by addition of 0.1 mL of H₂O, the mixture was stirred at 20° C. for 10 min, filtered and concentrated under reduced pressure to give crude [4-[1-(5-methoxybenzothiophen-2-yl)ethyl]-3-pyridyl]methanol.

### Step 8: 4-[1-(5-methoxybenzothiophen-2-yl)ethyl]pyridine-3-carbaldehyde

A mixture of [4-[1-(5-methoxybenzothiophen-2-yl)ethyl]-3-pyridyl]methanol (40 mg, 133.6 μmol, 1 eq), MnO₂ (58.1 mg, 668.0 μmol, 5 eq) in 1 mL of DCE was degassed and purged with N₂ for 3 times, and then the mixture was stirred at 40° C. for 12 hrs. LCMS showed the reaction was complete. The reaction mixture was filtered and concentrated under reduced pressure to give crude 4-[1-(5-methoxybenzothiophen-2-yl) ethyl]pyridine-3-carbaldehyde.

### Step 9: 9-methoxy-5-methyl-benzothiopheno[2,3-g]isoquinoline

A mixture of 4-[1-(5-methoxybenzothiophen-2-yl)ethyl]pyridine-3-carbaldehyde (40 mg, 134.5 μmol, 1 eq), indium (3+) trifluoromethanesulfonate (3.8 mg, 6.7 μmol, 0.05 eq) in 2 mL of DCE was stirred at 115° C. for 12 hrs. LCMS showed the reaction was complete. The reaction mixture was filtered and concentrated under reduced pressure to give a residue, which was purified by prep-HPLC (FA condition, column: 3-Phenomenex Luna C18 75*30 mm*3 um; mobile phase: [water(0.2% FA)-ACN]; B %: 30%-50%, 10 min) to give 9-methoxy-5-methyl-benzothiopheno[2,3-g]isoquinoline.

MS (ESI): m/z=280.00 [M+H]⁺

### Example 68

### N-(2-(dimethylamino)ethyl)-5,6-dimethyl-6H-pyrido[4,3-b]carbazole-9-carboxamide

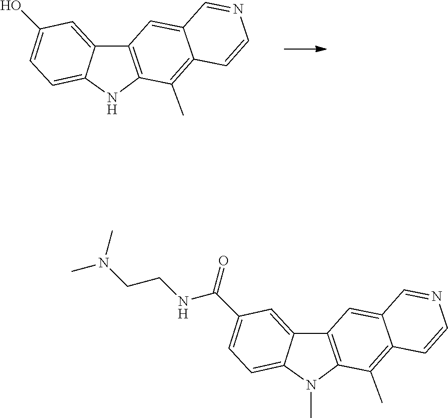 [SMILES: Cc1c2ccncc2cc2c1[nH]c1ccc(O)cc12 ; Cc1c2ccncc2cc2c3cc(C(=O)NCCN(C)C)ccc3n(C)c12]

To a solution of 5,6-dimethyl-6H-pyrido[4,3-b]carbazol-9-ol (52 mg, 0.20 mmol) in DCM (1 ml) and pyridine (25 uL, 0.30 mmol) was added trifluoromethanesulfonic anhydride (56 mg, 0.2 mmol) at 0° C. and warmed up to rt, then stirred overnight at rt. To the mixture was added water and EtOAc, and the org phase was washed with NaHCO₃ aq and brine, dried over Na2SO4 and concentrated. The residue was dissolved in toluene (1 ml) and Pd(dppf)C12 (10 mg, 0.01 mmol) and 2,4,6-trichlorophenylformate (45 mg, 0.2 mmol) were added. DBU (30 uL, 0.2 mmol) was then added and the mixture was heated at 80° C. overnight under N2, then added NH4Cl aq and DCM. The org phase was washed with brine, dried over Na2SO4 and concentrated in vacuo. The residue was dissolved in THF (1 ml) and 2-(dimethylamino)-ethylamine (88 mg, 1.0 mmol) was added and the mixture stirred overnight at rt. The crude product was purified with HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm.) to afford the title compound.

MS (ESI): 361.2 [M+H]⁺

### Examples 69-79

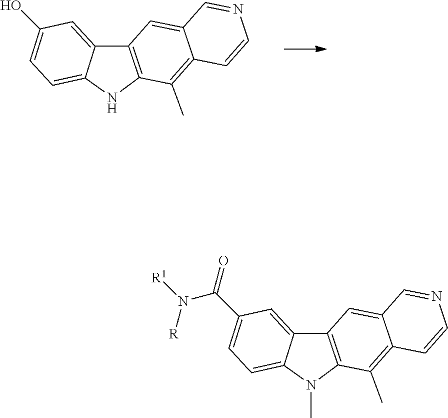

### General Procedure:

To a solution of 5,6-dimethyl-6H-pyrido[4,3-b]carbazol-9-ol (140 mg, 0.53 mmol) in DCM (1 ml) and pyridine (100 uL, 1.20 mmol) was added trifluoromethanesulfonic anhydride (280 mg, 1.0 mmol) at 0° C. and warmed up to rt, then stirred overnight at rt. To the mixture was added water and EtOAc, and the org phase was washed with NaHCO₃ aq and brine, dried over Na2SO4 and concentrated. The residue was dissolved in toluene (5 ml) and Pd(dppf)C12 (50 mg, 0.05 mmol) and 2,4,6-trichlorophenylformate (225 mg, 1.0 mmol) were added. DBU (150 uL, 1.0 mmol) was then added and the mixture was heated at 80° C. overnight under N2, then added NH4Cl aq and DCM. The org phase was washed with brine, dried over Na2SO4 and concentrated in vacuo. The residue was dissolved in THF (1 ml) and the appropriate amine R¹RNH (0.5 mmol) was added and the mixture stirred overnight at rt. The crude product was purified with HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm.) to afford the product as shown in the table below

| Example | | | |
|---|---|---|---|
| # | Compound Name | MS (ESI) | Structure |
| 69 | 5,6-dimethyl-6H-pyrido[4,3- b]carbazole-9-carboxamide | 290.1 [M + H]+ | 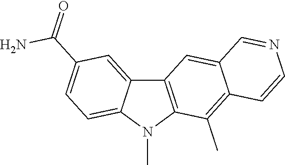 [SMILES: Cc1c2ccncc2cc2c3cc(C(N)=O)ccc3n(C)c12] |
| 70 | N,5,6-trimethyl-6H- pyrido[4,3-b]carbazole-9- carboxamide | 304.1 [M + H]+ | 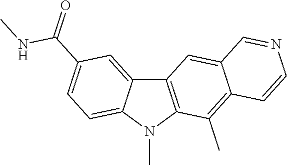 [SMILES: CNC(=O)c1ccc2c(c1)c1cc3cnccc3c(C)c1n2C] |
| 71 | N,N,5,6-tetramethyl-6H- pyrido[4,3-b]carbazole-9- carboxamide | 318.2 [M + H]+ | 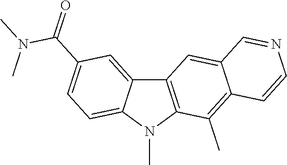 [SMILES: Cc1c2ccncc2cc2c3cc(C(=O)N(C)C)ccc3n(C)c12] |
| 72 | (5,6-dimethyl-6H-pyrido[4,3- b]carbazol-9-yl)(piperazin-1- yl)methanone | 358.2 [M + H]+ | 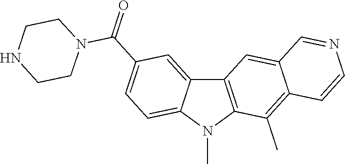 [SMILES: Cc1c2ccncc2cc2c3cc(C(=O)N4CCNCC4)ccc3n(C)c12] |
| 73 | 5,6-dimethyl-N-(2- (methylamino)ethyl)-6H- pyrido[4,3-b]carbazole-9- carboxamide | 347.2 [M + H]+ | 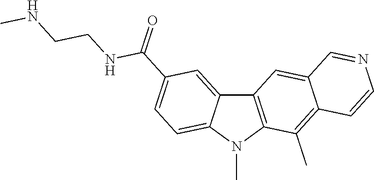 [SMILES: CNCCNC(=O)c1ccc2c(c1)c1cc3cnccc3c(C)c1n2C] |
| 74 | N-(2-aminoethyl)-5,6- dimethyl-6H-pyrido[4,3- b]carbazole-9-carboxamide | 332.2 [M + H]+ | 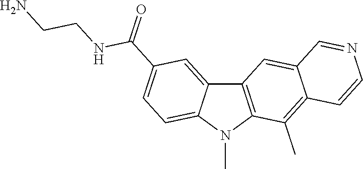 [SMILES: Cc1c2ccncc2cc2c3cc(C(=O)NCCN)ccc3n(C)c12] |
| 75 | 5,6-dimethyl-N-(2-(1- methylpiperidin-4-yl)ethyl)- 6H-pyrido[4,3-b]carbazole- 9-carboxamide | 415.2 [M + H]+ | 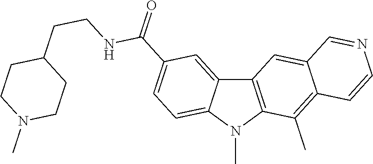 [SMILES: Cc1c2ccncc2cc2c3cc(C(=O)NCCC4CCN(C)CC4)ccc3n(C)c12] |
| 76 | (5,6-dimethyl-6H-pyrido[4,3- b]carbazol-9-yl)(4- methylpiperazin-1- yl)methanone | 373.2 [M + H]+ | 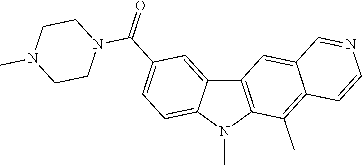 [SMILES: Cc1c2ccncc2cc2c3cc(C(=O)N4CCN(C)CC4)ccc3n(C)c12] |
| 77 | 5,6-dimethyl-N-(2-(piperidin- 1-yl)ethyl)-6H-pyrido[4,3- b]carbazole-9-carboxamide | 401.2 [M + H]+ | 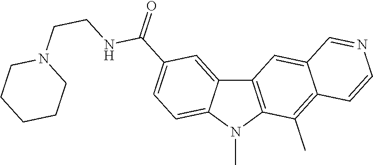 [SMILES: Cc1c2ccncc2cc2c3cc(C(=O)NCCN4CCCCC4)ccc3n(C)c12] |
| 78 | N-(2-hydroxyethyl)-5,6- dimethyl-6H-pyrido[4,3- b]carbazole-9-carboxamide | 334.5 [M + H]+ | 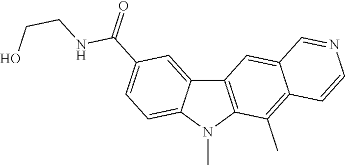 [SMILES: Cc1c2ccncc2cc2c3cc(C(=O)NCCO)ccc3n(C)c12] |
| 79 | N-(2-methoxyethyl)-5,6- dimethyl-6H-pyrido[4,3- b]carbazole-9-carboxamide | 334.1 [M + H]+ | 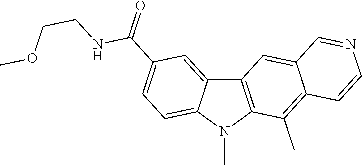 [SMILES: COCCNC(=O)c1ccc2c(c1)c1cc3cnccc3c(C)c1n2C] |

### Example 80

### 1-ethyl-9-methoxy-5,6-dimethyl-6H-pyrido[4,3-b]carbazole (80a) & 9-methoxy-5,6-dimethyl-1-(tetrahydrofuran-2-yl)-6H-pyrido[4,3-b]carbazole (80b)

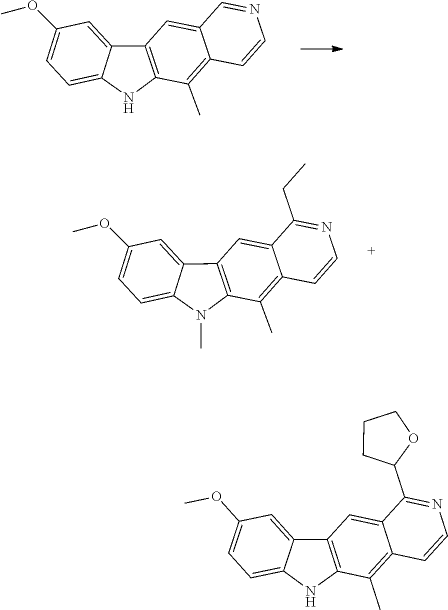 [SMILES: COc1ccc2[nH]c3c(C)c4ccncc4cc3c2c1 ; CCc1nccc2c(C)c3c(cc12)c1cc(OC)ccc1n3C ; COc1ccc2[nH]c3c(C)c4ccnc(C5CCCO5)c4cc3c2c1]

To a solution of 9-methoxy-5-methyl-6H-pyrido[4,3-b]carbazole (14 mg, 0.05 mmol) and Ir(ppy)2(dtbpy)PF6 (1.8 mg, 0.0002 mmol) in DMSO (0.1 ml) was added methyl thioglycolate (2 uL, 0.01 mmol), pTSA (38 mg, 0.2 mmol) and EtOH or THF (50 uL). The mixture was stirred at rt in front of blue LED light overnight. The resulting mixture was purified by HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm.) to afford 1-ethyl-9-methoxy-5,6-dimethyl-6H-pyrido[4,3-b]carbazole and 9-methoxy-5,6-dimethyl-1-(tetrahydrofuran-2-yl)-6H-pyrido[4,3-b]carbazole.

### Example 81

### 4-[2-(5-methylbenzothiopheno[2,3-g]isoquinolin-9-yl)oxyethyl]morpholine

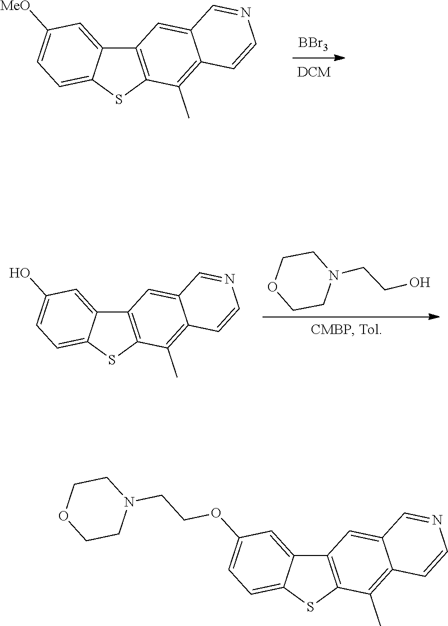

### Step 1. 5-methylbenzothiopheno[2,3-g]isoquinolin-9-ol

To a solution of 9-methoxy-5-methyl-benzothiopheno[2,3-g]isoquinoline (90 mg, 322.1 μmol, 1 eq) in DCM (3 mL) was added BBr3 (161.4 mg, 644.3 μmol, 2 eq) at 0° C. The mixture was stirred at 0° C. for 1 hr. LCMS showed the 9-methoxy-5-methyl-benzothiopheno [2,3-g]isoquinoline remained and the desired ms was detected then the mixture was stirred at 0° C. for another 3 hr, LCMS showed the reaction was complete. The reaction mixture was poured into 50 mL of sat·NaHCO₃ at 0° C., and then diluted with 50 mL of H2O and extracted with 300 mL of ethyl acetate. The combined organic layers were washed with 200 mL of brine, dried over Na2SO4, filtered and concentrated under reduced pressure to give 5-methylbenzothiopheno[2,3-g]isoquinolin-9-ol.

### Step 2. 4-[2-(5-methylbenzothiopheno[2,3-g]isoquinolin-9-yl)oxyethyl]morpholine

A mixture of 2-morpholinoethanol (49.44 mg, 376.89 μmol, 2 eq), 5-methylbenzothiopheno [2,3-g]isoquinolin-9-ol (50 mg, 188.45 μmol, 1 eq), 2-(tributyl-λ⁵-phosphanylidene) acetonitrile (68.22 mg, 282.67 μmol, 1.5 eq) in toluene (2 mL), and then the mixture was stirred at 100° C. for 12 hr. LCMS showed the reaction was completed. The reaction mixture was filtered and concentrated under reduced pressure to give a residue. The residue was purified by prep-HPLC (FA condition, column: 3-Phenomenex Luna C18 75*30 mm*3 um; mobile phase: [water(0.2% FA)-ACN]; B %: 5%-40%, 8 min) to give 4-[2-(5-methylbenzothiopheno[2,3-g]isoquinolin-9-yl)oxyethyl]morpholine.

MS (ESI): m/z=379.00 [M+H]⁺

### Example 82

### (R)—N-(1-(dimethylamino)propan-2-yl)-8-methoxy-5,6-dimethyl-6H-pyrido[4,3-b]carbazole-1-carboxamide

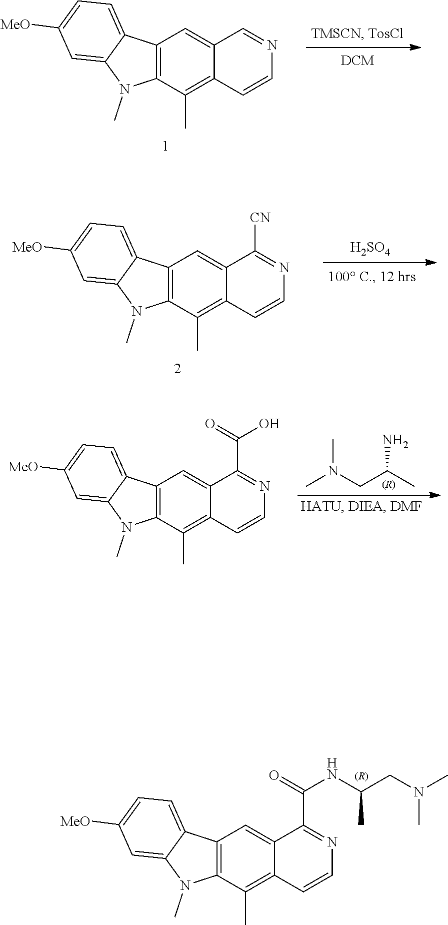

### Step 1: 8-methoxy-5,6-dimethyl-6H-pyrido[4,3-b]carbazole-1-carbonitrile

To a solution of 8-methoxy-5,6-dimethyl-pyrido[4,3-b]carbazole (0.6 g, 2.1 mmol, 1 eq) in DCM (20 mL) was added TMSCN (560.0 mg, 5.6 mmol, 706.2 uL, 2.6 eq). A solution of TosCl (786.5 mg, 4.1 mmol, 1.9 eq) in DCM (2 mL) was dropwsie added to the above mixture at 25° C. The mixture was stirred at 25° C. for 12 hr. 10 mL KOH (50% W/W) was added to the above mixture. The mixture was stirred at 25° C. for 2 hr. TLC showed the reaction was completely. The reaction was extracted with DCM (30 mL×3). The combined organic phase was dried over anhydrous Na₂SO₄, filtered and concentrated to give 8-methoxy-5,6-dimethyl-6H-pyrido[4,3-b]carbazole-1-carbonitrile.

### Step 2: 8-methoxy-5,6-dimethyl-6H-pyrido[4,3-b]carbazole-1-carboxylic acid

The mixture of 8-methoxy-5,6-dimethyl-pyrido[4,3-b]carbazole-1-carbonitrile (600 mg, 1.9 mmol, 1 eq) in H₂SO₄ (6 mL, 98% purity) and H₂O (6 mL) was stirred at 110° C. for 12 hr. LCMS showed some desired product was formed. The reaction was poured into saturated aqueous NaHCO₃ (80 mL) and filtered. The cake was dried to give 8-methoxy-5,6-dimethyl-6H-pyrido[4,3-b]carbazole-1-carboxylic acid.

### Step 3: (R)—N-(1-(dimethylamino)propan-2-yl)-8-methoxy-5,6-dimethyl-6H-pyrido[4,3-b]carbazole-1-carboxamide

To a solution of 8-methoxy-5,6-dimethyl-pyrido[4,3-b]carbazole-1-carboxylic acid (600 mg, 1.8 mmol, 1 eq) in DMF (20 mL) was added DIEA (1.2 g, 9.3 mmol, 1.6 mL, 5 eq) and (2R)—N1,N1-dimethylpropane-1,2-diamine (259.6 mg, 1.8 mmol, 1 eq, HCl). The mixture was cooled to 0° C. and HATU (2.1 g, 5.6 mmol, 3 eq) was added. The mixture was stirred at 25° C. for 12 hr. LCMS showed the reaction was completely. The reaction was concentrated to give a residue. The residue was purified by prep-HPLC (neutral condition, column: Kromasil C18 (250*50 mm*10 um); mobile phase: [water(10 mM NH4HCO3)-ACN]; B %: 35%-55%, 10 min) to afford (R)—N-(1-(dimethylamino)propan-2-yl)-8-methoxy-5,6-dimethyl-6H-pyrido[4,3-b]carbazole-1-carboxamide.

MS (ESI): m/z=405.2 [M+H]⁺

### Example 83

### 7-(4,5-dihydro-1H-imidazol-2-ylmethoxy)-10,11-dimethyl-pyrido[3,4-b]carbazole

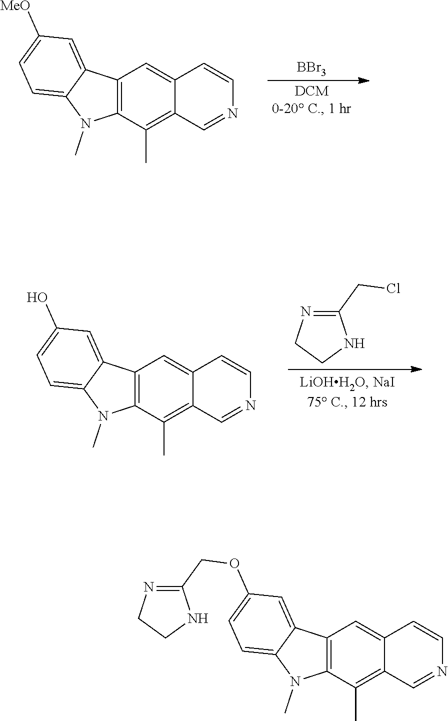 [SMILES: COc1ccc2c(c1)c1cc3ccncc3c(C)c1n2C ; "BBr3" ; "DCM0-20° C., 1 hr" ; Cc1c2cnccc2cc2c3cc(O)ccc3n(C)c12 ; ClCC1=NCCN1 ; Cc1c2cnccc2cc2c3cc(OCC4=NCCN4)ccc3n(C)c12]

### Step 1. 10,11-dimethyl-10H-pyrido[3,4-b]carbazol-7-ol

To a solution of 7-methoxy-10,11-dimethyl-pyrido[3,4-b]carbazole (0.06 g, 217.1 umol, 1 eq) in DCM (4 mL) was added BBr₃ (271.9 mg, 1.0 mmol, 104.6 uL, 5 eq) at 0° C. The mixture was stirred at 20° C. for 1 hr. LC-MS showed 7-methoxy-10,11-dimethyl-10H-pyrido[3,4-b]carbazole was consumed completely and desired mass was detected. The reaction mixture was quenched by addition NaHCO₃ 10 mL then concentrated under reduced pressure to give a residue. Then (DCM:MeOH=10:1) 100 mL was added, filtered and the filtrate was concentrated under reduced pressure to give 10,11-dimethyl-10H-pyrido[3,4-b]carbazol-7-ol.

### Step 2. 7-((4,5-dihydro-1H-imidazol-2-yl)methoxy)-10,11-dimethyl-10H-pyrido[3,4-b]carbazole

To a solution of 2-(chloromethyl)-1H-imidazole; hydrochloride (11.6 mg, 76.2 umol, 1 eq) in DMF (3 mL) was added LiOH·H₂O (12.8 mg, 304.9 umol, 4 eq) and 10,11-dimethylpyrido[3,4-b]carbazol-7-ol (20 mg, 76.2 umol, 1 eq), NaI (11.4 mg, 76.2 umol, 1 eq) was added. The mixture was stirred at 75° C. for 12 hrs. LC-MS showed 10,11-dimethyl-10H-pyrido[3,4-b]carbazol-7-ol was consumed and desired mass was detected. The reaction mixture was concentrated under reduced pressure to remove solvent. The residue was purified by prep-HPLC (FA condition; column: 3-Phenomenex Luna C18 75*30 mm*3 um; mobile phase: [water(0.2% FA)-ACN]; B %: 1%-30%, 8 min) to give 7-(4,5-dihydro-1H-imidazol-2-ylmethoxy)-10,11-dimethyl-pyrido[3,4-b]carbazole.

MS (ESI): m/z=345.0 [M+H]⁺

### Example 84

### 9-(4,5-dihydro-1H-imidazol-2-ylmethoxy)-6-isopropyl-5-methyl-pyrido[4,3-b]carbazole

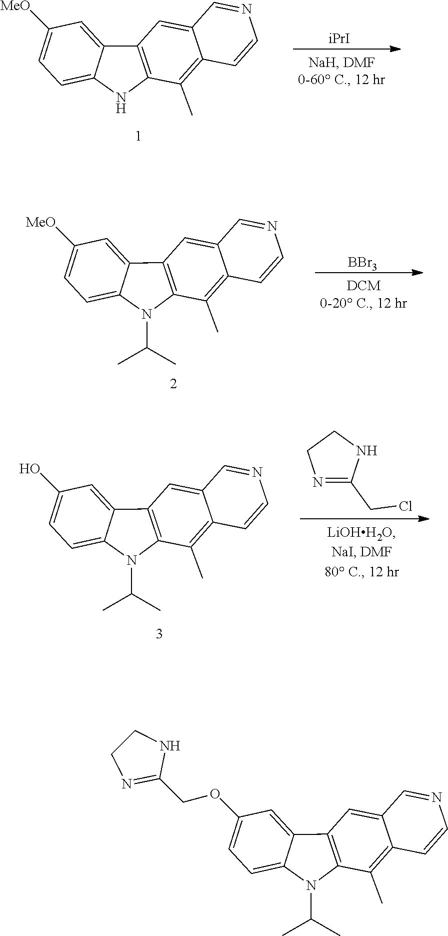

### Step 1: 6-isopropyl-9-methoxy-5-methyl-6H-pyrido[4,3-b]carbazole

To a solution of 9-methoxy-5-methyl-6H-pyrido[4,3-b]carbazole (300 mg, 1.1 mmol, 1 eq) in DMF (30 mL) was added NaH (91.5 mg, 2.3 mmol, 60% purity, 2 eq) at 0° C., then was added 2-iodopropane (583.2 mg, 3.4 mmol, 343.1 uL, 3 eq), the mixture was stirred at 60° C. for 12 hrs. LC-MS showed desired compound was detected. The reaction mixture was quenched by addition NH₄Cl 50 mL, and then extracted with Ethyl acetate (20 mL×3), dried over Na₂SO₄, filtered and concentrated under reduced pressure to give 6-isopropyl-9-methoxy-5-methyl-pyrido[4,3-b]carbazole.

### 6-isopropyl-5-methyl-6H-pyrido[4,3-b]carbazol-9-ol

To a solution of 6-isopropyl-9-methoxy-5-methyl-pyrido[4,3-b]carbazole (200 mg, 657.1 umol, 1 eq) in DCM (3 mL) was at 0° C. added BBr₃ (823.1 mg, 3.3 mmol, 316.5 uL, 5 eq). The mixture was stirred at 20° C. for 12 hrs. LC-MS showed desired compound was detected. The reaction mixture was quenched by addition NaHCO₃ 10 mL, and extracted with DCM (3 mL×3). The combined organic layers were washed with brine (10 mL×2), dried over Na₂SO₄, filtered and concentrated under reduced pressure to give 6-isopropyl-5-methyl-pyrido[4,3-b]carbazol-9-ol.

### Step 2: 9-((4,5-dihydro-1H-imidazol-2-yl)methoxy)-6-isopropyl-5-methyl-6H-pyrido[4,3-b]carbazole

To a solution of 6-isopropyl-5-methyl-pyrido[4,3-b]carbazol-9-ol (30 mg, 103.3 umol, 1 eq) in DMF (3 mL) were added LiOH·H₂O (17.3 mg, 413.3 umol, 4 eq), 2-(chloromethyl)-1H-imidazole (15.8 mg, 103.3 umol, 1 eq, HCl) and NaI (15.5 mg, 103.3 umol, 1 eq). The mixture was stirred at 80° C. for 12 hrs. LC-MS showed desired compound was detected. The residue was purified by prep-HPLC (FA condition; column: 3-Phenomenex Luna C18 75×30 mm×3 um; mobile phase: [water(0.2% FA)-ACN]; B %: 1%-30%, 8 min) to afford 9-(4,5-dihydro-1H-imidazol-2-ylmethoxy)-6-isopropyl-5-methyl-pyrido[4,3-b]carbazole.

MS (ESI): m/z=373.1 [M+H]⁺

### Synthesis of Compounds 194 and 195

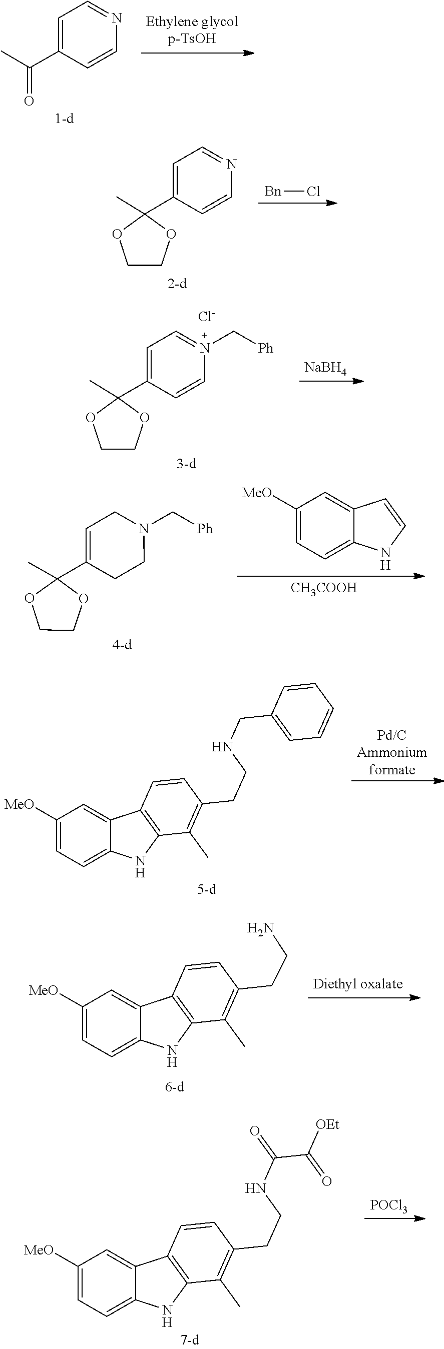

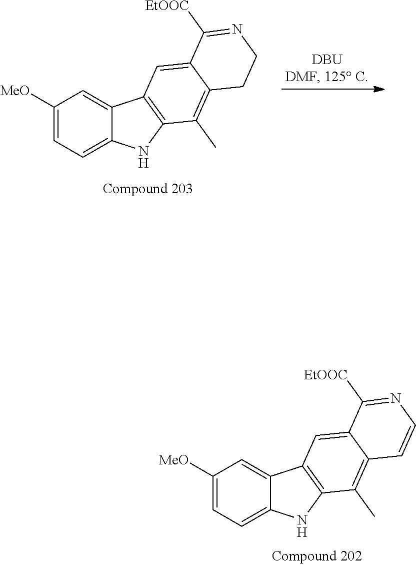 [SMILES: CCOC(=O)C1=NCCc2c1cc1c([nH]c3ccc(OC)cc31)c2C ; "DBUDMF, 125° C." ; CCOC(=O)c1nccc2c(C)c3[nH]c4ccc(OC)cc4c3cc12]

Compounds 1-d-4-d (Besselievre, R. et al. Tetrahedron 1981, 37, 241-246; Alvarez, M. et al. Tetrahedron 1987, 43 (11), 2513-2522) and 5-d, 7-d and Compound 203 (Jasztold-Howorko, R. et al. J Med Chem 1994, 37 (15), 2445-2452) were synthesized as previously reported.

2-(6-methoxy-1-methyl-9H-carbazol-2-yl)ethan-1-amine (6-d). Pd—C (10 wt. %, 192 mg, 0.18 mmol, 0.2 eq) and ammonium formate (255 mg, 4.05 mmol, 4.5 eq) were added to a solution of 5-d in MeOH (25 mL). The reaction mixture was heated to 70° C. and stirred for 1 hour before it was filtered over Celite and concentrated in vacuo. The crude product was purified by flash chromatography (DCM/MeOH 9:1, 1% NH₄OH) providing compound 6-d (167 mg) in 73% yield. ¹H NMR (400 MHz, DMSO) δ 10.85 (s, 1H), 7.82 (d, J=7.9 Hz, 1H), 7.59 (d, J=2.5 Hz, 1H), 7.38 (d, J=8.7 Hz, 1H), 6.98 (dd, J=2.5, 8.7 Hz, 1H), 6.94 (d, J=8.0 Hz, 1H), 3.84 (s, 3H), 2.89-2.83 (m, 2H), 2.82-2.75 (m, 2H), 2.49 (s, 3H). The spectral characteristics are identical to those previously reported (Jasztold-Howorko, R. (1994)).

### Ethyl-9-methoxy-5-methyl-6H-pyrido[4,3-b]carbazole-1-carboxylate (Compound 202)

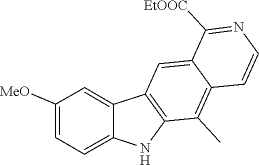 [SMILES: CCOC(=O)c1nccc2c(C)c3[nH]c4ccc(OC)cc4c3cc12]

DBU (131 μL, 1.41 mmol, 2.0 eq) was added to a solution of Compound 203 (250 mg, 0.705 mmol, 1.0 eq) in DMF (2.3 mL). The reaction mixture was heated to 125° C. and stirred for 90 min. The solvent was evaporated and the concentrated reaction mixture was diluted with DCM/MeOH 9:1 (20 mL) and H₂O (15 mL). The product was extracted with DCM/MeOH 9:1 three times (15 mL), the combined organic extracts were dried over Na₂SO₄, filtered and the solvent was removed under reduced pressure. The crude product was purified by flash chromatography (1-10% MeOH in DCM, linear gradient) to provide Compound 202 (105 mg) in 44% yield. ¹H NMR (700 MHz, MeOD) δ 9.26 (s, 1H), 8.30 (dd, J=1.9, 6.6 Hz, 1H), 8.19 (dd, J=1.9, 6.6 Hz, 1H), 7.29 (d, J=8.6 Hz, 1H), 7.16-7.11 (m, 1H), 4.75 (q, J=7.2 Hz, 1H), 3.92 (s, 3H), 2.68 (s, 3H), 1.64 (t, J=7.2 Hz, 3H). ¹³C NMR (176 MHz, MeOD) δ 161.24, 154.75, 143.93, 141.47, 137.32, 134.76, 129.05, 128.72, 122.14, 121.37, 118.80, 118.07, 117.31, 111.73, 111.65, 103.96, 63.79, 54.95, 13.05, 11.02.

### Ethyl-9-methoxy-5,6-dimethyl-6H-pyrido[4,3-b]carbazole-1-carboxylate (Compound 204)

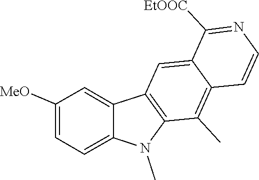 [SMILES: CCOC(=O)c1nccc2c(C)c3c(cc12)c1cc(OC)ccc1n3C]

Compound 202 (120 mg, 0.359 mmol, 1.0 eq) was dissolved in DMF (1.8 mL) and cooled to 0° C. After the addition of NaH (60% suspension in mineral oil, 19 mg, 0.467 mmol, 1.3 eq) the resulting suspension was stirred for 30 min at 0° C. before methyl iodide (25 μL, 0.395 mmol, 1.1 eq) was added. The cooling bath was removed and the reaction mixture was stirred at room temperature for 12 hours. After the dilution with DCM (15 mL) and H₂O (10 mL) the product was extracted three times with DCM (10 mL). The combined organic extracts were dried over Na2SO4, filtered and the solvent was removed under reduced pressure. The crude product was purified by flash chromatography (1-10% MeOH in DCM, linear gradient) providing Compound 204 (73 mg) in 58% yield. ¹H NMR (400 MHz, CDCl₃) δ 9.51 (s, 1H), 8.62 (d, J=6.5 Hz, 1H), 8.28 (d, J=6.5 Hz, 1H), 7.72 (d, J=2.4 Hz, 1H), 7.34 (d, J=8.8 Hz, 1H), 7.29-7.22 (m, 1H), 4.68 (q, J=7.2 Hz, 2H), 4.17 (s, 3H), 3.98 (s, 3H), 3.12 (s, 3H), 1.57 (t, J=7.2 Hz, 3H). ¹³C NMR (101 MHz, CDCl₃) δ 166.98, 154.21, 141.80, 139.71, 139.51, 135.02, 127.51, 122.97, 121.09, 119.06, 117.05, 115.41, 111.11, 109.45, 103.99, 62.15, 56.21, 33.61, 14.56, 13.85.

### (R)—N-(1-(dimethylamino)propan-2-yl)-9-methoxy-5-methyl-6H-pyrido[4,3-b]carbazole-1-carboxamide (Compound 194)

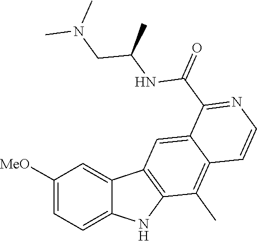 [SMILES: COc1ccc2[nH]c3c(C)c4ccnc(C(=O)N[C@H](C)CN(C)C)c4cc3c2c1]

To a solution of Compound 202 (25 mg, 0.075 mmol, 1.0 eq) in EtOH (1.5 mL) was added NaOH (225 μL, 0.171 mmol, 3.0 eq) and the mixture was refluxed for 90 min at 83° C. After neutralization with HCl (1M) the reaction mixture was concentrated in vacuo and lyophilized to give the corresponding acid in quantitative yield.

The acid (21.5 mg, 0.038 mmol, 1.0 eq) was dissolved in DMF (0.5 mL) and EDC (11 mg, 0.056 mmol, 1.5 eq), HOBt (7.6 mg, 0.056 mmol, 1.5 eq) and DIPEA (19 μL, 0.113 mmol, 3.0 eq) were added. The mixture was stirred for 5 min at room temperature before (R)—N¹,N¹-dimethylpropane-1,2-diamine (7.7 mg, 0.075 mmol, 2.0 eq) was added. After stirring for 14 hours at room temperature the solvent was evaporated and the product was purified by reversed phase flash chromatography (0-100% MeOH in H₂O, 0.1% TFA, linear gradient) providing Compound 194 (15 mg) in 79% yield. ¹H NMR (700 MHz, MeOD) δ 9.17 (s, 1H), 8.35 (d, J=7.1 Hz, 1H), 8.29 (d, J=6.7 Hz, 1H), 7.76 (dd, J=3.8, 7.3 Hz, 1H), 7.51-7.43 (m, 1H), 7.23 (d, J=8.6 Hz, 1H), 3.95 (s, 3H), 3.54-3.48 (m, 1H), 3.45-3.38 (m, 1H), 3.12 (s, 7H), 3.03-2.90 (m, 1H), 2.89-2.78 (m, 3H), 1.51 (d, J=6.6 Hz, 2H). ¹³C NMR (176 MHz, MeOD) δ 165.24, 154.69, 150.29, 143.72, 137.65, 134.06, 131.87, 128.07, 122.66, 119.60, 118.51, 117.64, 116.74, 111.68, 104.20, 61.87, 55.04, 41.64, 17.35, 10.94.

### (R)—N-(1-(dimethylamino)propan-2-yl)-9-methoxy-5,6-dimethyl-6H-pyrido[4,3-b]carbazole-1-carboxamide (Compound 212)

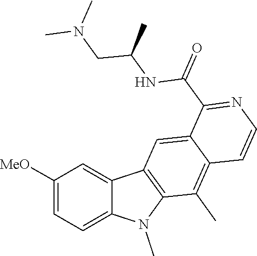 [SMILES: COc1ccc2c(c1)c1cc3c(C(=O)N[C@H](C)CN(C)C)nccc3c(C)c1n2C]

To a solution of Compound 204 (20 mg, 0.057 mmol, 1.0 eq) in EtOH (1.5 mL) was added NaOH (171 μL, 0.171 mmol, 3.0 eq) and the mixture was refluxed for 90 min at 83° C. After neutralization with HCl (1M) the reaction mixture was concentrated in vacuo and lyophilized to give the corresponding acid in quantitative yield. The acid (10 mg, 0.022 mmol, 1.0 eq) was dissolved in DMF (0.5 mL) and EDC (6.3 mg, 0.033 mmol, 1.5 eq), HOBt (4.4 mg, 0.033 mmol, 1.5 eq) and DIPEA (11 μL, 0.066 mmol, 3.0 eq) were added. The mixture was stirred for 5 min at room temperature before (R)—N¹,N¹-dimethylpropane-1,2-diamine (4.5 mg, 0.044 mmol, 2.0 eq) was added. After stirring for 20 hours at room temperature the solvent was evaporated and the product was purified by reversed phase flash chromatography (0-100% MeOH in H₂O, 0.1% TFA, linear gradient) providing Compound 212 (7.2 mg) in 63% yield. ¹H NMR (700 MHz, MeOD) δ 9.05 (s, 1H), 8.31 (d, J=6.0 Hz, 1H), 8.23 (d, J=6.1 Hz, 1H), 7.64 (s, 1H), 7.32 (dd, J=1.9, 8.8 Hz, 1H), 7.22-7.13 (m, 1H), 4.00 (s, 3H), 3.93 (s, 3H), 3.54-3.48 (m, 1H), 3.45-3.39 (m, 1H), 3.29-3.02 (m, 7H), 2.96 (s, 3H), 1.50 (d, J=6.8 Hz, 2H). ¹³C NMR (176 MHz, MeOD) δ 164.98, 154.84, 149.66, 143.20, 139.43, 135.16, 132.15, 128.38, 121.92, 119.65, 118.50, 117.23, 116.48, 112.16, 109.86, 103.80, 61.77, 55.03, 41.60, 32.53, 17.40, 12.57.

### (R)—N-(1-(dimethylamino)propan-2-yl)-9-hydroxy-5,6-dimethyl-6H-pyrido[4,3-b]carbazole-1-carboxamide (Compound 195)

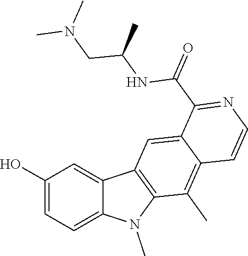 [SMILES: Cc1c2ccnc(C(=O)N[C@H](C)CN(C)C)c2cc2c3cc(O)ccc3n(C)c12]

A solution of Compound 212 (14 mg, 0.028 mmol, 1.0 eq) in DCM (1.2 mL) was cooled to −70° C. and BBr₃ (1M in DCM, 280 μL, 0.28 mmol, 10 eq) was added. The reaction mixture was allowed to come to room temperature over 5 hours and stirring was continued for further 12 hours. The reaction was quenched by pouring into ice-water (ca. 5 mL) and subsequent basification with NEt₃. After stirring for 3 hours at room temperature the phases were separated and the product was extracted with DCM (3×10 mL). The combined organic extracts were dried over Na₂SO₄, filtered and the solvent was removed under reduced pressure. The crude product was purified by preparative HPLC (linear gradient 10-100% acetonitrile/MeOH=1:1, 0.1% TFA, 10 min) providing Compound 195 (7.7 mg) in 55% yield. ¹H NMR (400 MHz, MeOD) δ 9.09 (s, 1H), 8.42 (d, J=6.6 Hz, 1H), 8.34 (d, J=6.6 Hz, 1H), 7.61 (d, J=2.4 Hz, 1H), 7.37 (d, J=8.8 Hz, 1H), 7.20 (dd, J=2.4, 8.8 Hz, 1H), 4.12 (s, 3H), 3.65-3.49 (m, 2H), 3.22 (bs, 7H), 3.10 (s, 3H), 1.60 (d, J=6.7 Hz, 3H).

Compounds 213-215

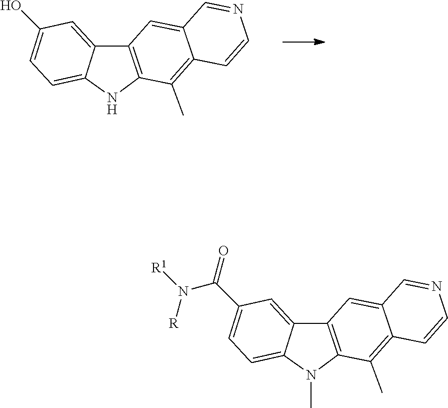

General Procedure

To a solution of 5,6-dimethyl-6H-pyrido[4,3-b]carbazol-9-ol (140 mg, 0.53 mmol) in DCM (1 ml) and pyridine (100 μL, 1.20 mmol) was added trifluoromethanesulfonic anhydride (280 mg, 1.0 mmol) at 0° C. The mixture was warmed up to room temperature, then stirred overnight at room temperature. To the mixture was added water and EtOAc, and the organic phase was washed with aqueous NaHCO₃ and brine, dried over Na₂SO₄ and concentrated. The residue was dissolved in toluene (5 ml) and Pd(dppf)Cl₂ (50 mg, 0.05 mmol) and 2,4,6-trichlorophenylformate (225 mg, 1.0 mmol) were added. DBU (150 μL, 1.0 mmol) was then added and the mixture was heated at 80° C. overnight under nitrogen. Aqueous NH₄Cl and DCM were then added and the organic phase was washed with brine, dried over Na₂SO₄ and concentrated in vacuo. The residue was dissolved in THF (1 ml) and the appropriate amine R¹RNH (0.5 mmol) was added and the mixture stirred overnight at room temperature. The crude product was purified with HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm.) to afford the title compounds as shown in the table below:

| Compound | | |
|---|---|---|
| # | Compound Name | Structure |
| 213 | 5,6-dimethyl-N-(2-(piperidin-1- yl)ethyl)-6H-pyrido[4,3- b]carbazole-9-carboxamide | 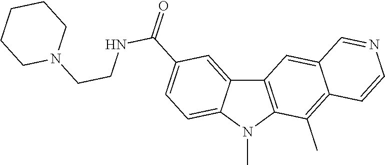 [SMILES: Cc1c2ccncc2cc2c3cc(C(=O)NCCN4CCCCC4)ccc3n(C)c12] |
| 214 | N-(2-hydroxyethyl)-5,6-dimethyl- 6H-pyrido[4,3-b]carbazole-9- carboxamide | 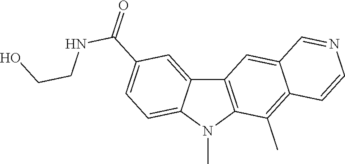 [SMILES: Cc1c2ccncc2cc2c3cc(C(=O)NCCO)ccc3n(C)c12] |
| 215 | N-(2-methoxyethyl)-5,6-dimethyl- 6H-pyrido[4,3-b]carbazole-9- carboxamide | 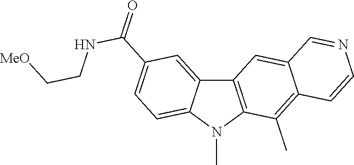 [SMILES: COCCNC(=O)c1ccc2c(c1)c1cc3cnccc3c(C)c1n2C] |

Analytical Data for Compounds 21-215:
213: MS (ESI): 401.2 [M+H]+
214: MS (ESI): 334.5 [M+H]+
215: MS (ESI): 334.1 [M+H]+

### N-(2-(dimethylamino)ethyl)-5,6-dimethyl-1-(tetrahydrofuran-2-yl)-6H-pyrido[4,3-b]carbazole-9-carboxamide (Compound 216)

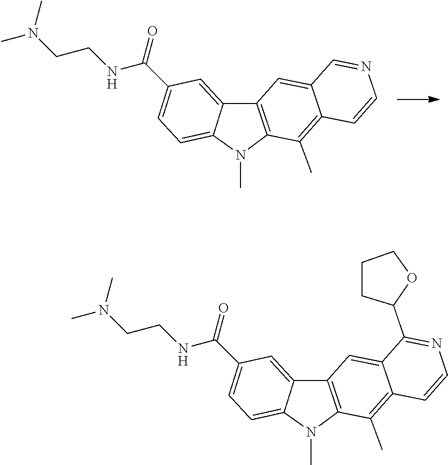 [SMILES: Cc1c2ccncc2cc2c3cc(C(=O)NCCN(C)C)ccc3n(C)c12 ; Cc1c2ccnc(C3CCCO3)c2cc2c3cc(C(=O)NCCN(C)C)ccc3n(C)c12]

To a solution of N-(2-(dimethylamino)ethyl)-5,6-dimethyl-6H-pyrido[4,3-b]carbazole-9-carboxamide (3.6 mg, 0.01 mmol) in water (0.1 ml) and MeCN (0.1 ml) was added NHS (11.5 mg, 0.1 mmol) and NH₄S₂O₈ (23 mg, 0.1 mmol) at room temperature and the mixture was stirred overnight. The residue was purified by HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H₂O and B=MeOH or ACN; Flow rate: 5 mL/min, Injection volume: 4 mL, Runtime: 60 min, gradient: variable concentrations of B in A; UV detection at 220 & 345 nm.) to afford the desired product.

MS (ESI): 431.2 [M+H]+

### 4-(2-((5,6-dimethyl-6H-pyrido[4,3-b]carbazol-9-yl)oxy)ethyl)thiomorpholine 1,1-dioxide (Compound 217)

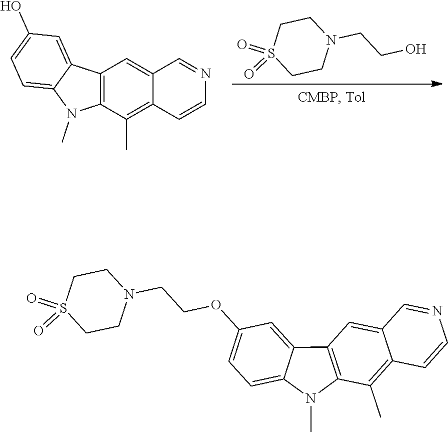

To a solution of 5,6-dimethylpyrido[4,3-b]carbazol-9-ol (50 mg, 190.6 μmol, 1 eq) in toluene (2 mL) was added 2-(1,1-dioxo-1,4-thiazinan-4-yl)ethanol (41.0 mg, 228.7 μmol, 1.2 eq) and 2-(tributyl-λ5-phosphanylidene)acetonitrile (55.2 mg, 228.7 μmol, 1.2 eq) under N₂. The mixture was stirred at 100° C. for 12 hr. LCMS showed the reaction was complete. The reaction was concentrated to give a residue, which was purified by prep-HPLC (TFA condition; column: Phenomenex Gemini-NX 150*30 mm*5 um; mobile phase: [water(0.1% TFA)-ACN]; B %: 15%-45%, 9 min) to afford 4-(2-((5,6-dimethyl-6H-pyrido[4,3-b]carbazol-9-yl)oxy)ethyl)thiomorpholine 1,1-dioxide.

MS (ESI): m/z=424.1 [M+H]⁺

### (S)-1-((5,6-dimethyl-6H-pyrido[4,3-b]carbazol-9-yl)oxy)propan-2-amine (Compound 218)

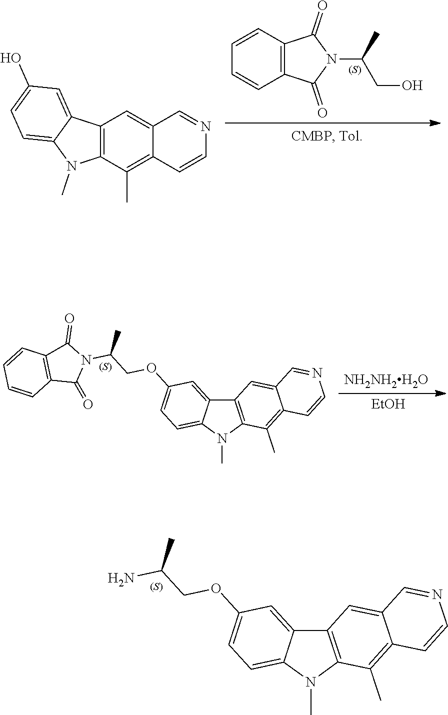

### Step 1: (S)-2-(1-((5,6-dimethyl-6H-pyrido[4,3-b]carbazol-9-yl)oxy)propan-2-yl)isoindoline-1,3-dione

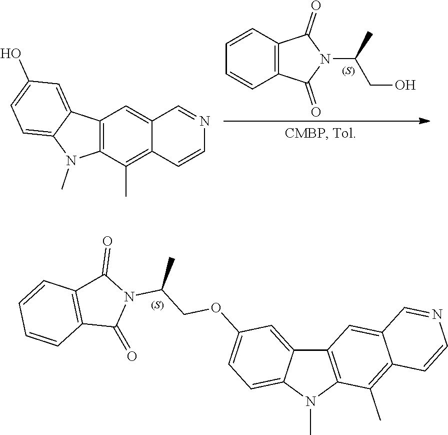

To a solution of 5,6-dimethylpyrido[4,3-b]carbazol-9-ol (106.5 mg, 406.0 μmol, 1 eq) in toluene (4 mL) was added 2-[(1S)-2-hydroxy-1-methyl-ethyl]isoindoline-1,3-dione (100 mg, 487.3 μmol, 1.2 eq) and 2-(tributyl-λ5-phosphanylidene)acetonitrile (117.6 mg, 487.3 μmol, 1.2 eq) under N₂ atmosphere. The mixture was stirred at 100° C. for 2 hr under N₂ atmosphere. LCMS showed the reaction was complete. The reaction mixture was concentrated under reduced pressure to remove solvent. The residue was purified by prep-TLC (SiO₂, DCM/MeOH=10/1) to afford (S)-2-(1-((5,6-dimethyl-6H-pyrido[4,3-b]carbazol-9-yl)oxy)propan-2-yl)isoindoline-1,3-dione.

### Step 2: (S)-1-((5,6-dimethyl-6H-pyrido[4,3-b]carbazol-9-yl)oxy)propan-2-amine

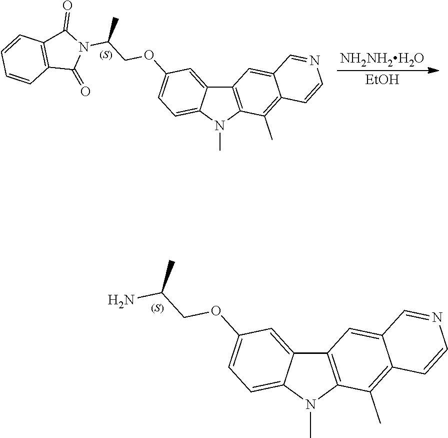

To a solution of (S)-2-(1-((5,6-dimethyl-6H-pyrido[4,3-b]carbazol-9-yl)oxy)propan-2-yl) isoindoline-1,3-dione (150 mg, 333.7 μmol, 1 eq) in EtOH (4 mL) was added N₂H₄·H₂O (34.0 mg, 667.4 μmol, 33.1 μL, 98% purity, 2 eq). The mixture was stirred at 80° C. for 12 hr. LCMS showed the reaction was complete. The reaction was concentrated to give a residue, which was dissolved in DCM (10 mL) and filtered. The filtrate was concentrated to give the crude product (140 mg) as a yellow solid. The residue (60 mg) was purified by prep-HPLC (TFA condition; column: Phenomenex Gemini-NX 150*30 mm*5 um; mobile phase: [water(0.1% TFA)-ACN]; B %: 4%-34%, 9 min) to afford (S)-1-((5,6-dimethyl-6H-pyrido[4,3-b]carbazol-9-yl)oxy)propan-2-amine.

MS (ESI): m/z=320.1 [M+H]⁺

### 9-(2-(azepan-1-yl)ethoxy)-5,6-dimethyl-6H-pyrido[4,3-b]carbazole (Compound 219)

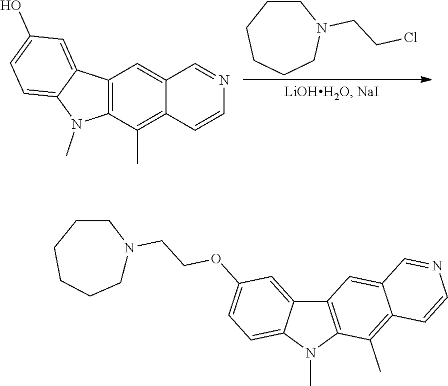

To a solution of 5,6-dimethylpyrido[4,3-b]carbazol-9-ol (50 mg, 190.6 μmol, 1 eq) in DMF (2 mL) was added 1-(2-chloroethyl)azepane (36.9 mg, 228.7 μmol, 1.2 eq), LiOH·H₂O (32.0 mg, 762.4 μmol, 4 eq) and NaI (28.5 mg, 190.6 μmol, 1 eq). The mixture was stirred at 80° C. for 12 hr. LCMS showed the reaction was complete. The reaction was concentrated to give a residue, which was purified by prep-HPLC (TFA condition; column: Phenomenex luna C18 100*40 mm*5 μm; mobile phase: [water(0.1% TFA)-ACN]; B %: 5%-38%, 8 min) to afford 9-(2-(azepan-1-yl)ethoxy)-5,6-dimethyl-6H-pyrido[4,3-b]carbazole.

MS (ESI): m/z=388.1 [M+H]⁺

### 5,6-dimethyl-6H-pyrido[4,3-b]carbazol-7-ol (Compound 220)

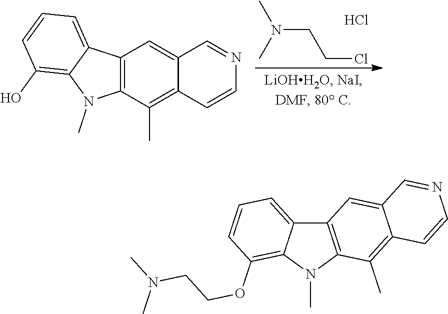

To a solution of 5,6-dimethyl-6H-pyrido[4,3-b]carbazol-7-ol (35 mg, 133.4 μmol, 1 eq) in DMF (5 mL) was added 2-chloro-N,N-dimethyl-ethanamine; hydrochloride (23.0 mg, 160.1 μmol, 1.2 eq), LiOH·H₂O (22.4 mg, 533.7 μmol, 4 eq) and NaI (20.0 mg, 133.4 μmol, 1 eq). The mixture was stirred at 80° C. for 12 hrs. LC-MS showed desired compound was detected. The reaction mixture was concentrated under reduced pressure to give a residue, which was purified by prep-HPLC (neutral condition; column: Waters Xbridge Prep OBD C18 150*40 mm*10 um; mobile phase: [water(0.05% NH₃H2O+10 mM NH₄HCO₃)-ACN]; B %: 30%-65%, 8 min.) to give 2-((5,6-dimethyl-6H-pyrido[4,3-b]carbazol-7-yl)oxy)-N,N-dimethylethanamine.

MS (ESI): m/z=334.1 [M+H]⁺

### 9-(2-(azetidin-1-yl)ethoxy)-5,6-dimethyl-6H-pyrido[4,3-b]carbazole (Compound 221)

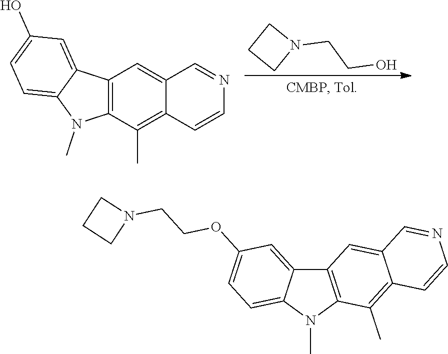

To a solution of 5,6-dimethylpyrido[4,3-b]carbazol-9-ol (50 mg, 190.6 μmol, 1 eq) in toluene (2 mL) was added 2-(azetidin-1-yl)ethanol (23.1 mg, 228.7 μmol, 1.2 eq) and 2-(tributyl-λ5-phosphanylidene)acetonitrile (55.2 mg, 228.7 μmol, 1.2 eq) under N₂. The mixture was stirred at 100° C. for 12 hr. LCMS showed the reaction was complete. The reaction was concentrated to give a residue. The residue was purified by prep-HPLC (TFA condition; column: Phenomenex luna C18 100*40 mm*5 um; mobile phase: [water(0.1% TFA)-ACN]; B %: 1%-40%, 8 min) to afford 9-(2-(azetidin-1-yl)ethoxy)-5,6-dimethyl-6H-pyrido[4,3-b]carbazole.

MS (ESI): m/z=346.1 [M+H]⁺

### (R)—N-(1-(dimethylamino)propan-2-yl)-5,6-dimethyl-8-(3-(4-methylpiperazin-1-yl)-propoxy)-6H-pyrido[4,3-b]carbazole-1-carboxamide (Compound 222)

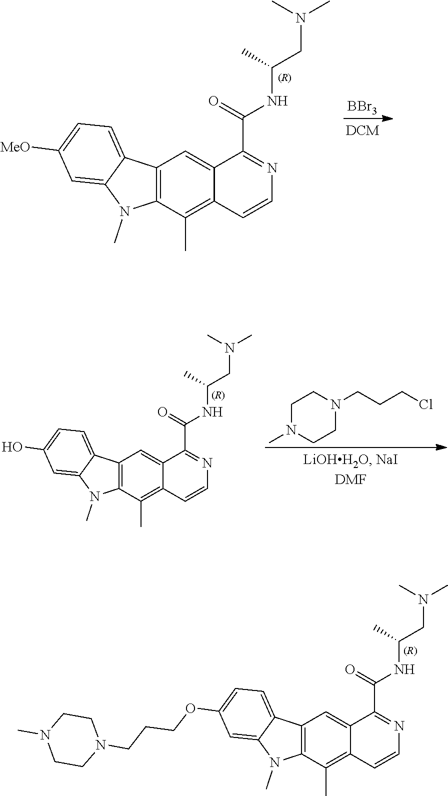

### Step 1. (R)—N-(1-(dimethylamino)propan-2-yl)-8-hydroxy-5,6-dimethyl-6H-pyrido[4,3-b]carbazole-1-carboxamide

To a solution of N-[(1R)-2-(dimethylamino)-1-methyl-ethyl]-8-methoxy-5,6-dimethyl-pyrido[4,3-b]carbazole-1-carboxamide (50 mg, 123.6 μmol, 1 eq) in DCM (2 mL) was added BBr₃ (154.8 mg, 618.0 μmol, 59.5 μL, 5 eq) at 0° C. The mixture was stirred at 25° C. for 1 hr. LCMS showed the reaction was complete. The reaction was poured into NH₃·H₂O (25% purity, 2 mL) and extracted with DCM (2 mL×3). The combined organic phase was dried over anhydrous Na₂SO₄, filtered and concentrated to give (R)—N-(1-(dimethylamino)propan-2-yl)-8-hydroxy-5,6-dimethyl-6H-pyrido[4,3-b]carbazole-1-carboxamide.

### Step 2. (R)—N-(1-(dimethylamino)propan-2-yl)-5,6-dimethyl-8-(3-(4-methylpiperazin-1-yl)propoxy)-6H-pyrido[4,3-b]carbazole-1-carboxamide

To a solution of (R)—N-(1-(dimethylamino)propan-2-yl)-8-hydroxy-5,6-dimethyl-6H-pyrido[4,3-b]carbazole-1-carboxamide (30 mg, 76.8 μmol, 1 eq) in DMF (2 mL) was added 1-(3-chloropropyl)-4-methyl-piperazine (23.0 mg, 92.1 μmol, 1.2 eq, 2 HCl), LiOH·H₂O (12.9 mg, 307.3 μmol, 4 eq) and NaI (11.5 mg, 76.8 μmol, 1 eq). The mixture was stirred at 80° C. for 12 hr. LCMS showed the reaction was complete. The reaction was concentrated to give a residue, which was purified by prep-HPLC (TFA condition; column: Phenomenex luna C18 100*40 mm*5 μm; mobile phase: [water(0.1% TFA)-ACN]; B %: 1%-40%, 8 min) to afford (R)—N-(1-(dimethylamino)propan-2-yl)-5,6-dimethyl-8-(3-(4-methylpiperazin-1-yl)propoxy)-6H-pyrido[4,3-b]carbazole-1-carboxamide.

MS (ESI): m/z=531.2 [M+H]⁺

### 9-(2-(4,4-dimethylpiperidin-1-yl)ethoxy)-5,6-dimethyl-6H-pyrido[4,3-b]carbazole (Compound 223)

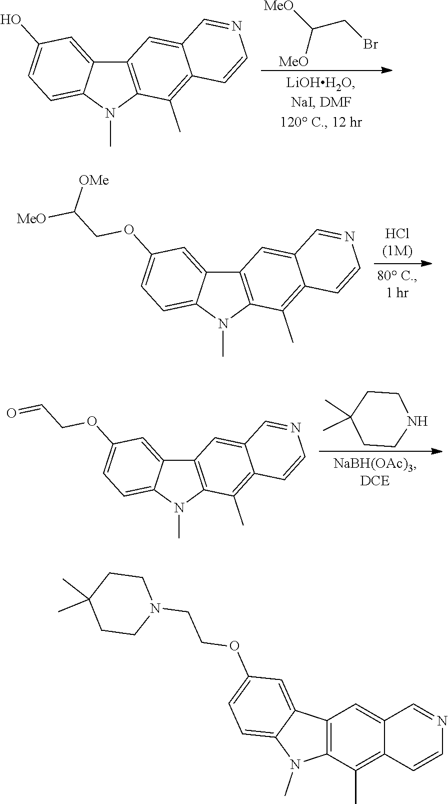

### Step 1: 9-(2,2-dimethoxyethoxy)-5,6-dimethyl-6H-pyrido[4,3-b]carbazole

To a solution of 5,6-dimethylpyrido[4,3-b]carbazol-9-ol (100 mg, 381.2 μmol, 1 eq) in DMF (4 mL) was added 2-bromo-1,1-dimethoxy-ethane (128.8 mg, 762.4 μmol, 89.4 μL, 2 eq), LiOH·H₂O (63.9 mg, 1.5 mmol, 4 eq) and NaI (57.1 mg, 381.2 μmol, 1 eq). The mixture was stirred at 120° C. for 12 hr. LCMS showed the desired product was formed. The reaction was concentrated to give a residue. The residue was purified by prep-TLC (SiO₂, Petroleum ether/Ethyl acetate=0/1) to afford 9-(2,2-dimethoxyethoxy)-5,6-dimethyl-6H-pyrido[4,3-b]carbazole.

### Step 2: 2-((5,6-dimethyl-6H-pyrido[4,3-b]carbazol-9-yl)oxy)acetaldehyde

A solution of 9-(2,2-dimethoxyethoxy)-5,6-dimethyl-pyrido[4,3-b]carbazole (60.0 mg, 171.2 μmol, 1 eq) in THF (1.5 mL) and HCl (1 M) (1 mL) was heated to 80° C. for 2 hr. LCMS showed the reaction was complete. The reaction was added to CH₃CN (10 mL) and yellow solid was precipitated. The solid was filtered and washed with CH₃CN (2 mL×3) to give 2-((5,6-dimethyl-6H-pyrido[4,3-b]carbazol-9-yl)oxy)acetaldehyde.

### Step 3: 9-(2-(4,4-dimethylpiperidin-1-yl)ethoxy)-5,6-dimethyl-6H-pyrido[4,3-b]carbazole

To a solution of 2-(5,6-dimethylpyrido[4,3-b]carbazol-9-yl)oxyacetaldehyde (40 mg, 131.4 μmol, 1 eq) in DCE (2 mL) was added 4,4-dimethylpiperidine (23.6 mg, 157.7 μmol, 25.5 μL, 1.2 eq, HCl) and adjusted pH to 7 with TEA, and then added AcOH to adjusted pH to 5. NaBH(OAc)₃ (111.4 mg, 525.7 μmol, 4 eq) was added to above mixture and the mixture was stirred at 45° C. for 12 hr. LCMS showed the reaction was complete. The reaction was concentrated to give a residue, which was purified by prep-HPLC (TFA condition; column: Phenomenex Gemini-NX 150*30 mm*5 μm; mobile phase: [water(0.1% TFA)-ACN]; B %: 4%-34%, 9 min). The residue was purified by prep-HPLC (neutral condition; column: Waters Xbridge BEH C18 100*25 mm*5 μm; mobile phase: [water(10 mM NH4HCO3)-ACN]; B %: 55%-90%, 10 min) to afford 9-(2-(4,4-dimethylpiperidin-1-yl)ethoxy)-5,6-dimethyl-6H-pyrido[4,3-b]carbazole.

MS (ESI): m/z=402.1 [M+H]⁺

### N,N-dimethyl-2-((5-methylbenzo[4,5]thieno[2,3-g]isoquinolin-9-yl)oxy)ethanamine (Compound 224)

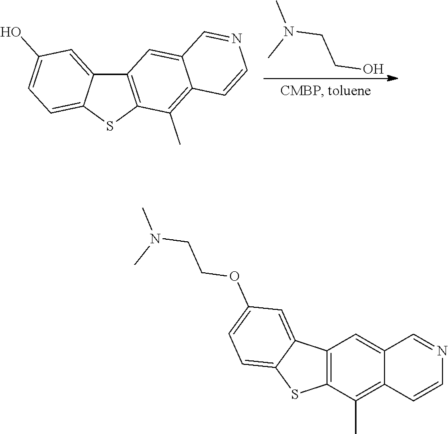

To a solution of 5-methylbenzothiopheno[2,3-g]isoquinolin-9-ol (50 mg, 188.4 μmol, 1 eq) in toluene (2 mL) was added 2-(dimethylamino)ethanol (20.1 mg, 226.1 μmol, 22.7 μL, 1.2 eq) and 2-(tributyl-λ5-phosphanylidene)acetonitrile (54.5 mg, 226.1 μmol, 1.2 eq) under N₂. The mixture was stirred at 100° C. for 12 hr. LCMS showed the reaction was complete. The reaction was concentrated to give a residue, which was purified by prep-HPLC (TFA condition; column: Phenomenex luna C18 100*40 mm*5 μm; mobile phase: [water(0.1% TFA)-ACN]; B %: 1%-36%, 8 min) to afford N,N-dimethyl-2-((5-methylbenzo[4,5]thieno[2,3-g]isoquinolin-9-yl)oxy)ethanamine.

MS (ESI): m/z=337.1 [M+H]⁺

### 4-(2-((6-methyl-6H-pyrido[3,2-b]carbazol-9-yl)oxy)ethyl)morpholine (Compound 225)

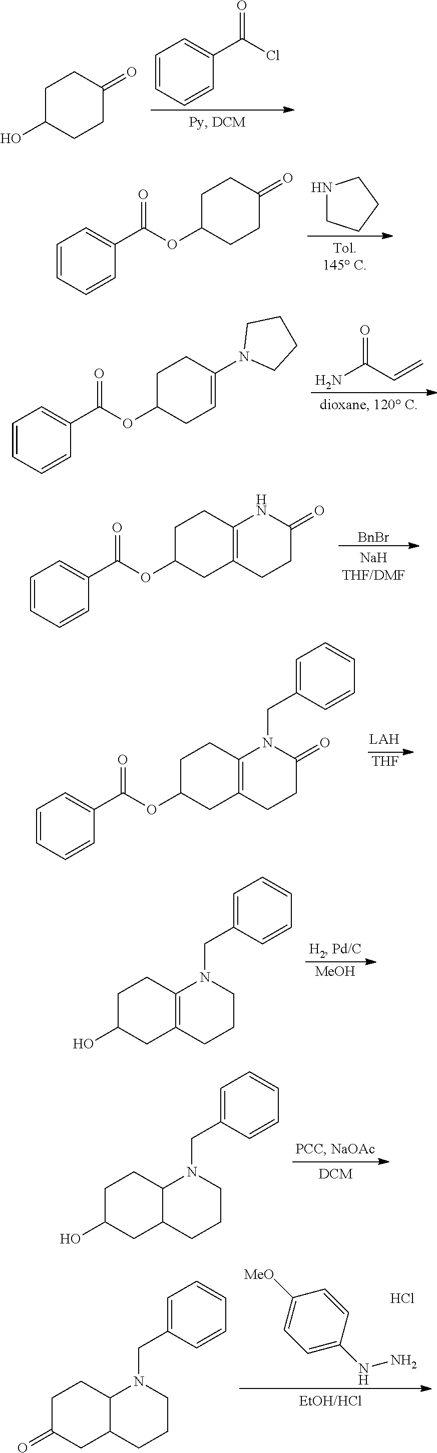

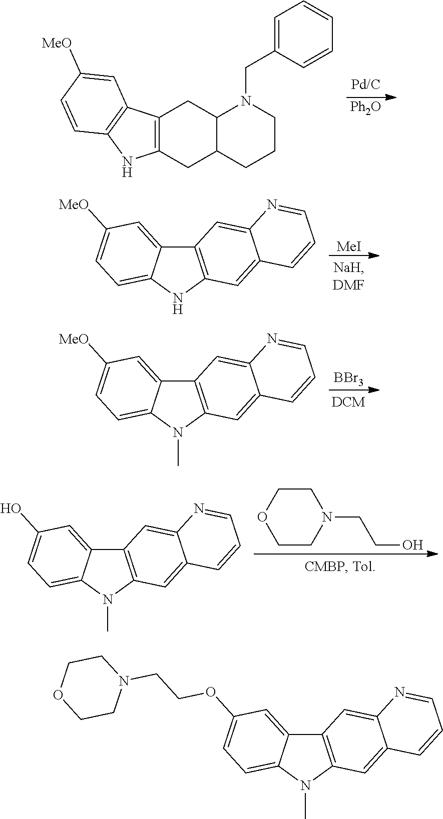

### Step 1: (4-oxocyclohexyl) benzoate

To a solution of 4-hydroxycyclohexanone (25 g, 219.0 mmol, 1 eq.) in 500 mL of DCM was added pyridine (41.6 g, 525.7 mmol, 42.4 mL, 2.4 eq.) and benzoyl chloride (37 g, 262.8 mmol, 30.5 mL, 1.2 eq), then the mixture was stirred at 20° C. for 12 hr. TLC (Petroleum ether/Ethyl acetate=1/1) indicated new spots formed. The reaction mixture was diluted with 300 mL of water and then extracted three times with 300 mL of DCM. The combined organic layers were washed with 200 mL of brine, dried over Na₂SO₄, filtered and concentrated under reduced pressure to give a residue. The residue was purified by column chromatography (SiO₂, eluting with a gradient of petroleum ether/ethyl acetate=100/1 to 4/1) to give 4-oxocyclohexyl) benzoate.

### Step 2: 4-(pyrrolidin-1-yl)cyclohex-3-en-1-yl benzoate

A mixture of (4-oxocyclohexyl) benzoate (21 g, 96.2 mmol, 1 eq.), pyrrolidine (10.3 g, 144.3 mmol, 12.0 mL, 1.5 eq), 4-methylbenzenesulfonic acid (1.7 g, 9.6 mmol, 0.1 eq.) in 20 mL of toluene was stirred at 145° C. for 5 hr. HNMR indicated desired compound. The reaction was concentrated under reduced pressure to give 4-(pyrrolidin-1-yl)cyclohex-3-en-1-yl benzoate.

### Step 3: 2-oxo-1,2,3,4,5,6,7,8-octahydroquinolin-6-yl benzoate

A mixture of 4-(pyrrolidin-1-yl)cyclohex-3-en-1-yl benzoate (26 g, 95.8 mmol, 1 eq.) in 260 mL of dioxane was added prop-2-enamide (17.0 g, 239.5 mmol, 16.5 mL, 2.5 eq.). The mixture was stirred at 120° C. for 12 hr. LCMS found the MS of desired compound. The reaction was concentrated under reduced pressure to give a residue. The residue was purified by column chromatography (SiO₂, eluting with a gradient of petroleum ether/ethyl acetate=100/1 to 3/1) to give 2-oxo-1,2,3,4,5,6,7,8-octahydroquinolin-6-yl benzoate.

### Step 4: 1-benzyl-2-oxo-1,2,3,4,5,6,7,8-octahydroquinolin-6-yl benzoate

To a solution of 2-oxo-1,2,3,4,5,6,7,8-octahydroquinolin-6-yl benzoate (10 g, 36.9 mmol, 1 eq) in 70 mL of THF and 70 mL of DMF was added NaH (1.5 g, 36.9 mmol, 60% purity, 1 eq) at 0° C. The mixture was stirred at 20° C. for 0.5 hr. A solution of BnBr (6.3 g, 36.9 mmol, 4.4 mL, 1 eq.) in 10 mL of THF was added dropwise, the mixture was stirred at 20° C. for 3 hr. LCMS found the MS of desired compound. The reaction was diluted with 200 mL of H₂O and then extracted three times with 300 mL of ethyl acetate, dried over Na₂SO₄, filtered and concentrated under reduced pressure to give a residue. The residue was purified by column chromatography (SiO₂, eluting with a gradient of petroleum ether/ethyl acetate=100/1 to 7/3) to give 1-benzyl-2-oxo-1,2,3,4,5,6,7,8-octahydroquinolin-6-yl benzoate.

### Step 5: 1-benzyl-1,2,3,4,5,6,7,8-octahydroquinolin-6-ol

To a solution of (1-benzyl-2-oxo-3,4,5,6,7,8-hexahydroquinolin-6-yl) benzoate (13 g, 36 mmol, 1 eq.) in 150 mL of THF was added LiAlH₄ (4.7 g, 123.8 mmol, 3.4 eq.) portion-wise at 0° C., the mixture was stirred at 70° C. for 4 hr. LCMS found the MS of desired compound. The reaction was diluted with 5 mL of H₂O, 5 mL of 15% NaOH (aq.) and 15 mL of H₂O, the reaction mixture was stirred 0.5 hr, then the reaction was filtered and concentrated under reduced pressure to give 1-benzyl-1,2,3,4,5,6,7,8-octahydroquinolin-6-ol.

### Step 6: 1-benzyldecahydroquinolin-6-ol

To a solution of 1-benzyl-1,2,3,4,5,6,7,8-octahydroquinolin-6-ol (5 g, 20.5 mmol, 1 eq) in MeOH (50 mL) was added Pd/C (0.5 g, 10% purity) under N₂. The suspension was degassed under vacuum and purged with H₂ several times. The mixture was stirred under H₂ (15 psi) at 25° C. for 2 hr. LCMS showed the reaction was completed and desired product was detected. The reaction mixture was filtered and concentrated under reduced pressure to give a residue. The residue was purified by flash silica gel chromatography (ISCO®; 20 g SepaFlash® Silica Flash Column, Eluent of 0˜60% Ethylacetate/Petroleum ether gradient @ 50 mL/min) to give 1-benzyldecahydroquinolin-6-ol.

### Step 7: 1-benzyloctahydroquinolin-6(2H)-one

To a solution of 1-benzyldecahydroquinolin-6-ol (4.2 g, 17.12 mmol, 1 eq) in DCM (50 mL) were added PCC (8.0 g, 37.4 mmol, 2.1 eq) and NaOAc (3.0 g, 37.5 mmol, 2.1 eq). The mixture was stirred at 25° C. for 12 hr. LCMS showed the reaction was completed. The reaction mixture was filtered and concentrated under reduced pressure to give a residue. The residue was purified by flash silica gel chromatography (ISCO®; 20 g SepaFlash® Silica Flash Column, Eluent of 0˜20% Ethyl acetate/Petroleum ether gradient @ 80 mL/min) to give 1-benzyloctahydroquinolin-6(2H)-one.

### Step 8: 1-benzyl-9-methoxy-2,3,4,4a, 5,6,11,11a-octahydro-1H-pyrido[3,2-b]carbazole

A mixture of (4-methoxyphenyl)hydrazine (387.5 mg, 2.2 mmol, 1 eq, HCl), 1-benzyloctahydroquinolin-6(2H)-one (540 mg, 2.2 mmol, 1 eq), HCl (12 M, 216.3 uL, 1.1 eq) in EtOH (10 mL) was stirred at 20° C. for 12 hr under N₂. LCMS showed the reaction was completed and desired product was detected. The mixture was filtered and washed with EtOH (10 mL×2) and collected the solid to get product. The product was diluted with NaHCO₃ (50 mL) and extracted with DCM (20 mL×3). The combined organic layers were washed with brine (20 mL×3), dried over Na₂SO₄, filtered and concentrated under reduced pressure to give a residue. The residue was purified by flash silica gel chromatography (ISCO®; 4 g SepaFlash® Silica Flash Column, Eluent of 0˜20% Ethyl acetate/Petroleum ether gradient @ 60 mL/min). The residue was purified by prep-TLC (SiO₂, Petroleum ether:Ethyl acetate=2:1) to afford 1-benzyl-9-methoxy-2,3,4,4a, 5,6,11,11a-octahydro-1H-pyrido[3,2-b]carbazole.

### Step 9: 9-methoxy-6H-pyrido[3,2-b]carbazole

To a solution of 1-benzyl-9-methoxy-2,3,4,4a, 5,6,11,11a-octahydro-1H-pyrido[3,2-b]carbazole (260.0 mg, 759.2 umol, 1 eq) in Ph₂O (5 mL) was added Pd/C (60 mg, 10% purity). The mixture was stirred at 220° C. for 1 hr under N₂. LCMS showed the reaction was completed and desired product was detected. The mixture was filtered and concentrated under reduced pressure to give a residue. The residue was purified by flash silica gel chromatography (ISCO®; 12 g SepaFlash® Silica Flash Column, Eluent of 0˜20% Ethyl acetate/Petroleum ether gradient to Eluent of 0˜10% Methanol/Dichloromethane@60 mL/min) to give 9-methoxy-6H-pyrido[3,2-b]carbazole.

### Step 10: 9-methoxy-6-methyl-pyrido[3,2-b]carbazole

To a solution of 9-methoxy-6H-pyrido[3,2-b]carbazole (130 mg, 523.6 umol, 1 eq) in THF (10 mL) was added NaH (62.8 mg, 1.5 mmol, 60% purity, 3 eq) and Mel (70.6 mg, 497.4 umol, 30.9 uL, 0.95 eq) at 0° C. Then the mixture was stirred at 25° C. for 2 hr. LCMS showed the reaction was completed and desired product was detected. The mixture was poured into NH₄Cl (10 mL) and extracted with EtOAc (50 mL×3). The combined organic layers were washed with brine (50 mL×3), dried over Na₂SO₄, filtered and concentrated under reduced pressure to give 9-methoxy-6-methyl-pyrido[3,2-b]carbazole.

### Step 11: 6-methyl-6H-pyrido[3,2-b]carbazol-9-ol

To a solution of 9-methoxy-6-methyl-pyrido[3,2-b]carbazole (140 mg, 533.7 umol, 1 eq) in DCM (10 mL) was added BBr₃ (267.4 mg, 1.0 mmol, 102.8 uL, 2 eq) at 0° C. Then the mixture was stirred at 0° C. for 2 hr. LCMS showed the reaction was completed and desired product was detected. The mixture was poured into NaHCO₃ (50 mL) and extracted with DCM (20 mL×3). The combined organic layers were washed with brine (20 mL×3), dried over Na₂SO₄, filtered and concentrated under reduced pressure to give 6-methyl-6H-pyrido[3,2-b]carbazol-9-ol.

### Step 12: 4-[2-(6-methylpyrido[3,2-b]carbazol-9-yl)oxyethyl]morpholine

To a solution of 6-methylpyrido[3,2-b]carbazol-9-ol (50 mg, 201.3 umol, 1 eq) in Tol. (2 mL) was added 2-morpholinoethanol (31.7 mg, 241.6 umol, 29.6 uL, 1.2 eq) and 2-(tributyl-λ5-phosphanylidene)acetonitrile (58.3 mg, 241.6 umol, 1.2 eq) under N2. The mixture was stirred at 100° C. for 12 hr. LCMS showed the reaction was completely. The reaction was concentrated to give a residue. The residue was purified by prep-HPLC (TFA condition; column: Phenomenex luna C18 100*40 mm*5 um; mobile phase: [water(0.1% TFA)-ACN]; B %: 1%-33%, 8 min) to afford 4-[2-(6-methylpyrido[3,2-b]carbazol-9-yl)oxyethyl]morpholine.

MS (ESI): m/z=362.1 [M+H]⁺

### N,N-dimethyl-2-((6-methyl-6H-pyrido[3,2-b]carbazol-9-yl)oxy)ethanamine (Compound 226)

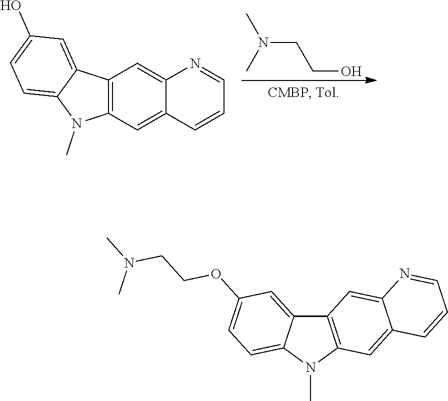

To a solution of 6-methylpyrido[3,2-b]carbazol-9-ol (50 mg, 201.3 μmol, 1 eq) in Tol. (2 mL) was added 2-(dimethylamino)ethanol (21.5 mg, 241.6 μmol, 24.2 μL, 1.2 eq) and 2-(tributyl-λ5-phosphanylidene)acetonitrile (72.9 mg, 302.0 μmol, 1.5 eq) under N₂. The mixture was stirred at 100° C. for 12 hr. LCMS showed the reaction was completely. The reaction was concentrated to give a residue. The residue was purified by prep-HPLC (TFA condition; column: Phenomenex luna C18 100*40 mm*5 μm; mobile phase: [water(0.1% TFA)-ACN]; B %: 1%-30%, 8 min) to afford N,N-dimethyl-2-(6-methylpyrido[3,2-b]carbazol-9-yl)oxyethanamine.

MS (ESI): m/z=320.1 [M+H]⁺

### 5,6-dimethyl-9-(2-(4-methylpiperidin-1-yl)ethoxy)-6H-pyrido[4,3-b]carbazole (Compound 227)

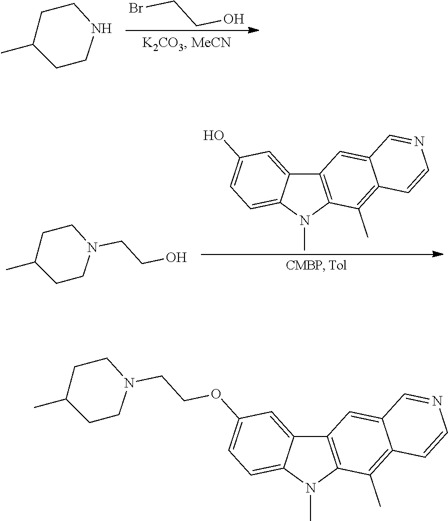

### Step 1: 2-(4-methylpiperidin-1-yl)ethanol

To a solution of 4-methylpiperidine (1 g, 10.0 mmol, 1.1 mL, 1 eq) in MeCN (10.0 mL) was added K₂CO₃ (4.1 g, 30.2 mmol, 3 eq) 2-bromoethanol (1.3 g, 11.0 mmol, 787.5 μL, 1.1 eq). The mixture was stirred at 25° C. for 12 hrs. LCMS showed the reaction was completed and desired product was detected. The reaction mixture was diluted with H₂O (20 mL) and extracted with EtOAc (20 mL×3). The combined organic layers were washed with brine (20 mL×3), dried over Na₂SO₄, filtered and concentrated under reduced pressure to give 2-(4-methylpiperidin-1-yl)ethanol.

### Step 2: 5,6-dimethyl-9-(2-(4-methylpiperidin-1-yl)ethoxy)-6H-pyrido[4,3-b]carbazole

To a solution of 5,6-dimethylpyrido[4,3-b]carbazol-9-ol (50 mg, 190.6 μmol, 1 eq) in toluene (2.0 mL) was added 2-(4-methylpiperidin-1-yl)ethanol (54.6 mg, 381.2 μmol, 2 eq) and 2-(tributyl-λ5-phosphanylidene)acetonitrile (55.2 mg, 228.7 μmol, 1.2 eq). The mixture was stirred at 100° C. for 12 hrs under N₂. LCMS showed the reaction was completed and desired product was detected. The mixture was concentrated to get crude residue. The residue was purified by prep-HPLC (TFA condition column: Phenomenex Gemini-NX C18 75*30 mm*3 μm; mobile phase: [water(0.1% TFA)-ACN]; B %: 5%-35%, 8 min) to afford 5,6-dimethyl-9-[2-(4-methyl-1-piperidyl)ethoxy]pyrido[4,3-b]carbazole.

MS (ESI): m/z=388.1 [M+H]⁺

### 1-(2-((5,6-dimethyl-6H-pyrido[4,3-b]carbazol-9-yl)oxy)ethyl)piperidin-4-ol (Compound 228)

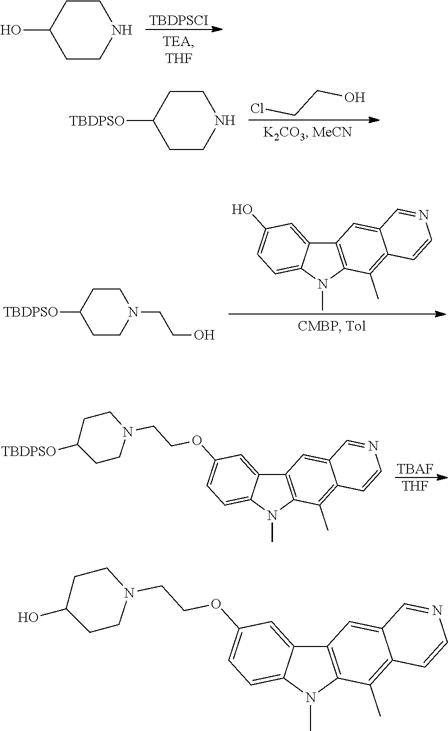

### Step 1: 4-((tert-butyldiphenylsilyl)oxy)piperidine

To a solution of piperidin-4-ol (2 g, 19.7 mmol, 1 eq) in THF (50.0 mL) was added TEA (6.0 g, 59.3 mmol, 8.2 mL, 3 eq), TBDPSCI (7.9 g, 29.0 mmol, 7.4 mL, 1.4 eq). The mixture was stirred at 25° C. for 12 hrs. LCMS showed the reaction was completed and desired product. The mixture was concentrated to get crude residue. The residue was purified by flash silica gel chromatography (ISCO®; 12 g SepaFlash® Silica Flash Column, Eluent of 0˜10% Ethyl acetate/Petroleum ether gradient @ 60 mL/min) to afford 4-((tert-butyldiphenylsilyl)oxy)piperidine.

### Step 2: 2-(4-((tert-butyldiphenylsilyl)oxy)piperidin-1-yl)ethanol

To a solution of 4-((tert-butyldiphenylsilyl)oxy)piperidine (2.3 g, 6.7 mmol, 1 eq) in MeCN (20.0 mL) was added K₂CO₃ (4.6 g, 33.8 mmol, 5 eq), 2-bromoethanol (1.6 g, 13.5 mmol, 961.9 uL, 2 eq). The mixture was stirred at 25° C. for 12 hrs. LCMS showed the reaction was completed and desired product. The reaction mixture was diluted with H₂O (20 mL) and extracted with EtOAc (20 mL×3). The combined organic layers were washed with brine (20 mL×3), dried over Na₂SO₄, filtered and concentrated under reduced pressure to give 2-(4-((tert-butyldiphenylsilyl)oxy) piperidin-1-yl)ethanol.

### Step 3: 9-(2-(4-((tert-butyldiphenylsilyl)oxy)piperidin-1-yl)ethoxy)-5,6-dimethyl-6H-pyrido[4,3-b]carbazole

To a solution of 5,6-dimethylpyrido[4,3-b]carbazol-9-ol (50 mg, 190.6 μmol, 1 eq) in toluene (4 mL) was added 2-(4-((tert-butyldiphenylsilyl)oxy) piperidin-1-yl)ethanol (146.2 mg, 381.2 μmol, 2 eq) and 2-(tributyl-λ5-phosphanylidene)acetonitrile (55.2 mg, 228.7 μmol, 1.2 eq). The mixture was stirred at 100° C. for 12 hrs under N₂. LCMS showed the reaction was completed and desired product was detected. The mixture was concentrated to get crude residue, which was purified by prep-TLC (SiO₂, DCM/MeOH=10/1) to afford 9-(2-(4-((tert-butyldiphenylsilyl) oxy)piperidin-1-yl)ethoxy)-5,6-dimethyl-6H-pyrido[4,3-b]carbazole.

### Step 4; 1-(2-((5,6-dimethyl-6H-pyrido[4,3-b]carbazol-9-yl)oxy)ethyl)piperidin-4-ol

To a solution of 9-(2-(4-((tert-butyldiphenylsilyl)oxy)piperidin-1-yl)ethoxy)-5,6-dimethyl-6H-pyrido[4,3-b]carbazole (100 mg, 159.2 μmol, 1 eq) in THF (2.0 mL) was added TBAF (1 M, 318.5 μL, 2 eq, in THF). The mixture was stirred at 50° C. for 12 hr. LCMS the reaction was completed and desired product was detected. The mixture was concentrated to get crude residue. The residue was purified by prep-HPLC (TFA condition, column: Phenomenex Gemini-NX C18 75*30 mm*3 μm; mobile phase: [water(0.1% TFA)-ACN]; B %: 10%-40%, 8 min) to afford 1-(2-((5,6-dimethyl-6H-pyrido[4,3-b]carbazol-9-yl)oxy)ethyl)piperidin-4-ol.

MS (ESI): m/z=390.1 [M+H]⁺

### 9-(2-(4-fluoropiperidin-1-yl)ethoxy)-5,6-dimethyl-6H-pyrido[4,3-b]carbazole (Compound 229)

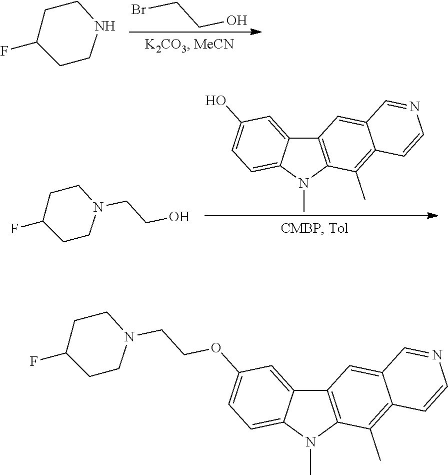

### Step 1: 2-(4-fluoropiperidin-1-yl)ethanol

To a solution of 4-fluoropiperidine (1.0 g, 7.1 mmol, 1 eq, HCl) in ACN (30 mL) was added K₂CO₃ (4.9 g, 35.8 mmol, 5 eq) and 2-bromoethanol (1.7 g, 14.3 mmol, 1.0 mL, 2 eq). The mixture was stirred at 25° C. for 12 hr. LCMS showed the reaction was complete. The reaction was concentrated to remove the solvent. The residue was dissolved in ethyl acetate (20 mL) and washed with H₂O (20 mL×3), dried over anhydrous Na₂SO₄, filtered and concentrated to give 2-(4-fluoropiperidin-1-yl)ethanol.

### 9-(2-(4-fluoropiperidin-1-yl)ethoxy)-5,6-dimethyl-6H-pyrido[4,3-b]carbazole

To a solution of 5,6-dimethylpyrido[4,3-b]carbazol-9-ol (50 mg, 190.6 μmol, 1 eq) in toluene (2 mL) was added 2-(4-fluoro-1-piperidyl)ethanol (33.6 mg, 228.7 μmol, 1.2 eq) and 2-(tributyl-λ5-phosphanylidene)acetonitrile (55.2 mg, 228.7 μmol, 1.2 eq) under N₂. The mixture was stirred at 100° C. for 12 hr. LCMS showed the reaction was complete. The reaction was concentrated to give residue. The residue was purified by prep-HPLC (TFA condition; column: Phenomenex Luna C18 100*30 mm*5 μm; mobile phase: [water(0.1% TFA)-ACN]; B %: 5%-40%, 8 min) to afford 9-(2-(4-fluoropiperidin-1-yl)ethoxy)-5,6-dimethyl-6H-pyrido[4,3-b]carbazole.

MS (ESI): m/z=392.1 [M+H]⁺

### (S)-1-((5,6-dimethyl-6H-pyrido[4,3-b]carbazol-9-yl)oxy)-N,N-dimethylpropan-2-amine (Compound 230)

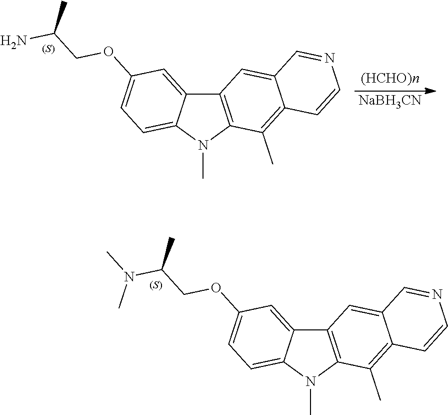

To a solution of (S)-1-((5,6-dimethyl-6H-pyrido[4,3-b]carbazol-9-yl)oxy)propan-2-amine (60 mg, 187.8 μmol, 1 eq) and (HCHO)n (90.1 mg, 939.2 μmol, 5 eq) in MeOH (2 mL) was added AcOH (11.2 mg, 187.8 μmol, 10.7 μL, 1 eq) to adjust pH to 5. Then NaBH₃CN (47.2 mg, 751.4 μmol, 4 eq) was added to the above mixture. The mixture was stirred at 40° C. for 12 hr. LCMS showed the reaction was complete. The reaction was concentrated to give a residue. The residue was purified by prep-HPLC (TFA condition; column: Phenomenex luna C18 100*40 mm*5 um; mobile phase: [water(0.1% TFA)-ACN]; B %: 1%-30%, 8 min). The residue was purified by prep-HPLC (basic condition; column: Waters Xbridge Prep OBD C18 150*40 mm*10 μm; mobile phase: [water(0.05% NH₃H2O+10 mM NH₄HCO₃)-ACN]; B %: 23%-53%, 8 min) to afford (S)-1-((5,6-dimethyl-6H-pyrido[4,3-b]carbazol-9-yl)oxy)-N,N-dimethylpropan-2-amine.

MS (ESI): m/z=348.1 [M+H]⁺

### 5-methyl-9-(2-morpholinoethoxy)benzofuro[2,3-g]isoquinoline (Compound 231)

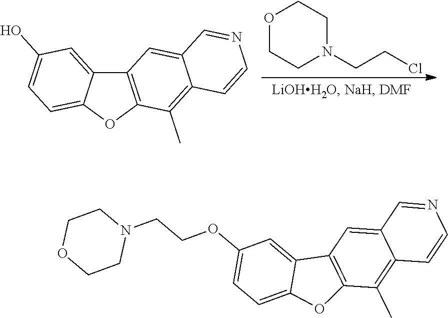

To a solution of 5-methylbenzofuro[2,3-g]isoquinolin-9-ol (70 mg, 280.8 μmol, 1 eq) in DMF (2 mL) was added 4-(2-chloroethyl)morpholine (46.2 mg, 308.9 μmol, 5.4 uL, 1.1 eq), LiOH·H₂O (47.1 mg, 1.1 mmol, 4 eq) and NaI (42.0 mg, 280.8 μmol, 1 eq). The mixture was stirred at 80° C. for 12 hrs. LCMS showed most desired product was formed. The mixture was filtered and concentrated under reduced pressure to give a residue. The residue was purified by prep-HPLC (FA condition, column: Phenomenex Luna C18 200*40 mm*10 um; mobile phase: [water(0.2% FA)-ACN]; B %: 1%-40%, 8 min) to give 5-methyl-9-(2-morpholinoethoxy)benzofuro[2,3-g]isoquinoline.

MS (ESI): m/z=363.1 [M+H]⁺

### 7-((4,5-dihydro-1H-imidazol-2-yl)methoxy)-5,6-dimethyl-6H-pyrido[4,3-b]carbazole (Compound 232)

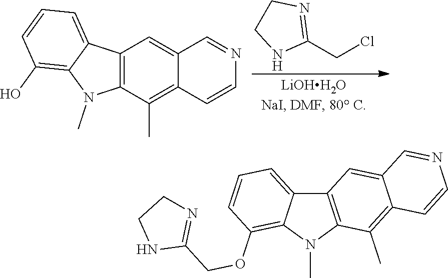

To a solution of 5,6-dimethyl-6H-pyrido[4,3-b]carbazol-7-ol (50 mg, 190.6 μmol, 1 eq) in DMF (5 mL) was added 2-(chloromethyl)-4,5-dihydro-1H-imidazole (35.4 mg, 228.7 μmol, 1.2 eq, HCl), LiOH·H₂O (32.0 mg, 762.4 μmol, 4 eq) and NaI (28.5 mg, 190.6 μmol, 1 eq), the mixture was stirred at 80° C. for 12 hrs. LC-MS showed desired compound was detected. The reaction mixture was concentrated under reduced pressure to give a residue. The residue was purified by prep-HPLC (TFA condition, column: Phenomenex luna C18 100*40 mm*5 μm; mobile phase: [water(0.1% TFA)-ACN]; B %: 1%-33%, 8 min) to give 7-((4,5-dihydro-1H-imidazol-2-yl)methoxy)-5,6-dimethyl-6H-pyrido[4,3-b]carbazole.

MS (ESI): m/z=345.1 [M+H]⁺

### 9-((4,5-dihydro-1H-imidazol-2-yl)methoxy)-5-methylbenzofuro[2,3-g]isoquinoline (Compound 233)

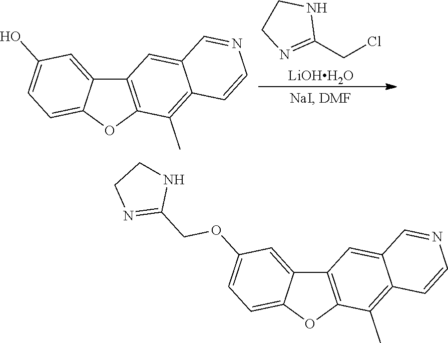

To a solution of 5-methylbenzofuro[2,3-g]isoquinolin-9-ol (70 mg, 280.8 μmol, 1 eq) in DMF (4 mL) was added 2-(chloromethyl)-4,5-dihydro-1H-imidazole (65.3 mg, 421.2 μmol, 5.4 uL, 1.5 eq, HCl), NaI (42.0 mg, 280.8 μmol, 1 eq) and LiOH·H₂O (47.1 mg, 1.1 mmol, 4 eq). The mixture was stirred at 80° C. for 12 hr. LCMS showed the reaction was completed. The reaction was concentrated under reduced pressure to give a residue. The residue was purified by prep-HPLC (FA condition, column: 3-Phenomenex Luna C18 75*30 mm*3 um; mobile phase: [water(0.2% FA)-ACN]; B %: 1%-20%, 10 min) to give 9-((4,5-dihydro-1H-imidazol-2-yl)methoxy)-5-methylbenzofuro[2,3-g]isoquinoline.

MS (ESI): m/z=332.0 [M+H]⁺

### 9-((4,5-dihydro-1H-imidazol-2-yl)methoxy)-6-methyl-6H-pyrido[4,3-b]carbazole (Compound 234)

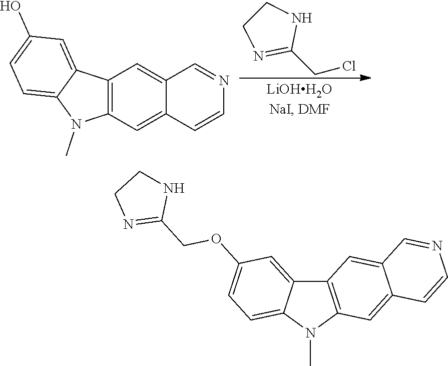

To a solution of 6-methylpyrido[4,3-b]carbazol-9-ol (50 mg, 201.3 μmol, 1 eq) in DMF (2.0 mL) were added LiOH·H₂O (33.8 mg, 805.5 μmol, 4 eq) and NaI (30.1 mg, 201.3 μmol, 1 eq) 2-(chloromethyl)-4,5-dihydro-1H-imidazole (46.8 mg, 302.0 μmol, 1.5 eq, HCl). The mixture was stirred at 70° C. for 12 hrs. LCMS showed the reaction was completed and desired product was detected. The mixture was concentrated to get crude residue. The residue was purified by prep-HPLC (TFA condition, column: Phenomenex Luna C18 100*30 mm*5 um; mobile phase: [water(0.1% TFA)-ACN]; B %: 15%-30%, 7 min) to afford 9-((4,5-dihydro-1H-imidazol-2-yl)methoxy)-6-methyl-6H-pyrido[4,3-b]carbazole.

MS (ESI): m/z=331.1 [M+H]⁺

### 4-(2-((6-isopropyl-5-methyl-6H-pyrido[4,3-b]carbazol-9-yl)oxy)ethyl)morpholine (Compound 235)

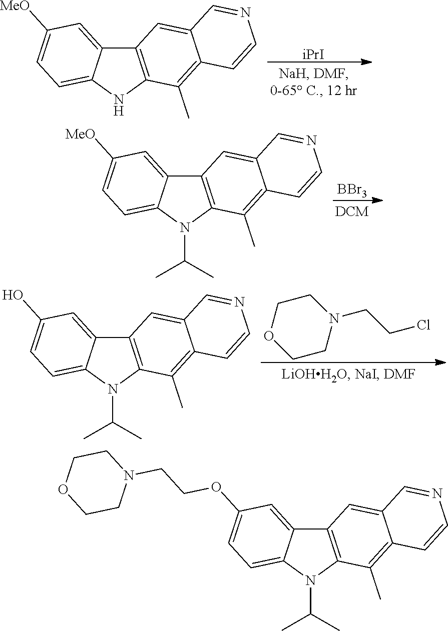

Step 1: 6-isopropyl-9-methoxy-5-methyl-6H-pyrido[4,3-b]carbazole

To a solution of 9-methoxy-5-methyl-6H-pyrido[4,3-b]carbazole (200 mg, 762.4 μmol, 1 eq) in DMF (5 mL) was added NaH (91.5 mg, 2.2 mmol, 60% purity, 3 eq) and 2-iodopropane (388.8 mg, 2.2 mmol, 228.7 μL, 3 eq) at 0° C. Then the mixture was stirred at 65° C. for 12 hrs. LCMS showed the reaction was completed and desired product was detected. The mixture was poured into NH₄Cl (10 mL) and extracted with EtOAc (10 mL×3). The combined organic layers were washed with brine (10 mL×3), dried over Na₂SO₄, filtered and concentrated under reduced pressure to give a residue. The residue was purified by prep-TLC (SiO₂, DCM:MeOH=10:1) to give 6-isopropyl-9-methoxy-5-methyl-6H-pyrido[4,3-b]carbazole.

### Step 2: 6-isopropyl-5-methyl-6H-pyrido[4,3-b]carbazol-9-ol

To a solution of 6-isopropyl-9-methoxy-5-methyl-6H-pyrido[4,3-b]carbazole (110 mg, 361.3 μmol, 1 eq) in DCM (4 mL) was added BBr₃ (452.6 mg, 1.8 mmol, 174.1 μL, 5 eq) at 0° C. The mixture was stirred at 20° C. for 12 hr. LC-MS showed the desired compound was detected. The reaction mixture was washed with NH₃·H₂O (20 mL, 25% purity) and filtered and the filter cake was washed with 10 mL of H₂O, dried in vacuum to give 6-isopropyl-5-methyl-6H-pyrido[4,3-b]carbazol-9-ol.

### Step 3: 4-(2-((6-isopropyl-5-methyl-6H-pyrido[4,3-b]carbazol-9-yl)oxy)ethyl)morpholine

To a solution of 6-isopropyl-5-methyl-6H-pyrido[4,3-b]carbazol-9-ol (50 mg, 172.2 μmol, 1 eq) in DMF (2 mL) was added 4-(2-chloroethyl)morpholine (38.4 mg, 206.6 μmol, 1.2 eq, HCl), LiOH·H₂O (28.9 mg, 688.8 μmol, 4 eq) and NaI (25.8 mg, 172.2 μmol, 1 eq). The mixture was stirred at 80° C. for 12 hr. LC-MS showed the desired compound was detected. The reaction mixture was filtered and the filtrate dried in vacuum to give a residue. The residue was purified by prep-HPLC (TFA condition, column: Phenomenex luna C18 100*40 mm*5 μm; mobile phase: [water(0.1% TFA)-ACN]; B %: 5%-40%, 8 min) to give 4-(2-((6-isopropyl-5-methyl-6H-pyrido[4,3-b]carbazol-9-yl)oxy)ethyl)morpholine.

MS (ESI): m/z=404.2 [M+H]⁺

### N,N-dimethyl-2-((5-methylbenzofuro[2,3-g]isoquinolin-9-yl)oxy)ethanamine (Compound 236)

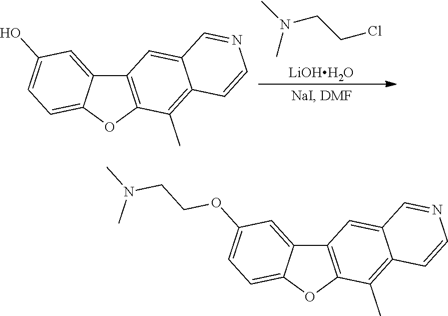

To a solution of 5-methylbenzofuro[2,3-g]isoquinolin-9-ol (60 mg, 240.7 μmol, 1 eq) in DMF (4 mL) was added 2-chloro-N,N-dimethyl-ethanamine (41.6 mg, 288.8 μmol, 5.4 μL, 1.2 eq, HCl) NaI (36.0 mg, 240.7 μmol, 1 eq) and LiOH·H₂O (40.4 mg, 962.8 μmol, 4 eq). The mixture was stirred at 80° C. for 12 hr. LCMS showed the reaction was completed. The reaction was concentrated under reduced pressure to give a residue. The residue was purified by prep-HPLC (FA condition, column: Phenomenex Luna C18 200*40 mm*10 um; mobile phase: [water(0.2% FA)-ACN]; B %: 1%-30%, 8 min) to give N,N-dimethyl-2-((5-methylbenzofuro[2,3-g]isoquinolin-9-yl)oxy)ethanamine.

MS (ESI): m/z=321.0 [M+H]⁺

### N,N-dimethyl-2-((6-methyl-6H-pyrido[4,3-b]carbazol-9-yl)oxy)ethanamine (Compound 237)

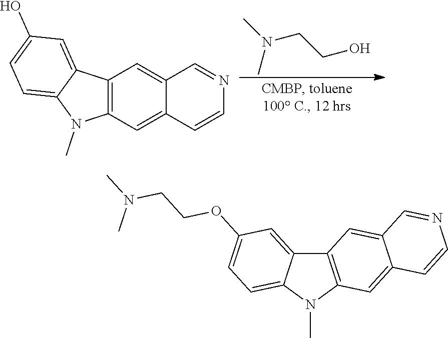

To a solution of 6-methylpyrido[4,3-b]carbazol-9-ol (70 mg, 281.9 μmol, 1 eq) in toluene (2.0 mL) were added 2-(dimethylamino)ethanol (25.1 mg, 281.9 μmol, 28.3 μL, 1.0 eq) and 2-(tributyl-λ5-phosphanylidene)acetonitrile (136.0 mg, 563.8 μmol, 2 eq. The mixture was stirred at 100° C. for 12 hrs. LCMS showed the reaction was completed and desired product was detected. The mixture was concentrated to get crude residue. The residue was purified by prep-HPLC (TFA condition, column: Phenomenex luna C18 100*40 mm*5 μm; mobile phase: [water(0.1% TFA)-ACN]; B %: 1%-22%, 8 min) to afford N,N-dimethyl-2-(6-methylpyrido[4,3-b] carbazol-9-yl)oxyethanamine.

MS (ESI): m/z=320.1 [M+H]⁺

### N-(2-(dimethylamino)ethyl)-1-isopropyl-5,6-dimethyl-6H-pyrido[4,3-b]carbazole-9-carboxamide (Compound 238)

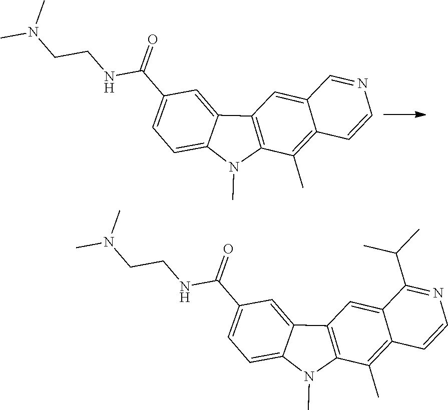 [SMILES: Cc1c2ccncc2cc2c3cc(C(=O)NCCN(C)C)ccc3n(C)c12 ; Cc1c2ccnc(C(C)C)c2cc2c3cc(C(=O)NCCN(C)C)ccc3n(C)c12]

To the solution of N-(2-(dimethylamino)ethyl)-5,6-dimethyl-6H-pyrido[4,3-b]carbazole-9-carboxamide (18 mg, 0.05 mmol) oxalate isopropyl ester (0.1 mmol) in DMSO (0.5 ml) and water (0.1 ml) was added (NH4)S2O8 (22 mg, 0.1 mmol) and the mixture was heated at 50° C. overnight and purified by HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm) to afford the product.

MS (ESI): 403.5 [M+H]⁺

### 2-(dimethylamino)ethyl 5,6-dimethyl-6H-pyrido[4,3-b]carbazole-9-carboxylate (Compound 239)

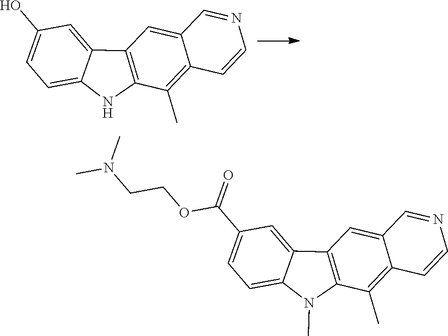

To a solution of 5,6-dimethyl-6H-pyrido[4,3-b]carbazol-9-ol (26.4 mg, 0.1 mmol) in DCM (0.2 ml) and pyridine (20 uL, 0.23 mmol) was added trifluoromethanesulfonic anhydride (53 mg, 0.2 mmol) at 0° C. The mixture was warmed up to room temperature, then stirred overnight at room temperature. To the mixture was added water and EtOAc, and the org phase was washed with aqueous NaHCO₃ and brine, dried over Na2SO4 and concentrated. The residue was dissolved in toluene (1 ml) and Pd(dppf)C12 (9.5 mg, 0.01 mmol) and 2,4,6-trichlorophenylformate (42 mg, 0.2 mmol) were added. DBU (28 uL, 0.2 mmol) was then added and the mixture was heated at 80° C. overnight under nitrogen. Aqueous NH4Cl and DCM were then added and the org phase was washed with brine, dried over Na2SO4 and concentrated in vacuo. The residue was dissolved in water/EtOH (1:1) and LiOH·H2O (42 mg, 1.0 mmol) was added. The mixture was stirred at 60° C. overnight and then purified with RP-HPLC. The resulting carboxylic acid intermediate was dissolved in dichloromethane (1 ml). 2-dimethylaminoethanol (20 uL, 0.2 mmol), DMAP (12 mg, 0.1 mmol) and EDC (38 mg, 0.2 mmol) were added. The mixture was stirred overnight at room temperature and then purified by HPLC (Waters 1525 binary preparatory HPLC system with a gradient elution of mobile phases A and B of the following specifications. Guard column: Atlantis T3 Prep Guard Cartridge (5 μm, 19 mm×10 mm) Column: Atlantis T3 Prep OBD (100 Å, 5 μm, 19 mm×150 mm) Mobile phase: A=0.1% TFA in H2O and B=MeOH or ACN; Flow rate: 5 mL/min, Injection volume: 4 mL, Runtime: 60 min, gradient: variable concentrations of B in A; UV detection at 220 & 345 nm) to afford the title compound.

MS (ESI): 362.2 [M+H]⁺

### (R)—N-(1-(dimethylamino)propan-2-yl)-7-methoxy-5,6-dimethyl-6H-pyrido[4,3-b]carbazole-1-carboxamide (Compound 240)

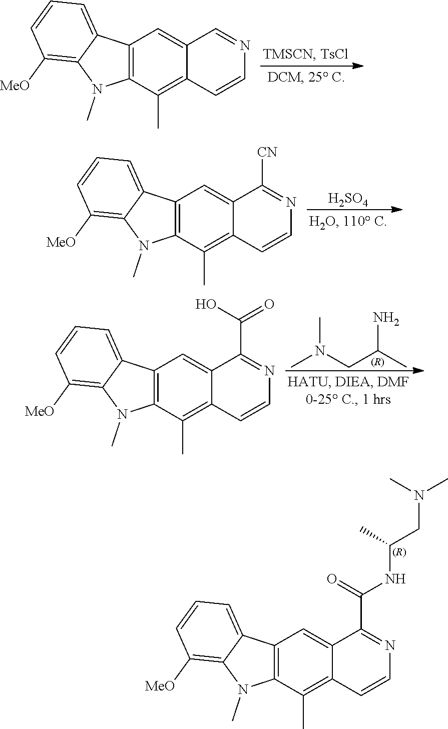

### Step 1: 7-methoxy-5,6-dimethyl-6H-pyrido[4,3-b]carbazole-1-carbonitrile

To a solution of 7-methoxy-5,6-dimethyl-6H-pyrido[4,3-b]carbazole (200 mg, 723.7 μmol, 1 eq) in DCM (6 mL) was added trimethylsilylformonitrile (186.6 mg, 1.8 mmol, 235.4 μL, 2.6 eq), then TosCl (262.1 mg, 1.3 mmol, 1.9 eq) in DCM (6 mL) was dropwsie added to the above mixture at 25° C. The mixture was stirred at 25° C. for 12 hrs. Then LCMS showed intermediate was detected and 2 mL KOH (50% W/W) was added. The mixture was stirred at 25° C. for 2 hrs. LC-MS showed desired compound was detected. The reaction mixture was partitioned between H₂O 15 mL and CH₂Cl₂ 15 mL. The aqueous phase was separated, washed with CH₂Cl₂ (10 mL×2), the organic was phase dried over Na₂SO₄, filtered and concentrated under reduced pressure to give 7-methoxy-5,6-dimethyl-6H-pyrido[4,3-b]carbazole-1-carbonitrile.

### Step 2: 7-methoxy-5,6-dimethyl-6H-pyrido[4,3-b]carbazole-1-carboxylic acid

A solution of 7-methoxy-5,6-dimethyl-6H-pyrido[4,3-b]carbazole-1-carbonitrile (200 mg, 663.7 μmol, 1 eq) in H₂SO₄ (2 mL, 98% purity) and H₂O (2 mL) was stirred at 110° C. for 12 hrs. LC-MS showed desired compound was detected. The mixture was poured into NaHCO₃ (100 mL) and yellow solid was separate out. Then the mixture was filtered and collect the solid to afford 7-methoxy-5,6-dimethyl-6H-pyrido[4,3-b]carbazole-1-carboxylic acid.

### Step 3: (R)—N-(1-(dimethylamino)propan-2-yl)-7-methoxy-5,6-dimethyl-6H-pyrido[4,3-b]carbazole-1-carboxamide

To a solution of 7-methoxy-5,6-dimethyl-6H-pyrido[4,3-b]carbazole-1-carboxylic acid (50 mg, 156.0 μmol, 1 eq) in DMF (4 mL) was added (2R)—N1,N1-dimethylpropane-1,2-diamine (81.9 mg, 468.2 μmol, 3 eq, 2 HCl), DIEA (100.8 mg, 780.4 μmol, 135.9 μL, 5 eq) and HATU (118.70 mg, 312.17 μmol, 2 eq) at 0° C. The mixture was stirred at 25° C. for 1 hr. LC-MS showed desired compound was detected. The reaction mixture was concentrated under reduced pressure to give a residue. The residue was purified by prep-HPLC (neutral condition, column: Waters Xbridge BEH C18 100*30 mm*10 um; mobile phase: [water(10 mM NH4HCO3)-ACN]; B %: 40%-70%, 10 min) to give (R)—N-(1-(dimethylamino)propan-2-yl)-7-methoxy-5,6-dimethyl-6H-pyrido[4,3-b]carbazole-1-carboxamide.

MS (ESI): m/z=405.1 [M+H]⁺

### (R)—N-(1-(dimethylamino)propan-2-yl)-5,6-dimethyl-10-(3-(4-methylpiperazin-1-yl)propoxy)-6H-pyrido[4,3-b]carbazole-1-carboxamide (Compound 241)

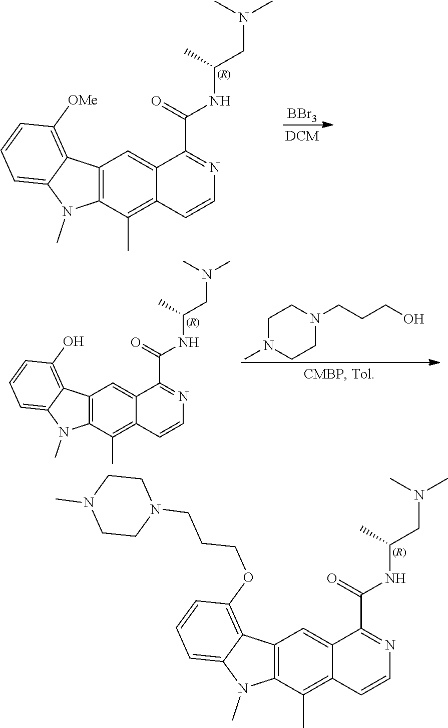

### Step 1: (R)—N-(1-(dimethylamino)propan-2-yl)-10-hydroxy-5,6-dimethyl-6H-pyrido[4,3-b]carbazole-1-carboxamide

To a solution of (R)—N-(1-(dimethylamino)propan-2-yl)-10-methoxy-5,6-dimethyl-6H-pyrido[4,3-b]carbazole-1-carboxamide (50 mg, 123.6 umol, 1 eq) in DCM (2.5 mL) was added BBr3 (154.8 mg, 618.0 umol, 59.5 uL, 5 eq) at 0° C. The mixture was stirred at 20° C. for 2 hr. LC-MS showed the desired compound was detected. The reaction mixture was add to NH3·H2O (10 mL, 25% purity) and filtered. The filter cake was washed with 10 mL of H2O, dried in vacuum to give (R)—N-(1-(dimethylamino)propan-2-yl)-10-hydroxy-5,6-dimethyl-6H-pyrido[4,3-b]carbazole-1-carboxamide.

### Step 2: (R)—N-(1-(dimethylamino)propan-2-yl)-5,6-dimethyl-10-(3-(4-methylpiperazin-1-yl)propoxy)-6H-pyrido[4,3-b]carbazole-1-carboxamide

To a solution of (R)—N-(1-(dimethylamino)propan-2-yl)-10-hydroxy-5,6-dimethyl-6H-pyrido[4,3-b]carbazole-1-carboxamide (30 mg, 76.8 umol, 1 eq) in Tol. (2 mL) was added 3-(4-methylpiperazin-1-yl)propan-1-ol (14.5 mg, 92.1 umol, 1.2 eq) and 2-(tributyl-λ5-phosphanylidene)acetonitrile (37.0 mg, 153.6 umol, 2 eq) under N2. The mixture was stirred at 100° C. for 12 hr. LC-MS showed the desired compound was detected. The reaction mixture was concentrated under reduced pressure to give a residue. The residue was purified by prep-HPLC (TFA condition, column: Phenomenex Gemini-NX 150*30 mm*5 um; mobile phase: [water(0.1% TFA)-ACN]; B %: 5%-35%, 9 min) to give (R)—N-(1-(dimethylamino)propan-2-yl)-5,6-dimethyl-10-(3-(4-methylpiperazin-1-yl)propoxy)-6H-pyrido[4,3-b]carbazole-1-carboxamide.

MS (ESI): m/z=531.3M+H]⁺

### (R)-1-((5,6-dimethyl-6H-pyrido[4,3-b]carbazol-9-yl)oxy)-N,N-dimethylpropan-2-amine (Compound 242)

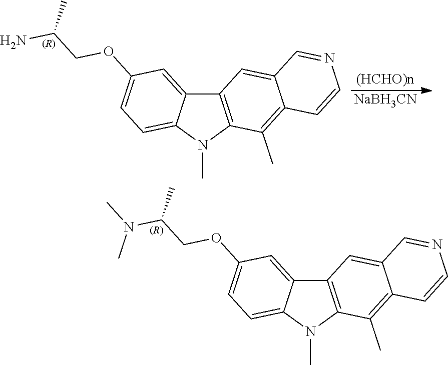

To a solution of (R)-1-((5,6-dimethyl-6H-pyrido[4,3-b]carbazol-9-yl)oxy)propan-2-amine (80 mg, 250.4 umol, 1 eq) and (HCHO)n (120.2 mg, 1.25 mmol, 5 eq) in MeOH (4 mL) was added AcOH (15.0 mg, 250.4 μmol, 14.3 μL, 1 eq) to adjusted pH to 5. Then NaBH₃CN (62.9 mg, 1.0 mmol, 4 eq) was added to the above mixture. The mixture was stirred at 25° C. for 12 hr. LC-MS showed the desired compound was detected. The reaction mixture was filtered and the filtrate dried in vacuum to give crude product. The residue was purified by prep-HPLC (basic condition, column: Waters Xbridge Prep OBD C18 150*40 mm*10 um; mobile phase: [water(0.1% TFA)-ACN]; B %: 30%-50%, 8 min) to give (R)-1-((5,6-dimethyl-6H-pyrido[4,3-b]carbazol-9-yl)oxy)-N,N-dimethylpropan-2-amine.

MS (ESI): m/z=348.1[M+H]⁺

### 5,6-dimethyl-9-(4-methylpiperazin-1-yl)-6H-pyrido[4,3-b]carbazole (Compound 243)

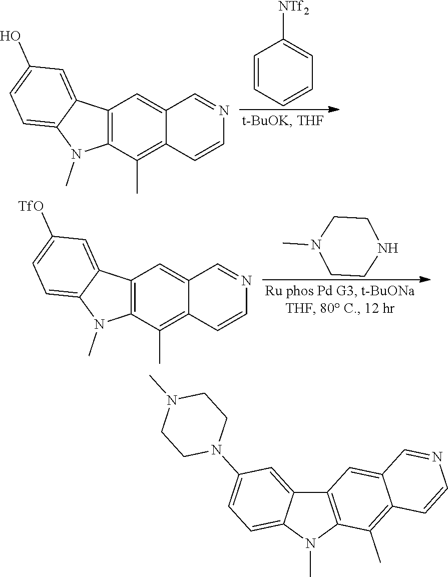

### Step 1: 5,6-dimethyl-6H-pyrido[4,3-b]carbazol-9-yl trifluoromethanesulfonate

To a solution of 5,6-dimethyl-6H-pyrido[4,3-b]carbazol-9-ol (269 mg, 1.0 mmol, 1 eq) in THF (7 mL) was added t-BuOK (230.1 mg, 2.0 mmol, 2 eq) at 0 C. The mixture was stirred at 0° C. for 10 min. And then 1,1,1-trifluoro-N-phenyl-N-((trifluoromethyl)sulfonyl)-methanesulfonamide (439.6 mg, 1.2 mmol, 1.2 eq) was added to the above mixture. The mixture was stirred at 25° C. for 12 hr. LCMS showed desired compound was detected. The reaction mixture was partitioned between H₂O 100 mL and EtOAc (50 mL×3). And the filtrate was concentrated under reduced pressure to give a residue. The residue was purified by flash silica gel chromatography (ISCO®; 4 g SepaFlash® Silica Flash Column, Eluent of 0˜40% Ethyl acetate/Petroleum ether gradient @75 mL/min) to give 5,6-dimethyl-6H-pyrido[4,3-b]carbazol-9-yl trifluoromethanesulfonate.

### Step 2: 5,6-dimethyl-9-(4-methylpiperazin-1-yl)-6H-pyrido[4,3-b]carbazole

A mixture of 5,6-dimethyl-6H-pyrido[4,3-b]carbazol-9-yl trifluoromethanesulfonate (80 mg, 202.8 umol, 1 eq), 1-methylpiperazine (40.6 mg, 405.7 umol, 45.0 uL, 2 eq), Cs₂CO₃ (132.1 mg, 405.7 umol, 2 eq), RuPhos Pd G3 (16.9 mg, 20.2 umol, 0.1 eq) in THF (2 mL) was degassed and purged with N₂ for 3 times, and then the mixture was stirred at 80° C. for 12 hr under N₂ atmosphere. LCMS showed desired compound formed. The reaction mixture was concentrated under reduced pressure to remove solvent. The residue was purified by prep-HPLC (TFA condition column: Phenomenex Gemini-NX 150*30 mm*5 um; mobile phase: [water(0.1% TFA)-ACN]; B %: 1%-30%, 9 min) to give 5,6-dimethyl-9-(4-methylpiperazin-1-yl)-6H-pyrido[4,3-b]carbazole.

MS (ESI): m/z=345.1 [M+H]⁺

### 5,6-dimethyl-9-(piperazin-1-yl)-6H-pyrido[4,3-b]carbazole (Compound 244)

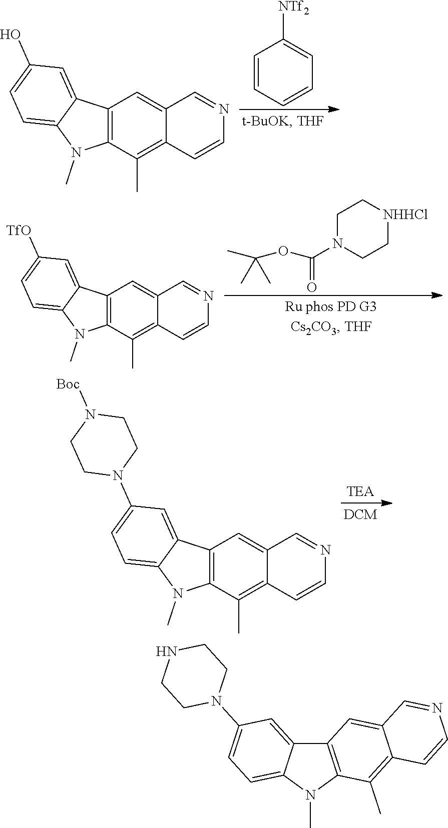

### Step 1: 5,6-dimethyl-6H-pyrido[4,3-b]carbazol-9-yl trifluoromethanesulfonate

To a solution of 5,6-dimethyl-6H-pyrido[4,3-b]carbazol-9-ol (269 mg, 1.0 mmol, 1 eq) in THF (7 mL) was added t-BuOK (230.1 mg, 2.0 mmol, 2 eq) at 0° C. The mixture was stirred at 0° C. for 10 min. And then 1,1,1-trifluoro-N-phenyl-N-((trifluoromethyl)sulfonyl)methanesulfonamide (439.6 mg, 1.2 mmol, 1.2 eq) was added to the above mixture. The mixture was stirred at 25° C. for 12 hr. LCMS showed desired compound was detected. The reaction mixture was partitioned between H₂O 100 mL and EtOAc (50 mL×3). And the filtrate was concentrated under reduced pressure to give a residue. The residue was purified by flash silica gel chromatography (ISCO®; 4 g SepaFlash® Silica Flash Column, Eluent of 0˜40% Ethyl acetate/Petroleum ether gradient @ 75 mL/min) to give 5,6-dimethyl-6H-pyrido[4,3-b]carbazol-9-yl trifluoromethanesulfonate.

### Step 2: tert-butyl 4-(5,6-dimethyl-6H-pyrido[4,3-b]carbazol-9-yl)piperazine-1-carboxylate

A mixture of 5,6-dimethyl-6H-pyrido[4,3-b]carbazol-9-yl trifluoromethanesulfonate (80 mg, 202.8 umol, 1 eq), tert-butyl piperazine-1-carboxylate hydrochloride (90.3 mg, 405.7 umol, 2 eq), Cs₂CO₃ (132.1 mg, 405.7 umol, 2 eq), Ru phos Pd G3 (16.9 mg, 20.2 umol, 0.1 eq) in THF (3 mL) was degassed and purged with N₂ for 3 times, and then the mixture was stirred at 80 C for 12 hr under N₂ atmosphere. LCMS showed desired compound formed. The reaction mixture was concentrated under reduced pressure to remove solvent. The residue was purified by prep-TLC (SiO2, DCM:MeOH=10:1) to give tert-butyl 4-(5,6-dimethyl-6H-pyrido[4,3-b]carbazol-9-yl)piperazine-1-carboxylate.

### Step 3: 5,6-dimethyl-9-(piperazin-1-yl)-6H-pyrido[4,3-b]carbazole

To a solution of tert-butyl 4-(5,6-dimethyl-6H-pyrido[4,3-b]carbazol-9-yl)piperazine-1-carboxylate (71 mg, 164.9 umol, 1 eq) in TFA (1 mL) and DCM (2 mL). The mixture was stirred at 25 C for 1 hr. LCMS showed desired compound formed. The reaction mixture was concentrated under reduced pressure to remove solvent. The residue was purified by prep-HPLC (TFA condition column: Phenomenex luna C18 100*40 mm*5 um; mobile phase: [water(0.1% TFA)-ACN]; B %: 5%-33%, 8 min) to give 5,6-dimethyl-9-(piperazin-1-yl)-6H-pyrido[4,3-b]carbazole.

MS (ESI): m/z=331.1 [M+H]⁺

### 2-((6-methyl-6H-pyrido[4,3-b]carbazol-9-yl)oxy)acetimidamide (Compound 245)

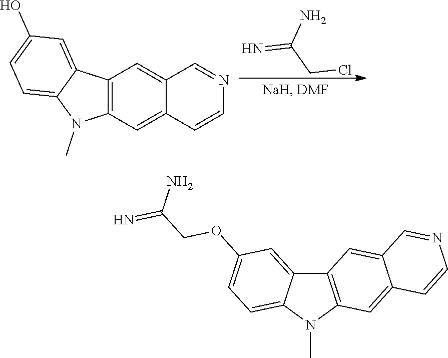

To a solution of 6-methylpyrido[4,3-b]carbazol-9-ol (100 mg, 402.7 μmol, 1 eq) in DMF (4.0 mL) were added NaH (77.3 mg, 1.93 mmol, 60% purity, 4.8 eq) and 2-chloroacetamidine (77.9 mg, 604.1 μmol, 1.5 eq, HCl). The mixture was stirred at 70° C. for 12 hrs. LCMS showed the reaction was completed and desired product was detected. The mixture was poured into NH₄Cl (10 mL) and extracted with EtOAc (20 mL×3). The combined organic layers were washed with brine (20 mL×3), dried over Na₂SO₄, filtered and concentrated under reduced pressure to give a residue. The residue was purified by prep-HPLC (TFA condition: Phenomenex Luna C18 100*30 mm*5 μm; mobile phase: [water(0.1% TFA)-ACN]; B %: 15%-30%, 7 min) to afford 2-((6-methyl-6H-pyrido[4,3-b]carbazol-9-yl)oxy).

MS (ESI): m/z=305.0 [M+H]⁺

### 5,6-dimethyl-9-(4,7-diazaspiro[2.5]octan-7-yl)-6H-pyrido[4,3-b]carbazole (Compound 246)

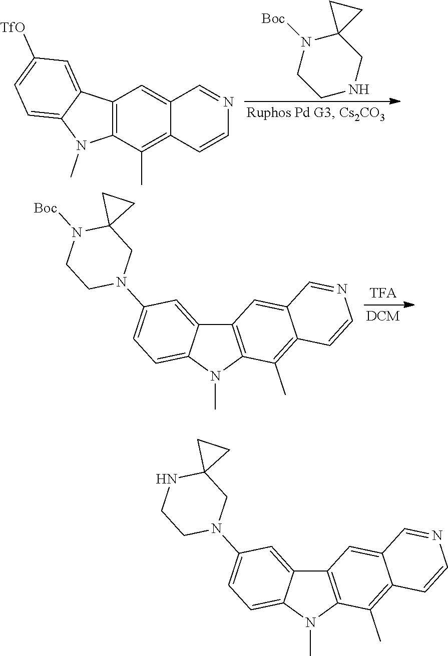

### Step 1: tert-butyl 7-(5,6-dimethyl-6H-pyrido[4,3-b]carbazol-9-yl)-4,7-diazaspiro[2.5]octane-4-carboxylate

A mixture of (5,6-dimethylpyrido[4,3-b]carbazol-9-yl) trifluoromethanesulfonate (100 mg, 253.5 umol, 1 eq), tert-butyl 4,7-diazaspiro[2.5]octane-4-carboxylate (161.4 mg, 760.7 umol, 3 eq), Cs₂CO₃ (247.8 mg, 760.7 umol, 3 eq) and RuPhos Pd G3 (42.4 mg, 50.7 umol, 0.2 eq) in THF (4 mL) was degassed and purged with N₂ for 3 times, and then the mixture was stirred at 80° C. for 12 hr under N₂ atmosphere. LCMS showed the reaction was completely. The reaction was concentrated to give a residue. The residue was purified by prep-TLC (SiO₂, Petroleum ether/Ethyl acetate=0/1) to afford tert-butyl 7-(5,6-dimethyl-6H-pyrido[4,3-b]carbazol-9-yl)-4,7-diazaspiro[2.5]octane-4-carboxylate.

### Step 2; 5,6-dimethyl-9-(4,7-diazaspiro[2.5]octan-7-yl)-6H-pyrido[4,3-b]carbazole

To a solution of tert-butyl 7-(5,6-dimethyl-6H-pyrido[4,3-b]carbazol-9-yl)-4,7-diazaspiro[2.5]octane-4-carboxylate (70 mg, 153.3 umol, 1 eq) in DCM (2 mL) was added TFA (1 mL). The mixture was stirred at 25° C. for 0.5 hr. LCMS showed the reaction was completely. The reaction was concentrated to give a residue. The residue was purified by prep-HPLC (TFA condition; column: Phenomenex luna C18 80*40 mm*3 um; mobile phase: [water(0.1% TFA)-ACN]; B %: 3%-27%, 7 min) to afford 5,6-dimethyl-9-(4,7-diazaspiro[2.5]octan-7-yl)-6H-pyrido[4,3-b]carbazole.

MS (ESI): m/z=357.1 [M+H]⁺

### 4-(2-((5-methyl-6-neopentyl-6H-pyrido[4,3-b]carbazol-9-yl)oxy)ethyl)morpholine (Compound 247)

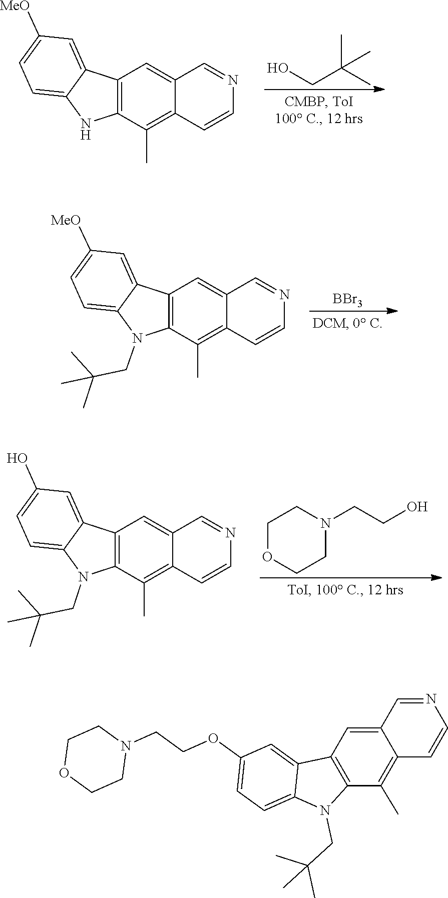

### Step 1: 9-methoxy-5-methyl-6-neopentyl-6H-pyrido[4,3-b]carbazole

To a solution of 9-methoxy-5-methyl-6H-pyrido[4,3-b]carbazole (400 mg, 1.5 mmol, 1 eq) in Tol. (2 mL) was added 2,2-dimethylpropan-1-ol (268.8 mg, 3.0 mmol, 328.7 μL, 2 eq) and 2-(tributyl-λ5-phosphanylidene)acetonitrile (552.0 mg, 2.2 mmol, 1.5 eq). The mixture was stirred at 100° C. for 12 hrs under N₂. LC-MS showed desired compound was detected. The reaction mixture was concentrated under reduced pressure to give a residue. The residue was purified by flash silica gel chromatography (ISCO®; 12 g SepaFlash® Silica Flash Column, Eluent of 0˜80% Ethyl acetate/Petroleum ether gradient @ 70 mL/min) to give 9-methoxy-5-methyl-6-neopentyl-6H-pyrido[4,3-b]carbazole.

### Step 2: 5-methyl-6-neopentyl-6H-pyrido[4,3-b]carbazol-9-ol

To a solution of 9-methoxy-5-methyl-6-neopentyl-6H-pyrido[4,3-b]carbazole (450 mg, 1.35 mmol, 1 eq) in DCM (10 mL) was added BBr₃ (1.7 g, 6.7 mmol, 652.1 μL, 5 eq). The mixture was stirred at 0° C. for 2 hrs. LC-MS showed desired compound was detected. The reaction was poured into saturated aqueous NaHCO₃ (30 mL). The aqueous phase was washed with H₂O (10 mL×3), filtered and concentrated under reduced pressure to give 5-methyl-6-neopentyl-6H-pyrido[4,3-b]carbazol-9-ol

### Step 3: 4-(2-((5-methyl-6-neopentyl-6H-pyrido[4,3-b]carbazol-9-yl)oxy)ethyl)morpholine

To a solution of 5-methyl-6-neopentyl-6H-pyrido[4,3-b]carbazol-9-ol (80 mg, 251.25 μmol, 1 eq) in Tol. (3 mL) was added 2-morpholinoethanol (32.9 mg, 251.2 μmol, 30.8 μL, 1 eq) and 2-(tributyl-λ5-phosphanylidene)acetonitrile (121.2 mg, 502.4 μmol, 2 eq). The mixture was stirred at 100° C. for 12 hrs under N₂. LC-MS showed desired compound was detected. The reaction mixture was concentrated under reduced pressure to give a residue. The residue was purified by prep-HPLC (TFA condition, column: Phenomenex luna C18 100*40 mm*5 um; mobile phase: [water(0.1% TFA)-ACN]; B %: 5%-34%, 8 min) to give 4-(2-((5-methyl-6-neopentyl-6H-pyrido[4,3-b]carbazol-9-yl)oxy)ethyl)morpholine.

MS (ESI): m/z=432.2 [M+H]⁺

### 4-(2-((6-isopentyl-5-methyl-6H-pyrido[4,3-b]carbazol-9-yl)oxy)ethyl)morpholine (Compound 248)

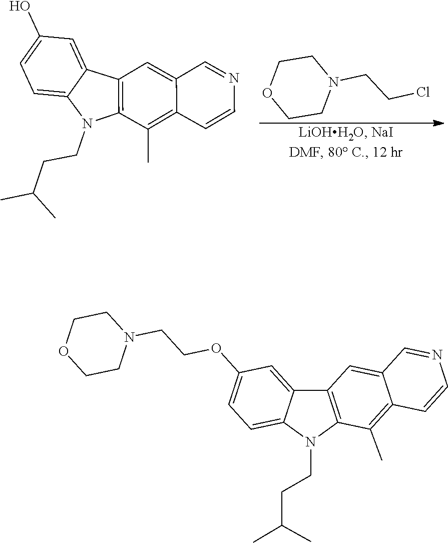

To a solution of 6-isopentyl-5-methyl-6H-pyrido[4,3-b]carbazol-9-ol (80 mg, 251.2 umol, 1 eq) in DMF (4 mL) was added 4-(2-chloroethyl)morpholine (111.8 mg, 502.4 umol, 2 eq, HCl), LiOH·H₂O (42.1 mg, 1.0 mmol, 4 eq) and NaI (37.6 mg, 251.2 umol, 1 eq). The mixture was stirred at 80° C. for 12 hr. LC-MS showed desired compound was detected. The reaction mixture was concentrated under reduced pressure to give a residue. The residue was purified by prep-HPLC (TFA condition, column: Phenomenex luna C18 100*40 mm*5 um; mobile phase: [water (0.1% TFA)-ACN]; B %: 5%-38%, 8 min) to give 4-[2-(6-isopentyl-5-methyl-pyrido[4,3-b]carbazol-9-yl)oxyethyl]morpholine.

MS (ESI): m/z=432.3 [M+H]⁺

### 9-(4,5-dihydro-1H-imidazol-2-ylmethoxy)-6-isopentyl-5-methyl-pyrido[4,3-b]carbazole (Compound 249)

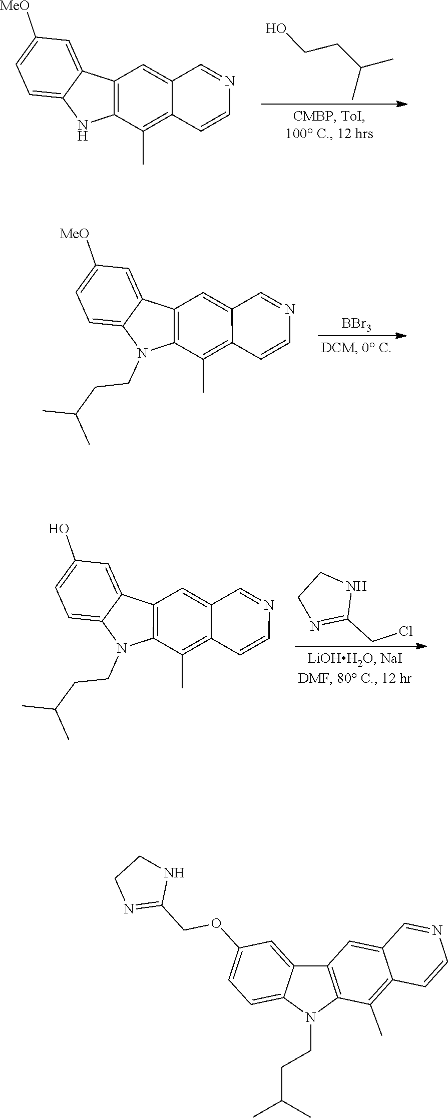

### Step 1: 6-isopentyl-9-methoxy-5-methyl-6H-pyrido[4,3-b]carbazole

To a solution of 9-methoxy-5-methyl-6H-pyrido[4,3-b]carbazole (400 mg, 1.5 mmol, 1 eq) in Tol (2 mL) was added 3-methylbutan-1-ol (134.4 mg, 1.5 mmol, 166.0 uL, 1 eq) and 2-(tributyl-λ5-phosphanylidene)acetonitrile (441.6 mg, 1.83 mmol, 1.2 eq). The mixture was stirred at 100° C. for 12 hrs under N₂. LC-MS showed desired compound was detected. The reaction mixture was concentrated under reduced pressure to give a residue. The residue was purified by flash silica gel chromatography (ISCO®; 12 g SepaFlash® Silica Flash Column, Eluent of 0˜80% Ethyl acetate/Petroleum ether gradient @ 70 mL/min) to give

### 6-isopentyl-9-methoxy-5-methyl-pyrido[4,3-b]carbazole

### Step 2; 6-isopentyl-5-methyl-6H-pyrido[4,3-b]carbazol-9-ol

To a solution of 6-isopentyl-9-methoxy-5-methyl-pyrido[4,3-b]carbazole (410 mg, 1.2 mmol, 1 eq) in DCM (8 mL) was added BBr₃ (1.5 g, 6.1 mmol, 594.1 uL, 5 eq). The mixture was stirred at 0° C. 2 hrs. LC-MS showed desired compound was detected. The reaction was slowly added to NH₃·H₂O (25% purity, 10 mL) at 0° C. The mixture was filtered and the cake was washed with H₂O (10 mL×3). The combined aqueous was washed with DCM (10 mL×3). The organic phase was dried over anhydrous Na₂SO₄ and concentrated to give 6-isopentyl-5-methyl-6H-pyrido[4,3-b]carbazol-9-ol.

### Step 3; 9-((4,5-dihydro-1H-imidazol-2-yl)methoxy)-6-isopentyl-5-methyl-6H-pyrido[4,3-b]carbazole

To a solution of 6-isopentyl-5-methyl-6H-pyrido[4,3-b]carbazol-9-ol (80 mg, 251.2 umol, 1 eq) in DMF (4 mL) was added 2-(chloromethyl)-4,5-dihydro-1H-imidazole (46.7 mg, 301.5 umol, 1.2 eq, HCl) and LiOH·H₂O (42.1 mg, 1.0 mmol, 4 eq) and NaI (37.6 mg, 251.2 umol, 1 eq) was added in the mixture was stirred at 80° C. for 12 hrs. LC-MS showed desired compound was detected. The reaction mixture was concentrated under reduced pressure to give a residue. The residue was purified by prep-HPLC (TFA condition, column: Phenomenex luna C18 100*40 mm*5 um; mobile phase: [water(0.1% TFA)-ACN]; B %: 5%-38%, 8 min) to give 9-(4,5-dihydro-1H-imidazol-2-ylmethoxy)-6-isopentyl-5-methyl-pyrido[4,3-b]carbazole.

MS (ESI): m/z=401.1 [M+H]⁺

### 4-(2-((5-methyl-6H-pyrido[4,3-b]carbazol-9-yl)oxy)ethyl)morpholine (Compound 250)

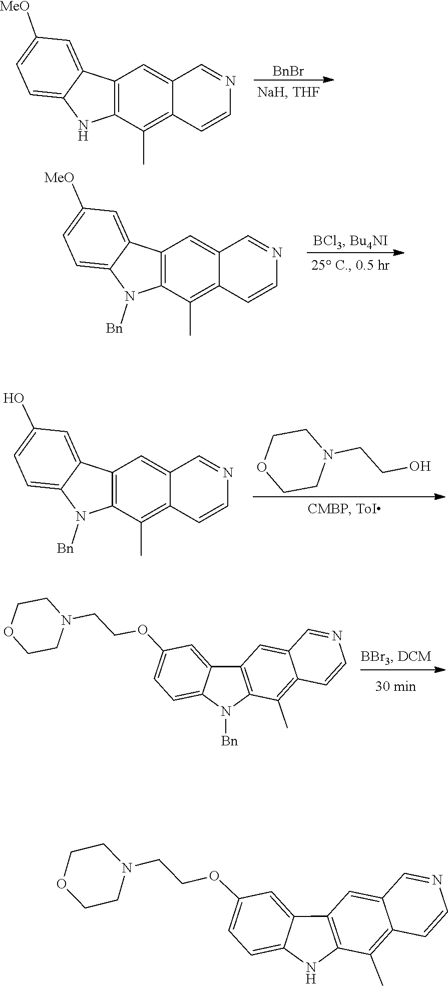

### Step 1: 6-benzyl-9-methoxy-5-methyl-6H-pyrido[4,3-b]carbazole

To a solution of 9-methoxy-5-methyl-6H-pyrido[4,3-b]carbazole (500 mg, 1.9 mmol, 1 eq) in THF (15 mL) was added NaH (228.7 mg, 5.7 mmol, 60% purity, 3 eq) at 0° C. and stirred for 30 min. Bromomethylbenzene (326.0 mg, 1.9 mmol, 226.4 uL, 1 eq) was added to the mixture at 0° C. and stirred at 25° C. for 12 hr. LCMS showed the desired compound was detected. The reaction mixture was partitioned between NH₄Cl (50 mL) and EtOAc (50 mL×3). The organic phase was separated, washed with brine (30 mL×3), dried over Na₂SO₄, filtered and concentrated under reduced pressure to give a residue. The residue was purified by flash silica gel chromatography (ISCO®; 4 g SepaFlash® Silica Flash Column, Eluent of 0˜25% Ethyl acetate/Petroleum ether gradient @ 75 mL/min) to give 6-benzyl-9-methoxy-5-methyl-pyrido[4,3-b]carbazole.

### Step 2: 6-benzyl-5-methyl-6H-pyrido[4,3-b]carbazol-9-ol

6-benzyl-9-methoxy-5-methyl-pyrido[4,3-b]carbazole (90 mg, 255.3 umol, 1 eq) and Bu₄NI (188.6 mg, 510.7 umol, 2 eq) were added to BCl₃ (5 mL) at 25° C. The mixture was stirred at 25° C. for 30 min. LC-MS showed the desired compound was detected. The reaction mixture was added to NaHCO₃ (10 mL) and filtered and the filter cake was washed with 10 mL of H₂O, dried in vacuum to give 6-benzyl-5-methyl-6H-pyrido[4,3-b]carbazol-9-ol.

### Step 3: 4-(2-((6-benzyl-5-methyl-6H-pyrido[4,3-b]carbazol-9-yl)oxy)ethyl)morpholine

To a solution of 6-benzyl-5-methyl-pyrido[4,3-b]carbazol-9-ol (100 mg, 295.5 umol, 1 eq) in Tol. (4 mL) was added 2-morpholinoethanol (46.5 mg, 354.6 umol, 43.4 uL, 1.2 eq) and 2-(tributyl-λ⁵-phosphanylidene)acetonitrile (142.6 mg, 591.0 umol, 2 eq) under N₂. The mixture was stirred at 100° C. for 12 hr. LC-MS showed the desired compound was detected. The reaction mixture was concentrated under reduced pressure to give a residue. The residue was purified by prep-TLC (SiO₂, DCM:MeOH=10:1) to give 4-[2-(6-benzyl-5-methyl-pyrido[4,3-b]carbazol-9-yl)oxyethyl]morpholine.

### Step 4: 4-(2-((5-methyl-6H-pyrido[4,3-b]carbazol-9-yl)oxy)ethyl)morpholine

To a solution of 4-[2-(6-benzyl-5-methyl-pyrido[4,3-b]carbazol-9-yl)oxyethyl]morpholine (30 mg, 66.4 umol, 1 eq) in DCM (1 mL) was added BBr₃ (83.2 mg, 332.1 umol, 32.0 uL, 5 eq) at −20° C. The mixture was stirred at −20° C. for 30 min. LC-MS showed the desired compound was detected. The reaction was added to NH₃·H₂O (5 mL). The reaction mixture was partitioned between H₂O 5 mL and CH₂Cl₂ (5 mL×3). The organic phase was separated, dried over Na₂SO₄, filtered and concentrated under reduced pressure to give a residue. The residue was purified by prep-HPLC (TFA condition column: Phenomenex luna C18 100*40 mm*5 um; mobile phase: [water(0.1% TFA)-ACN]; B %: 1%-25%, 8 min) to give 4-(2-((5-methyl-6H-pyrido[4,3-b]carbazol-9-yl)oxy)ethyl)morpholine.

MS (ESI): m/z=362.1 [M+H]⁺

### 9-((1H-imidazol-2-yl)methoxy)-5,6-dimethyl-6H-pyrido[4,3-b]carbazole (Compound 251)

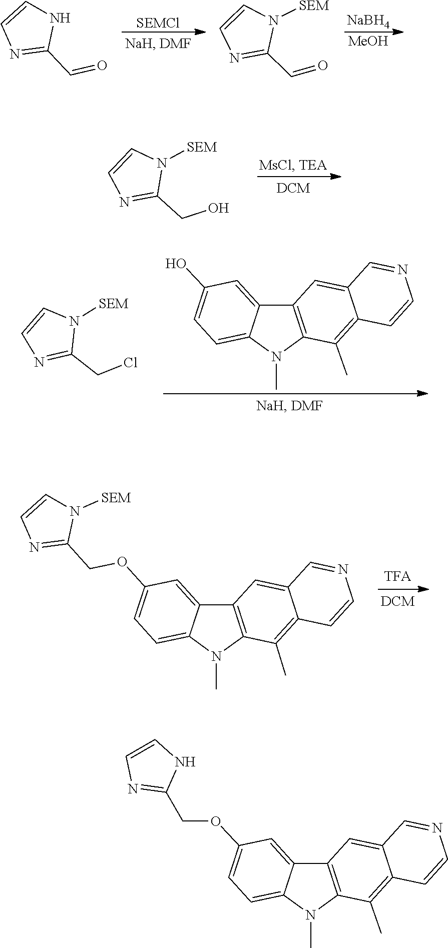

### Step 1: 1-((2-(trimethylsilyl)ethoxy)methyl)-1H-imidazole-2-carbaldehyde

To a solution of 1H-imidazole-2-carbaldehyde (1.0 g, 10.4 mmol, 1 eq) in DMF (30 mL) was added NaH (499.5 mg, 12.4 mmol, 60% purity, 1.2 eq) at 25° C. After 1.5 hr, SEM-CI (1.9 g, 11.4 mmol, 2.0 mL, 1.1 eq) was added to the above mixture and the mixture was stirred at 25° C. for 12 hr. LCMS showed the reaction was completely. The reaction was quenched with saturated NH₄Cl (15 mL) and extracted with ethyl acetate (30 mL×3). The combined organic phase was washed with brine (20 mL×3), dried over anhydrous Na₂SO₄, filtered and concentrated to give a residue. The residue was purified by column chromatography (SiO₂, Petroleum ether/Ethyl acetate=0/1 to 3/1) to afford 1-(2-trimethylsilylethoxymethyl)imidazole-2-carbaldehyde.

### Step 2: (1-((2-(trimethylsilyl)ethoxy)methyl)-1H-imidazol-2-yl)methanol

To a solution of 1-(2-trimethylsilylethoxymethyl)imidazole-2-carbaldehyde (1.0 g, 4.4 mmol, 1 eq) in MeOH (15 mL) was added NaBH₄ (200.5 mg, 5.3 mmol, 1.2 eq) at −20° C. The mixture was stirred at 25° C. for 12 hr. LCMS showed the reaction was completely. The reaction was concentrated to remove MeOH, and then dissolved in ethyl acetate (25 mL), poured into saturated aqueous NaHCO₃ (15 mL) and then separated. The organic phase was washed with brine (20 mL×3), dried over anhydrous Na₂SO₄, filtered and concentrated to give a residue. The residue was purified by flash silica gel chromatography (ISCO®; 4 g SepaFlash® Silica Flash Column, Eluent of 0˜100% Ethyl acetate/Petroleum ether gradient @ 50 mL/min) to give (1-((2-(trimethylsilyl)ethoxy)methyl)-1H-imidazol-2-yl)methanol.

### Step 3: 2-(chloromethyl)-1-((2-(trimethylsilyl)ethoxy)methyl)-1H-imidazole

To a solution of [1-(2-trimethylsilylethoxymethyl)imidazol-2-yl]methanol (500 mg, 2.1 mmol, 1 eq) in DCM (20 mL) was added TEA (664.6 mg, 6.5 mmol, 914.2 uL, 3 eq) and methanesulfonyl chloride (400.0 mg, 3.4 mmol, 270.2 uL, 1.5 eq). The mixture was stirred at 25° C. for 2.5 hr. LCMS showed desired compound formed. The reaction was quenched with saturated H₂O (15 mL) and extracted with DCM (30 mL×3), dried over anhydrous Na₂SO₄, filtered and concentrated to give 2-[[2-(chloromethyl)imidazol-1-yl]methoxy]ethyl-trimethyl-silane.

### Step 4: 5,6-dimethyl-9-((1-((2-(trimethylsilyl)ethoxy)methyl)-1H-imidazol-2-yl)methoxy)-6H-pyrido[4,3-b]carbazole

To a solution of 5,6-dimethylpyrido[4,3-b]carbazol-9-ol (100 mg, 381.2 umol, 1 eq) in DMF (2 mL) was added NaH (45.7 mg, 1.1 mmol, 60% purity, 3 eq) at 0° C. and the mixture was stirred for 0.5 hr. 2-[[2-(chloromethyl)imidazol-1-yl]methoxy]ethyl-trimethyl-silane (103.5 mg, 419.3 umol, 10.7 uL, 1.1 eq) and NaI (57.1 mg, 381.2 umol, 1 eq) was added to the above mixture. The mixture was stirred at 25° C. for 12 hr. LCMS showed the reaction was completely. The reaction was concentrated to give a residue. The residue was purified by prep-TLC (SiO₂, DCM:MeOH=10:1) to afford 5,6-dimethyl-9-((1-((2-(trimethylsilyl)ethoxy)methyl)-1H-imidazol-2-yl)methoxy)-6H-pyrido[4,3-b]carbazole.

### Step 5: 9-((1H-imidazol-2-yl)methoxy)-5,6-dimethyl-6H-pyrido[4,3-b]carbazole

To a solution of 5,6-dimethyl-9-((1-((2-(trimethylsilyl)ethoxy)methyl)-1H-imidazol-2-yl)methoxy)-6H-pyrido[4,3-b]carbazole (60 mg, 126.9 umol, 1 eq) in DCM (2 mL) was added TFA (1 mL). The mixture was stirred at 25° C. for 0.5 hr. LCMS showed the reaction was completely. The reaction was concentrated to give a residue. The residue was purified by prep-HPLC (TFA condition; Phenomenex luna C18 80*40 mm*3 um; mobile phase: [water(0.1% TFA)-ACN]; B %: 1%-35%, 7 min) to afford 9-((1H-imidazol-2-yl)methoxy)-5,6-dimethyl-6H-pyrido[4,3-b]carbazole.

MS (ESI): m/z=343.0 [M+H]⁺

### 9-((4,5-dihydro-1H-imidazol-2-yl)methoxy)-5-methyl-6-neopentyl-6H-pyrido[4,3-b]carbazole (Compound 252)

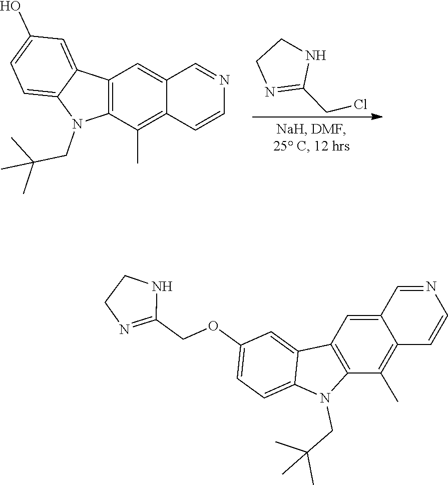

To a solution of 5-methyl-6-neopentyl-6H-pyrido[4,3-b]carbazol-9-ol (25 mg, 78.5 umol, 1 eq) in DMF (3 mL) was added 2-(chloromethyl)-4,5-dihydro-1H-imidazole (11.1 mg, 94.2 umol, 1.2 eq), NaH (12.5 mg, 314.0 umol, 60% purity, 4 eq) was added in the mixture was stirred at 25° C. for 12 hrs. LC-MS showed desired compound was detected. The reaction mixture was concentrated under reduced pressure to give a residue. The residue was purified by prep-HPLC (TFA condition; column: Phenomenex luna C18 100*40 mm*5 um; mobile phase: [water(0.1% TFA)-ACN]; B %: 13%-30%, 8 min) to give 9-((4,5-dihydro-1H-imidazol-2-yl)methoxy)-5-methyl-6-neopentyl-6H-pyrido[4,3-b]carbazole.

MS (ESI): m/z=401.1[M+H]⁺

### (R)—N-(1-(dimethylamino)propan-2-yl)-5,6-dimethyl-7-(3-(4-methylpiperazin-1-yl)propoxy)-6H-pyrido[4,3-b]carbazole-1-carboxamide (Compound 253)

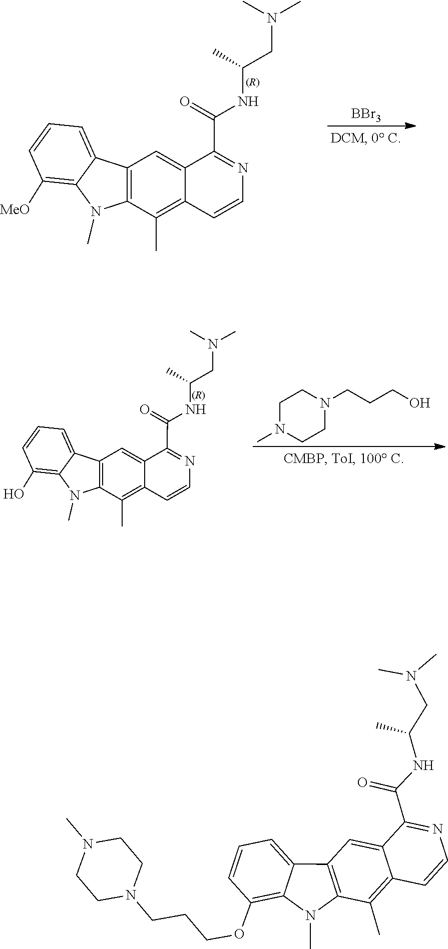

### Step 1: (R)—N-(1-(dimethylamino)propan-2-yl)-7-hydroxy-5,6-dimethyl-6H-pyrido[4,3-b]carbazole-1-carboxamide

To a solution of (R)—N-(1-(dimethylamino)propan-2-yl)-7-methoxy-5,6-dimethyl-6H-pyrido[4,3-b]carbazole-1-carboxamide (30 mg, 74.1 umol, 1 eq) in DCM (3 mL) was added BBr₃ (92.9 mg, 370.8 umol, 35.7 uL, 5 eq). The mixture was stirred at 0° C. 2 hrs. LC-MS showed desired product was detected. The reaction mixture was quenched by addition NH₃·H₂O 10 mL at 0° C., and extracted with DCM (10 mL×3). The combined organic layers were washed with brine (15 mL×1), dried over Na₂SO₄, filtered and concentrated under reduced pressure to give (R)—N-(1-(dimethylamino)propan-2-yl)-7-hydroxy-5,6-dimethyl-6H-pyrido[4,3-b] carbazole-1-carboxamide.

### Step 2: (R)—N-(1-(dimethylamino)propan-2-yl)-5,6-dimethyl-7-(3-(4-methylpiperazin-1-yl)propoxy)-6H-pyrido[4,3-b]carbazole-1-carboxamide

To a solution of (R)—N-(1-(dimethylamino)propan-2-yl)-7-hydroxy-5,6-dimethyl-6H-pyrido[4,3-b]carbazole-1-carboxamide (25 mg, 64.0 umol, 1 eq) in Tol. (4 mL) was added 3-(4-methylpiperazin-1-yl)propan-1-ol (12.1 mg, 76.8 umol, 1.2 eq) and 2-(tributyl-λ5-phosphanylidene)acetonitrile (46.3 mg, 192.0 umol, 3 eq). The mixture was stirred at 100° C. for 12 hrs. LC-MS showed desired compound was detected. The reaction mixture was concentrated under reduced pressure to give a residue. The residue was purified by prep-HPLC (neutral condition; column: Phenomenex Gemini-NX C18 75*30 mm*3 um; mobile phase: [water(0.05% NH₃H2O+10 mM NH₄HCO₃)-ACN]; B %: 20%-65%, 8 min) to give (R)—N-(1-(dimethylamino)propan-2-yl)-5,6-dimethyl-7-(3-(4-methylpiperazin-1-yl)propoxy)-6H-pyrido[4,3-b]carbazole-1-carboxamide.

MS (ESI): m/z=531.3 [M+H]⁺

### 9-((4,5-dihydro-1H-imidazol-2-yl)methoxy)-7-fluoro-5,6-dimethyl-6H-pyrido[4,3-b]carbazole (Compound 254)

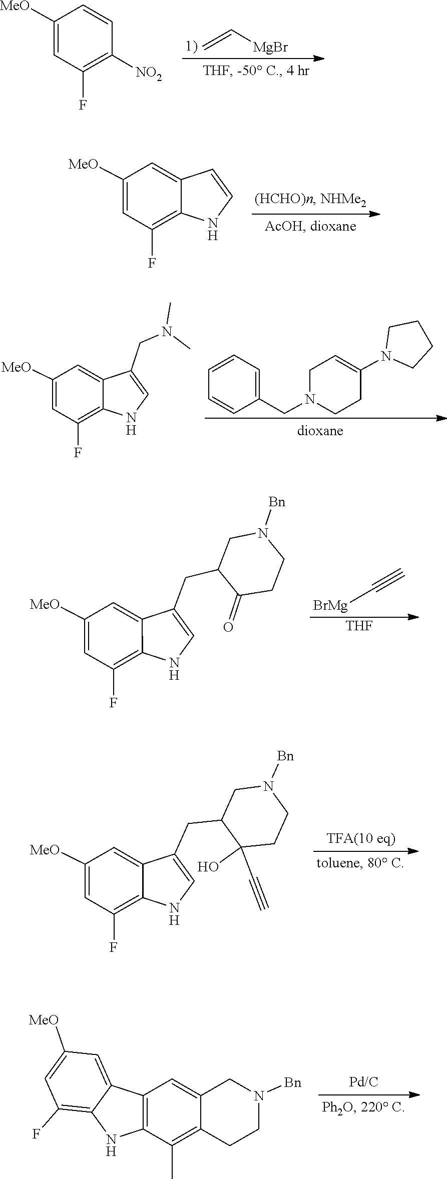

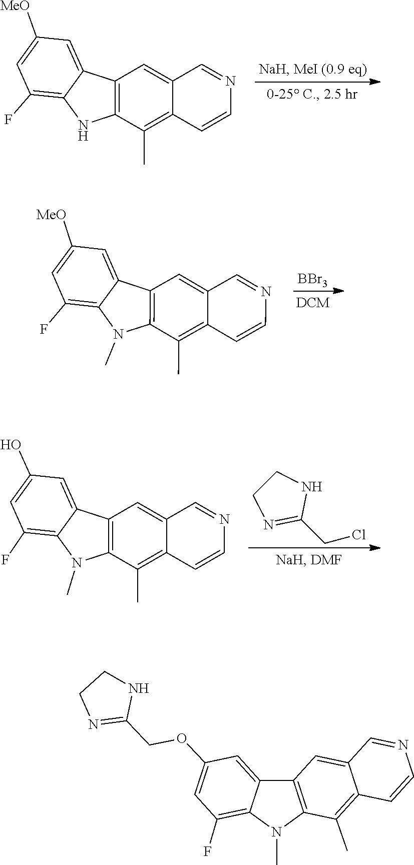

### Step 1: 7-fluoro-5-methoxy-1H-indole

To a solution of 2-fluoro-4-methoxy-1-nitro-benzene (30 g, 175.3 mmol, 1 eq) in THF (600 mL) was added dropwise bromo(vinyl)magnesium (1 M, 876.5 mL, 5 eq) at −50° C. over 2 hr. Then the mixture was stirred at −50° C. for 2 hr. LC-MS showed the desired compound was detected. The reaction mixture was partitioned between NH₄Cl 800 mL and EtOAc 1000 mL. The water phase was separated, extracted with EtOAc (1000 mL×3). The combined organic phase was washed with brine (1000 mL×3), dried over Na₂SO₄, filtered and concentrated under reduced pressure to give a residue. The residue was purified by flash silica gel chromatography (ISCO®; 80 g SepaFlash® Silica Flash Column, Eluent of 0˜10% Ethyl acetate/Petroleum ether gradient @ 150/min) to give 7-fluoro-5-methoxy-1H-indole.

### Step 2: 1-(7-fluoro-5-methoxy-1H-indol-3-yl)-N,N-dimethylmethanamine

To a solution of 7-fluoro-5-methoxy-1H-indole (4.2 g, 25.43 mmol, 1 eq) in dioxane (40 mL) and H₂O (40 mL) was added N-methylmethanamine; hydrochloride (2.2 g, 27.9 mmol, 1.1 eq), formaldehyde (2.2 g, 27.9 mmol, 2.0 mL, 37% purity, 1.1 eq), TEA (2.8 g, 27.9 mmol, 3.8 mL, 1.1 eq) and AcOH (2.2 g, 38.1 mmol, 2.1 mL, 1.5 eq). The mixture was stirred at 30° C. for 12 hr. LC-MS showed the desired compound was detected. The mixture was poured into ice NaOH (400 mL, 2N) and white solid was separate out. The mixture was filtered and washed with H₂O (200 mL) and the cake was collected to give 1-(7-fluoro-5-methoxy-1H-indol-3-yl)-N,N-dimethyl-methanamine.

### Step 3: benzyl-3-((7-fluoro-5-methoxy-1H-indol-3-yl)methyl)piperidin-4-one

To a solution of 1-(7-fluoro-5-methoxy-1H-indol-3-yl)-N,N-dimethyl-methanamine (4 g, 18.0 mmol, 1 eq) in dioxane (55 mL) was added 1-benzyl-4-pyrrolidin-1-yl-3,6-dihydro-2H-pyridine (10.9 g, 44.9 mmol, 2.5 eq). The mixture was stirred at 120° C. for 12 hr. LC-MS showed the desired compound was detected. The reaction mixture was concentrated under reduced pressure to give a residue. The residue was purified by flash silica gel chromatography (ISCO®; 25 g SepaFlash® Silica Flash Column, Eluent of 0˜15% Ethyl acetate/Petroleum ether gradient @ 150 mL/min) to give 1-benzyl-3-[(7-fluoro-5-methoxy-1H-indol-3-yl)methyl]piperidin-4-one.

### Step 4: 1-benzyl-4-ethynyl-3-((7-fluoro-5-methoxy-1H-indol-3-yl)methyl)piperidin-4-ol

To a solution of 1-benzyl-3-[(7-fluoro-5-methoxy-1H-indol-3-yl)methyl]piperidin-4-one (7.6 g, 20.8 mmol, 1 eq) in THF (150 mL) was added bromo(ethynyl)magnesium (0.5 M, 167.0 mL, 4 eq) at 0 C. The mixture was stirred at 0° C. for 1 hr. LC-MS showed the desired compound was detected. The reaction mixture was partitioned between NH₄Cl 300 mL and EtOAc 300 mL. The water phase was separated, extracted with EtOAc (300 mL×3). The combined organic phase was washed with brine (200 mL×3), dried over Na₂SO₄, filtered and concentrated under reduced pressure to give 1-benzyl-4-ethynyl-3-[(7-fluoro-5-methoxy-1H-indol-3-yl)methyl]piperidin-4-ol.

### Step 5: 2-benzyl-7-fluoro-9-methoxy-5-methyl-2,3,4,6-tetrahydro-1H-pyrido[4,3-b]carbazole

To a solution of 1-benzyl-4-ethynyl-3-[(7-fluoro-5-methoxy-1H-indol-3-yl)methyl]piperidin-4-ol (6.8 g, 17.4 mmol, 1 eq) in Tol. (120 mL) was added TFA (19.9 g, 174.7 mmol, 12.9 mL, 10 eq). The mixture was stirred at 80° C. for 12 hr. LC-MS showed the desired compound was detected. The mixture was concentrated to get crude residue and diluted with EtOAc 100 mL and adjusted pH=8 with sat NaHCO₃, extracted with EtOAc (200 mL×3). The combined organic layers were washed with brine (100 mL×3), dried over Na₂SO₄, filtered and concentrated under reduced pressure to give a residue. The residue was purified by flash silica gel chromatography (ISCO®; 25 g SepaFlash® Silica Flash Column, Eluent of 0˜50% Ethyl acetate/Petroleum ether gradient @ 150 mL/min) to give 2-benzyl-7-fluoro-9-methoxy-5-methyl-1,3,4,6-tetrahydropyrido[4,3-b]carbazole.

### Step 6: 7-fluoro-9-methoxy-5-methyl-6H-pyrido[4,3-b]carbazole

To a solution of Pd/C (800 mg, 10% purity) in Ph₂O (7 mL) was added 2-benzyl-7-fluoro-9-methoxy-5-methyl-1,3,4,6-tetrahydropyrido[4,3-b]carbazole (500 mg, 1.3 mmol, 1 eq). The mixture was stirred at 220° C. for 1 hr under N₂. LC-MS showed the desired compound was detected. The reaction mixture was filtered and the filtrate dried in vacuum to give crude product. The residue was purified by flash silica gel chromatography (ISCO®; 25 g SepaFlash® Silica Flash Column, Eluent of 0˜10% CH₂Cl₂/MeOH @ 150 mL/min) to give 7-fluoro-9-methoxy-5-methyl-6H-pyrido[4,3-b]carbazole.

### Step 7: 7-fluoro-9-methoxy-5,6-dimethyl-6H-pyrido[4,3-b]carbazole

To a solution of 7-fluoro-9-methoxy-5-methyl-6H-pyrido[4,3-b]carbazole (480 mg, 1.7 mmol, 1 eq) in THF (8 mL) was added NaH (205.4 mg, 5.1 mmol, 60% purity, 3 eq) at 0° C. and stirred at 0° C. for 30 min, and Mel (230.9 mg, 1.6 mmol, 101.2 uL, 0.9 eq) was added to the mixture at 0° C. The mixture was stirred at 25° C. for 2 hr. TLC indicated Reactant was consumed completely and one major new spot with larger polarity was detected. The reaction mixture was partitioned between NH₄Cl 30 mL and EtOAc 50 mL. The water phase was separated, extracted with EtOAc (50 mL×3). The combined organic phase was washed with brine (20 mL×3), dried over Na₂SO₄, filtered and concentrated under reduced pressure to give a residue to give 7-fluoro-9-methoxy-5,6-dimethyl-pyrido[4,3-b]carbazole.

### Step 8: 7-fluoro-5,6-dimethyl-6H-pyrido[4,3-b]carbazol-9-ol

To a solution of 7-fluoro-9-methoxy-5,6-dimethyl-pyrido[4,3-b]carbazole (500 mg, 1.7 mmol, 1 eq) in DCM (20 mL) was added BBr₃ (851.1 mg, 3.4 mmol, 327.3 uL, 2 eq) at 0° C. The mixture was stirred at 0° C. for 2 hr. LC-MS showed the desired compound was detected. The reaction mixture was added to NH₃·H₂O (purity 25%, 10 mL) and filtered and the filter cake was washed with 20 mL of H₂O, dried in vacuum to give crude product to give 7-fluoro-5,6-dimethyl-pyrido[4,3-b]carbazol-9-ol.

### Step 9: 9-((4,5-dihydro-1H-imidazol-2-yl)methoxy)-7-fluoro-5,6-dimethyl-6H-pyrido[4,3-b]carbazole

To a solution of 7-fluoro-5,6-dimethyl-pyrido[4,3-b]carbazol-9-ol (40 mg, 142.7 umol, 1 eq) in DMF (2 mL) were added NaH (57.0 mg, 1.4 mmol, 60% purity, 10 eq) and 2-(chloromethyl)-4,5-dihydro-1H-imidazole (33.8 mg, 285.4 umol, 2 eq). The mixture was stirred at 25° C. for 1 hr. LCMS showed the reaction was completed and desired product was detected. The reaction mixture was quenched by addition HCOOH 1 mL at 25° C. and concentrated under reduced pressure to give a residue. The residue was purified by prep-HPLC (TFA condition; column: Phenomenex Gemini-NX 150*30 mm*5 um; mobile phase: [water(0.1% TFA)-ACN]; B %: 1%-20%, 9 min) to give 9-(4,5-dihydro-1H-imidazol-2-ylmethoxy)-7-fluoro-5,6-dimethyl-pyrido[4,3-b]carbazole.

MS (ESI): m/z=363.1[M+H]⁺

### 9-(4,5-dihydro-1H-imidazol-2-ylmethoxy)-6-ethyl-pyrido[4,3-b]carbazole (Compound 255)

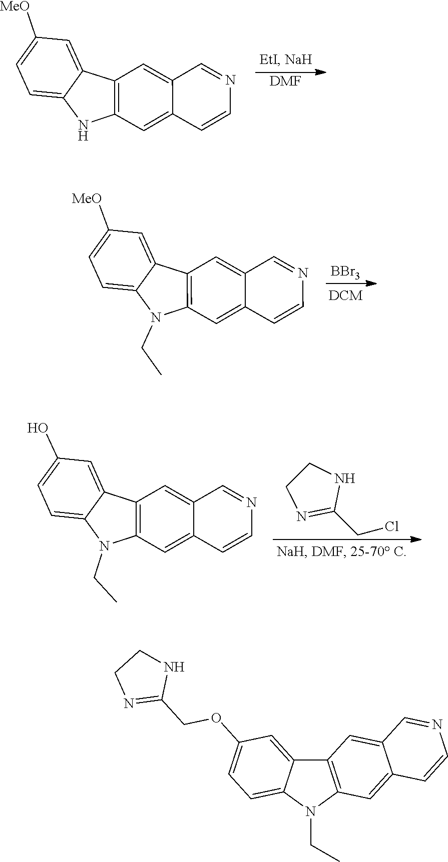

### Step 1: 6-ethyl-9-methoxy-pyrido[4,3-b]carbazole

To a solution of 9-methoxy-6H-pyrido[4,3-b]carbazole (50 mg, 201.3 umol, 1 eq) in DMF (2 mL) was added NaH (16.1 mg, 402.8 umol, 60% purity, 2 eq) and the mixture was stirred at 0° C. for 30 min. Etl (37.7 mg, 241.7 umol, 19.3 uL, 1.2 eq) was added to the mixture and stirred at 25° C. for 2 hr. LC-MS showed the desired compound was detected. The mixture was added to the NH₄Cl (20 mL). The residue was extracted with EtOAc (30 mL×3). The combined organic layers were washed with brine (60 mL×1), dried over Na₂SO₄, filtered and concentrated under reduced pressure to give 6-ethyl-9-methoxy-pyrido[4,3-b]carbazole.

### Step 2: 6-ethylpyrido[4,3-b]carbazol-9-ol

To a solution of 6-ethyl-9-methoxy-pyrido[4,3-b]carbazole (190 mg, 687.6 umol, 1 eq) in DCM (3 mL) was added BBr₃ (861.3 mg, 3.4 mmol, 331.3 uL, 5 eq) at 0° C. The mixture was stirred at 25° C. for 2 hr. LC-MS showed the desired compound was detected. The reaction mixture was added to the NH₃·H₂O (40 mL, 25%) at 0° C. The mixture was filtered and the filter cake was washed with 30 mL of H₂O, concentrated under reduced pressure to give 6-ethylpyrido[4,3-b]carbazol-9-ol.

### Step 3: 9-(4,5-dihydro-1H-imidazol-2-ylmethoxy)-6-ethyl-pyrido[4,3-b]carbazole

To a solution of 6-ethylpyrido[4,3-b]carbazol-9-ol (40 mg, 152.5 umol, 1 eq) in DMF (3 mL) was added NaH (24.4 mg, 609.9 umol, 60% purity, 4 eq) at 25° C. and stirred for 30 min. 2-(chloromethyl)-4,5-dihydro-1H-imidazole (28.4 mg, 183.0 umol, 1.2 eq, HCl) and NaI (22.9 mg, 152.5 umol, 1 eq) was added to the mixture and stirred at 70° C. for 1 hr. LC-MS showed the desired compound was detected. The reaction mixture was concentrated under reduced pressure to remove solvent. The residue was purified by prep-HPLC (TFA condition, column: Phenomenex luna C18 100×40 mm×5 um; mobile phase: [water(0.1% TFA)-ACN]; B %: 1%-30%, 8 min) to give 9-(4,5-dihydro-1H-imidazol-2-ylmethoxy)-6-ethyl-pyrido[4,3-b]carbazole.

MS (ESI): m/z=345.1[M+H]⁺

### 9-(4,5-dihydro-1H-imidazol-2-ylmethoxy)-6-isopropyl-pyrido[4,3-b]carbazole (Compound 256)

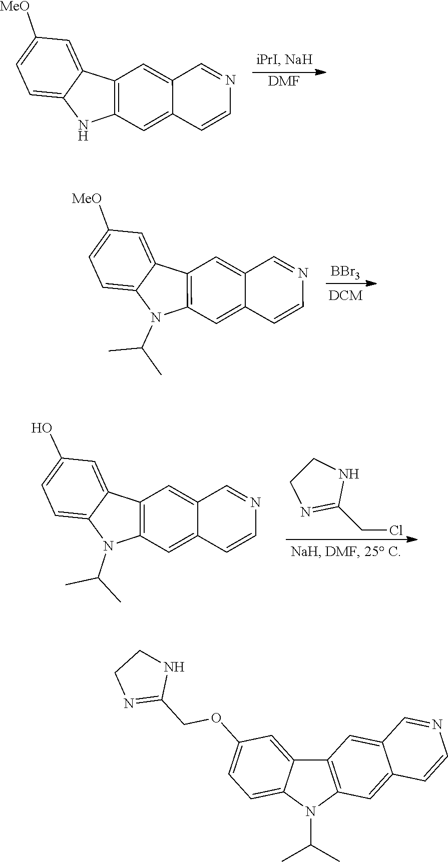

### Step 1: 6-isopropyl-9-methoxy-6H-pyrido[4,3-b]carbazole

To a solution of 9-methoxy-6H-pyrido[4,3-b]carbazole (150 mg, 604.1 umol, 1 eq) in DMF (6 mL) was added NaH (72.4 mg, 1.8 mmol, 60% purity, 3 eq) at 0° C. and stirred for 30 min. 2-iodopropane (308.1 mg, 1.8 mmol, 181.2 uL, 3 eq) was added to the mixture and stirred for 2 hr at 65° C. LC-MS showed the desired compound was detected. The reaction mixture was partitioned between NH₄Cl 20 mL and EtOAc 50 mL. The water phase was separated, extracted with EtOAc (50 mL×3). The combined organic phase was washed with brine (30 mL×3), dried over Na₂SO₄, filtered and concentrated under reduced pressure to give 6-isopropyl-9-methoxy-pyrido[4,3-b]carbazole.

### Step 2: 6-isopropyl-6H-pyrido[4,3-b]carbazol-9-ol

To a solution of 6-isopropyl-9-methoxy-pyrido[4,3-b]carbazole (300 mg, 1.0 mmol, 1 eq) in DCM (5 mL) was added BBr₃ (1.2 g, 5.1 mmol, 497.7 uL, 5 eq) at 0° C. The mixture was stirred at 25° C. for 1 hr. LC-MS showed the desired compound was detected. The reaction mixture was added to NH₃·H₂O (purity 25%, 8 mL), filtered and the filter cake was washed with 10 mL of H₂O, dried in vacuum to give 6-isopropylpyrido[4,3-b]carbazol-9-ol.

### Step 3: 9-((4,5-dihydro-1H-imidazol-2-yl)methoxy)-6-isopropyl-6H-pyrido[4,3-b]carbazole

To a solution of 6-isopropylpyrido[4,3-b]carbazol-9-ol (60 mg, 217.13 umol, 1 eq) in DMF (3 mL) was added NaH (26.0 mg, 651.3 umol, 60% purity, 3 eq) at 25° C. and stirred for 30 min, 2-(chloromethyl)-4,5-dihydro-1H-imidazole (40.3 mg, 260.5 umol, 1.2 eq, HCl) and NaI (32.5 mg, 217.1 umol, 1 eq) was added to the mixture. The mixture was stirred at 25° C. for 2 hr. LC-MS showed the desired compound was detected. The reaction mixture was concentrated under reduced pressure to give a residue. The residue was purified by prep-HPLC (TFA condition, column: Phenomenex Gemini-NX 150*30 mm*5 um; mobile phase: [water(0.1% TFA)-ACN]; B %: 1%-30%, 9 min) to give 9-(4,5-dihydro-1H-imidazol-2-ylmethoxy)-6-isopropyl-pyrido[4,3-b]carbazole.

MS (ESI): m/z=359.1[M+H]⁺

### 9-((4,5-dihydro-1H-imidazol-2-yl)methoxy)-5-methyl-6H-pyrido[4,3-b]carbazole (Compound 257)

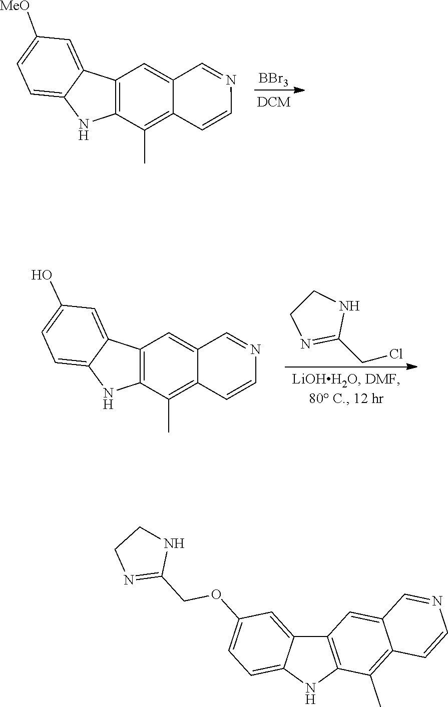

### Step 1: 5-methyl-6H-pyrido[4,3-b]carbazol-9-ol

To a solution of 9-methoxy-5-methyl-6H-pyrido[4,3-b]carbazole (100 mg, 381.2 umol, 1 eq) in DCM (2 mL) was added BBr₃ (477.5 mg, 1.9 mmol, 183.6 uL, 5 eq) at 0° C. The mixture was stirred at 25° C. for 1 hr. LC-MS showed the desired compound was detected. The reaction mixture was added to NH₃·H₂O (purity 25%, 4 mL) and filtered and the filter cake was washed with 10 mL of H₂O, dried in vacuum to give 5-methyl-6H-pyrido[4,3-b]carbazol-9-ol.

### Step 2: 9-((4,5-dihydro-1H-imidazol-2-yl)methoxy)-5-methyl-6H-pyrido[4,3-b]carbazole

To a solution of 5-methyl-6H-pyrido[4,3-b]carbazol-9-ol (60 mg, 241.6 umol, 1 eq) in DMF (3 mL) was added 2-(chloromethyl)-4,5-dihydro-1H-imidazole (44.9 mg, 290.0 umol, 1.2 eq, HCl), LiOH·H₂O (30.4 mg, 724.9 umol, 3 eq) and NaI (36.2 mg, 241.6 umol, 1 eq). The mixture was stirred at 80° C. for 12 hr. LC-MS showed the desired compound was detected. The reaction mixture was concentrated under reduced pressure to give a residue. The residue was purified by prep-HPLC (TFA condition, column: Phenomenex luna C18 100*40 mm*5 um; mobile phase: [water(0.1% TFA)-ACN]; B %: 5%-36%, 8 min) to give 9-(4,5-dihydro-1H-imidazol-2-ylmethoxy)-5-methyl-6H-pyrido[4,3-b]carbazole.

MS (ESI): m/z=331.1M+H]⁺

### 2-((7-fluoro-5,6-dimethyl-6H-pyrido[4,3-b]carbazol-9-yl)oxy)-N,N-dimethylethanamine (Compound 258)

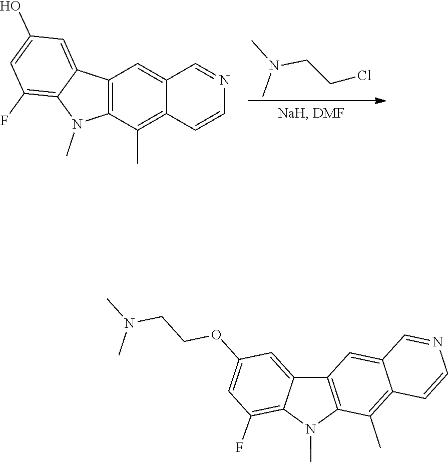

To a solution of 7-fluoro-5,6-dimethyl-pyrido[4,3-b]carbazol-9-ol (80 mg, 285.4 umol, 1 eq) in DMF (3 mL) was added NaH (34.2 mg, 856.2 umol, 60% purity, 3 eq) 2-chloro-N,N-dimethyl-ethanamine (49.3 mg, 342.5 umol, 1.2 eq, HCl) the mixture was stirred at 25° C. for 30 min, 2-chloro-N,N-dimethyl-ethanamine (49.3 mg, 342.5 umol, 1.2 eq, HCl) was added to the mixture. The mixture was stirred at 70° C. for 2 hr. LC-MS showed the desired compound was detected. The reaction mixture was concentrated under reduced pressure to give a residue. The residue was purified by prep-HPLC (TFA condition column: Phenomenex luna C18 100*40 mm*5 um; mobile phase: [water(0.1% TFA)-ACN]; B %: 5%-30%, 8 min) to give 2-(7-fluoro-5,6-dimethyl-pyrido[4,3-b]carbazol-9-yl)oxy-N,N-dimethyl-ethanamine.

MS (ESI): m/z=352.1[M+H]⁺

### 4-(2-((7-fluoro-5,6-dimethyl-6H-pyrido[4,3-b]carbazol-9-yl)oxy)ethyl)morpholine (Compound 259)

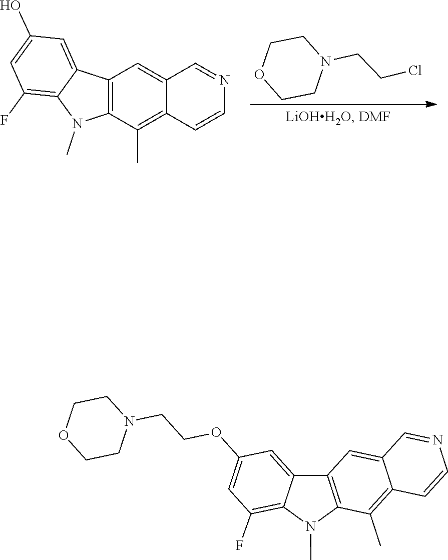

To a solution of 7-fluoro-5,6-dimethyl-pyrido[4,3-b]carbazol-9-ol (80 mg, 285.4 umol, 1 eq) in DMF (2 mL) was added 4-(2-chloroethyl)morpholine (63.7 mg, 342.5 umol, 1.2 eq, HCl), LiOH·H₂O (35.9 mg, 856.2 umol, 3 eq) and NaI (42.7 mg, 285.4 umol, 1 eq). The mixture was stirred at 80° C. for 12 hr. LC-MS showed the desired compound was detected. The reaction mixture was concentrated under reduced pressure to give a residue. The residue was purified by prep-HPLC (TFA condition, column: Phenomenex luna C18 100*40 mm*5 um; mobile phase: [water(0.1% TFA)-ACN]; B %: 5%-30%, 8 min) to give 4-[2-(7-fluoro-5,6-dimethyl-pyrido[4,3-b]carbazol-9-yl)oxyethyl]morpholine.

MS (ESI): m/z=394.2[M+H]⁺

### 9-((4,5-dihydro-1H-imidazol-2-yl)methoxy)-6-methyl-6H-pyrido[3,2-b]carbazole (Compound 260)

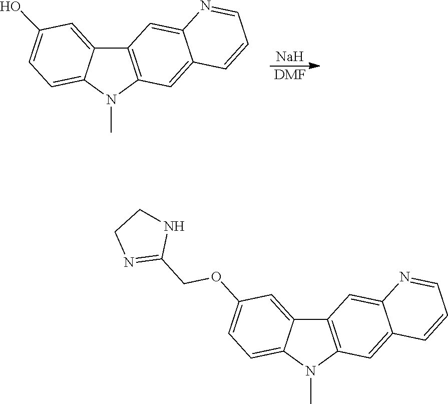

To a solution of 6-methyl-6H-pyrido[3,2-b]carbazol-9-ol (40 mg, 161.1 umol, 1 eq) in DMF (2 mL) were added NaH (64.4 mg, 1.6 mmol, 60% purity, 10 eq) and 2-(chloromethyl)-4,5-dihydro-1H-imidazole (49.9 mg, 322.2 umol, 2 eq, HCl). The mixture was stirred at 25° C. for 1 hr. LCMS showed the reaction was completed and desired product was detected. The reaction mixture was quenched by addition HCOOH (1 mL) at 25° C. and concentrated under reduced pressure to give a residue. The residue was purified by prep-HPLC (TFA condition, column: Phenomenex luna C18 100*40 mm*5 um; mobile phase: [water(0.1% TFA)-ACN]; B %: 5%-25%, 8 min) to give 9-((4,5-dihydro-1H-imidazol-2-yl)methoxy)-6-methyl-6H-pyrido[3,2-b]carbazole.

MS (ESI): m/z=331.0 [M+H]⁺

### 2-((6-methyl-6H-pyrido[3,2-b]carbazol-9-yl)oxy)acetimidamide (Compound 261)

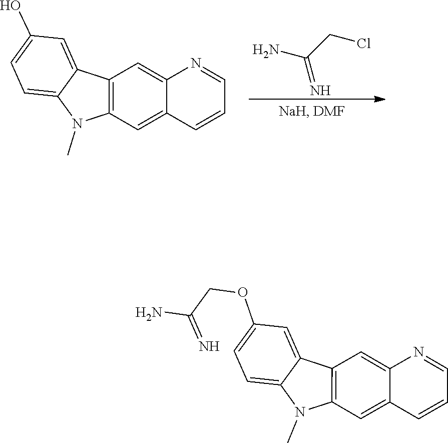 [SMILES: Cn1c2ccc(O)cc2c2cc3ncccc3cc21 ; N=C(N)CCl ; Cn1c2ccc(OCC(=N)N)cc2c2cc3ncccc3cc21]

To a solution of 6-methylpyrido[3,2-b]carbazol-9-ol (46 mg, 185.2 umol, 1 eq) in DMF (2 mL) were added NaH (74.1 mg, 1.8 mmol, 60% purity, 10 eq) and 2-chloroacetamidine (47.8 mg, 370.5 umol, 2 eq, HCl). The mixture was stirred at 25° C. for 1 hr. LCMS showed the reaction was completed and desired product was detected. The reaction mixture was quenched by addition HCOOH (1 mL) at 25° C. and concentrated under reduced pressure to give a residue. The residue was purified by prep-HPLC (TFA condition, column: Phenomenex luna C18 100*40 mm*5 um; mobile phase: [water(0.1% TFA)-ACN]; B %: 5%-25%, 8 min) to afford 2-(6-methylpyrido[3,2-b]carbazol-9-yl)oxyacetamidine.

MS (ESI): m/z=305.1 [M+H]⁺

### 9-((4,5-dihydro-1H-imidazol-2-yl)methoxy)-6H-pyrido[4,3-b]carbazole (Compound 262)

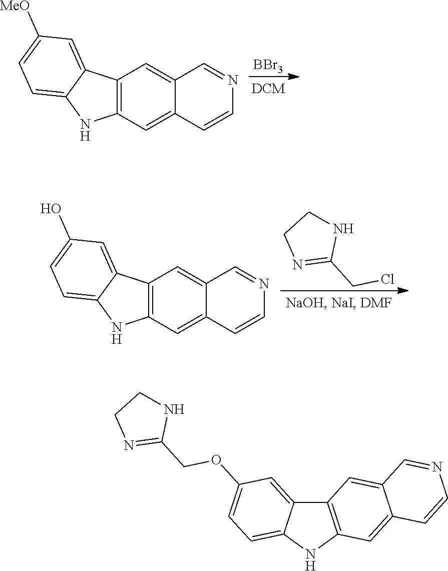

### Step 1: 6H-pyrido[4,3-b]carbazol-9-ol

To a solution of 9-methoxy-6H-pyrido[4,3-b]carbazole (400 mg, 1.6 mmol, 1 eq) in DCM (15 mL) was added BBr₃ (2.0 g, 8.0 mmol, 776.1 uL, 5 eq) at 0° C. The mixture was stirred at 25° C. for 2 hr. LCMS showed the reaction was completely. The reaction was slowly added to NH₃·H₂O (25% purity, 30 mL) at 0° C. The mixture was filtered, and the collected solid was washed with H₂O (20 mL×3). The combined aqueous was extracted with DCM (20 mL×3). The collected solid and the organic extracts were combined and dried over anhydrous Na₂SO₄ and concentrated to give 6H-pyrido[4,3-b]carbazol-9-ol.

### Step 2: 9-((4,5-dihydro-1H-imidazol-2-yl)methoxy)-6H-pyrido[4,3-b]carbazole

To a solution of 6H-pyrido[4,3-b]carbazol-9-ol (50 mg, 213.4 umol, 1 eq) in DMF (2 mL) was added 2-(chloromethyl)-4,5-dihydro-1H-imidazole; hydrochloride (39.7 mg, 256.1 umol, 1.2 eq), NaOH (25.6 mg, 640.3 umol, 3 eq) and sodium iodide (31.9 mg, 213.4 umol, 1 eq). The mixture was stirred at 70° C. for 12 hr. The reaction was concentrated to give a residue. The residue was purified by prep-HPLC (TFA condition; column: Phenomenex Gemini-NX 150*30 mm*5 um; mobile phase: [water(0.1% TFA)-ACN]; B %: 1%-23%, 9 min) to afford 9-((4,5-dihydro-1H-imidazol-2-yl)methoxy)-6H-pyrido[4,3-b]carbazole.

MS (ESI): m/z=317.1 [M+H]⁺

### 2-[(5-methyl-6H-pyrido[4,3-b]carbazol-9-yl)oxy]ethanamine (Compound 263)

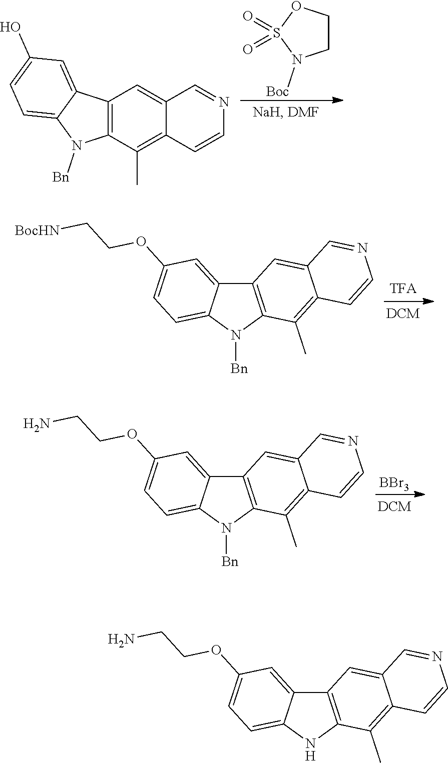

### Step 1: tert-butyl (2-((6-benzyl-5-methyl-6H-pyrido[4,3-b]carbazol-9-yl)oxy)ethyl)carbamate

To a solution of 6-benzyl-5-methyl-pyrido[4,3-b]carbazol-9-ol (60 mg, 177.3 umol, 1 eq) in DMF (5 mL) was added NaH (21.2 mg, 531.9 umol, 60% purity, 3 eq) and the solution was stirred at 25° C. for 30 min. Tert-butyl 2,2-dioxooxathiazolidine-3-carboxylate (47.5 mg, 212.7 umol, 1.2 eq) was added to the mixture and stirred at 70° C. for 12 hr. The reaction mixture was partitioned between NH₄Cl 5 mL and EtOAc 5 mL. The water phase was separated, extracted with EtOAc (5 mL×3). The combined organic phase was washed with brine (5 mL×3), dried over Na₂SO₄, filtered, and concentrated under reduced pressure to give tert-butyl N-[2-(6-benzyl-5-methyl-pyrido[4,3-b]carbazol-9-yl)oxyethyl]carbamate.

### Step 2: 2-((6-benzyl-5-methyl-6H-pyrido[4,3-b]carbazol-9-yl)oxy)ethanamine

To a solution of tert-butyl N-[2-(6-benzyl-5-methyl-pyrido[4,3-b]carbazol-9-yl)oxyethyl]carbamate (50 mg, 103.8 umol, 1 eq) in DCM (5 mL) was added TFA (35.5 mg, 311.4 umol, 23.0 uL, 3 eq). The mixture was stirred at 25° C. for 1 hr. The reaction mixture was concentrated under reduced pressure to give 2-(6-benzyl-5-methyl-pyrido[4,3-b]carbazol-9-yl)oxyethanamine.

### Step 3: 2-((5-methyl-6H-pyrido[4,3-b]carbazol-9-yl)oxy)ethanamine

To a solution of 2-(6-benzyl-5-methyl-pyrido[4,3-b]carbazol-9-yl)oxyethanamine (50 mg, 131.0 umol, 1 eq) in DCM (3 mL) was added BBr₃ (164.1 mg, 655.3 umol, 63.1 uL, 5 eq) at −20° C. The mixture was stirred at −20° C. for 30 min. The reaction mixture was added to ice NH₃·H₂O (purity 25%, 4 mL) and filtered. The collected solid was washed with 5 mL of H₂O and dried in vacuum to give a residue. The residue was purified by prep-HPLC (TFA condition, column: 3-Phenomenex Luna C18 75*30 mm*3 um; mobile phase: [water(0.1% TFA)-ACN]; B %: 1%-30%, 10 min) to give 2-[(5-methyl-6H-pyrido[4,3-b]carbazol-9-yl)oxy]ethanamine.

MS (ESI): m/z=292.1 [M+H]⁺

### 2-(7-fluoro-5,6-dimethyl-pyrido[4,3-b]carbazol-9-yl)oxyacetamide (Compound 264)

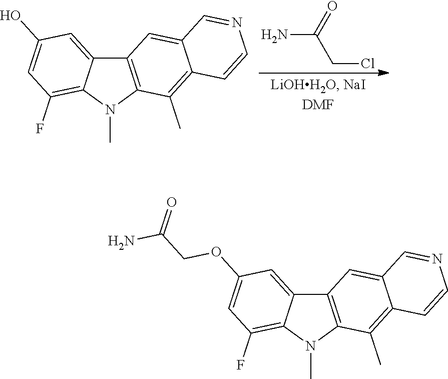

To a solution of 7-fluoro-5,6-dimethyl-pyrido[4,3-b]carbazol-9-ol (30 mg, 107.0 umol, 1 eq) in DMF (2 mL) was added 2-chloroacetamide (12.0 mg, 128.4 umol, 1.2 eq), LiOH·H₂O (17.9 mg, 428.1 umol, 4 eq) and NaI (16.0 mg, 107.0 umol, 1 eq). The mixture was stirred at 80° C. for 12 hr. The reaction mixture was concentrated under reduced pressure to give a residue. The residue was purified by prep-HPLC (TFA condition; column: Phenomenex luna C18 100*40 mm*5 um; mobile phase: [water(0.1% TFA)-ACN]; B %: 5%-30%, 8 min) to give 2-(7-fluoro-5,6-dimethyl-pyrido[4,3-b]carbazol-9-yl)oxyacetamide.

MS (ESI): m/z=338.1 [M+H]⁺

### (R)-7-fluoro-5,6-dimethyl-9-(pyrrolidin-2-ylmethoxy)-6H-pyrido[4,3-b]carbazole (Compound 265)

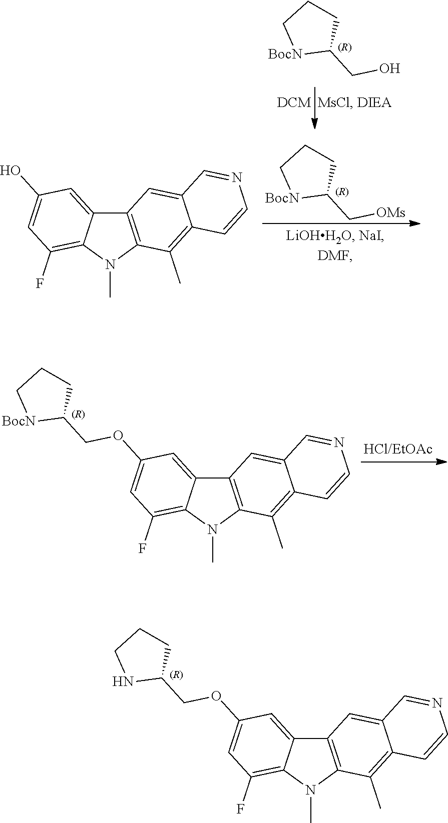

### Step 1: (R)-tert-butyl 2-(((methylsulfonyl)oxy)methyl)pyrrolidine-1-carboxylate

To a solution of tert-butyl (2R)-2-(hydroxymethyl)pyrrolidine-1-carboxylate (100 mg, 496.8 umol, 1 eq) in DCM (3 mL) was added DIEA (192.6 mg, 1.4 mmol, 259.6 uL, 3 eq) and MsCl (113.8 mg, 993.7 umol, 76.9 uL, 2 eq) at 0° C. The mixture was stirred at 25° C. for 1 hr. The reaction mixture diluted with NaHCO₃ 5 mL and extracted with DCM (5 mL×2). The combined organic layers were washed with brine (10 mL×1), dried over Na₂SO₄, filtered, and concentrated under reduced pressure to give tert-butyl (R)-tert-butyl 2-(((methylsulfonyl)oxy)methyl)pyrrolidine-1-carboxylate.

### Step 2: (R)-tert-butyl 2-(((7-fluoro-5,6-dimethyl-6H-pyrido[4,3-b]carbazol-9-yl)oxy)methyl)pyrrolidine-1-carboxylate

To a solution of 7-fluoro-5,6-dimethyl-pyrido[4,3-b]carbazol-9-ol (45 mg, 160.5 umol, 1 eq) in DMF (2 mL) was added (R)-tert-butyl 2-(((methylsulfonyl)oxy)methyl)pyrrolidine-1-carboxylate (42.3 mg, 192.6 umol, 1.2 eq), LiOH·H₂O (26.9 mg, 642.1 umol, 4 eq) and NaI (24.0 mg, 160.5 umol, 1 eq). The mixture was stirred at 80° C. for 12 hr. The reaction mixture was partitioned between H₂O 5 mL and EtOAc 10 mL. The water phase was separated, extracted with EtOAc (10 mL×3). The combined organic phase was washed with brine (10 mL×3), dried over Na₂SO₄, filtered, and concentrated under reduced pressure to give (R)-tert-butyl 2-(((7-fluoro-5,6-dimethyl-6H-pyrido[4,3-b]carbazol-9-yl)oxy)methyl)pyrrolidine-1-carboxylate.

### Step 3: (R)-7-fluoro-5,6-dimethyl-9-(pyrrolidin-2-ylmethoxy)-6H-pyrido[4,3-b]carbazole

A mixture of (R)-tert-butyl 2-(((7-fluoro-5,6-dimethyl-6H-pyrido[4,3-b]carbazol-9-yl)oxy)methyl)pyrrolidine-1-carboxylate (100 mg, 215.7 umol, 1 eq) in HCl/EtOAc (2 mL) (4N) was stirred at 25° C. for 2 hr. The reaction mixture was concentrated under reduced pressure to give a residue. The residue was purified by prep-HPLC (HCl condition; column: Welch Xtimate C18 100*25 mm*3 um; mobile phase:[water(0.05% HCl)-ACN]; B %: 1%-25%, 8 min) to give (R)-7-fluoro-5,6-dimethyl-9-(pyrrolidin-2-ylmethoxy)-6H-pyrido[4,3-b]carbazole.

MS (ESI): m/z=364.1 [M+H]⁺

### 5,6-dimethyl-9-(3-(piperidin-1-yl)propoxy)-6H-pyrido[4,3-b]carbazole (Compound 266)

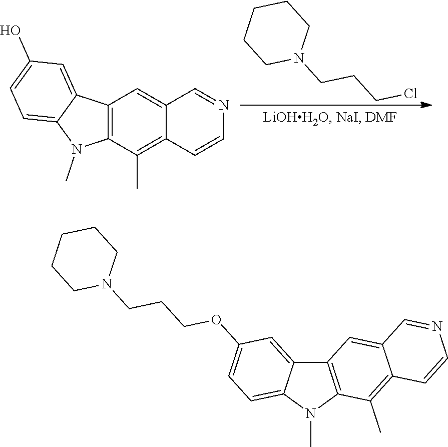 [SMILES: Cc1c2ccncc2cc2c3cc(O)ccc3n(C)c12 ; ClCCCN1CCCCC1 ; Cc1c2ccncc2cc2c3cc(OCCCN4CCCCC4)ccc3n(C)c12]

To a solution of 1-(3-chloropropyl)piperidine (30.2 mg, 152.4 umol, 1.0 eq, HCl) in DMF (2 mL) was added 5,6-dimethylpyrido[4,3-b]carbazol-9-ol (40 mg, 152.4 umol, 1 eq), LiOH·H₂O (25.5 mg, 609.9 umol, 4 eq) and NaI (22.8 mg, 152.4 umol, 1 eq). The mixture was stirred at 80° C. for 12 hr. The reaction was concentrated, and the residue was purified by prep-HPLC (HCl condition; column: Welch Xtimate C18 100*25 mm*3 um; mobile phase: [water(0.05% HCl)-MeOH]; B %: 25%-55%, 8 min) to afford 5,6-dimethyl-9-(3-(piperidin-1-yl)propoxy)-6H-pyrido[4,3-b]carbazole.

MS (ESI): m/z=388.1 [M+H]⁺

### (R)-4-((5,6-dimethyl-6H-pyrido[4,3-b]carbazol-9-yl)oxy)butan-2-amine (Compound 267)

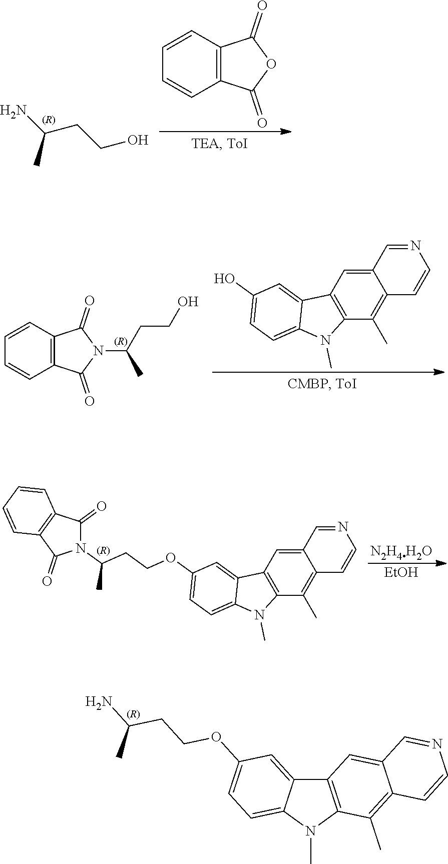

### Step 1: (R)-2-(4-hydroxybutan-2-yl)isoindoline-1,3-dione

To a solution of (R)-3-aminobutan-1-ol (1.6 g, 11.2 mmol, 1 eq) in Tol (10 mL) was added (3R)-3-aminobutan-1-ol (1 g, 11.2 mmol, 1 eq) and TEA (385.9 mg, 3.8 mmol, 530.9 uL, 0.3 eq). The mixture was stirred at 110° C. for 12 hr. The reaction mixture was concentrated under reduced pressure to give a residue. The residue was purified by flash silica gel chromatography (ISCO®; 12 g SepaFlash® Silica Flash Column, Eluent of 0˜100% Petroleum ether gradient/Ethyl acetate @ 80 mL/min) to give (R)-2-(4-hydroxybutan-2-yl)isoindoline-1,3-dione.

### Step 2: (R)-2-(4-((5,6-dimethyl-6H-pyrido[4,3-b]carbazol-9-yl)oxy)butan-2-yl)isoindoline-1,3-dione

To a solution of (R)-2-(4-hydroxybutan-2-yl)isoindoline-1,3-dione (49.8 mg, 190.0 umol, 1 eq) in Tol (3 mL) was added 5,6-dimethyl-6H-pyrido[4,3-b]carbazol-9-ol (50 mg, 228.0 umol, 1.2 eq) and 2-(tributyl-λ⁵-phosphanylidene)acetonitrile (229.3 mg, 950.2 umol, 5 eq) under N₂. The mixture was stirred at 100° C. for 12 hr under N₂. The reaction mixture was concentrated under reduced pressure to remove solvent. The residue was purified by prep-TLC (SiO₂, DCM/MeOH=10/1) to give (R)-2-(4-((5,6-dimethyl-6H-pyrido[4,3-b]carbazol-9-yl)oxy)butan-2-yl)isoindoline-1,3-dione.

### Step 3: (R)-4-((5,6-dimethyl-6H-pyrido[4,3-b]carbazol-9-yl)oxy)butan-2-amine

To a solution of (R)-2-(4-((5,6-dimethyl-6H-pyrido[4,3-b]carbazol-9-yl)oxy)butan-2-yl)isoindoline-1,3-dione (30 mg, 64.7 umol, 1 eq) in EtOH (2 mL) was added N₂H₄·H₂O (66.1 mg, 1.2 mmol, 64.2 uL, 98% purity, 20 eq). The mixture was stirred at 80° C. for 12 hr. The reaction mixture was diluted with H₂O (10 mL) and extracted with EtOAc (5 mL×3). The combined organic layers were washed with brine (5 mL×2), dried over Na₂SO₄, filtered and the filtrate was concentrated under reduced pressure to give a residue. The residue was purified by prep-HPLC (TFA condition, column: Waters Xbridge BEH C18 100*30 mm*10 um; mobile phase: [water(0.1% TFA)-ACN]; B %: 10%-40%, 10 min) to give (R)-4-((5,6-dimethyl-6H-pyrido[4,3-b]carbazol-9-yl)oxy)butan-2-amine.

MS (ESI): m/z=334.1 [M+H]⁺

### ((5,6-dimethyl-6H-pyrido[4,3-b]carbazol-9-yl)oxy)butan-2-amine (Compound 268)

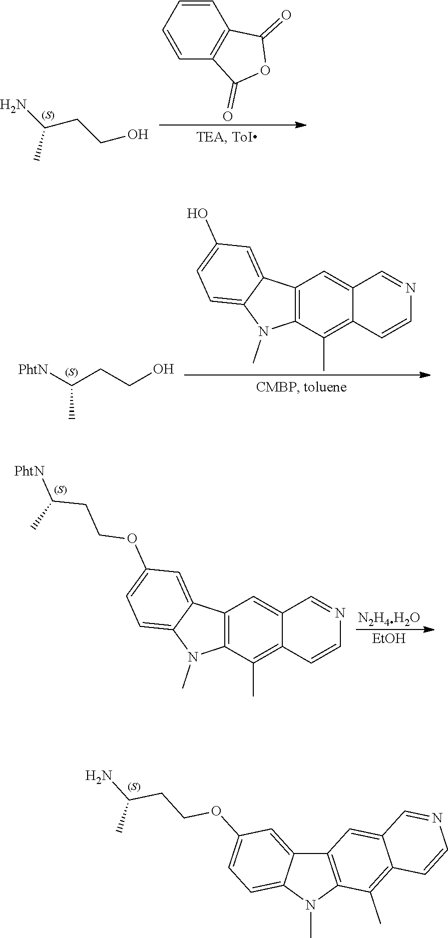

### Step 1: (S)-2-(4-hydroxybutan-2-yl)isoindoline-1,3-dione

To a solution of isobenzofuran-1,3-dione (1.6 g, 11.2 mmol, 1 eq) and (3S)-3-aminobutan-1-ol (1.0 g, 11.2 mmol, 1 eq) in Tol. (20 mL) was added TEA (385.9 mg, 3.8 mmol, 530.9 uL, 0.3 eq). The mixture was stirred at 110° C. for 12 hr. The reaction mixture was concentrated under reduced pressure to remove solvent. The residue was purified by flash silica gel chromatography (ISCO®; 12 g SepaFlash® Silica Flash Column, Eluent of 0˜40% Ethyl acetate/Petroleum ether gradient @ 60 mL/min) to give (S)-2-(4-hydroxybutan-2-yl)isoindoline-1,3-dione.

### Step 2: (S)-2-(4-((5,6-dimethyl-6H-pyrido[4,3-b]carbazol-9-yl)oxy)butan-2-yl)isoindoline-1,3-dione

A mixture of 5,6-dimethyl-6H-pyrido[4,3-b]carbazol-9-ol (100 mg, 381.2 umol, 1 eq), (S)-2-(4-hydroxybutan-2-yl)isoindoline-1,3-dione (100.3 mg, 457.4 umol, 1.2 eq), 2-(tributyl-λ5-phosphanylidene) acetonitrile (184.0 mg, 762.4 umol, 2 eq) in Tol. (5 mL) was stirred at 100° C. for 12 hr under N₂ atmosphere. The reaction mixture was concentrated under reduced pressure to remove solvent. The residue was purified by prep-TLC (DCM: MeOH=10/1) to give (S)-2-(4-((5,6-dimethyl-6H-pyrido[4,3-b]carbazol-9-yl)oxy)butan-2-yl)isoindoline-1,3-dione.

### Step 3: (S)-4-((5,6-dimethyl-6H-pyrido[4,3-b]carbazol-9-yl)oxy)butan-2-amine

To a solution of (S)-2-(4-((5,6-dimethyl-6H-pyrido[4,3-b]carbazol-9-yl)oxy)butan-2-yl)isoindoline-1,3-dione (120 mg, 258.8 umol, 1 eq) in EtOH (5 mL) was added N₂H₄·H₂O (264.4 mg, 5.1 mmol, 256.7 uL, 98% purity, 20 eq). The mixture was stirred at 80° C. for 12 hr. The reaction mixture was filtered to give filtrate. The filtrate was purified by prep-HPLC (TFA condition, column: Phenomenex Luna C18 150*30 mm*5 um; mobile phase: [water(0.1% TFA)-ACN]; B %: 7%-37%, 9 min) to give (S)-4-((5,6-dimethyl-6H-pyrido[4,3-b]carbazol-9-yl)oxy)butan-2-amine.

MS (ESI): m/z=334.1 [M+H]⁺

### (S)—N-(1-((5,6-dimethyl-6H-pyrido[4,3-b]carbazol-9-yl)oxy)propan-2-yl)pyridin-2-amine (Compound 269)

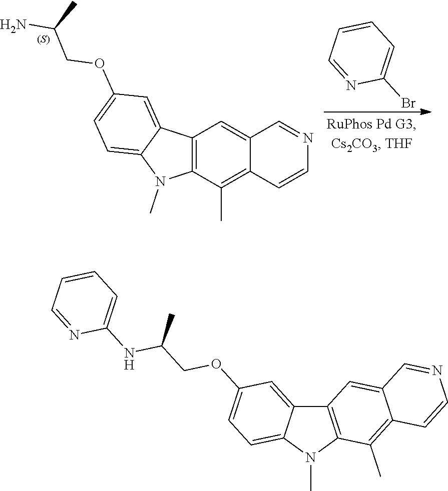

To a solution of (2S)-1-(5,6-dimethylpyrido[4,3-b]carbazol-9-yl)oxypropan-2-amine (50 mg, 115.3 umol, 1 eq, TFA) in MeOH (5 mL) was added basic resin to adjusted pH=8. Then the mixture was filtered and concentrated under reduced pressure to give a residue. The residue was dissolve with THF (2 mL) and added 2-bromopyridine (36.4 mg, 230.7 umol, 21.9 uL, 2 eq), Cs₂CO₃ (112.7 mg, 346.0 umol, 3 eq) and RuPhos Pd G3 (9.6 mg, 11.5 umol, 0.1 eq). The mixture was stirred at 100° C. for 12 hr under N₂. The mixture was concentrated, and the residue was purified by prep-HPLC (TFA condition; column: Phenomenex Luna C18 150*30 mm*5 um; mobile phase: [water(0.1% TFA)-ACN]; B %: 10%-40%, 9 min) to afford (S)—N-(1-((5,6-dimethyl-6H-pyrido[4,3-b]carbazol-9-yl)oxy)propan-2-yl)pyridin-2-amine.

MS (ESI): m/z=397.1 [M+H]⁺

### 9-(2-amino-2-oxo-ethoxy)-N-[(1R)-2-(dimethylamino)-1-methyl-ethyl]-5,6-dimethyl-pyrido[4,3-b]carbazole-1-carboxamide (Compound 270)

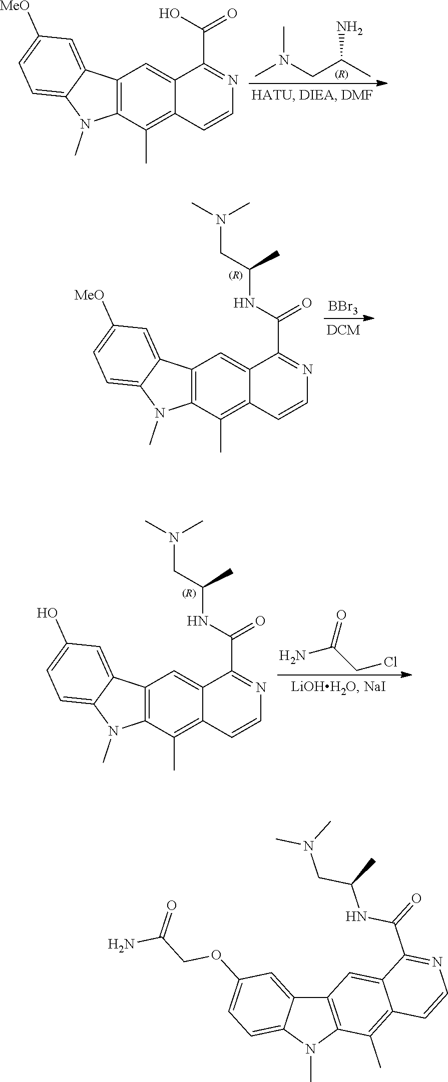

### Step 1: N-[(1R)-2-(dimethylamino)-1-methyl-ethyl]-9-methoxy-5,6-dimethyl-pyrido[4,3-b]carbazole-1-carboxamide

To a solution of 9-methoxy-5,6-dimethyl-pyrido[4,3-b]carbazole-1-carboxylic acid (350 mg, 1.0 mmol, 1 eq) in DMF (5 mL) was added (2R)—N1,N1-dimethylpropane-1,2-diamine (573.9 mg, 3.2 mmol, 3 eq, 2 HCl), DIEA (706.0 mg, 5.4 mmol, 951.5 uL, 5 eq). Then HATU (830.8 mg, 2.1 mmol, 2 eq) was added to the mixture at 0° C. The mixture was warmed to 25° C. for 1 hr. The reaction mixture was concentrated under reduced pressure to give a residue. The residue was purified by prep-HPLC (TFA condition) to give N-[(1R)-2-(dimethylamino)-1-methyl-ethyl]-9-methoxy-5,6-dimethyl-pyrido[4,3-b]carbazole-1-carboxamide.

### Step 2: (R)—N-(1-(dimethylamino)propan-2-yl)-9-hydroxy-5,6-dimethyl-6H-pyrido[4,3-b]carbazole-1-carboxamide

To a solution of N-[(1R)-2-(dimethylamino)-1-methyl-ethyl]-9-methoxy-5,6-dimethyl-pyrido[4,3-b]carbazole-1-carboxamide (100 mg, 247.2 umol, 1 eq) in DCM (3 mL) was added BBr₃ (309.6 mg, 1.2 mmol, 119.1 uL, 5 eq) at 0° C. The mixture was stirred at 45° C. for 1 hr. The reaction mixture was added to NH₃·H₂O (5 mL, purity 25%) at 0° C. The water phase was separated, extracted with CH₂Cl₂ (5 mL×3). The combined organic phase was dried over Na₂SO₄, filtered, and concentrated under reduced pressure to give N-[(1R)-2-(dimethylamino)-1-methyl-ethyl]-9-hydroxy-5,6-dimethyl-pyrido[4,3-b]carbazole-1-carboxamide.

### Step 3: (R)-9-(2-amino-2-oxoethoxy)-N-(1-(dimethylamino)propan-2-yl)-5,6-dimethyl-6H-pyrido[4,3-b]carbazole-1-carboxamide

To a solution of N-[(1R)-2-(dimethylamino)-1-methyl-ethyl]-9-hydroxy-5,6-dimethyl-pyrido[4,3-b]carbazole-1-carboxamide (50 mg, 128.0 umol, 1 eq) in DMF (2 mL) was added 2-chloroacetamide (10.7 mg, 115.2 umol, 0.9 eq), LiOH·H₂O (16.1 mg, 384.1 umol, 3 eq) and NaI (19.1 mg, 128.0 umol, 1 eq). The mixture was stirred at 80° C. for 12 hr. The reaction mixture was concentrated under reduced pressure to give a residue. The residue was purified by prep-HPLC (TFA condition; column: Phenomenex Luna C18 100*30 mm*5 um; mobile phase: [water(0.1% TFA)-ACN]; B %: 10%-40%, 10 min) to give 9-(2-amino-2-oxo-ethoxy)-N-[(1R)-2-(dimethylamino)-1-methyl-ethyl]-5,6-dimethyl-pyrido[4,3-b]carbazole-1-carboxamide.

MS (ESI): m/z=448.2 [M+H]⁺

### 9-[(2S)-2-aminopropoxy]-N-[(1R)-2-(dimethylamino)-1-methyl-ethyl]-5,6-dimethyl-pyrido[4,3-b]carbazole-1-carboxamide (Compound 271)

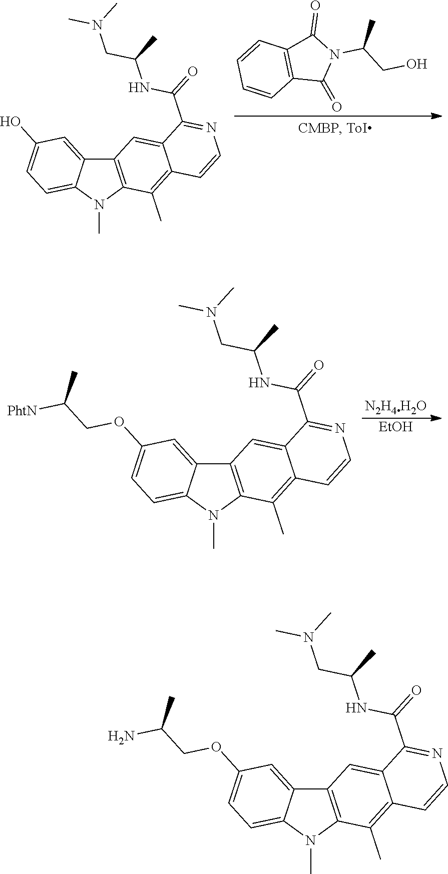

### Step 1: N-[(1R)-2-(dimethylamino)-1-methyl-ethyl]-9-[(2S)-2-(1,3-dioxoisoindolin-2-yl)propoxy]-5,6-dimethyl-pyrido[4,3-b]carbazole-1-carboxamide

To a solution of N-[(1R)-2-(dimethylamino)-1-methyl-ethyl]-9-hydroxy-5,6-dimethyl-pyrido[4,3-b]carbazole-1-carboxamide (50 mg, 128.0 umol, 1 eq) in Tol. (3 mL) was added 2-[(1S)-2-hydroxy-1-methyl-ethyl]isoindoline-1,3-dione (31.5 mg, 153.6 umol, 1.2 eq) and 2-(tributyl-λ⁵-phosphanylidene)acetonitrile (61.8 mg, 256.1 umol, 2 eq) under N₂. The mixture was stirred at 100° C. for 12 hr under N₂. The reaction mixture was concentrated under reduced pressure to give a residue. The residue was purified by prep-TLC (SiO₂, DCM:MeOH=10:1) to give N-[(1R)-2-(dimethylamino)-1-methyl-ethyl]-9-[(2S)-2-(1,3-dioxoisoindolin-2-yl)propoxy]-5,6-dimethyl-pyrido[4,3-b]carbazole-1-carboxamide.

### Step 2: 9-((S)-2-aminopropoxy)-N—((R)-1-(dimethylamino)propan-2-yl)-5,6-dimethyl-6H-pyrido[4,3-b]carbazole-1-carboxamide

To a solution of N-[(1R)-2-(dimethylamino)-1-methyl-ethyl]-9-[(2S)-2-(1,3-dioxoisoindolin-2-yl)propoxy]-5,6-dimethyl-pyrido[4,3-b]carbazole-1-carboxamide (20 mg, 34.6 umol, 1 eq) in EtOH (2 mL) was added N₂H₄·H₂O (8.8 mg, 173.1 umol, 8.5 uL, 98% purity, 5 eq). The mixture was stirred at 70° C. for 12 hr. The reaction mixture was concentrated under reduced pressure. The residue was diluted with NH₄Cl 5 mL and extracted with EtOAc (5 mL×3). The combined organic layers were washed with brine (5 mL×3), dried over Na₂SO₄, filtered, and concentrated under reduced pressure to give a residue. The residue was purified by prep-HPLC (TFA condition, column: Phenomenex Gemini-NX C18 75*30 mm*3 um; mobile phase: [water(0.1% TFA)-ACN]; B %: 1%-35%, 10 min) to give 9-[(2S)-2-aminopropoxy]-N-[(1R)-2-(dimethylamino)-1-methyl-ethyl]-5,6-dimethyl-pyrido[4,3-b]carbazole-1-carboxamide.

MS (ESI): m/z=448.2 [M+H]⁺

### (S)—N-(1-((5,6-dimethyl-6H-pyrido[4,3-b]carbazol-9-yl)oxy)propan-2-yl)acetamide (Compound 272)

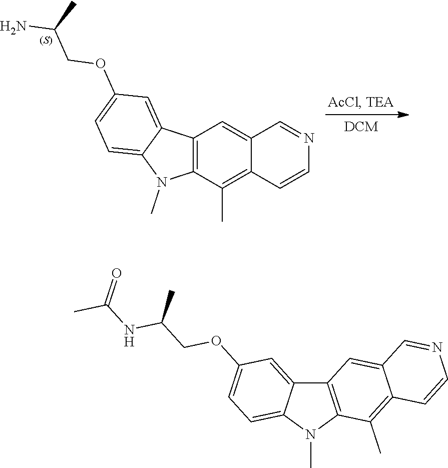

To a solution of (2S)-1-(5,6-dimethylpyrido[4,3-b]carbazol-9-yl)oxypropan-2-amine (50 mg, 115.3 umol, 1 eq, TFA) in DCM (3 mL) was added TEA (35.0 mg, 346.0 umol, 48.1 uL, 3 eq) and acetyl chloride (10.8 mg, 138.4 umol, 9.8 uL, 1.2 eq) at 0° C. The mixture was stirred at 25° C. for 1 hr. The reaction was concentrated to give a residue. The residue was purified by prep-HPLC (TFA condition; column: Phenomenex Luna C18 150*30 mm*5 um; mobile phase: [water(0.1% TFA)-ACN]; B %: 13%-43%, 9 min) to afford (S)—N-(1-((5,6-dimethyl-6H-pyrido[4,3-b]carbazol-9-yl)oxy)propan-2-yl)acetamide.

MS (ESI): m/z=362.2 [M+H]⁺

### (S)-1-(1-((5,6-dimethyl-6H-pyrido[4,3-b]carbazol-9-yl)oxy)propan-2-yl)urea (Compound 273)

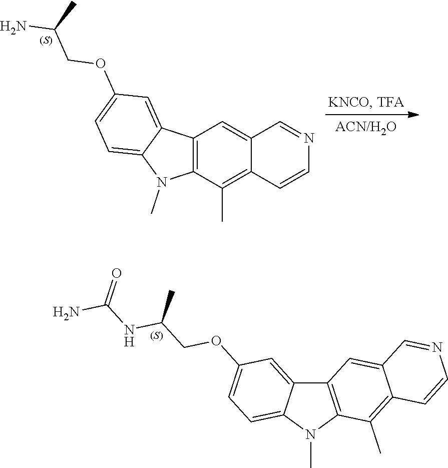

To a solution of (2S)-1-(5,6-dimethylpyrido[4,3-b]carbazol-9-yl)oxypropan-2-amine (50 mg, 115.3 umol, 1 eq, TFA) in ACN (1 mL)/H₂O (1 mL) was added KNCO (29.4 mg, 346.0 umol, 3 eq) and TFA (13.1 mg, 115.3 umol, 8.5 uL, 1 eq). The mixture was stirred at 40° C. for 12 hr. The reaction was concentrated to give a residue. The residue was purified by prep-HPLC (TFA condition; column: Phenomenex luna C18 100*40 mm*5 um; mobile phase: [water(0.1% TFA)-ACN]; B %: 1%-35%, 8 min) to afford (S)-1-(1-((5,6-dimethyl-6H-pyrido[4,3-b]carbazol-9-yl)oxy)propan-2-yl)urea.

MS (ESI): m/z=363.1 [M+H]⁺

### 4-((5,6-dimethyl-6H-pyrido[4,3-b]carbazol-9-yl)oxy)-2-methylbutan-2-amine (Compound 274)

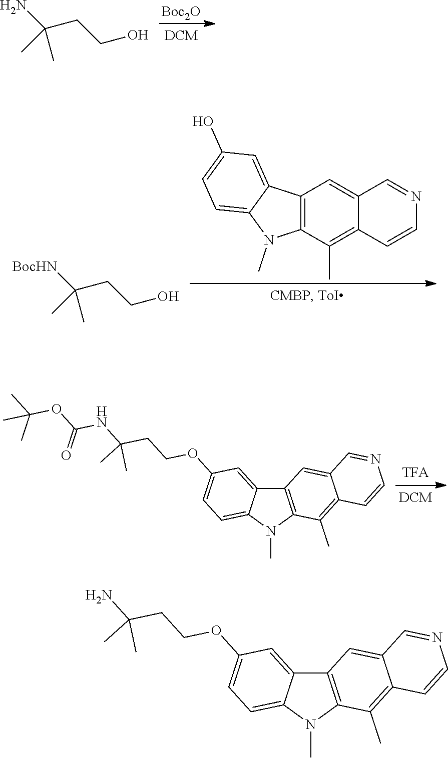

### Step 1: tert-butyl (4-hydroxy-2-methylbutan-2-yl)carbamate

tert-Butoxycarbonyl tert-butyl carbonate (1.3 g, 5.8 mmol, 1.3 mL, 1.2 eq) and 3-amino-3-methyl-butan-1-ol (0.5 g, 4.8 mmol, 1 eq) in DCM (15 mL) was stirred at 25° C. for 12 hr. The reaction mixture was concentrated under reduce pressure to give tert-butyl (4-hydroxy-2-methylbutan-2-yl)carbamate

### Step 2: tert-butyl(4-((5,6-dimethyl-6H-pyrido[4,3-b]carbazol-9-yl)oxy)-2-methylbutan-2-yl)-carbamate

To a solution of 5,6-dimethylpyrido[4,3-b]carbazol-9-ol (50 mg, 190.6 umol, 1 eq) in toluene (2 mL) was added tert-butyl (4-hydroxy-2-methylbutan-2-yl)carbamate (46.5 mg, 228.7 umol, 1.2 eq), and 2-(tributyl-λ⁵-phosphanylidene)acetonitrile (92.0 mg, 381.2 umol, 2 eq). The reaction mixture was stirred at 100° C. for 2 hr under N₂. The reaction mixture was concentrated under reduced pressure to remove solvent. The residue was diluted with DCM (2 mL). The residue was purified by prep-TLC (SiO₂, DCM:Methanol=10:1) to give tert-butyl (4-((5,6-dimethyl-6H-pyrido[4,3-b]carbazol-9-yl)oxy)-2-methylbutan-2-yl)carbamate.

### Step 3: 4-((5,6-dimethyl-6H-pyrido[4,3-b]carbazol-9-yl)oxy)-2-methylbutan-2-amine

To a solution of tert-butyl N-[3-(5,6-dimethylpyrido[4,3-b]carbazol-9-yl)oxy-1,1-dimethyl-propyl]carbamate (82 mg, 183.2 umol, 1 eq) in DCM (2 mL) was added TFA (1.5 g, 13.5 mmol, 1 mL, 73.7 eq). The reaction mixture was stirred at 25° C. for 3 hr. The reaction mixture was concentrated under reduced pressure to remove solvent. The residue was diluted with methanol (2 mL). The residue was purified by prep-HPLC (TFA condition; column: Phenomenex luna C18 100*40 mm*5 um; mobile phase: [water(0.1% TFA)-ACN]; B %: 5%-25%, 8 min) to give 4-((5,6-dimethyl-6H-pyrido[4,3-b]carbazol-9-yl)oxy)-2-methylbutan-2-amine.

MS (ESI): m/z=348.1 [M+H]⁺

### (2S)-1-(5,6-dimethylpyrido[4,3-b]carbazol-9-yl)oxy-N-ethyl-propan-2-amine (Compound 275)

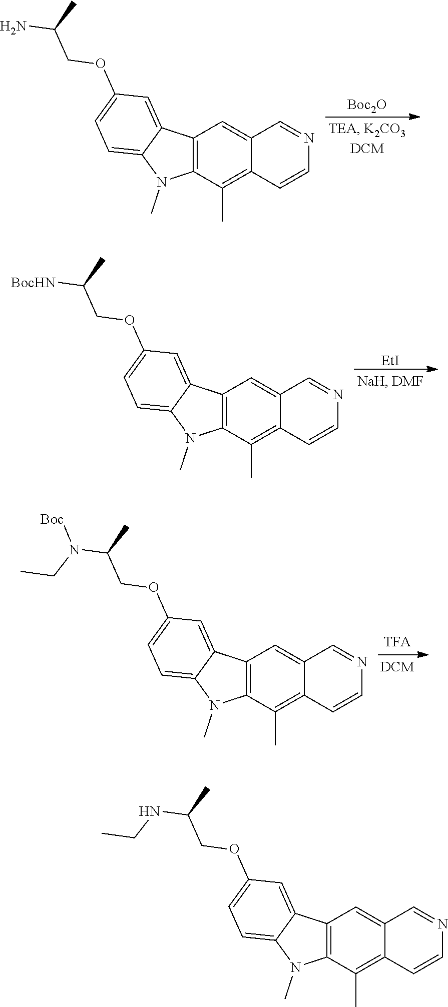

### Step 1: tert-butylN-[(1S)-2-(5,6-dimethylpyrido[4,3-b]carbazol-9-yl)oxy-1-methyl-ethyl]carbamate

To a solution of (2S)-1-(5,6-dimethylpyrido[4,3-b]carbazol-9-yl)oxypropan-2-amine (200 mg, 626.1 umol, 1 eq) in DCM (10 mL) was added TEA (190.0 mg, 1.8 mmol, 261.4 uL, 3 eq) and Boc₂O (273.3 mg, 1.2 mmol, 287.7 uL, 2 eq). The mixture was stirred at 25° C. for 12 hr. The mixture was concentrated to get crude residue and diluted with MeOH 10 mL. Then K₂CO₃ (259.6 mg, 1.8 mmol, 3 eq) was added. Then the mixture was stirred at 50° C. for 12 hr. The mixture was concentrated to get crude residue and then diluted with H₂O 10 mL and extracted with EtOAc (20 mL×3). The combined organic layers were washed with brine (10 mL×3), dried over Na₂SO₄, filtered and concentrated under reduced pressure to give a residue. The residue was purified by flash silica gel chromatography (ISCO®; 4 g SepaFlash® Silica Flash Column, Eluent of 0˜10% Dichloromethane:Methanol gradient @ 60 mL/min) to afford tert-butyl N-[(1S)-2-(5,6-dimethylpyrido[4,3-b]carbazol-9-yl)oxy-1-methyl-ethyl]carbamate.

### Step 2: tert-butyl N-[(1S)-2-(5,6-dimethylpyrido[4,3-b]carbazol-9-yl)oxy-1-methyl-ethyl]-N-ethyl-carbamate

To a solution of tert-butyl N-[(1S)-2-(5,6-dimethylpyrido[4,3-b]carbazol-9-yl)oxy-1-methyl-ethyl]carbamate (30 mg, 71.51 umol, 1 eq) in DMF (2 mL) was added NaH (8.5 mg, 214.5 umol, 60% purity, 3 eq). The mixture was stirred at 0° C. for 10 min. Then iodoethane (11.1 mg, 71.5 umol, 5.7 uL, 1 eq) was added to the mixture and stirred at 25° C. for 1 hr. The mixture was quenched with TFA (0.2 mL) and concentrated to afford tert-butyl-N-[(1S)-2-(5,6-dimethylpyrido[4,3-b]carbazol-9-yl) oxy-1-methyl-ethyl]-N-ethyl-carbamate.

### Step 3: (2S)-1-(5,6-dimethylpyrido[4,3-b]carbazol-9-yl)oxy-N-ethyl-propan-2-amine

To a solution of tert-butyl N-[(1S)-2-(5,6-dimethylpyrido[4,3-b]carbazol-9-yl)oxy-1-methyl-ethyl]-N-ethyl-carbamate (30 mg, 67.03 umol, 1 eq) in DCM (2 mL) was added TFA (1 mL). The mixture was stirred at 25° C. for 1 hr. The mixture was concentrated to get crude residue. The residue was purified by prep-HPLC (TFA condition, column: Phenomenex Luna C18 150*30 mm*5 um; mobile phase: [water(0.1% TFA)-ACN]; B %: 8%-38%, 9 min) to afford (2S)-1-(5,6-dimethylpyrido[4,3-b]carbazol-9-yl)oxy-N-ethyl-propan-2-amine.

MS (ESI): m/z=348.1 [M+H]⁺

### (S)-1-((5,6-dimethyl-6H-pyrido[4,3-b]carbazol-9-yl)oxy)-N-methylpropan-2-amine (Compound 276)

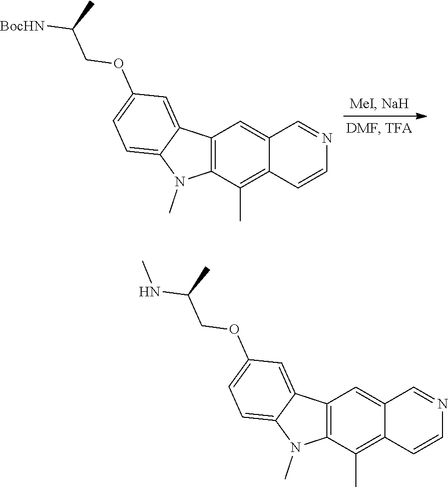 [SMILES: Cc1c2ccncc2cc2c3cc(OC[C@H](C)NC(=O)OC(C)(C)C)ccc3n(C)c12 ; CN[C@@H](C)COc1ccc2c(c1)c1cc3cnccc3c(C)c1n2C]

To a solution of tert-butyl N-[(1S)-2-(5,6-dimethylpyrido[4,3-b]carbazol-9-yl)oxy-1-methyl-ethyl]carbamate (50 mg, 119.1 umol, 1 eq) in DMF (2 mL) was added NaH (14.3 mg, 357.5 umol, 60% purity, 3 eq). The mixture was stirred at 0° C. for 30 min, and then Mel (16.9 mg, 119.1 umol, 7.4 uL, 1 eq) was added. The mixture was stirred at 25° C. for 12 hr. The mixture was quenched with TFA (1.0 mL) and concentrated to get crude residue. The residue was purified by prep-HPLC (TFA condition; column: Phenomenex luna C18 100*40 mm*5 um; mobile phase: [water(0.1% TFA)-ACN]; B %: 1%-25%, 8 min) to afford (S)-1-((5,6-dimethyl-6H-pyrido[4,3-b]carbazol-9-yl)oxy)-N-methylpropan-2-amine.

MS (ESI): m/z=334.1 [M+H]⁺

### (2S)-1-(5,6-dimethylpyrido[4,3-b]carbazol-9-yl)oxy-N-propyl-propan-2-amine (Compound 277)

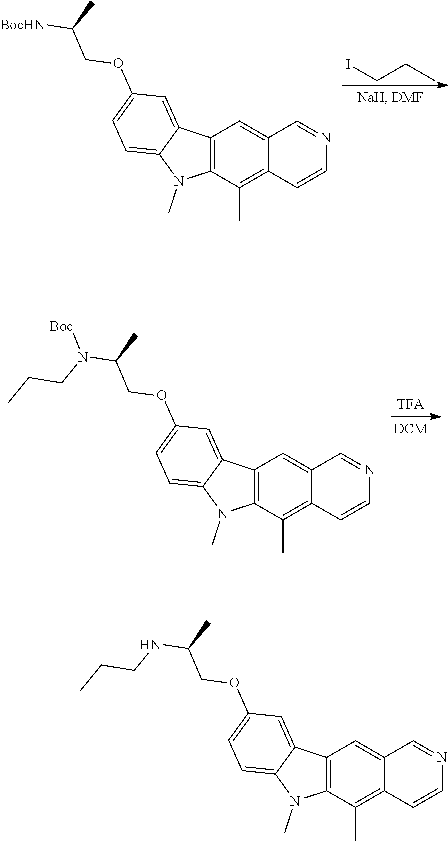

### Step 1: tert-butylN-[(1S)-2-(5,6-dimethylpyrido[4,3-b]carbazol-9-yl)oxy-1-methyl-ethyl]-N-propyl-carbamate

To a solution of tert-butyl N-[(1S)-2-(5,6-dimethylpyrido[4,3-b]carbazol-9-yl)oxy-1-methyl-ethyl]carbamate (50 mg, 119.1 umol, 1 eq) in DMF (2 mL) was added NaH (14.3 mg, 357.5 umol, 60% purity, 3 eq). The mixture was stirred at 0° C. for 10 min. Then 1-iodopropane (20.2 mg, 119.1 umol, 11.6 ul, 1 eq) was added to the mixture and stirred at 25° C. for 1 hr. The reaction mixture was poured into NH₄Cl (10 mL) and extracted with EtOAc (20 mL×3). The combined organic layers were washed with brine (20 mL×3), dried over Na₂SO₄, filtered and concentrated under reduced pressure to give a residue. The residue was purified by prep-TLC (SiO₂, DCM:MeOH=10:1) to give tert-butylN-[(1S)-2-(5,6-dimethylpyrido[4,3-b]carbazol-9-yl)oxy-1-methyl-ethyl]-N-propyl-carbamate.

### Step 2: (2S)-1-(5,6-dimethylpyrido[4,3-b]carbazol-9-yl)oxy-N-propyl-propan-2-amine

To a solution of tert-butyl N-[(1S)-2-(5,6-dimethylpyrido[4,3-b]carbazol-9-yl)oxy-1-methyl-ethyl]-N-propyl-carbamate (60 mg, 129.98 umol, 1 eq) in DCM (2 mL) was added TFA (1 mL). The mixture was stirred at 25° C. for 1 hr. The reaction mixture was concentrated under reduced pressure to give a residue. The residue was purified by prep-HPLC (TFA condition, column: Phenomenex Luna C18 150*30 mm*5 um; mobile phase: [water(0.1% TFA)-ACN]; B %: 10%-40%, 9 min) to give (2S)-1-(5,6-dimethylpyrido[4,3-b]carbazol-9-yl)oxy-N-propyl-propan-2-amine.

MS (ESI): m/z=362.2 [M+H]⁺

### (2S)-1-(5,6-dimethylpyrido[4,3-b]carbazol-9-yl)oxy-N-(2-methoxyethyl)propan-2-amine (Compound 278)

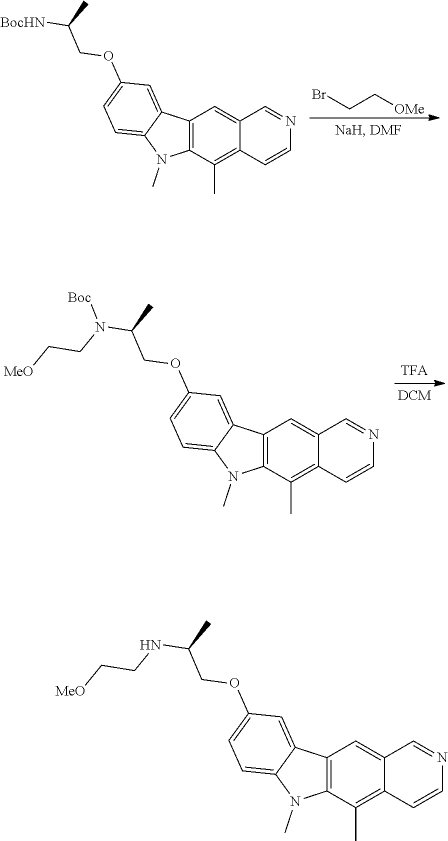

### Step 1: tert-butyl N-[(1S)-2-(5,6-dimethylpyrido[4,3-b]carbazol-9-yl)oxy-1-methyl-ethyl]-N-(2-methoxyethyl)carbamate

To a solution of tert-butyl N-[(1S)-2-(5,6-dimethylpyrido[4,3-b]carbazol-9-yl)oxy-1-methyl-ethyl]carbamate (30 mg, 71.5 umol, 1 eq) in DMF (1 mL) was added NaH (8.5 mg, 214.5 umol, 60% purity, 3 eq). The mixture was stirred at 0° C. for 10 min. Then 1-bromo-2-methoxy-ethane (10.9 mg, 78.6 umol, 7.3 ul, 1.1 eq) was added to the mixture and stirred at 25° C. for 1 hr. The mixture was quenched with TFA (0.2 mL) and concentrated to afford tert-butyl N-[(1S)-2-(5,6-dimethylpyrido[4,3-b]carbazol-9-yl)oxy-1-methyl-ethyl]-N-(2-methoxyethyl)carbamate.

### Step 2: (S)-1-((5,6-dimethyl-6H-pyrido[4,3-b]carbazol-9-yl)oxy)-N-(2-methoxyethyl)propan-2-amine

To a solution of tert-butyl N-[(1S)-2-(5,6-dimethylpyrido[4,3-b]carbazol-9-yl)oxy-1-methyl-ethyl]-N-(2-methoxyethyl)carbamate (30 mg, 62.8 umol, 1 eq) in DCM (2 mL) was added TFA (1 mL). The mixture was stirred at 25° C. for 1 hr. The mixture was concentrated to get crude residue. The residue was purified by prep-HPLC (TFA condition, column: Phenomenex Luna C18 150*30 mm*5 um; mobile phase: [water(0.1% TFA)-ACN]; B %: 8%-38%, 9 min) to afford (2S)-1-(5,6-dimethylpyrido[4,3-b]carbazol-9-yl)oxy-N-(2-methoxyethyl)propan-2-amine.

MS (ESI): m/z=378.1 [M+H]⁺

### (2S)—N-(2,2-difluoroethyl)-1-(5,6-dimethylpyrido[4,3-b]carbazol-9-yl)oxy-propan-2-amine (Compound 279)

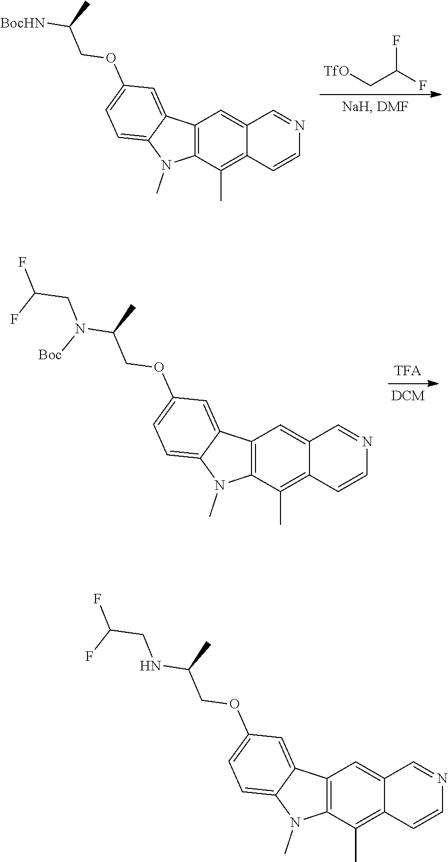 [SMILES: Cc1c2ccncc2cc2c3cc(OC[C@H](C)NC(=O)OC(C)(C)C)ccc3n(C)c12 ; O=S(=O)(OCC(F)F)C(F)(F)F ; Cc1c2ccncc2cc2c3cc(OC[C@H](C)N(CC(F)F)C(=O)OC(C)(C)C)ccc3n(C)c12 ; Cc1c2ccncc2cc2c3cc(OC[C@H](C)NCC(F)F)ccc3n(C)c12]

### Step 1: tert-butylN-(2,2-difluoroethyl)-N-[(1S)-2-(5,6-dimethylpyrido[4,3-b]carbazol-9-yl)oxy-1-methyl-ethyl]carbamate

To a solution of tert-butyl N-[(1S)-2-(5,6-dimethylpyrido[4,3-b]carbazol-9-yl)oxy-1-methyl-ethyl]carbamate (50 mg, 119.1 umol, 1 eq) in DMF (2 mL) was added NaH (14.3 mg, 357.5 umol, 60% purity, 3 eq). The mixture was stirred at 0° C. for 10 min. Then 2,2-difluoroethyl trifluoromethanesulfonate (25.5 mg, 119.1 umol, 5.7 uL, 1 eq) was added to the mixture and stirred at 25° C. for 1 hr. The mixture was poured into NH₄Cl (5 mL) and extracted with EtOAc (10 mL×3). The combined organic layers were washed with brine (5 mL×3), dried over Na₂SO₄, filtered and concentrated under reduced pressure to give a residue. The residue was purified by prep-TLC (SiO₂, DCM:MeOH=10:1) to give tert-butyl N-(2,2-difluoroethyl)-N-[(1S)-2-(5,6-dimethylpyrido[4,3-b]carbazol-9-yl)oxy-1-methyl-ethyl]carbamate.

### Step 2: (2S)—N-(2,2-difluoroethyl)-1-(5,6-dimethylpyrido[4,3-b]carbazol-9-yl)oxy-propan-2-amine

To a solution of tert-butyl N-(2,2-difluoroethyl)-N-[(1S)-2-(5,6-dimethylpyrido[4,3-b]carbazol-9-yl)oxy-1-methyl-ethyl]carbamate (20 mg, 41.3 umol, 1 eq) in DCM (2 mL) was added TFA (1.5 g, 13.5 mmol, 1 mL, 326.5 eq). The mixture was stirred at 25° C. for 1 hr. The reaction mixture was concentrated to dryness. The residue was purified by prep-HPLC to give (2S)—N-(2,2-difluoroethyl)-1-(5,6-dimethylpyrido[4,3-b]carbazol-9-yl)oxy-propan-2-amine.

MS (ESI): m/z=384.2[M+H]⁺

### (S)—N-(2-aminopropyl)-5,6-dimethyl-6H-pyrido[4,3-b]carbazole-9-carboxamide (Compound 280)

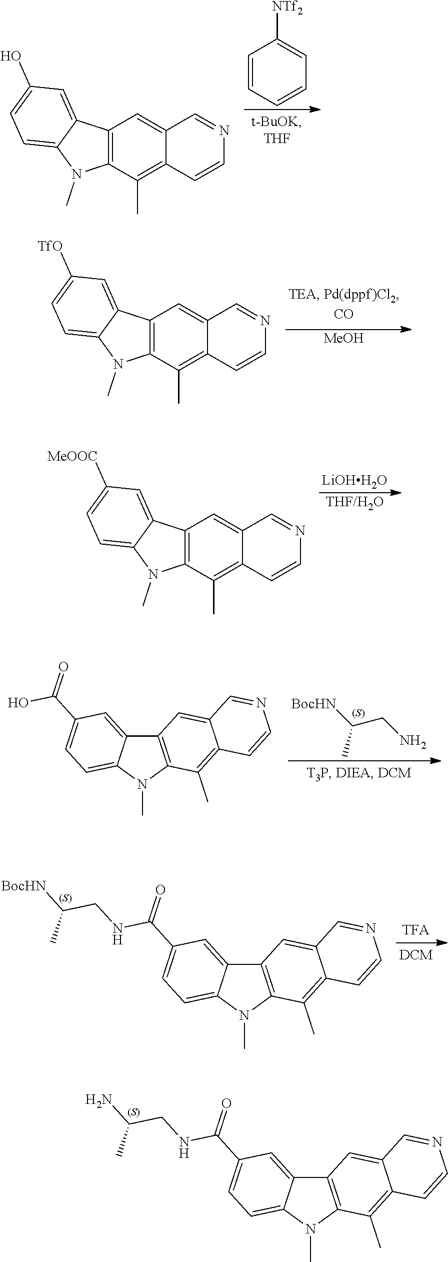

### Step 1: 5,6-dimethyl-6H-pyrido[4,3-b]carbazol-9-yl trifluoromethanesulfonate

To a solution of 5,6-dimethylpyrido[4,3-b]carbazol-9-ol (500 mg, 1.9 mmol, 1 eq) in THF (20 mL) was added t-BuOK (1 M, 3.8 mL, 2 eq) at 0° C. The mixture was stirred at 0° C. for 10 min. 1,1,1-trifluoro-N-phenyl-N-(trifluoromethylsulfonyl) methanesulfonamide (817.1 mg, 2.3 mmol, 1.2 eq) was added to the mixture. The mixture was stirred at 25° C. for 12 hr. The reaction mixture was concentrated under reduced pressure to give a residue. The residue was purified by flash silica gel chromatography (ISCO®; 10 g SepaFlash® Silica Flash Column, Eluent of 0˜10% Ethyl acetate/Petroleum ether gradient @ 80 mL/min) to give 5,6-dimethyl-6H-pyrido[4,3-b]carbazol-9-yl trifluoromethanesulfonate.

### Step 2: methyl 5,6-dimethyl-6H-pyrido[4,3-b]carbazole-9-carboxylate

To a solution of (5,6-dimethylpyrido[4,3-b]carbazol-9-yl) trifluoromethanesulfonate (570 mg, 1.5 mmol, 1 eq) in MeOH (15 mL) was added TEA (438.8 mg, 4.3 mmol, 603.5 uL, 3 eq) and Pd(dppf)Cl₂ (105.8 mg, 144.5 umol, 0.1 eq). The mixture was stirred at 75° C. for 4 hr under CO (50 psi). The reaction mixture was concentrated under reduced pressure to remove solvent. The residue was diluted with H₂O 30 mL and extracted with DCM (30 mL×3). The combined organic layers were dried over Na₂SO₄, filtered and concentrated under reduced pressure to give a residue. The residue was purified by flash silica gel chromatography (ISCO®; 10 g SepaFlash® Silica Flash Column, Eluent of 0˜10% Ethyl acetate/Petroleum ether gradient @ 80 mL/min) to give methyl 5,6-dimethyl-6H-pyrido[4,3-b]carbazole-9-carboxylate.

### Step 3: 5,6-dimethyl-6H-pyrido[4,3-b]carbazole-9-carboxylic acid

To a solution of methyl 5,6-dimethylpyrido[4,3-b]carbazole-9-carboxylate (200 mg, 657.2 umol, 1 eq) in THF (1.5 mL)/H₂O (0.5 mL) was added LiOH·H₂O (110.3 mg, 2.63 mmol, 4 eq). The reaction mixture was stirred at 60° C. for 12 hr. The pH was adjusted to 5 by addition HCl (1 M), then filtered and concentrated under reduced pressure to give 5,6-dimethylpyrido [4,3-b]carbazole-9-carboxylic acid.

### Step 4: (S)-tert-butyl(1-(5,6-dimethyl-6H-pyrido[4,3-b]carbazole-9-carboxamido)propan-2-yl)carbamate

To a solution of 5,6-dimethylpyrido[4,3-b]carbazole-9-carboxylic acid (44 mg, 151.6 umol, 1 eq) in DCM (2 mL) was added tert-butyl N-[(1S)-2-amino-1-methyl-ethyl]carbamate (52.8 mg, 303.1 umol, 2 eq), T₃P (192.9 mg, 303.1 umol, 180.3 uL, 50% purity, 2 eq) and DIEA (39.2 mg, 303.1 umol, 52.8 uL, 2 eq) at 0° C. under N₂. The reaction mixture was stirred at 25° C. for 1 hr. The reaction mixture was concentrated under reduced pressure to give a residue. The residue was purified by prep-TLC (SiO₂, DCM:MeOH=10:1) to give (S)-tert-butyl(1-(5,6-dimethyl-6H-pyrido[4,3-b]carbazole-9-carboxamido)propan-2-yl)carbamate.

### Step 5: (S)—N-(2-aminopropyl)-5,6-dimethyl-6H-pyrido[4,3-b]carbazole-9-carboxamide

To a solution of tert-butyl N-[(1S)-2-[(5,6-dimethylpyrido[4,3-b]carbazole-9-carbonyl) amino]-1-methyl-ethyl]carbamate (40 mg, 89.6 umol, 1 eq) in DCM (2 mL) was added TFA (10.2 mg, 89.6 umol, 6.6 uL, 1 eq). The reaction mixture was stirred at 25° C. for 1 hr. The reaction mixture was concentrated under reduced pressure to remove solvent. The residue was diluted with methanol (2 mL). The residue was purified by prep-HPLC (TFA condition; column: Phenomenex Luna C18 150×30 mm×5 um; mobile phase: [water(0.1% TFA)-ACN]; B %: 1%-28%, 9 min) to give (S)—N-(2-aminopropyl)-5,6-dimethyl-6H-pyrido[4,3-b]carbazole-9-carboxamide.

MS (ESI): m/z=347.1 [M+H]⁺

### (S)—N-(1-aminopropan-2-yl)-5,6-dimethyl-6H-pyrido[4,3-b]carbazole-9-carboxamide (Compound 281)

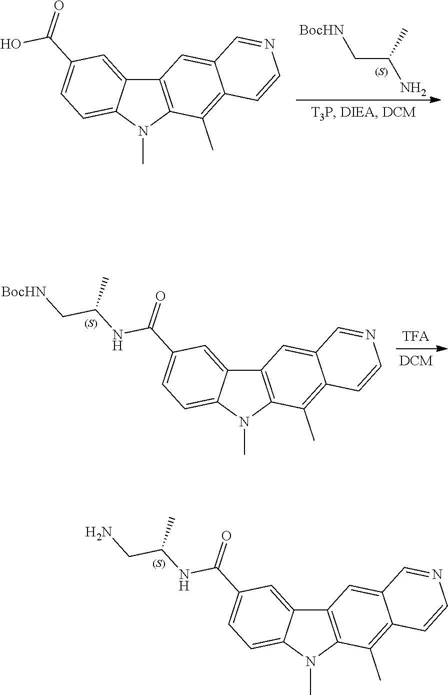

### Step 1: (S)-tert-butyl (2-(5,6-dimethyl-6H-pyrido[4,3-b]carbazole-9-carboxamido)propyl)carbamate

To a solution of 5,6-dimethyl-6H-pyrido[4,3-b]carbazole-9-carboxylic acid (40 mg, 137.7 umol, 1 eq) in DCM (2 mL) was added tert-butyl N-[(2S)-2-aminopropyl]carbamate (48.0 mg, 275.5 umol, 2 eq), T₃P (175.3 mg, 275.5 umol, 163.8 uL, 50% purity, 2 eq) and DIEA (35.6 mg, 275.5 umol, 48.0 uL, 2 eq) at 0° C. under N₂. The reaction mixture was stirred at 25° C. for 1 hr. T₃P (87.6 mg, 137.7 umol, 81.9 uL, 50% purity, 1 eq) and DIEA (17.8 mg, 137.7 umol, 24.0 uL, 1 eq) was added to the solution and the reaction was stirred at 25° C. for 12 hr. The reaction mixture was concentrated under reduced pressure to remove solvent. The residue was purified by prep-TLC (SiO₂, DCM:MeOH=10:1) to give (S)-tert-butyl (2-(5,6-dimethyl-6H-pyrido[4,3-b]carbazole-9-carboxamido)propyl)carbamate.

### Step 2: (S)—N-(1-aminopropan-2-yl)-5,6-dimethyl-6H-pyrido[4,3-b]carbazole-9-carboxamide

To a solution of (S)-tert-butyl (2-(5,6-dimethyl-6H-pyrido[4,3-b]carbazole-9-carboxamido)propyl)carbamate (55 mg, 123.1 umol, 1 eq) in DCM (2 mL) was added TFA (1 mL). The mixture was stirred at 25° C. for 1 hr. The reaction mixture was concentrated under reduced pressure to remove solvent. The residue was purified by prep-HPLC (TFA condition, column: Phenomenex Luna C18 150*30 mm*5 um; mobile phase: [water(0.1% TFA)-ACN]; B %: 1%-28%, 9 min). The residue was purified by prep-HPLC (TFA condition, column: Phenomenex luna C18 100*40 mm*5 um; mobile phase: [water(0.1% TFA)-ACN]; B %: 1%-35%, 8 min) to give (S)—N-(1-aminopropan-2-yl)-5,6-dimethyl-6H-pyrido[4,3-b]carbazole-9-carboxamide.

MS (ESI): m/z=347.1 [M+H]⁺

### N-[(1R)-2-amino-1-methyl-ethyl]-5,6-dimethyl-pyrido[4,3-b] carbazole-9-carboxamide (Compound 282)

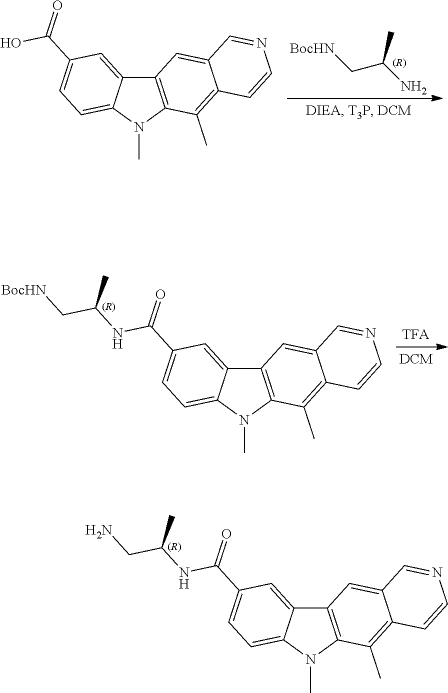

### Step 1: tert-butyl N-[(2R)-2-[(5,6-dimethylpyrido[4,3-b]carbazole-9-carbonyl)amino]propyl]carbamate

To a solution of 5,6-dimethylpyrido[4,3-b]carbazole-9-carboxylic acid (30 mg, 103.3 umol, 1 eq) and tert-butyl N-[(2R)-2-aminopropyl]carbamate (45.0 mg, 258.3 umol, 2.5 eq) in DCM (3 mL) was added T₃P (131.5 mg, 206.6 umol, 122.9 uL, 50% purity, 2 eq) cooled to 0° C. Then DIEA (40.0 mg, 310.0 umol, 54.0 uL, 3 eq) was added. The mixture was stirred at 25° C. for 12 hr under N₂. The reaction mixture was concentrated under reduced pressure to remove solvent. The residue was purified by prep-TLC (SiO₂, Dichloromethane:Methanol=10/1) to give tert-butyl N-[(2R)-2-[(5,6-dimethylpyrido[4,3-b]carbazole-9-carbonyl)amino]propyl]carbamate.

### Step 2: N-[(1R)-2-amino-1-methyl-ethyl]-5,6-dimethyl-pyrido[4,3-b]carbazole-9-carboxamide

To a solution of tert-butyl N-[(2R)-2-[(5,6-dimethylpyrido[4,3-b]carbazole-9-carbonyl) amino]propyl]carbamate (40 mg, 89.5 umol, 1 eq) in DCM (2 mL) was added TFA (1.5 g, 13.5 mmol, 1 mL, 150.7 eq). The mixture was stirred at 25° C. for 2 hr. The reaction mixture was concentrated under reduced pressure to remove solvent. The residue was purified by prep-HPLC (TFA condition, column: Phenomenex luna C18 80*40 mm*3 um; mobile phase: [water(0.1% TFA)-ACN]; B %: 5%-25%, 7 min) to give N-[(1R)-2-amino-1-methyl-ethyl]-5,6-dimethyl-pyrido[4,3-b] carbazole-9-carboxamide.

MS (ESI): m/z=347.1 [M+H]⁺

### (S)-1-((5,6-dimethyl-6H-pyrido[4,3-b]carbazol-9-yl)oxy)-N-isopropylpropan-2-amine (Compound 283)

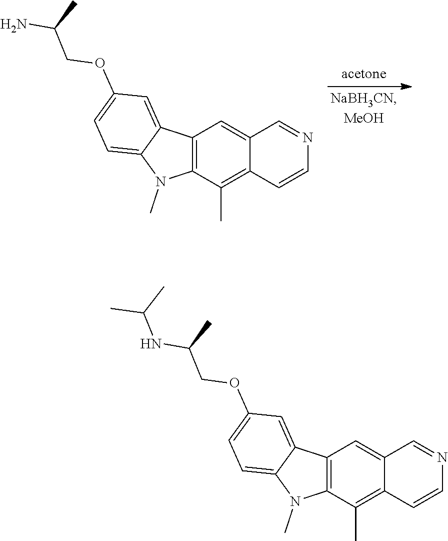 [SMILES: Cc1c2ccncc2cc2c3cc(OC[C@H](C)N)ccc3n(C)c12 ; Cc1c2ccncc2cc2c3cc(OC[C@H](C)NC(C)C)ccc3n(C)c12]

To a solution of (2S)-1-(5,6-dimethylpyrido[4,3-b]carbazol-9-yl)oxypropan-2-amine (40 mg, 112.4 umol, 1 eq, HCl) in MeOH (1 mL) was added TEA adjusted pH to 8 and then acetone (9.7 mg, 168.6 umol, 12.4 uL, 1.5 eq) was added. AcOH was added to the above mixture was adjusted pH to 5 and then NaBH₃CN (28.2 mg, 449.6 umol, 4 eq) was added. The mixture was stirred at 30° C. for 12 hr. The reaction was filtered and the filtrated was concentrated to give a residue. The residue was purified by prep-HPLC (TFA condition; column: Phenomenex luna C18 100*40 mm*5 um; mobile phase: [water(0.1% TFA)-ACN]; B %: 1%-25%, 8 min) to afford (S)-1-((5,6-dimethyl-6H-pyrido[4,3-b]carbazol-9-yl)oxy)-N-isopropylpropan-2-amine.

MS (ESI): m/z=362.2 [M+H]⁺

### (R)—N-(2-aminopropyl)-5,6-dimethyl-6H-pyrido[4,3-b]carbazole-9-carboxamide (Compound 284)

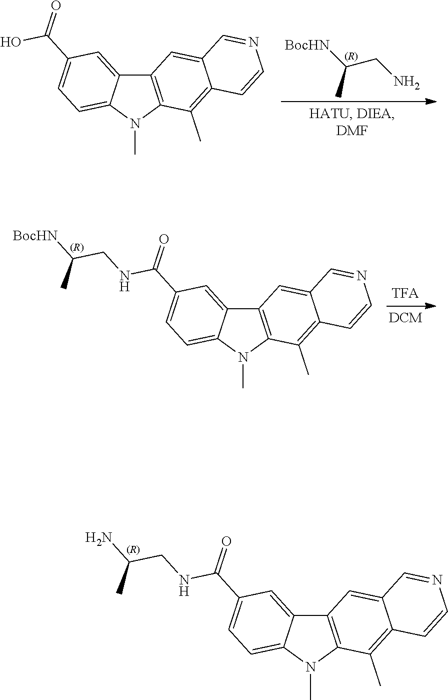

### Step 1: (R)-tert-butyl-(1-(5,6-dimethyl-6H-pyrido[4,3-b]carbazole-9-carboxamido)propan-2-yl)carbamate

To a solution of 5,6-dimethylpyrido[4,3-b]carbazole-9-carboxylic acid (30 mg, 103.3 umol, 1 eq) in DMF (2 mL) was added DIEA (40.1 mg, 310.0 umol, 54.0 uL, 3 eq) and HATU (58.9 mg, 155.0 umol, 1.5 eq). The reaction mixture was stirred at 25° C. for 30 min. tert-Butyl N-[(1R)-2-amino-1-methyl-ethyl]carbamate (36.0 mg, 206.7 umol, 2 eq) was added to the mixture. The mixture was stirred at 25° C. for 3 hr. The reaction mixture was concentrated under reduced pressure to remove DMF. The residue was diluted with H₂O (15 mL) and extracted with ethyl acetate (15 mL×3). The combined organic layers were washed with brine (15 mL×2), dried over Na₂SO₄, filtered and concentrated under reduced pressure to give a residue. The residue was purified by prep-TLC (SiO₂, DCM:MeOH=10:1) to give (R)-tert-butyl-(1-(5,6-dimethyl-6H-pyrido[4,3-b]carbazole-9-carboxamido)propan-2-yl)carbamate.

### Step 2: (R)—N-(2-aminopropyl)-5,6-dimethyl-6H-pyrido[4,3-b]carbazole-9-carboxamide

To solution of tert-butyl N-[(1R)-2-[(5,6-dimethylpyrido[4,3-b]carbazole-9-carbonyl) amino]-1-methyl-ethyl]carbamate (40 mg, 89.58 umol, 1 eq) in DCM (2 mL) was added TFA (102.1 mg, 895.8 umol, 66.3 uL, 10 eq). The reaction mixture was stirred at 25° C. for 2 hr. The reaction mixture was concentrated under reduced pressure to remove solvent. The residue was purified by prep-HPLC (TFA condition; column: Phenomenex Luna C18 150*30 mm*5 um; mobile phase: [water(0.1% TFA)-ACN]; B %: 1%-30%, 9 min) to give (R)—N-(2-aminopropyl)-5,6-dimethyl-6H-pyrido[4,3-b]carbazole-9-carboxamide.

MS (ESI): m/z=347.1 [M+H]⁺

### (R)—N-(2-hydroxypropyl)-5,6-dimethyl-6H-pyrido[4,3-b]carbazole-9-carboxamide (Compound 285)

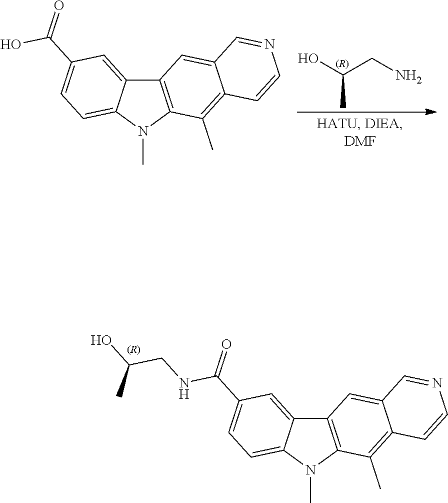

To a solution of 5,6-dimethylpyrido[4,3-b]carbazole-9-carboxylic acid (50 mg, 172.2 umol, 1 eq) in DMF (2 mL) was added (2R)-1-aminopropan-2-ol (25.8 mg, 344.4 umol, 27.1 uL, 2 eq), DIEA (66.7 mg, 516.6 umol, 90.0 uL, 3 eq) and HATU (98.2 mg, 258.3 umol, 1.5 eq) at 0° C. under N₂. The reaction mixture was stirred at 25° C. for 12 hr. The reaction mixture was concentrated under reduced pressure to give a residue. The residue was purified by prep-HPLC (TFA condition; column: Phenomenex Luna C18 150*30 mm*5 um; mobile phase: [water(0.1% TFA)-ACN]; B %: 1%-35%, 9 min) to give (R)—N-(2-hydroxypropyl)-5,6-dimethyl-6H-pyrido[4,3-b]carbazole-9-carboxamide.

MS (ESI): m/z=348.1 [M+H]⁺

### N-[(1R)-2-(dimethylamino)-1-methyl-ethyl]-5,6-dimethyl-pyrido[4,3-b]carbazole-9-carboxamide (Compound 286)

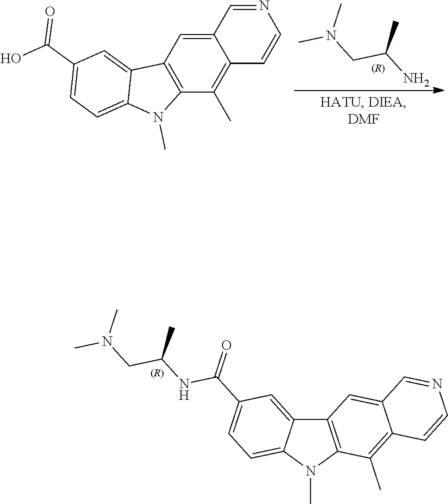

To a solution of 5,6-dimethylpyrido[4,3-b]carbazole-9-carboxylic acid (30 mg, 103.3 umol, 1 eq) in DMF (3 mL) was added DIEA (33.3 mg, 258.3 umol, 45.0 uL, 2.5 eq) and HATU (39.2 mg, 103.3 umol, 1 eq) and stirred at 25° C. for 30 min. The mixture was added (2R)—N1,N1-dimethylpropane-1,2-diamine (21.7 mg, 124.0 umol, 1.2 eq, 2 HCl) and stirred at 25° C. for 2 hr. The reaction mixture was filtered. The residue was purified by prep-HPLC (TFA condition; column: Phenomenex Luna C18 150*30 mm*5 um; mobile phase: [water(0.1% TFA)-ACN]; B %: 1%-30%, 9 min) to give N-[(1R)-2-(dimethylamino)-1-methyl-ethyl]-5,6-dimethyl-pyrido[4,3-b]carbazole-9-carboxamide.

MS (ESI): m/z=375.1 [M+H]⁺

### N-[(2S)-2-hydroxypropyl]-5,6-dimethyl-pyrido[4,3-b]carbazole-9-carboxamide (Compound 287)

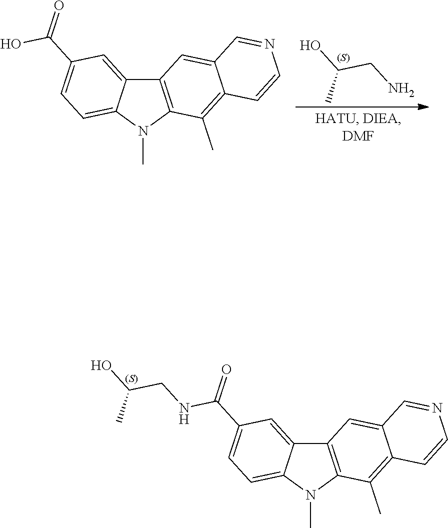

To a solution of 5,6-dimethylpyrido[4,3-b]carbazole-9-carboxylic acid (30 mg, 103.3 umol, 1 eq) in DMF (3 mL) was added DIEA (33.3 mg, 258.3 umol, 45.0 uL, 2.5 eq) and HATU (39.2 mg, 103.3 umol, 1 eq) and stirred at 25° C. for 30 min. (2S)-1-aminopropan-2-ol (9.3 mg, 124.0 umol, 9.7 uL, 1.2 eq) was added to the above mixture and the mixture was stirred at 25° C. for 12 hr. The reaction mixture was filtered. The residue was purified by prep-HPLC (TFA condition; column: Phenomenex Luna C18 150*30 mm*5 um; mobile phase: [water(0.1% TFA)-ACN]; B %: 5%-35%, 9 min) to give N-[(2S)-2-hydroxypropyl]-5,6-dimethyl-pyrido[4,3-b]carbazole-9-carboxamide.

MS (ESI): m/z=348.0 [M+H]⁺

### (S)—N-(1-methoxypropan-2-yl)-5,6-dimethyl-6H-pyrido[4,3-b]carbazole-9-carboxamide (Compound 288)

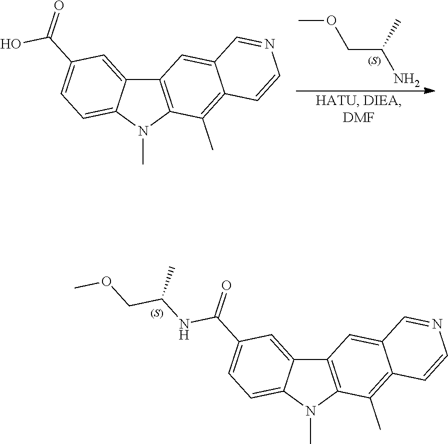

To a solution of 5,6-dimethylpyrido[4,3-b]carbazole-9-carboxylic acid (50 mg, 172.2 umol, 1 eq) in DMF (2 mL) was added DIEA (66.7 mg, 516.6 umol, 90.0 uL, 3 eq) and HATU (98.2 mg, 258.3 umol, 1.5 eq) and stirred for 30 min. (2S)-1-methoxypropan-2-amine (30.7 mg, 344.4 umol, 2 eq) was added to the mixture. The mixture was stirred at 25° C. for 12 hr. The reaction mixture was concentrated under reduced pressure to remove solvent. The residue was purified by prep-HPLC (TFA condition, column: Phenomenex luna C18 100*40 mm*5 um; mobile phase: [water(0.1% TFA)-ACN]; B %: 5%-37%, 8 min) to give (S)—N-(1-methoxypropan-2-yl)-5,6-dimethyl-6H-pyrido[4,3-b]carbazole-9-carboxamide.

MS (ESI): m/z=362.2 [M+H]⁺

### (R)—N-(1-hydroxypropan-2-yl)-5,6-dimethyl-6H-pyrido[4,3-b]carbazole-9-carboxamide (Compound 289)

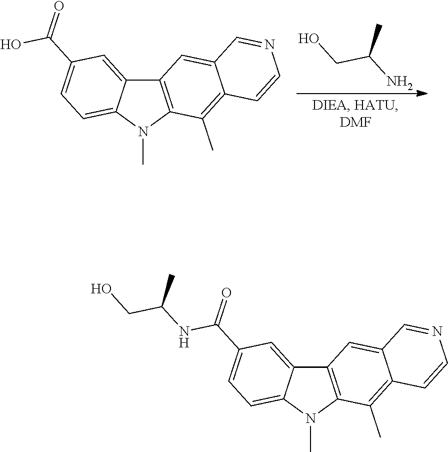

To a solution of 5,6-dimethylpyrido[4,3-b]carbazole-9-carboxylic acid (30 mg, 103.3 umol, 1 eq) in DMF (2 mL) was added DIEA (40.0 mg, 310.0 umol, 54.0 uL, 3 eq), HATU (58.9 mg, 155.0 umol, 1.5 eq) and stirred for 30 min. (2R)-2-aminopropan-1-ol (15.5 mg, 206.6 umol, 16.1 uL, 2 eq) was added to the mixture. The mixture was stirred at 25° C. for 12 hr. The reaction mixture was concentrated under reduced pressure to give a residue. The residue was purified by prep-HPLC (TFA condition, column: Phenomenex luna C18 100*40 mm*5 um; mobile phase: [water(0.1% TFA)-ACN]; B %: 5%-30%, 8 min) to give (R)—N-(1-hydroxypropan-2-yl)-5,6-dimethyl-6H-pyrido[4,3-b]carbazole-9-carboxamide.

MS (ESI): m/z=348.2 [M+H]⁺

### N-(2-amino-2-oxoethyl)-5,6-dimethyl-6H-pyrido[4,3-b]carbazole-1-carboxamide (Compound 290)

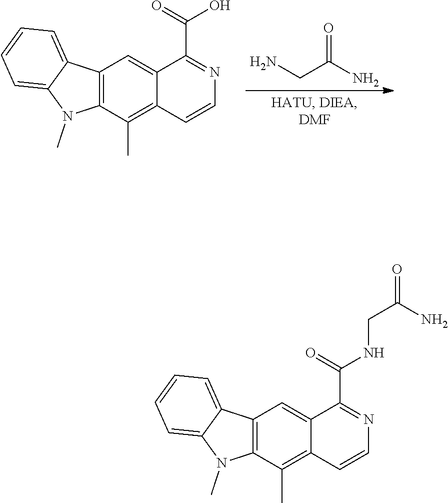 [SMILES: Cc1c2ccnc(C(=O)O)c2cc2c3ccccc3n(C)c12 ; NCC(N)=O ; Cc1c2ccnc(C(=O)NCC(N)=O)c2cc2c3ccccc3n(C)c12]

To a solution of 5,6-dimethylpyrido[4,3-b]carbazole-1-carboxylic acid (50 mg, 172.2 umol, 1 eq) in DMF (2 mL) was added DIEA (66.7 mg, 516.6 umol, 90.0 uL, 3 eq), HATU (98.2 mg, 258.3 umol, 1.5 eq) and stirred for 30 min. 2-aminoacetamide (38.0 mg, 344.4 umol, 2 eq, HCl) was added to the solution and stirred at 25° C. for 12 hr. The reaction mixture was concentrated under reduced pressure to remove solvent. The residue was purified by prep-HPLC (TFA condition, column: Phenomenex Luna C18 100*30 mm*5 um; mobile phase: [water(0.1% TFA)-ACN]; B %: 15%-45%, 10 min) to give N-(2-amino-2-oxoethyl)-5,6-dimethyl-6H-pyrido[4,3-b]carbazole-1-carboxamide.

MS (ESI): m/z=347.1 [M+H]⁺

### N-[(1S)-2-hydroxy-1-methyl-ethyl]-5,6-dimethyl-pyrido[4,3-b]carbazole-9-carboxamide (Compound 291)

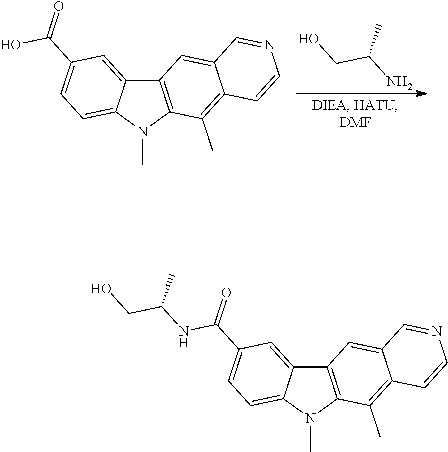

### N-[(1S)-2-hydroxy-1-methyl-ethyl]-5,6-dimethyl-pyrido[4,3-b]carbazole-9-carboxamide

To a solution of 5,6-dimethylpyrido[4,3-b]carbazole-9-carboxylic acid (30 mg, 103.3 umol, 1 eq) in DMF (2 mL) was added DIEA (40.0 mg, 310.0 umol, 54.0 uL, 3 eq) and HATU (58.9 mg, 155.0 umol, 1.5 eq) and stirred for 30 min. (2S)-2-aminopropan-1-ol (9.3 mg, 124.0 umol, 9.6 ul, 1.2 eq) was added to the mixture. The mixture was stirred at 25° C. for 6 hr. The reaction mixture was concentrated to dryness. The crude product was purified by reversed-phase HPLC (0.1% NH₃·H₂O condition column: Phenomenex Gemini-NX C18 75*30 mm*3 um; mobile phase: [water(0.05% NH₃H2O+10 mM NH₄HCO₃)-ACN]; B %: 25%-55%, 8 min) to give N-[(1S)-2-hydroxy-1-methyl-ethyl]-5,6-dimethyl-pyrido[4,3-b]carbazole-9-carboxamide (12.3 mg, 35.0 umol, 33.9% yield, 99.29% purity) as a yellow solid.

¹HNMR (400 MHz, METHANOL-d₄) δ 9.32 (s, 1H), 8.81 (d, J=1.6 Hz, 1H), 8.75 (s, 1H), 8.39 (br d, J=6.4 Hz, 1H), 8.18-8.07 (m, 2H), 7.59 (d, J=8.6 Hz, 1H), 4.34-4.27 (m, 1H), 4.24 (s, 3H), 3.70 (dq, J=5.8, 10.8 Hz, 2H), 3.15 (s, 3H), 1.35 (d, J=6.8 Hz, 3H)

MS (ESI): m/z=348.2 [M+H]⁺

### (R)—N-(1-(dimethylamino)propan-2-yl)-5,6-dimethyl-6H-pyrido[4,3-b]carbazole-1-carboxamide (Compound 292)

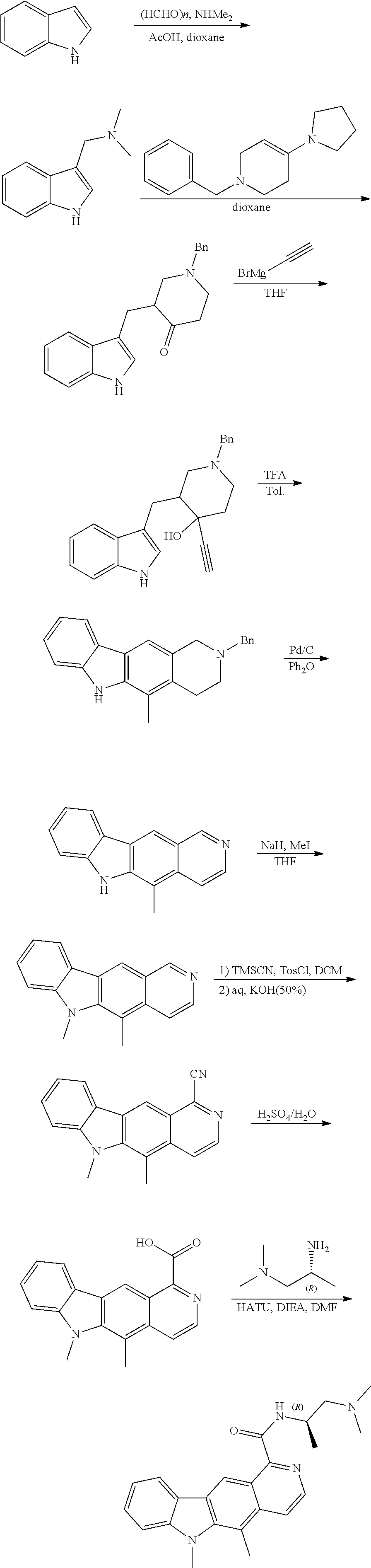

### Step 1: 1-(1H-indol-3-yl)-N,N-dimethylmethanamine

To a solution of indole (25 g, 213.4 mmol, 1 eq) in dioxane (283 mL) and H₂O (283 mL) was added dimethylamine hydrochloride (19.1 g, 234.7 mmol, 1.1 eq), formaldehyde (19.0 g, 234.7 mmol, 17.4 mL, 37% purity, 1.1 eq), TEA (23.7 g, 234.7 mmol, 32.6 mL, 1.1 eq) and AcOH (19.2 g, 320.1 mmol, 18.3 mL, 1.5 eq). The mixture was stirred at 30° C. for 12 hr. The mixture was concentrated to remove the dioxane and poured into NaOH (1000 mL, 2N) and solid was separated out. The mixture was filtered and wash with H₂O (500 mL) and the cake was collected to give 1-(1H-indol-3-yl)-N,N-dimethyl-methanamine.

### Step 2: 3-((1H-indol-3-yl)methyl)-1-benzylpiperidin-4-one

To a solution of 1-(1H-indol-3-yl)-N,N-dimethyl-methanamine (15 g, 86.0 mmol, 1 eq) in dioxane (400 mL) was added 1-benzyl-4-pyrrolidin-1-yl-3,6-dihydro-2H-pyridine (52.1 g, 215.2 mmol, 2.5 eq). The mixture was stirred at 120° C. for 12 hr. The mixture was concentrated to get crude residue. The residue was purified by flash silica gel chromatography (ISCO®; 330 g SepaFlash® Silica Flash Column, Eluent of 0˜30% Ethyl acetate/Petroleum ether gradient @ 60 mL/min) to give 3-((1H-indol-3-yl)methyl)-1-benzylpiperidin-4-one.

### Step 3: 3-((1H-indol-3-yl)methyl)-1-benzyl-4-ethynylpiperidin-4-ol

To a solution of 3-((1H-indol-3-yl)methyl)-1-benzylpiperidin-4-one (19 g, 59.6 mmol, 1 eq) in THF (150 mL) was added bromo(ethynyl)magnesium (0.5 M, 477.3 mL, 4 eq). The mixture was stirred at 0° C. for 1 hr. The reaction mixture was quenched by addition NH₄Cl (500 mL) at 25° C., and extracted with EtOAc (100 mL×3). The combined organic layers were washed with brine (50 mL×3), dried over Na₂SO₄, filtered and concentrated under reduced pressure to give 3-((1H-indol-3-yl)methyl)-1-benzyl-4-ethynylpiperidin-4-ol.

### Step 4: 2-benzyl-5-methyl-2,3,4,6-tetrahydro-1H-pyrido[4,3-b]carbazole

To a solution of 3-((1H-indol-3-yl)methyl)-1-benzyl-4-ethynylpiperidin-4-ol (20.5 g, 59.5 mmol, 1 eq) in toluene (440 mL) was added TFA (67.8 g, 595.1 mmol, 44.0 mL, 10 eq). The mixture was stirred at 80° C. for 12 hr. The mixture was concentrated to get crude residue and diluted with EtOAc 500 mL and adjusted pH=8 with sat NaHCO₃ extracted with EtOAc (500 mL×3). The combined organic layers were washed with brine (500 mL×3), dried over Na₂SO₄, filtered and concentrated under reduced pressure to give a residue. The mixture was diluted with EtOAc 500 mL and filtered and collect the solid to afford 2-benzyl-5-methyl-2,3,4,6-tetrahydro-1H-pyrido[4,3-b]carbazole.

### Step 5: 5-methyl-6H-pyrido[4,3-b]carbazole

To a solution of Pd/C (2 g, 10% purity) in Ph₂O (50 mL) was added 2-benzyl-5-methyl-2,3,4,6-tetrahydro-1H-pyrido[4,3-b]carbazole (4 g, 12.2 mmol, 1 eq). The mixture was stirred at 220° C. for 4 hr. The reaction mixture was filtered and washed with the solvent (DCM:MeOH=10:1, 200 mL×3) and the filtrate dried in vacuum to give crude product. The residue was purified by flash silica gel chromatography (ISCO®; 20 g SepaFlash® Silica Flash Column, Eluent of 0˜10% MeOH/DCM @ 150 mL/min) to give 5-methyl-6H-pyrido[4,3-b]carbazole.

### Step 6: 5,6-dimethyl-6H-pyrido[4,3-b]carbazole

To a solution of 5-methyl-6H-pyrido[4,3-b]carbazole (2 g, 8.6 mmol, 1 eq) in THF (30 mL) was added NaH (1.0 g, 25.8 mmol, 60% purity, 3 eq) at 0° C. and stirred for 30 min. Mel (1.2 g, 8.6 mmol, 536.0 uL, 1 eq) was added to the mixture. The mixture was stirred at 25° C. for 2 hr. The reaction mixture was partitioned between NH₄Cl 50 mL and EtOAc 50 mL. The water phase was separated, extracted with EtOAc (100 mL×3). The combined organic phase was washed with brine (100 mL×3), dried over Na₂SO₄, filtered and concentrated under reduced pressure to give a residue. The residue was purified by flash silica gel chromatography (ISCO®; 4 g SepaFlash® Silica Flash Column, Eluent of 0˜10% MeOH/DCM gradient @100 mL/min) to give 5,6-dimethyl-6H-pyrido[4,3-b]carbazole.

### Step 7: 5,6-dimethyl-6H-pyrido[4,3-b]carbazole-1-carbonitrile

To a solution of 5,6-dimethyl-6H-pyrido[4,3-b]carbazole (600 mg, 2.7 mmol, 1 eq) in DCM (40 mL) was added TMSCN (712.1 mg, 7.1 mmol, 897.9 uL, 2.6 eq). To this mixture a solution of TosCl (1.0 g, 5.2 mmol, 1.9 eq) in DCM (10 mL) was added at 25° C. Then the mixture was stirred at 30° C. for 12 hr. Then, 10 mL KOH (50% W/W) was added and NH₄HSO₄ (15 mg). The mixture was stirred at 25° C. for 2 hr. LCMS showed the reaction was completed and desired product. The mixture was extracted with DCM (100 mL×3). The combined organic layers were washed with brine (100 mL×3), dried over Na₂SO₄, filtered, and concentrated under reduced pressure to give 5,6-dimethyl-6H-pyrido[4,3-b]carbazole-1-carbonitrile.

### Step 8: 5,6-dimethyl-6H-pyrido[4,3-b]carbazole-1-carboxylic acid

A mixture of 5,6-dimethyl-6H-pyrido[4,3-b]carbazole-1-carbonitrile (100 mg, 368.5 umol, 1 eq) in H₂O (2 mL) and H₂SO₄ (1 mL, 98% purity) was stirred at 110° C. for 40 hr. The reaction mixture was added to NaHCO₃ (100 mL) and concentrated under reduced pressure to give 5,6-dimethyl-6H-pyrido[4,3-b]carbazole-1-carboxylic acid.

### Step 9: (R)—N-(1-(dimethylamino)propan-2-yl)-5,6-dimethyl-6H-pyrido[4,3-b]carbazole-1-carboxamide

To a solution of 5,6-dimethyl-6H-pyrido[4,3-b]carbazole-1-carboxylic acid (50 mg, 172.2 umol, 1 eq) in DMF (2 mL) was added DIEA (66.7 mg, 516.6 umol, 90.0 uL, 3 eq), HATU (98.2 mg, 258.3 umol, 1.5 eq) and stirred for 30 min. (2R)—N1,N1-dimethylpropane-1,2-diamine (60.3 mg, 344.4 umol, 2 eq, 2 HCl) was added to the mixture. The mixture was stirred at 25° C. for 12 hr. The reaction mixture was concentrated under reduced pressure to give a residue. The residue was purified by prep-HPLC (TFA condition, column: Phenomenex Luna C18 150*30 mm*5 um; mobile phase: [water(0.1% TFA)-ACN]; B %: 15%-35%, 9 min) to give (R)—N-(1-(dimethylamino)propan-2-yl)-5,6-dimethyl-6H-pyrido[4,3-b]carbazole-1-carboxamide.

MS (ESI): m/z=375.2 [M+H]⁺

### N-(2-aminoethyl)-5,6-dimethyl-6H-pyrido[4,3-b]carbazole-1-carboxamide (Compound 293)

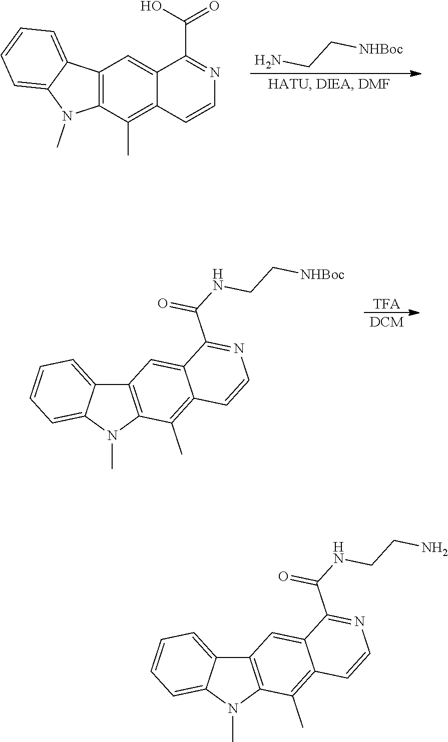

### Step 1: tert-butyl (2-(5,6-dimethyl-6H-pyrido[4,3-b]carbazole-1-carboxamido)ethyl)carbamate

To a solution of 5,6-dimethylpyrido[4,3-b]carbazole-1-carboxylic acid (80 mg, 275.5 umol, 1 eq) in DMF (3 mL) was added DIEA (106.8 mg, 826.6 umol, 143.9 uL, 3 eq), HATU (157.1 mg, 413.3 umol, 1.5 eq) and stirred for 30 min. tert-butyl N-(2-aminoethyl)carbamate (88.3 mg, 551.1 umol, 86.5 uL, 2 eq) was added to the mixture. The mixture was stirred at 25° C. for 12 hr. The reaction was added to H₂O (20 mL) and filtered, and the filter cake was washed with 10 mL of H₂O, dried in vacuum to give tert-butyl (2-(5,6-dimethyl-6H-pyrido[4,3-b]carbazole-1-carboxamido) ethyl)carbamate.

### Step 2: N-(2-aminoethyl)-5,6-dimethyl-6H-pyrido[4,3-b]carbazole-1-carboxamide

A mixture of tert-butyl (2-(5,6-dimethyl-6H-pyrido[4,3-b]carbazole-1-carboxamido)ethyl) carbamate (60 mg, 138.7 umol, 1 eq) in TFA (2 mL) and DCM (1 mL) was stirred at 25° C. for 2 hr. The reaction mixture was concentrated under reduced pressure to give a residue. The residue was purified by prep-HPLC (TFA condition, column: Phenomenex luna C18 100*40 mm*5 um; mobile phase: [water(0.1% TFA)-ACN]; B %: 1%-30%, 8 min) to give N-(2-aminoethyl)-5,6-dimethyl-6H-pyrido[4,3-b]carbazole-1-carboxamide.

MS (ESI): m/z=333.1 [M+H]⁺

### N-[(2R)-2-(dimethylamino)propyl]-5,6-dimethyl-pyrido[4,3-b]carbazole-9-carboxamide (Compound 294)

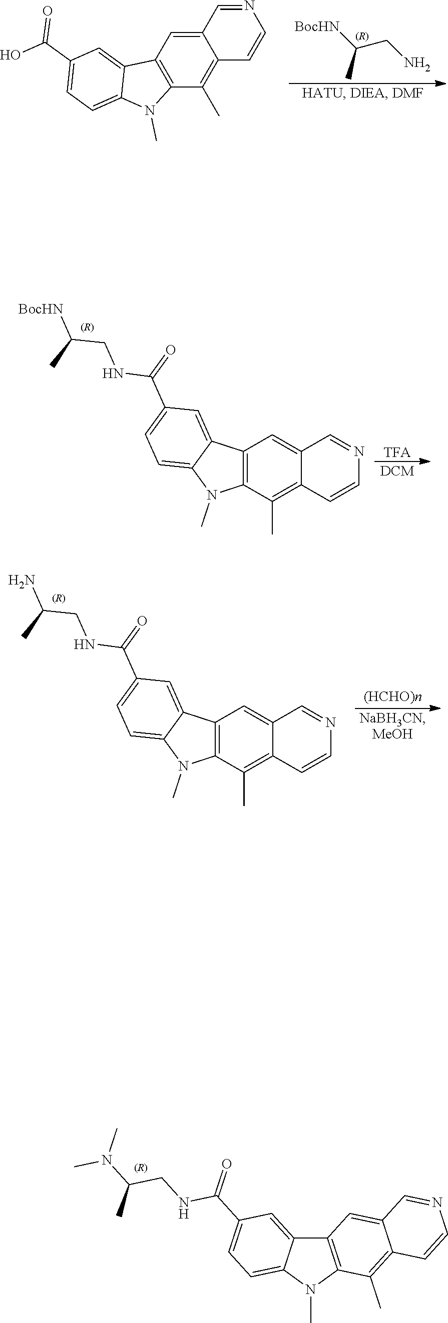

### Step 1: tert-butylN-[(1R)-2-[(5,6-dimethylpyrido[4,3-b]carbazole-9-carbonyl)amino]-1-methyl-ethyl]carbamate

To a solution of 5,6-dimethylpyrido[4,3-b]carbazole-9-carboxylic acid (200 mg, 688.9 umol, 1 eq) in DMF (4 mL) was added DIEA (89.0 mg, 688.9 umol, 119.9 uL, 1 eq) and HATU (261.9 mg, 688.9 umol, 1 eq), the mixture was stirred for 30 min. tert-butyl N-[(1R)-2-amino-1-methyl-ethyl]carbamate (240.0 mg, 1.3 mmol, 2 eq) was added to the mixture. The mixture was stirred at 25° C. for 12 hr. The residue was partitioned between H₂O (5 mL) and Dichloromethane (5 mL). The mixture was extracted with Dichloromethane (5 mL×3). The separated organic layer was washed with brine (5 mL×3), dried over Na₂SO₄ and evaporated to dryness. The residue was purified by prep-TLC (SiO₂, Dichloromethane:Methanol=10/1) to give tert-butyl N-[(1R)-2-[(5,6-dimethylpyrido[4,3-b]carbazole-9-carbonyl)amino]-1-methyl-ethyl]carbamate.

### Step 2: N-[(2R)-2-aminopropyl]-5,6-dimethyl-pyrido[4,3-b]carbazole-9-carboxamide

To a solution of tert-butyl N-[(1R)-2-[(5,6-dimethylpyrido[4,3-b]carbazole-9-carbonyl)amino]-1-methyl-ethyl]carbamate (70 mg, 156.76 umol, 1 eq) in DCM (2 mL) and was added TFA (1.5 g, 13.5 mmol, 1 mL, 86.1 eq). The mixture was stirred at 25° C. for 2 hr. The reaction mixture was concentrated to dryness to give N-[(2R)-2-aminopropyl]-5,6-dimethyl-pyrido[4,3-b]carbazole-9-carboxamide.

### Step 3: N-[(2R)-2-(dimethylamino)propyl]-5,6-dimethyl-pyrido[4,3-b]carbazole-9-carboxamide

To a solution of N-[(2R)-2-aminopropyl]-5,6-dimethyl-pyrido[4,3-b]carbazole-9-carboxamide (54 mg, 155.8 umol, 1 eq) in MeOH (2 mL) was added TEA to the above mixture was adjusted pH to 8 and was added (HCHO)n (29.9 mg, 311.7 umol, 2 eq). AcOH was added to the above mixture was adjusted pH to 5. NaBH₃CN (19.5 mg, 311.7 umol, 2 eq) was added to the mixture. The mixture was stirred at 25° C. for 12 hr. The reaction mixture was concentrated to dryness. The crude product was purified by reversed-phase HPLC (TFA condition column: Phenomenex luna C18 100*40 mm*5 um; mobile phase: [water(0.1% TFA)-ACN]; B %: 7%-27%, 8 min) to give N-[(2R)-2-(dimethylamino)propyl]-5,6-dimethyl-pyrido[4,3-b]carbazole-9-carboxamide.

MS (ESI): m/z=375.2 [M+H]⁺

### N-[(1S)-2-(dimethylamino)-1-methyl-ethyl]-5,6-dimethyl-pyrido[4,3-b]carbazole-9-carboxamide (Compound 295)

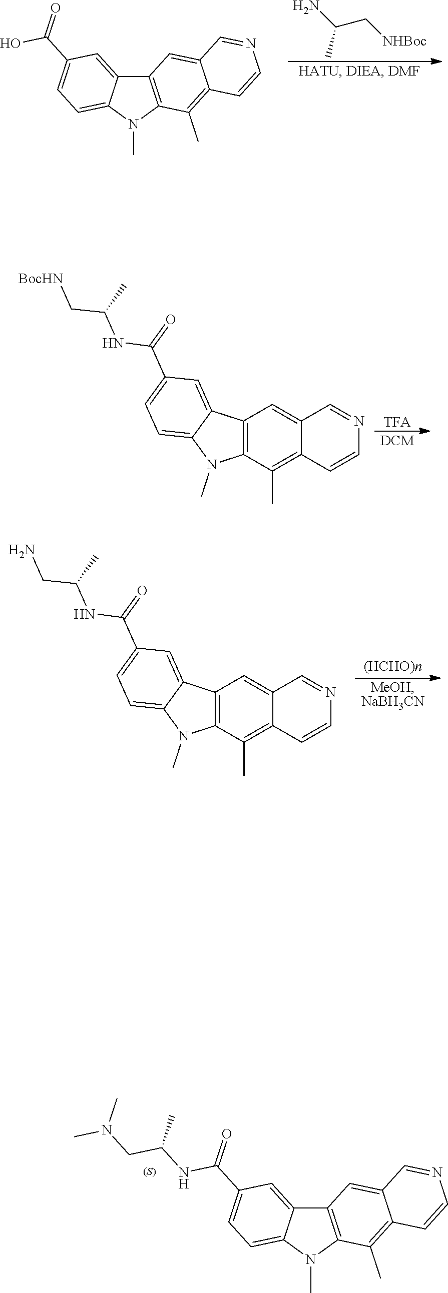

### Step 1: tert-butyl N-[(2S)-2-[(5,6-dimethylpyrido[4,3-b]carbazole-9-carbonyl)amino]propyl]carbamate

To a solution of 5,6-dimethylpyrido[4,3-b]carbazole-9-carboxylic acid (50 mg, 172.2 umol, 1 eq) in DMF (2 mL) was added DIEA (66.7 mg, 516.6 umol, 90.0 uL, 3 eq) and HATU (98.2 mg, 258.3 umol, 1.5 eq) and the mixture was stirred at 25° C. for 30 min. tert-butyl N-[(2S)-2-aminopropyl]carbamate (60.0 mg, 344.4 umol, 2 eq) was added to the mixture and stirred at 25° C. for 12 hr. The reaction mixture was partitioned between H₂O 5 mL and CH₂Cl₂ 5 mL. The water phase was separated, extracted with CH₂Cl₂ (5 mL×3). The combined organic phase was washed with brine (10 mL×3), dried over Na₂SO₄, filtered, and concentrated under reduced pressure to give a residue. The residue was purified by prep-TLC (SiO₂, DCM:MeOH=10:1) to give tert-butyl N-[(2S)-2-[(5,6-dimethylpyrido[4,3-b]carbazole-9-carbonyl)amino]propyl]carbamate.

### Step 2: N-[(1S)-2-amino-1-methyl-ethyl]-5,6-dimethyl-pyrido[4,3-b]carbazole-9-carboxamide

The mixture of tert-butyl N-[(2S)-2-[(5,6-dimethylpyrido[4,3-b]carbazole-9-carbonyl)amino]propyl]carbamate (50 mg, 111.9 umol, 1 eq) in DCM (2 mL) and TFA (1 mL) was stirred at 25° C. for 2 hr. The reaction mixture was concentrated under reduced pressure to remove solvent to give N-[(1S)-2-amino-1-methyl-ethyl]-5,6-dimethyl-pyrido[4,3-b]carbazole-9-carboxamide.

### Step 3: N-[(1S)-2-(dimethylamino)-1-methyl-ethyl]-5,6-dimethyl-pyrido[4,3-b]carbazole-9-carboxamide

To a solution of N-[(1S)-2-amino-1-methyl-ethyl]-5,6-dimethyl-pyrido[4,3-b]carbazole-9-carboxamide (50 mg, 144.3 umol, 1 eq) in MeOH (3 mL) was added TEA to adjust PH to 8, (HCHO)n (55.4 mg, 577.3 umol, 4 eq), AcOH to adjust PH to 5. The mixture was stirred at 25° C. for 30 min. NaBH₃CN (18.1 mg, 288.6 umol, 2 eq) was added to the mixture and the mixture stirred at 25° C. for 12 hr. The reaction mixture was filtered. The residue was purified by prep-HPLC (TFA condition; column: Phenomenex Luna C18 150*30 mm*5 um; mobile phase: [water(0.1% TFA)-ACN]; B %: 1%-35%, 9 min). The residue was purified by prep-HPLC (neutral condition; column: Waters Xbridge BEH C18 100*30 mm*10 um; mobile phase: [water(10 mM NH₄HCO₃)-ACN]; B %: 15%-45%, 8 min) to give N-[(1S)-2-(dimethylamino)-1-methyl-ethyl]-5,6-dimethyl-pyrido[4,3-b]carbazole-9-carboxamide.

MS (ESI): m/z=375.1 [M+H]⁺

### N-(2-amino-2-oxoethyl)-5,6-dimethyl-6H-pyrido[4,3-b]carbazole-9-carboxamide (Compound 296)

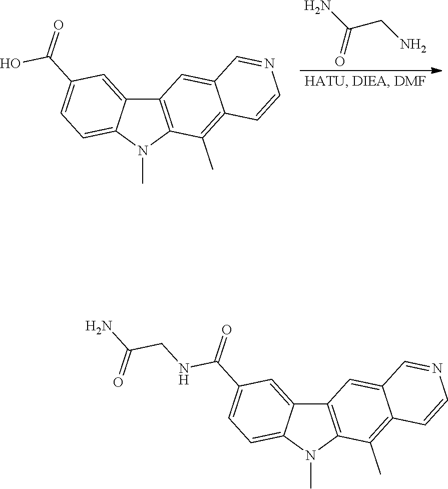

To a solution of 5,6-dimethylpyrido[4,3-b]carbazole-9-carboxylic acid (40 mg, 137.8 umol, 1 eq) in DMF (2 mL) was added DIEA (53.4 mg, 413.3 umol, 72.0 uL, 3 eq) and HATU (78.6 mg, 206.7 umol, 1.5 eq). The reaction mixture was stirred at 25° C. for 30 min. 2-aminoacetamide (22.9 mg, 206.7 umol, 1.5 eq, HCl) was added to the mixture. The reaction mixture was stirred at 25° C. for 3 hr. The residue was filtered, and the filtrate was obtained as brown liquid and purified by prep-HPLC (TFA condition; column: Phenomenex luna C18 100*40 mm*5 um; mobile phase: water (0.1% TFA)-ACN]; B %: 1%-30%, 8 min) to give N-(2-amino-2-oxoethyl)-5,6-dimethyl-6H-pyrido[4,3-b]carbazole-9-carboxamide.

MS (ESI): m/z=347.1 [M+H]⁺

### (S)—N-(1-amino-1-oxopropan-2-yl)-5,6-dimethyl-6H-pyrido[4,3-b]carbazole-9-carboxamide (Compound 297)

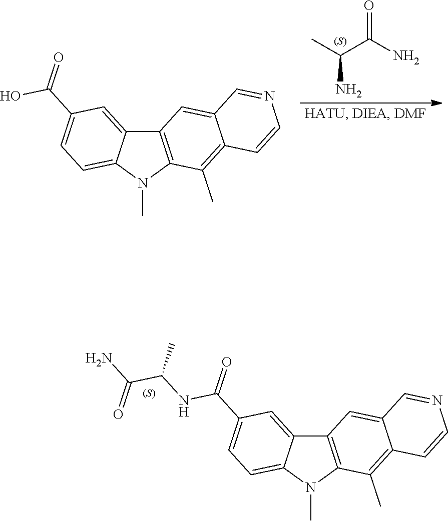

To a solution of 5,6-dimethylpyrido[4,3-b]carbazole-9-carboxylic acid (40 mg, 137.8 umol, 1 eq) in DMF (2 mL) added HATU (78.6 mg, 206.7 umol, 1.5 eq), DIEA (53.4 mg, 413.3 umol, 72.0 uL, 3 eq), (2S)-2-aminopropanamide; hydrochloride (25.7 mg, 206.7 umol, 1.5 eq). The reaction mixture was stirred at 25° C. for 2 hr. The reaction mixture was filtered. The residue was purified by prep-HPLC (TFA condition, column: Phenomenex luna C18 100×40 mm×5 um; mobile phase: [water(0.1% TFA)-ACN]; B %: 1%-30%, 8 min) to afford (S)—N-(1-amino-1-oxopropan-2-yl)-5,6-dimethyl-6H-pyrido[4,3-b]carbazole-9-carboxamide.

MS (ESI): m/z=361.1[M+H]⁺

### (R)—N-(1-amino-1-oxopropan-2-yl)-5,6-dimethyl-6H-pyrido[4,3-b]carbazole-9-carboxamide (Compound 298)

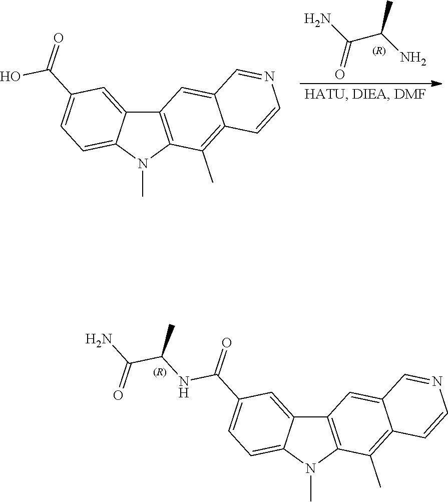

To a solution of 5,6-dimethylpyrido[4,3-b]carbazole-9-carboxylic acid (50 mg, 172.2 umol, 1 eq) in DMF (2 mL) was added DIEA (66.8 mg, 516.7 umol, 90.0 uL, 3 eq) and HATU (98.2 mg, 258.3 umol, 1.5 eq). The reaction mixture was stirred at 25° C. for 30 min. (2R)-2-aminopropanamide; hydrochloride (32.2 mg, 258.3 umol, 1.5 eq) was added to the mixture. The reaction mixture was stirred at 25° C. for 3 hr. The residue was filtered, and the filtrate was obtained as brown liquid and purified by prep-HPLC (TFA condition; column: Phenomenex luna C18 100*40 mm*5 um; mobile phase: [water(0.1% TFA)-ACN]; B %: 1%-30%, 8 min) to give (R)—N-(1-amino-1-oxopropan-2-yl)-5,6-dimethyl-6H-pyrido[4,3-b]carbazole-9-carboxamide.

MS (ESI): m/z=361.1 [M+H]⁺

### N-(2-methoxy-2-methylpropyl)-5,6-dimethyl-6H-pyrido[4,3-b]carbazole-9-carboxamide (Compound 299)

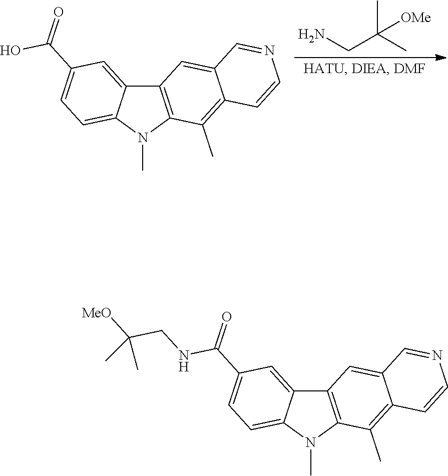

To a solution of 5,6-dimethyl-6H-pyrido[4,3-b]carbazole-9-carboxylic acid (50 mg, 172.2 umol, 1 eq) in DMF (2 mL) was added DIEA (66.7 mg, 516.6 umol, 90.0 uL, 3 eq) and HATU (98.2 mg, 258.3 umol, 1.5 eq) and stirred for 30 min. 2-methoxy-2-methylpropan-1-amine (21.3 mg, 206.6 umol, 1.2 eq) was added to the mixture. The mixture was stirred at 25° C. for 6 hr. The reaction mixture was concentrated to dryness. The crude product was purified by reversed-phase HPLC (TFA condition column: Phenomenex Luna C18 150*30 mm*5 um; mobile phase: [water(0.1% TFA)-ACN]; B %: 5%-40%, 9 min) to give N-(2-methoxy-2-methylpropyl)-5,6-dimethyl-6H-pyrido[4,3-b]carbazole-9-carboxamide.

MS (ESI): m/z=376.2 [M+H]⁺

### (S)—N-(2-(dimethylamino)propyl)-5,6-dimethyl-6H-pyrido[4,3-b]carbazole-9-carboxamide (Compound 300)

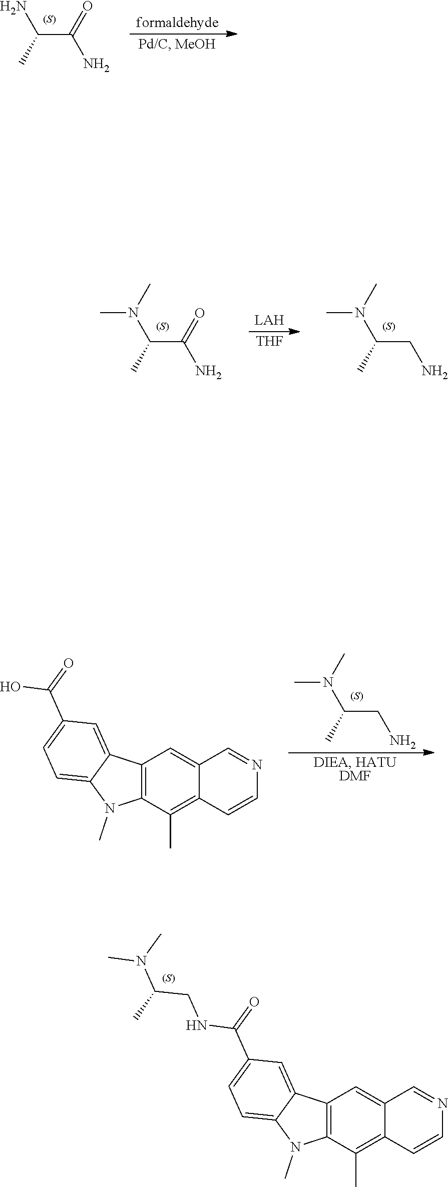

### Step 1: (S)-2-(dimethylamino)propanamide

To a solution of (S)-2-aminopropanamide (1 g, 8.0 mmol, 1 eq, HCl) and formaldehyde (1.4 g, 17.6 mmol, 1.3 mL, 37% purity, 2.2 eq) in MeOH (15 mL) was added Pd/C (0.4 g, 10% purity). The mixture was stirred at 40° C. for 5 hr under H₂ (15 psi). The mixture was filtered and concentrated under reduced pressure to give (S)-2-(dimethylamino)propanamide.

### Step 2: (S)—N2,N2-dimethylpropane-1,2-diamine

To a solution of (S)-2-(dimethylamino)propanamide (1.3 g, 8.5 mmol, 1 eq, HCl) in THF (20 mL) was added LAH (711.2 mg, 18.7 mmol, 2.2 eq). The mixture was stirred at 25° C. for 1 hr. Then the mixture was stirred at 80° C. for 12 hr under N₂. The reaction mixture was quenched by addition 0.7 mL Sat. MgSO₄, filtered and concentrated under reduced pressure to give (S)—N2,N2-dimethylpropane-1,2-diamine.

### Step 3: (S)—N-(2-(dimethylamino)propyl)-5,6-dimethyl-6H-pyrido[4,3-b]carbazole-9-carboxamide

To a solution of 5,6-dimethyl-6H-pyrido[4,3-b]carbazole-9-carboxylic acid (50 mg, 172.2 umol, 1 eq) in DMF (2 mL) was added DIEA (66.7 mg, 516.6 umol, 90.0 uL, 3 eq), HATU (98.2 mg, 258.3 umol, 1.5 eq) and stirred for 30 min. (S)—N2,N2-dimethylpropane-1,2-diamine (35.2 mg, 344.4 umol, 2 eq) was added to the solution and stirred at 25° C. for 2 hr. The reaction mixture was concentrated under reduced pressure to remove solvent. The residue was purified by prep-HPLC (TFA condition, column: Phenomenex luna C18 100*40 mm*5 um; mobile phase: [water(0.1% TFA)-ACN]; B %: 10%-30%, 8 min) to give (S)—N-(2-(dimethylamino)propyl)-5,6-dimethyl-6H-pyrido[4,3-b]carbazole-9-carboxamide.

MS (ESI): m/z=375.2 [M+H]⁺

### N-(2-(tert-butoxy)ethyl)-5,6-dimethyl-6H-pyrido[4,3-b]carbazole-9-carboxamide (Compound 301)

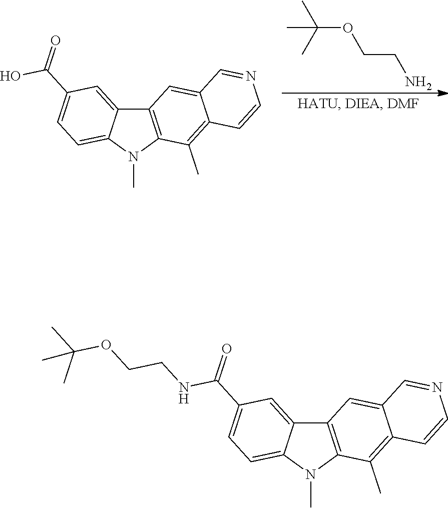

To a solution of 5,6-dimethyl-6H-pyrido[4,3-b]carbazole-9-carboxylic acid (50 mg, 172.2 umol, 1 eq) in DMF (2 mL) was added DIEA (66.7 mg, 516.6 umol, 90.0 uL, 3 eq), HATU (98.2 mg, 258.3 umol, 1.5 eq) and stirred at 25° C. for 30 min. 2-tert-butoxyethanamine; hydrochloride (52.9 mg, 344.4 umol, 2 eq) was added to the solution and stirred at 25° C. for 12 hr. The reaction mixture was concentrated under reduced pressure to remove solvent. The residue was purified by prep-HPLC (TFA condition, column: Phenomenex luna C18 100*40 mm*5 um; mobile phase: [water(0.1% TFA)-ACN]; B %: 10%-40%, 8 min) to give N-(2-(tert-butoxy)ethyl)-5,6-dimethyl-6H-pyrido[4,3-b]carbazole-9-carboxamide.

MS (ESI): m/z=390.2 [M+H]⁺

### N-(1-methoxy-2-methylpropan-2-yl)-5,6-dimethyl-6H-pyrido[4,3-b]carbazole-9-carb-oxamide (Compound 302)

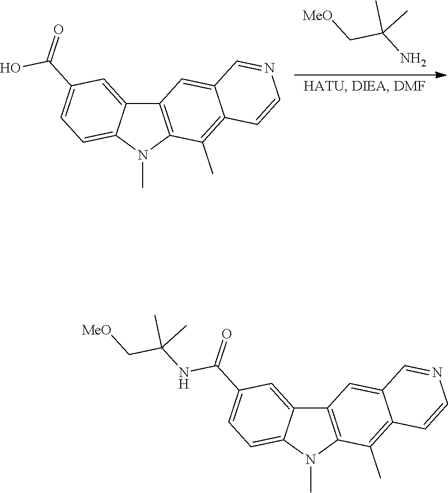

To a solution of 5,6-dimethylpyrido[4,3-b]carbazole-9-carboxylic acid (80 mg, 275.6 umol, 1 eq) in DMF (2 mL) was added DIEA (106.8 mg, 826.7 umol, 143.9 uL, 3 eq) and HATU (157.2 mg, 413.3 umol, 1.5 eq). The reaction mixture was stirred at 25° C. for 30 min. 1-Methoxy-2-methyl-propan-2-amine (42.6 mg, 413.3 umol, 1.5 eq) was added to the mixture. The reaction mixture was stirred at 25° C. for 3 hr. The residue was filtered, and the filtrate was obtained as brown liquid and purified by prep-HPLC (TFA condition; column: Phenomenex luna C18 100*40 mm*5 um; mobile phase: [water(0.1% TFA)-ACN]; B %: 1%-36%, 8 min) to give N-(1-methoxy-2-methylpropan-2-yl)-5,6-dimethyl-6H-pyrido[4,3-b]carbazole-9-carb-oxamide.

MS (ESI): m/z=376.2 [M+H]⁺

### 4-((5,6-dimethyl-6H-pyrido[4,3-b]carbazol-9-yl)oxy)pyridin-2-amine (Compound 303)

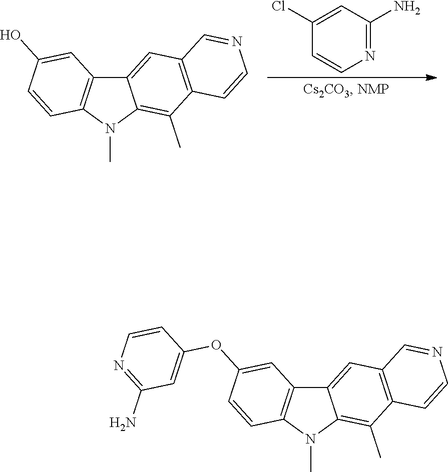

To a solution of 5,6-dimethyl-6H-pyrido[4,3-b]carbazol-9-ol (100 mg, 381.2 umol, 1 eq) in NMP (3 mL) was added 4-chloropyridin-2-amine (58.8 mg, 457.4 umol, 1.2 eq) and Cs₂CO₃ (149.0 mg, 457.4 umol, 1.2 eq). The mixture was stirred at 220° C. for 1 hr under microwave. The reaction mixture was concentrated under reduced pressure to remove solvent. The residue was purified by prep-HPLC (TFA condition; column: Phenomenex luna C18 100*40 mm*5 um; mobile phase: [water(0.1% TFA)-ACN]; B %: 5%-40%, 8 min) to give 4-((5,6-dimethyl-6H-pyrido[4,3-b]carbazol-9-yl)oxy)pyridin-2-amine.

MS (ESI): m/z=355.1 [M+H]⁺

### 6-((5,6-dimethyl-6H-pyrido[4,3-b]carbazol-9-yl)oxy)pyridin-2-amine (Compound 304)

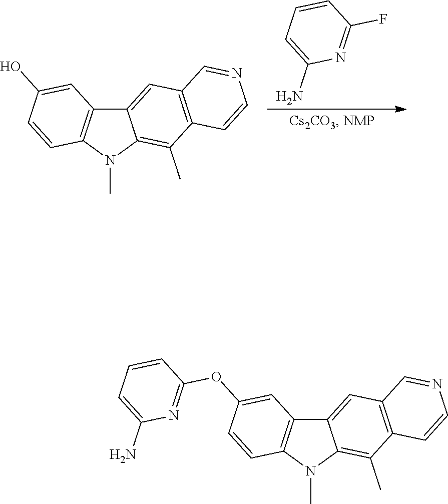

To a solution of 5,6-dimethylpyrido[4,3-b]carbazol-9-ol (100 mg, 381.2 umol, 1 eq) in NMP (3 mL) was added 6-fluoropyridin-2-amine (51.2 mg, 457.4 umol, 1.2 eq) and Cs₂CO₃ (149.0 mg, 457.4 umol, 1.2 eq). The mixture was stirred at 220° C. for 1 hr under microwave. The reaction mixture was filtered, and the filtrate dried in vacuum to give crude product. The residue was purified by prep-HPLC (basic condition, column: Phenomenex Gemini-NX C18 75*30 mm*3 um; mobile phase: [water(0.05% NH₃H2O+10 mM NH₄HCO₃)-ACN]; B %: 35%-65%, 8 min) to give 6-((5,6-dimethyl-6H-pyrido[4,3-b]carbazol-9-yl)oxy)pyridin-2-amine.

MS (ESI): m/z=355.1 [M+H]⁺

### 5,6-dimethyl-9-(pyridin-2-yloxy)-6H-pyrido[4,3-b]carbazole (Compound 305)

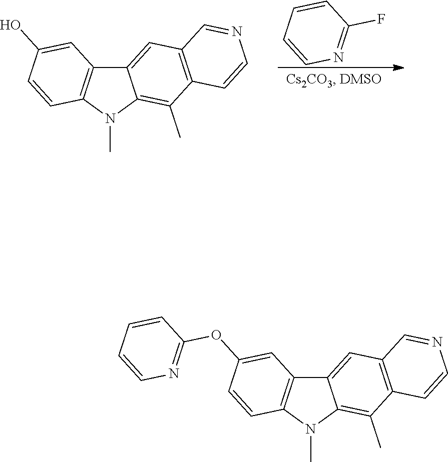

To a solution of 5,6-dimethyl-6H-pyrido[4,3-b]carbazol-9-ol (70 mg, 266.8 umol, 1 eq) in DMSO (1 mL) was added 2-fluoropyridine (31.0 mg, 320.2 umol, 27.5 uL, 1.2 eq) and Cs₂CO₃ (260.8 mg, 800.5 umol, 3 eq). The mixture was stirred at 120° C. for 12 hr. The reaction mixture was concentrated to dryness. The residue was purified by prep-HPLC (0.05% NH₃·H₂O column: Waters Xbridge Prep OBD C18 150*40 mm*10 um; mobile phase: [water(0.05% NH₃H2O+10 mM NH₄HCO₃)-ACN]; B %: 35%-65%, 8 min) to give 5,6-dimethyl-9-(pyridin-2-yloxy)-6H-pyrido[4,3-b]carbazole.

MS (ESI): m/z=340.1 [M+H]⁺

### 5,6-dimethyl-9-(pyridin-4-yloxy)-6H-pyrido[4,3-b]carbazole (Compound 306)

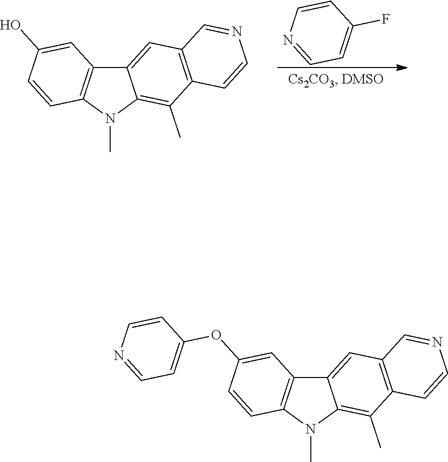

To a solution of 5,6-dimethylpyrido[4,3-b]carbazol-9-ol (60 mg, 228.7 umol, 1 eq) in DMSO (2 mL) was added 4-fluoropyridine (36.6 mg, 274.4 umol, 1.2 eq, HCl) and Cs₂CO₃ (223.5 mg, 686.2 umol, 3 eq). The mixture was stirred at 120° C. for 12 hr. The reaction mixture was concentrated under reduced pressure to give a residue. The residue was purified by prep-HPLC (TFA condition, column: Phenomenex Luna C18 150*30 mm*5 um; mobile phase: [water(0.1% TFA)-ACN]; B %: 1%-30%, 9 min) to give 5,6-dimethyl-9-(pyridin-4-yloxy)-6H-pyrido[4,3-b]carbazole.

MS (ESI): m/z=340.1 [M+H]⁺

### (R)—N-(1-(dimethylamino)propan-2-yl)-8,9-difluoro-5,6-dimethyl-6H-pyrido[4,3-b]carbazole-1-carboxamide (Compound 307)

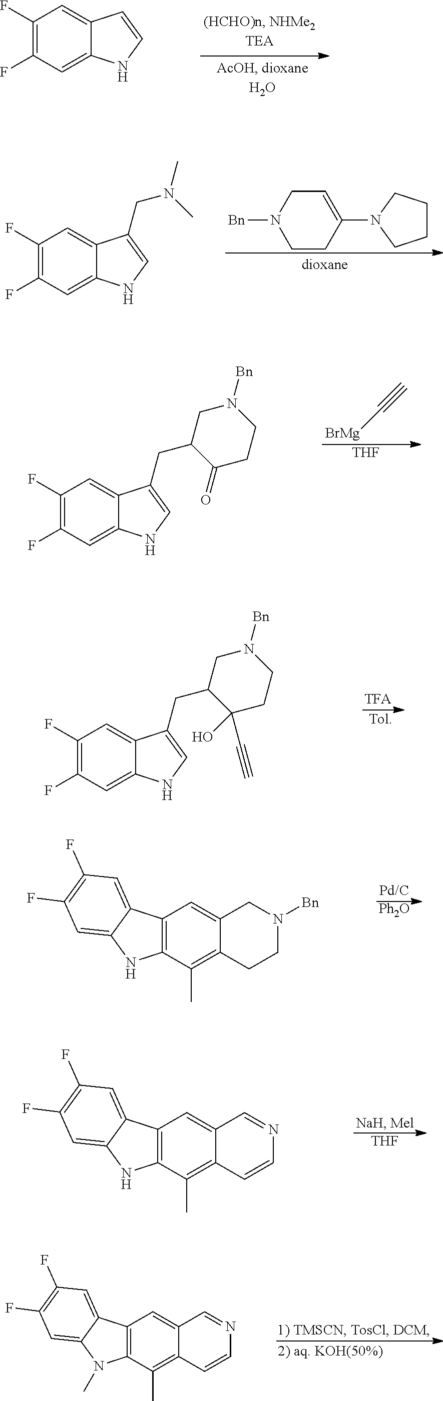

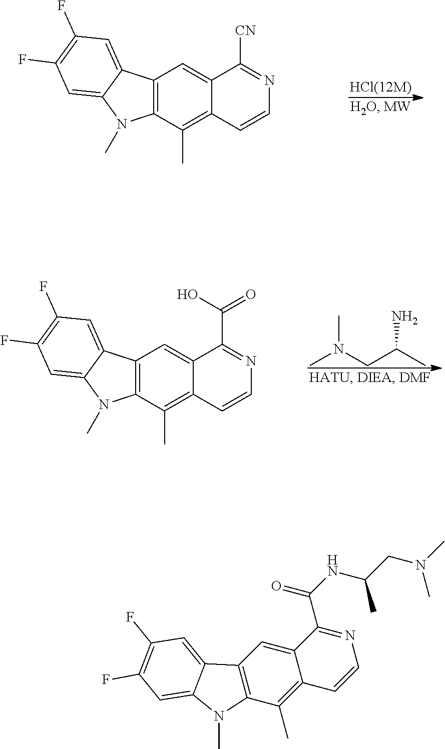

### Step 1: 1-(5,6-difluoro-1H-indol-3-yl)-N,N-dimethylmethanamine

To a solution of 5,6-difluoro-1H-indole (5 g, 32.6 mmol, 1 eq) in dioxane (56 mL) and H₂O (56 mL) was added dimethylamine hydrochloride (2.9 g, 35.9 mmol, 1.1 eq), formaldehyde (2.9 g, 35.9 mmol, 2.6 mL, 37% purity, 1.1 eq), TEA (3.6 g, 35.9 mmol, 5.0 mL, 1.1 eq), AcOH (2.9 g, 48.9 mmol, 2.8 mL, 1.5 eq). The mixture was stirred at 30° C. for 12 hr. The mixture was concentrated to remove the dioxane and poured into NaOH (200 mL, 2N). The precipitate was collected by filtration and washed with H₂O (300 mL) to give 1-(5,6-difluoro-1H-indol-3-yl)-N,N-dimethylmethanamine.

### Step 2: 1-benzyl-3-((5,6-difluoro-1H-indol-3-yl)methyl)piperidin-4-one

To a solution of 1-(5,6-difluoro-1H-indol-3-yl)-N,N-dimethylmethanamine (6 g, 28.5 mmol, 1 eq) in dioxane (70 mL) was added 1-benzyl-4-(pyrrolidin-1-yl)-1,2,3,6-tetrahydropyridine (17.2 g, 71.3 mmol, 2.5 eq). The mixture was stirred at 120° C. for 12 hr. The reaction mixture was concentrated under reduced pressure to give a residue. The residue was purified by flash silica gel chromatography (ISCO®; 40 g SepaFlash® Silica Flash Column, Eluent of 0˜100% Ethyl acetate/Petroleum ether gradient @ 120 mL/min) to give 1-benzyl-3-[(5,6-difluoro-1H-indol-3-yl)methyl]piperidin-4-one.

### Step 3: 1-benzyl-3-((5,6-difluoro-1H-indol-3-yl)methyl)-4-ethynylpiperidin-4-ol

To a solution of 1-benzyl-3-[(5,6-difluoro-1H-indol-3-yl)methyl]piperidin-4-one (5.2 g, 14.6 mmol, 1 eq) in THF (40 mL) was added bromo(ethynyl)magnesium (0.5 M, 117.3 mL, 4 eq) at 0° C. The mixture was stirred at 0° C. for 1 hr. The reaction mixture was quenched by addition NH₄Cl (200 mL) at 25° C., and extracted with EtOAc (200 mL×3). The combined organic layers were washed with brine (200 mL×3), dried over Na₂SO₄, filtered, and concentrated under reduced pressure to give 1-benzyl-3-((5,6-difluoro-1H-indol-3-yl)methyl)-4-ethynylpiperidin-4-ol.

### Step 4: 2-benzyl-8,9-difluoro-5-methyl-2,3,4,6-tetrahydro-1H-pyrido[4,3-b]carbazole

To a solution of 1-benzyl-3-[(5,6-difluoro-1H-indol-3-yl)methyl]-4-ethynylpiperidin-4-ol (6 g, 15.7 mmol, 1 eq) in Tol. (117 mL) was added TFA (17.9 g, 157.7 mmol, 11.6 mL, 10 eq). The mixture was stirred at 80° C. for 12 hr. The reaction mixture was partitioned between NaHCO₃ 300 mL and EtOAc 300 mL. The water phase was separated, extracted with EtOAc (300 mL×3). The combined organic phase was washed with brine (300 mL×3), dried over Na₂SO₄, filtered and concentrated under reduced pressure to give a residue. The residue was purified by flash silica gel chromatography (ISCO®; 20 g SepaFlash® Silica Flash Column, Eluent of 0˜30% Ethyl acetate/Petroleum ether gradient @ 120 mL/min) to give 2-benzyl-8,9-difluoro-5-methyl-2,3,4,6-tetrahydro-1H-pyrido[4,3-b]carbazole.

### Step 5: 8,9-difluoro-5-methyl-6H-pyrido[4,3-b]carbazole

To a solution of 2-benzyl-8,9-difluoro-5-methyl-2,3,4,6-tetrahydro-1H-pyrido[4,3-b]carbazole (4 g, 11.0 mmol, 1 eq) in Ph₂O (50 mL) was added Pd/C (2 g, 10% purity). The mixture was stirred at 220° C. for 4 hr. The reaction mixture was filtered and washed with the solvent (DCM:MeOH=10:1) (300 mL×3) and the filtrate was dried in vacuum to give crude product. The residue was purified by flash silica gel chromatography (ISCO®; 20 g SepaFlash® Silica Flash Column, Eluent of 0˜30% Ethyl acetate/Petroleum ether gradient @ 120 mL/min) to give 8,9-difluoro-5-methyl-6H-pyrido[4,3-b]carbazole.

### Step 6: 8,9-difluoro-5,6-dimethyl-6H-pyrido[4,3-b]carbazole

To a solution of 8,9-difluoro-5-methyl-6H-pyrido[4,3-b]carbazole (2.9 g, 11.0 mmol, 1 eq) in THF (30 mL) was added NaH (1.3 g, 33.1 mmol, 60% purity, 3 eq) at 0° C. and stirred for 30 min. Then Mel (1.5 g, 11.0 mmol, 686.9 uL, 1 eq) was added to the mixture and stirred at 25° C. for 2 hr. The residue was diluted with NH₄Cl 200 mL and extracted with EtOAc (200 mL×3). The combined organic layers were washed with brine (200 mL×2), dried over Na₂SO₄, filtered, and concentrated under reduced pressure to give a residue. The residue was purified by flash silica gel chromatography (ISCO®; 25 g SepaFlash® Silica Flash Column, Eluent of 0˜10% MeOH/CH₂Cl₂@ 120 mL/min) to give 8,9-difluoro-5,6-dimethyl-pyrido[4,3-b]carbazole.

### Step 7: 8,9-difluoro-5,6-dimethyl-6H-pyrido[4,3-b]carbazole-1-carbonitrile

To a solution of 8,9-difluoro-5,6-dimethyl-pyrido[4,3-b]carbazole (600 mg, 2.1 mmol, 1 eq) in DCM (10 mL) was added trimethylsilylcyanide (548.2 mg, 5.5 mmol, 691.3 uL, 2.6 eq). A solution of TosCl (769.9 mg, 4.0 mmol, 1.9 eq) in DCM (5 mL) was added to the mixture at 25° C. Then the mixture was stirred at 30° C. for 12 hr. Then, 10 mL KOH (50% W/W) was added to the reaction. The mixture was stirred at 30° C. for 2 hr. The reaction mixture was filtered, and the filter cake was washed with 20 mL of H₂O, dried in vacuum to give 8,9-difluoro-5,6-dimethyl-6H-pyrido[4,3-b]carbazole-1-carbonitrile.

### Step 8: 8,9-difluoro-5,6-dimethyl-6H-pyrido[4,3-b]carbazole-1-carboxylic acid

A mixture of 8,9-difluoro-5,6-dimethyl-6H-pyrido[4,3-b]carbazole-1-carbonitrile (100 mg, 325.4 umol, 1 eq) in HCl (2 mL) (12 M) was stirred at 130° C. for 4 hr under microwave. The reaction mixture was added to NaHCO₃ (10 mL) and filtered, and the filter cake was washed with 10 mL of H₂O, dried in vacuum to give 8,9-difluoro-5,6-dimethyl-6H-pyrido[4,3-b]carbazole-1-carboxylic acid.

### Step 9: (R)—N-(1-(dimethylamino)propan-2-yl)-8,9-difluoro-5,6-dimethyl-6H-pyrido[4,3-b]carbazole-1-carboxamide

To a solution of 8,9-difluoro-5,6-dimethyl-6H-pyrido[4,3-b]carbazole-1-carboxylic acid (30 mg, 91.9 umol, 1 eq) in DMF (1 mL) was added DIEA (35.6 mg, 275.8 umol, 48.0 uL, 3 eq) and HATU (52.4 mg, 137.9 umol, 1.5 eq) and stirred for 30 min. (2R)—N1,N1-dimethylpropane-1,2-diamine (32.2 mg, 183.8 umol, 2 eq, 2 HCl) was added to the mixture. The mixture was stirred at 25° C. for 12 hr. The reaction mixture was filtered, and the filtrate dried in vacuum to give crude product. The residue was purified by prep-HPLC (TFA condition, column: Phenomenex Luna C18 150*30 mm*5 um; mobile phase: [water(0.1% TFA)-ACN]; B %: 15%-45%, 9 min) to give (R)—N-(1-(dimethylamino)propan-2-yl)-8,9-difluoro-5,6-dimethyl-6H-pyrido[4,3-b]carbazole-1-carboxamide.

MS (ESI): m/z=411.2 [M+H]⁺

### N-(2-aminoethyl)-9-fluoro-5,6-dimethyl-6H-pyrido[4,3-b]carbazole-1-carboxamide (Compound 308)

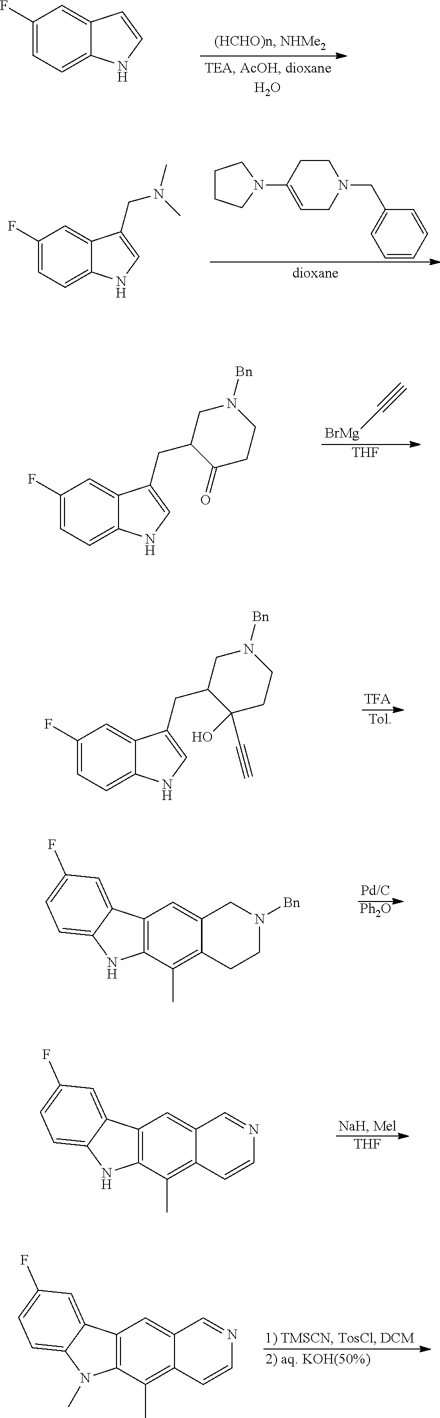

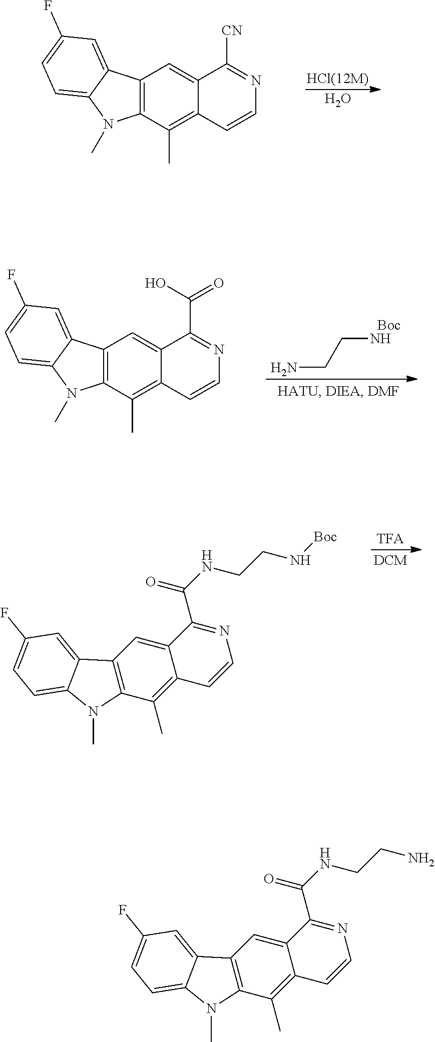

### Step 1: 1-(5-fluoro-1H-indol-3-yl)-N,N-dimethylmethanamine

To a solution of 5-fluoro-1H-indole (20 g, 148.0 mmol, 1 eq) in dioxane (283 mL) and H₂O (283 mL) was added dimethylamine hydrochloride (13.2 g, 162.8 mmol, 1.1 eq), formaldehyde (13.2 g, 162.8 mmol, 12.1 mL, 37% purity, 1.1 eq), TEA (16.4 g, 162.8 mmol, 22.6 mL, 1.1 eq) and AcOH (13.3 g, 222.0 mmol, 12.7 mL, 1.5 eq). The mixture was stirred at 30° C. for 12 hr. The mixture was concentrated to remove the dioxane and poured into NaOH (1000 mL, 2N) and white solid was separated out. The mixture was filtered and wash with H₂O (500 mL) to give 1-(5-fluoro-1H-indol-3-yl)-N,N-dimethylmethanamine.

### Step 2: 1-benzyl-3-((5-fluoro-1H-indol-3-yl)methyl)piperidin-4-one

To a solution of 1-(5-fluoro-1H-indol-3-yl)-N,N-dimethylmethanamine (15 g, 78.0 mmol, 1 eq) in dioxane (120 mL) was added 1-benzyl-4-(pyrrolidin-1-yl)-1,2,3,6-tetrahydropyridine (47.2 g, 195.0 mmol, 2.5 eq). The mixture was stirred at 120° C. for 12 hr. The reaction mixture was concentrated under reduced pressure to give a residue. The residue was purified by flash silica gel chromatography (ISCO®; 330 g SepaFlash® Silica Flash Column, Eluent of 0˜100% Ethyl acetate/Petroleum ether gradient @ 100 mL/min) to give 1-benzyl-3-((5-fluoro-1H-indol-3-yl)methyl)piperidin-4-one.

### Step 3: 1-benzyl-4-ethynyl-3-((5-fluoro-1H-indol-3-yl)methyl)piperidin-4-ol

To a solution of 1-benzyl-3-((5-fluoro-1H-indol-3-yl)methyl)piperidin-4-one (2 g, 5.9 mmol, 1 eq) in THF (15 mL) was added bromo(ethynyl)magnesium (0.5 M, 47.5 mL, 4 eq) at 0° C. The mixture was stirred at 0° C. for 1 hr. The reaction mixture was quenched by addition NH₄Cl (50 mL) at 25° C., and extracted with EtOAc (50 mL×3). The combined organic layers were washed with brine (50 mL×3), dried over Na₂SO₄, filtered and concentrated under reduced pressure to give 1-benzyl-4-ethynyl-3-((5-fluoro-1H-indol-3-yl)methyl)piperidin-4-ol.

### Step 4: 2-benzyl-9-fluoro-5-methyl-2,3,4,6-tetrahydro-1H-pyrido[4,3-b]carbazole

To a solution of 1-benzyl-4-ethynyl-3-((5-fluoro-1H-indol-3-yl)methyl)piperidin-4-ol (2 g, 5.5 mmol, 1 eq) in Tol. (40 mL) was added TFA (6.2 g, 55.1 mmol, 4.0 mL, 10 eq). The mixture was stirred at 80° C. for 12 hr. The reaction mixture was partitioned between NaHCO₃ 100 mL and EtOAc 100 mL. The water phase was separated, extracted with EtOAc (100 mL×3). The combined organic phase was washed with brine (100 mL×3), dried over Na₂SO₄, filtered and concentrated under reduced pressure to give a residue. The residue was purified by flash silica gel chromatography (ISCO®; 12 g SepaFlash® Silica Flash Column, Eluent of 0˜20% Ethyl acetate/Petroleum ether gradient @ 80 mL/min) to give 2-benzyl-9-fluoro-5-methyl-2,3,4,6-tetrahydro-1H-pyrido[4,3-b]carbazole.

### Step 5: 9-fluoro-5-methyl-6H-pyrido[4,3-b]carbazole

To a solution of 2-benzyl-9-fluoro-5-methyl-2,3,4,6-tetrahydro-1H-pyrido[4,3-b]carbazole (1 g, 2.9 mmol, 1 eq) in Ph₂O (10 mL) was added Pd/C (1 g, 10% purity). The mixture was stirred at 220° C. for 4 hr. The reaction mixture was filtered and washed with the solvent (DCM:MeOH=10:1) (100 mL×3) and the filtrate was dried in vacuum to give crude product. The residue was purified by flash silica gel chromatography (ISCO®; 12 g SepaFlash® Silica Flash Column, Eluent of 0˜10% MeOH/DCM @ 80 mL/min) to give 9-fluoro-5-methyl-6H-pyrido[4,3-b]carbazole.

### Step 6: 9-fluoro-5,6-dimethyl-6H-pyrido[4,3-b]carbazole

To a solution of 9-fluoro-5-methyl-6H-pyrido[4,3-b]carbazole (320 mg, 1.2 mmol, 1 eq) in THF (10 mL) was added NaH (153.4 mg, 3.8 mmol, 60% purity, 3 eq) at 0° C. and stirred for 30 min. Then Mel (181.4 mg, 1.2 mmol, 79.6 uL, 1 eq) was added to mixture and stirred at 25° C. for 2 hr. TLC indicated one new spot formed. The residue was diluted with NH₄Cl 50 mL and extracted with EtOAc (20 mL×3). The combined organic layers were washed with brine (10 mL×2), dried over Na₂SO₄, filtered, and concentrated under reduced pressure to give 9-fluoro-5,6-dimethyl-6H-pyrido[4,3-b]carbazole as a yellow solid.

### Step 7: 9-fluoro-5,6-dimethyl-6H-pyrido[4,3-b]carbazole-1-carbonitrile

To a mixture of 9-fluoro-5,6-dimethyl-6H-pyrido[4,3-b]carbazole (430 mg, 1.6 mmol, 1 eq) in DCM (10 mL) was added trimethylsilylcyanide (419.6 mg, 4.2 mmol, 529.2 uL, 2.6 eq). To this mixture a solution of TosCl (589.3 mg, 3.0 mmol, 1.9 eq) in DCM (2 mL) was added at 25° C. Then the mixture was stirred at 30° C. for 12 hr. LCMS showed the intermediate was detected and 10 mL KOH (50% W/W) was added to the reaction. The mixture was stirred at 30° C. for 2 hr. The reaction mixture was diluted with H₂O 20 mL and extracted with DCM (10 mL×3). The combined organic layers were washed with brine (10 mL×2), dried over Na₂SO₄, filtered and the filtrate was concentrated under reduced pressure to give 9-fluoro-5,6-dimethyl-6H-pyrido[4,3-b]carbazole-1-carbonitrile.

### Step 8: 9-fluoro-5,6-dimethyl-6H-pyrido[4,3-b]carbazole-1-carboxylic acid

The solution of 9-fluoro-5,6-dimethyl-6H-pyrido[4,3-b]carbazole-1-carbonitrile (140 mg, 483.9 umol, 1 eq) in HCl (5 mL) (12 M) was stirred at 130° C. for 4 hr. The reaction mixture was diluted with NaHCO₃ 15 mL, filtered and concentrated under reduced pressure to give 9-fluoro-5,6-dimethyl-6H-pyrido[4,3-b]carbazole-1-carboxylic acid.

### Step 9: tert-butyl (2-(9-fluoro-5,6-dimethyl-6H-pyrido[4,3-b]carbazole-1-carboxamido)ethyl)carbamate

To a solution of 9-fluoro-5,6-dimethyl-6H-pyrido[4,3-b]carbazole-1-carboxylic acid (60 mg, 194.6 umol, 1 eq) in DMF (2 mL) was added DIEA (75.4 mg, 583.8 umol, 101.6 uL, 3 eq) and HATU (111.0 mg, 291.9 umol, 1.5 eq) at 25° C., then the reaction was stirred at 25° C. for 30 min. tert-butyl N-(2-aminoethyl)carbamate (62.3 mg, 389.2 umol, 61.1 uL, 2 eq) was added to the mixture and stirred at 25° C. for 12 hr. The reaction mixture was diluted with H₂O 5 mL and the product was precipitated out, the filtrate cake was concentrated under reduced pressure to give tert-butyl (2-(9-fluoro-5,6-dimethyl-6H-pyrido[4,3-b]carbazole-1-carboxamido)ethyl)carbamate.

### Step 10: N-(2-aminoethyl)-9-fluoro-5,6-dimethyl-6H-pyrido[4,3-b]carbazole-1-carboxamide

To a solution of tert-butyl (2-(9-fluoro-5,6-dimethyl-6H-pyrido[4,3-b]carbazole-1-carboxamido)ethyl)carbamate (60 mg, 133.1 umol, 1 eq) in DCM (2 mL) was added TFA (1.5 g, 13.5 mmol, 1 mL, 101.4 eq). The mixture was stirred at 25° C. for 1 hr. The reaction mixture was concentrated under reduced pressure to remove solvent. The residue was purified by prep-HPLC (TFA condition, column: Phenomenex Luna C18 150*30 mm*5 um; mobile phase: [water(0.1% TFA)-ACN]; B %: 20%-50%, 8 min) to give N-(2-aminoethyl)-9-fluoro-5,6-dimethyl-6H-pyrido[4,3-b]carbazole-1-carboxamide.

MS (ESI): m/z=351.1 [M+H]⁺

### N-(2-aminoethyl)-8,9-difluoro-5,6-dimethyl-6H-pyrido[4,3-b]carbazole-1-carboxamide (Compound 309)

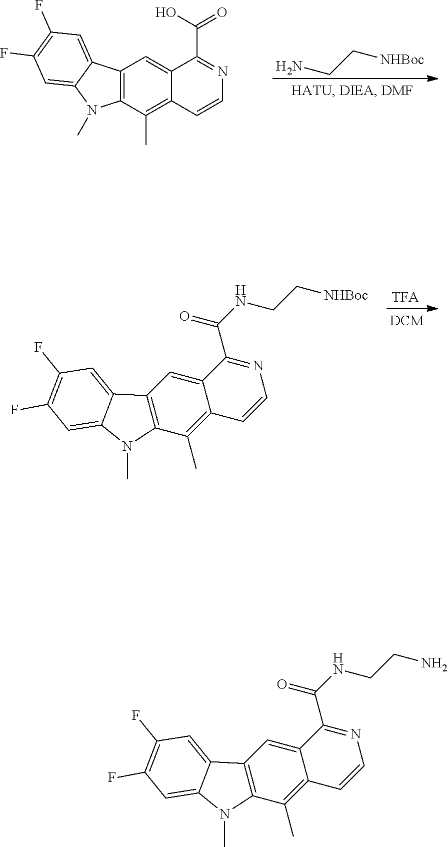 [SMILES: Cc1c2ccnc(C(=O)O)c2cc2c3cc(F)c(F)cc3n(C)c12 ; CC(C)(C)OC(=O)NCCN ; Cc1c2ccnc(C(=O)NCCNC(=O)OC(C)(C)C)c2cc2c3cc(F)c(F)cc3n(C)c12 ; Cc1c2ccnc(C(=O)NCCN)c2cc2c3cc(F)c(F)cc3n(C)c12]

### Step 1: tert-butyl (2-(8,9-difluoro-5,6-dimethyl-6H-pyrido[4,3-b]carbazole-1-carboxamido)ethyl)carbamate

To a solution of 8,9-difluoro-5,6-dimethyl-pyrido[4,3-b]carbazole-1-carboxylic acid (100 mg, 306.4 umol, 1 eq) in DMF (3 mL) was added DIEA (118.8 mg, 919.4 umol, 160.1 uL, 3 eq), HATU (174.7 mg, 459.7 umol, 1.5 eq) and stirred for 30 min. tert-butyl N-(2-aminoethyl)carbamate (98.2 mg, 612.9 umol, 96.2 uL, 2 eq) was added to the mixture. The mixture was stirred at 25° C. for 12 hr. The reaction mixture was added to H₂O (3 mL) and filtered, and the filter cake was washed with 2 mL of H₂O, dried in vacuum to give tert-butyl (2-(8,9-difluoro-5,6-dimethyl-6H-pyrido[4,3-b]carbazole-1-carboxamido)ethyl)carbamate.

### Step 2: N-(2-aminoethyl)-8,9-difluoro-5,6-dimethyl-6H-pyrido[4,3-b]carbazole-1-carboxamide

A mixture of tert-butyl (2-(8,9-difluoro-5,6-dimethyl-6H-pyrido[4,3-b]carbazole-1-carboxamido)ethyl)carbamate (90 mg, 192.1 umol, 1 eq) in TFA (1 mL) and DCM (2 mL) was stirred at 25° C. for 1 hr. The reaction mixture was concentrated under reduced pressure to give a residue. The residue was purified by prep-HPLC (TFA condition, column: Phenomenex Luna C18 150*30 mm*5 um; mobile phase: [water(0.1% TFA)-ACN]; B %: 20%-50%, 8 min) to give N-(2-aminoethyl)-8,9-difluoro-5,6-dimethyl-6H-pyrido[4,3-b]carbazole-1-carboxamide.

MS (ESI): m/z=369.1 [M+H]⁺

### (R)—N-(1-(dimethylamino)propan-2-yl)-9-fluoro-5,6-dimethyl-6H-pyrido[4,3-b]carbazole-1-carboxamide (Compound 310)

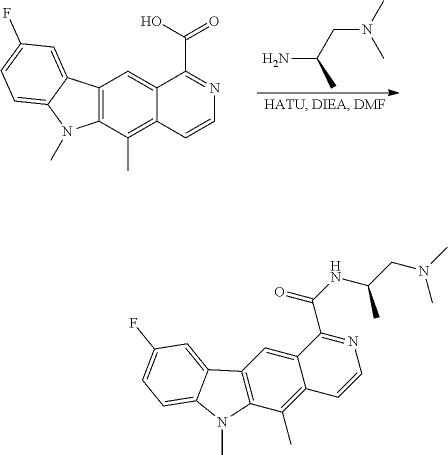

To a solution of 9-fluoro-5,6-dimethyl-6H-pyrido[4,3-b]carbazole-1-carboxylic acid (50 mg, 162.1 umol, 1 eq) in DMF (2 mL) was added DIEA (62.8 mg, 486.5 umol, 84.7 uL, 3 eq) and HATU (92.5 mg, 243.2 umol, 1.5 eq) at 25° C., then the reaction was stirred at 25° C. for 30 min. (2R)—N1,N1-dimethylpropane-1,2-diamine (56.7 mg, 324.3 umol, 2 eq, 2 HCl) was added to the mixture and stirred at 25° C. for 12 hr. (2R)—N1,N1-dimethylpropane-1,2-diamine (14.2 mg, 81.0 umol, 0.5 eq, 2 HCl) was added to the reaction and stirred at 25° C. for 2 hr. The reaction mixture was concentrated under reduced pressure to remove solvent. The residue was purified by prep-HPLC (TFA condition, column: Phenomenex Luna C18 100*30 mm*5 um; mobile phase: [water(0.1% TFA)-MeOH]; B %: 30%-60%, 10 min) to give (R)—N-(1-(dimethylamino)propan-2-yl)-9-fluoro-5,6-dimethyl-6H-pyrido[4,3-b]carbazole-1-carboxamide.

MS (ESI): m/z=393.2 [M+H]⁺

### 5,6-dimethyl-9-(pyridin-3-yloxy)-6H-pyrido[4,3-b]carbazole (Compound 311)

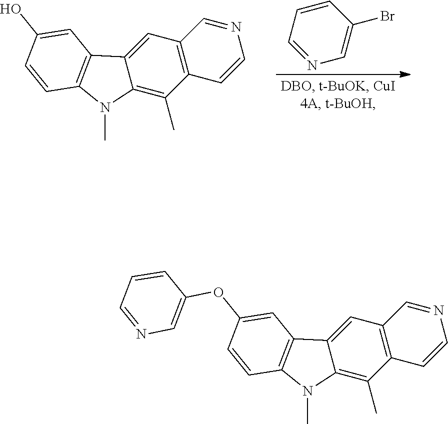 [SMILES: Cc1c2ccncc2cc2c3cc(O)ccc3n(C)c12 ; Brc1cccnc1 ; Cc1c2ccncc2cc2c3cc(Oc4cccnc4)ccc3n(C)c12]

To a solution of 5,6-dimethylpyrido[4,3-b]carbazol-9-ol (199.2 mg, 759.5 umol, 1.2 eq) in t-BuOH (2 mL) was added 3-bromopyridine (100 mg, 632.9 umol, 60.9 uL, 1 eq), CuI (1.2 mg, 6.3 umol, 0.01 eq), N,N-dibenzyloxamide (3.4 mg, 12.7 umol, 0.02 eq), t-BuOK (142.0 mg, 1.2 mmol, 2 eq) and 4 A molecular sieve (300 mg) under N₂. The reaction mixture was stirred at 100° C. for 12 hr. The reaction mixture was concentrated under reduced pressure to remove solvent and diluted with dimethyl sulfoxide (2 mL). The residue was purified by prep-HPLC (TFA condition; column: Waters Xbridge BEH C18 100*25 mm*5 um; mobile phase: [water(10 mM NH₄HCO₃)-ACN]; B %: 35%-65%, 10 min) to give 5,6-dimethyl-9-(pyridin-3-yloxy)-6H-pyrido[4,3-b]carbazole.

MS (ESI): m/z=340.1 [M+H]⁺

### N,N-dimethyl-2-(5-methyl-6H-pyrido[4,3-b]carbazol-6-yl)ethanamine (Compound 312)

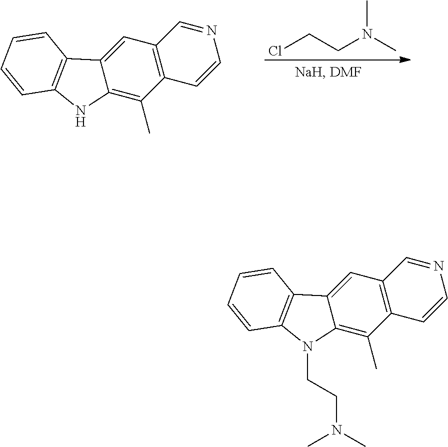

To a solution of 5-methyl-6H-pyrido[4,3-b]carbazole (80 mg, 344.4 umol, 1 eq) in DMF (2 mL) was added NaH (41.3 mg, 1.0 mmol, 60% purity, 3 eq) at 0° C. and stirred for 30 min. 2-chloro-N,N-dimethyl-ethanamine (59.5 mg, 413.3 umol, 1.2 eq, HCl) was added to the mixture and stirred at 25° C. for 12 hr. The reaction was stirred at 70° C. for 12 hr. The reaction mixture was diluted with FA 1 mL, and then filtered and the filtrate was concentrated under reduced pressure to give a residue. The residue was purified by prep-HPLC (TFA condition, column: Phenomenex Luna C18 150*30 mm*5 um; mobile phase: [water(0.1% TFA)-ACN]; B %: 1%-25%, 8 min) to give N,N-dimethyl-2-(5-methyl-6H-pyrido[4,3-b]carbazol-6-yl)ethanamine.

MS (ESI): m/z=304.1 [M+H]⁺

### N-(2-(dimethylamino)ethyl)-8,9-difluoro-5,6-dimethyl-6H-pyrido[4,3-b]carbazole-1-carboxamide (Compound 313)

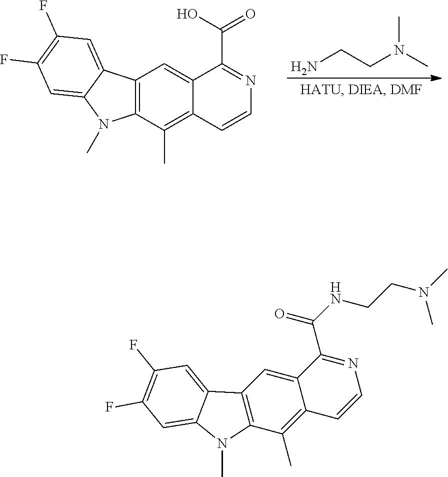

To a solution of 8,9-difluoro-5,6-dimethyl-6H-pyrido[4,3-b]carbazole-1-carboxylic acid (80 mg, 245.1 umol, 1 eq) in DMF (2 mL) was added DIEA (95.0 mg, 735.5 umol, 128.1 uL, 3 eq) and HATU (139.8 mg, 367.7 umol, 1.5 eq) at 25° C., then the reaction was stirred at 25° C. for 30 min. N,N-dimethylethane-1,2-diamine (43.2 mg, 490.3 umol, 53.5 uL, 2 eq) was added to the mixture and stirred at 25° C. for 12 hr. The reaction mixture was concentrated under reduced pressure to remove solvent. The residue was purified by prep-HPLC (TFA condition, column: Phenomenex luna C18 100*40 mm*5 um; mobile phase: [water(0.1% TFA)-ACN]; B %: 10%-45%, 8 min) to give N-(2-(dimethylamino)ethyl)-8,9-difluoro-5,6-dimethyl-6H-pyrido[4,3-b]carbazole-1-carboxamide.

MS (ESI): m/z=397.1 [M+H]⁺

### 5-((5,6-dimethyl-6H-pyrido[4,3-b]carbazol-9-yl)oxy)pyridine-2-amine (Compound 314)

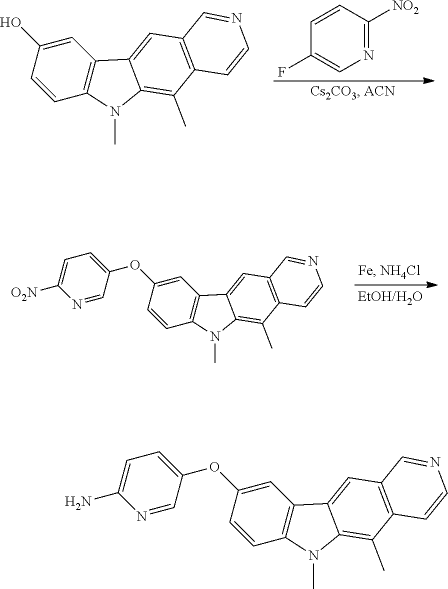

### Step 1: 5,6-dimethyl-9-((6-nitropyridin-3-yl)oxy)-6H-pyrido[4,3-b]carbazole

To a solution of 5,6-dimethylpyrido[4,3-b]carbazol-9-ol (221.5 mg, 844.5 umol, 1.2 eq) in ACN (2 mL) was added 5-fluoro-2-nitro-pyridine (100 mg, 703.7 umol, 12.2 uL, 1 eq) and Cs₂CO₃ (458.6 mg, 1.4 mmol, 2. eq) under N₂. The reaction mixture was stirred at 80° C. for 12 hr. The reaction mixture was concentrated filtered. The filter cake was washed with H₂O (50 mL) and concentrated under reduced pressure to give 5,6-dimethyl-9-((6-nitropyridin-3-yl)oxy)-6H-pyrido[4,3-b]carbazole.

### Step 2: 5-((5,6-dimethyl-6H-pyrido[4,3-b]carbazol-9-yl)oxy)pyridin-2-amine

To a solution of 5,6-dimethyl-9-[(6-nitro-3-pyridyl)oxy]pyrido[4,3-b]carbazole (200 mg, 520.3 umol, 1 eq) in EtOH (4 mL)/H₂O (1 mL) was added Fe (145.2 mg, 2.6 mmol, 5 eq) and NH₄Cl (139.1 mg, 2.6 mmol, 5 eq). The reaction mixture was stirred at 80° C. for 12 hr. The reaction mixture was concentrated under reduced pressure to remove solvent. The residue was diluted with DMF (2 mL) and DMSO (2 mL). The residue was purified by prep-HPLC (TFA condition; column: Phenomenex luna C18 100*40 mm*5 um; mobile phase: [water(0.1% TFA)-ACN]; B %: 3%-38%, 8 min) to give 5-((5,6-dimethyl-6H-pyrido[4,3-b]carbazol-9-yl)oxy)pyridine-2-amine.

MS (ESI): m/z=355.1 [M+H]⁺

### N-(2-(dimethylamino)ethyl)-9-fluoro-5,6-dimethyl-6H-pyrido[4,3-b]carbazole-1-carboxamide (Compound 315)

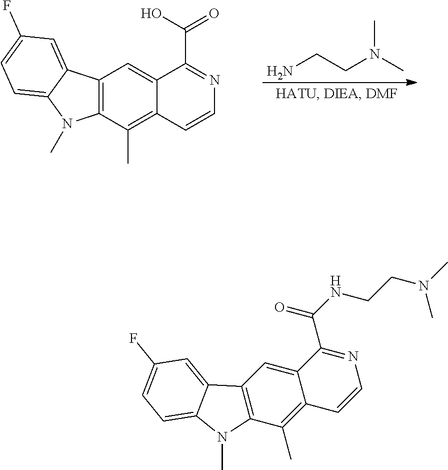

To a solution of 9-fluoro-5,6-dimethyl-6H-pyrido[4,3-b]carbazole-1-carboxylic acid (80 mg, 259.4 umol, 1 eq) in DMF (2 mL) was added DIEA (100.6 mg, 778.4 umol, 135.5 uL, 3 eq) and HATU (147.9 mg, 389.2 umol, 1.5 eq) at 25° C., then the reaction was stirred at 25° C. for 30 min. N,N-dimethylethane-1,2-diamine (45.7 mg, 518.9 umol, 56.6 uL, 2 eq) was added to the mixture and stirred at 25° C. for 12 hr. The reaction mixture was concentrated under reduced pressure to remove solvent. The residue was purified by prep-HPLC (TFA condition, column: Phenomenex luna C18 100*40 mm*5 um; mobile phase: [water(0.1% TFA)-ACN]; B %: 5%-50%, 8 min) to give N-(2-(dimethylamino)ethyl)-9-fluoro-5,6-dimethyl-6H-pyrido[4,3-b]carbazole-1-carboxamide.

MS (ESI): m/z=379.1 [M+H]⁺

### 9-methoxy-5-methyl-6-(2-(piperidin-1-yl)ethyl)-6H-pyrido[4,3-b]carbazole (Compound 316)

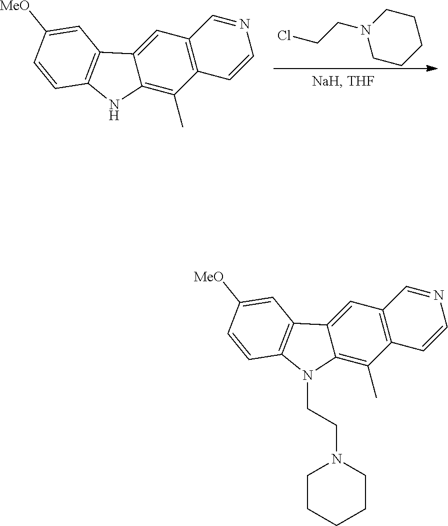

To a solution of 9-methoxy-5-methyl-6H-pyrido[4,3-b]carbazole (100 mg, 381.2 umol, 1 eq) in DMF (2 mL) was added NaH (45.7 mg, 1.1 mmol, 60% purity, 3 eq) at 0° C. The reaction mixture was stirred at 0° C. for 30 min. 1-(2-chloroethyl)piperidine (112.6 mg, 611.5 umol, 1.6 eq, HCl) was added to the mixture. The mixture was stirred at 25° C. for 12 hr. The pH of reaction mixture was adjusted to 7 by addition of formic acid. The reaction mixture was filtered. The residue was purified by prep-HPLC (TFA condition; column: Phenomenex Luna C18 150*30 mm*5 um; mobile phase: [water(0.1% TFA)-ACN]; B %: 10%-40%, 8 min) to give 9-methoxy-5-methyl-6-(2-(piperidin-1-yl)ethyl)-6H-pyrido[4,3-b]carbazole.

MS (ESI): m/z=374.1 [M+H]⁺

### 6-((4,5-dihydro-1H-imidazol-2-yl)methyl)-5-methyl-6H-pyrido[4,3-b]carbazole (Compound 317)

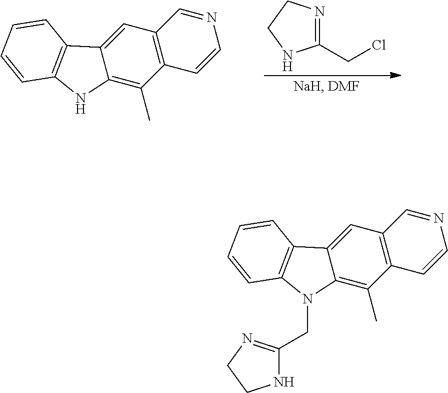 [SMILES: Cc1c2ccncc2cc2c1[nH]c1ccccc12 ; ClCC1=NCCN1 ; Cc1c2ccncc2cc2c3ccccc3n(CC3=NCCN3)c12]

To a solution of 5-methyl-6H-pyrido[4,3-b]carbazole (80 mg, 344.4 umol, 1 eq) in DMF (2 mL) was added NaH (68.8 mg, 1.7 mmol, 60% purity, 5 eq) and 2-(chloromethyl)-4,5-dihydro-1H-imidazole (106.7 mg, 688.8 umol, 2 eq, HCl). The mixture was stirred at 70° C. for 2 hr. The reaction mixture was concentrated under reduced pressure to give a residue. The residue was purified by prep-HPLC (TFA condition, column: Phenomenex Luna 80*30 mm*3 um; mobile phase: [water(0.1% TFA)-ACN]; B %: 5%-35%, 8 min) to give 6-((4,5-dihydro-1H-imidazol-2-yl)methyl)-5-methyl-6H-pyrido[4,3-b]carbazole.

MS (ESI): m/z=315.1 [M+H]⁺

### 5-methyl-6-(2-(piperidin-1-yl)ethyl)-6H-pyrido[4,3-b]carbazole (Compound 318)

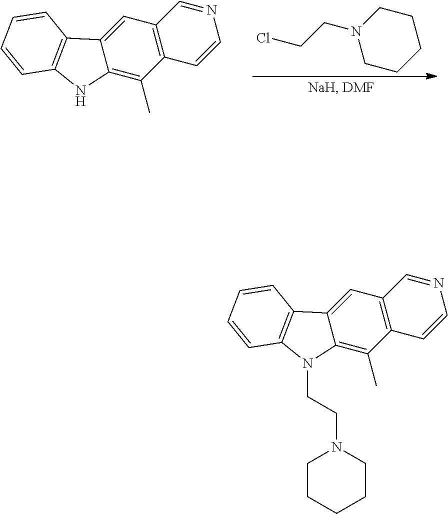 [SMILES: Cc1c2ccncc2cc2c1[nH]c1ccccc12 ; ClCCN1CCCCC1 ; Cc1c2ccncc2cc2c3ccccc3n(CCN3CCCCC3)c12]

To a solution of 5-methyl-6H-pyrido[4,3-b]carbazole (50 mg, 215.2 umol, 1 eq) in DMF (2 mL) was added NaH (25.8 mg, 645.7 umol, 60% purity, 3 eq). The mixture was stirred at 25° C. for 10 min. 1-(2-chloroethyl)piperidine (63.5 mg, 430.5 umol, 2 eq) was added to the mixture. The mixture was stirred at 25° C. for 2 hr. The reaction mixture was partitioned between NH₄Cl 5 mL and EtOAc 5 mL. The water phase was separated, extracted with EtOAc (5 mL×3). The combined organic phase was washed with brine (5 mL×3), dried over Na₂SO₄, filtered and concentrated under reduced pressure to give a residue. The residue was purified by prep-HPLC (FA condition; column: Phenomenex Luna C18 150*30 mm*5 um; mobile phase: [water(0.2% FA)-ACN]; B %: 2%-30%, 8 min) to give 5-methyl-6-(2-(piperidin-1-yl)ethyl)-6H-pyrido[4,3-b]carbazole.

MS (ESI): m/z=344.1 [M+H]⁺

### 4-(2-(5-methyl-6H-pyrido[4,3-b]carbazol-6-yl)ethyl)morpholine (Compound 319)

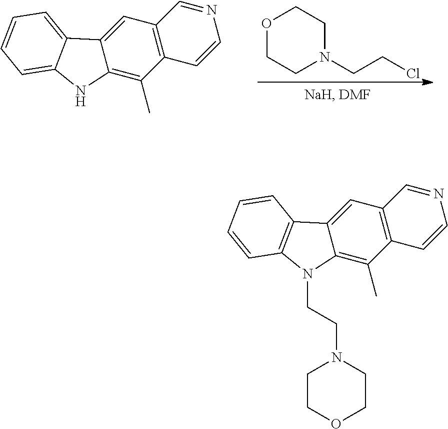 [SMILES: Cc1c2ccncc2cc2c1[nH]c1ccccc12 ; ClCCN1CCOCC1 ; Cc1c2ccncc2cc2c3ccccc3n(CCN3CCOCC3)c12]

To a solution of 5-methyl-6H-pyrido[4,3-b]carbazole (80 mg, 344.4 umol, 1 eq) in DMF (2 mL) was added NaH (41.3 mg, 1.0 mmol, 60% purity, 3 eq) at 0° C. and stirred for 30 min. 4-(2-chloroethyl)morpholine (128.1 mg, 688.8 umol, 2 eq, HCl) was added to the mixture. The mixture was stirred at 25° C. for 12 hr. The mixture was stirred at 70° C. for 12 hr. The reaction mixture was concentrated under reduced pressure to give a residue. The residue was purified by prep-HPLC (TFA condition, column: Phenomenex luna C18 100*40 mm*5 um; mobile phase: [water(0.1% TFA)-ACN]; B %: 1%-20%, 8 min) to give 4-(2-(5-methyl-6H-pyrido[4,3-b] carbazol-6-yl)ethyl)morpholine.

MS (ESI): m/z=346.1[M+H]⁺

### 4-(2-(9-methoxy-5-methyl-6H-pyrido[4,3-b]carbazol-6-yl)ethyl)morpholine (Compound 320)

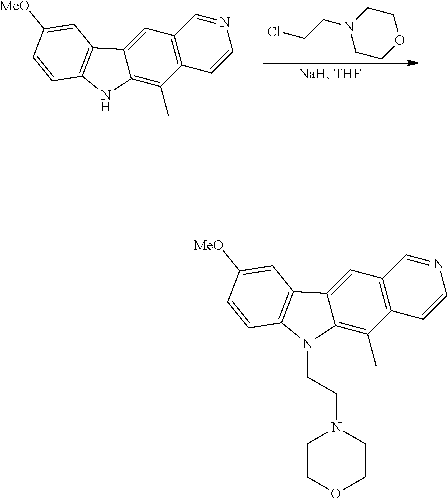 [SMILES: COc1ccc2[nH]c3c(C)c4ccncc4cc3c2c1 ; ClCCN1CCOCC1 ; COc1ccc2c(c1)c1cc3cnccc3c(C)c1n2CCN1CCOCC1]

To a solution of 9-methoxy-5-methyl-6H-pyrido[4,3-b]carbazole (100 mg, 381.2 umol, 1 eq) in DMF (2 mL) was added NaH (45.7 mg, 1.1 mmol, 60% purity, 3 eq) at 0° C. The reaction mixture was stirred at 0° C. for 30 min. 4-(2-chloroethyl)morpholine (113.8 mg, 611.5 umol, 1.6 eq, HCl) was Added to the mixture. The reaction mixture was stirred at 25° C. for 12 hr. The pH of reaction mixture was adjusted to 7 by addition formic acid. The reaction mixture was filtered. The residue was purified by prep-HPLC (TFA condition; column: Phenomenex Luna C18 150*30 mm*5 um; mobile phase: [water(0.1% TFA)-ACN]; B %: 10%-40%, 8 min) to give 4-(2-(9-methoxy-5-methyl-6H-pyrido[4,3-b]carbazol-6-yl)ethyl)morpholine.

MS (ESI): m/z=376.1 [M+H]⁺

### 2-((5,6-dimethyl-6H-pyrido[4,3-b]carbazol-9-yl)oxy)-N-methylethanamine (Compound 321)

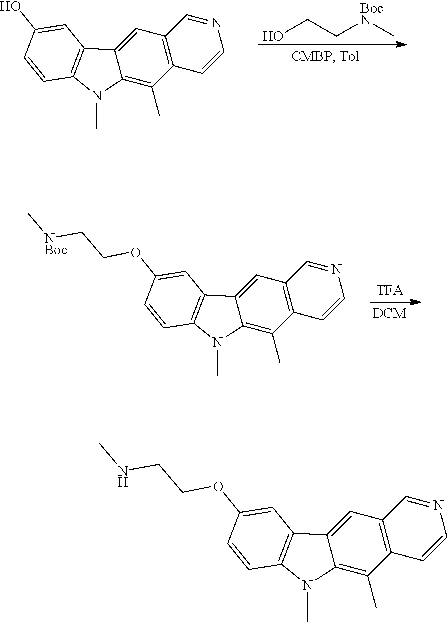 [SMILES: Cc1c2ccncc2cc2c3cc(O)ccc3n(C)c12 ; CN(CCO)C(=O)OC(C)(C)C ; Cc1c2ccncc2cc2c3cc(OCCN(C)C(=O)OC(C)(C)C)ccc3n(C)c12 ; CNCCOc1ccc2c(c1)c1cc3cnccc3c(C)c1n2C]

### Step 1: tert-butyl (2-((5,6-dimethyl-6H-pyrido[4,3-b]carbazol-9-yl)oxy)ethyl)(methyl)carbamate

To a solution of 5,6-dimethyl-6H-pyrido[4,3-b]carbazol-9-ol (100 mg, 381.2 umol, 1 eq) in toluene (3 mL) was added tert-butyl N-(2-hydroxyethyl)-N-methyl-carbamate (66.8 mg, 381.24 umol, 1 eq) and 2-(tributyl-λ⁵-phosphanylidene)acetonitrile (9.2 mg, 38.1 umol, 0.1 eq). The reaction mixture was stirred at 100° C. for 12 hr. The reaction mixture was concentrated under reduced pressure to remove solvent. The residue was diluted with (DCM 2 mL). The residue was purified by prep-TLC (SiO₂, DCM:MeOH=10:1) to give tert-butyl (2-((5,6-dimethyl-6H-pyrido[4,3-b]carbazol-9-yl)oxy)ethyl)(methyl)carbamate.

### Step 2; 2-((5,6-dimethyl-6H-pyrido[4,3-b]carbazol-9-yl)oxy)-N-methylethanamine

To a solution of tert-butyl (2-((5,6-dimethyl-6H-pyrido[4,3-b]carbazol-9-yl)oxy)ethyl)(methyl)carbamate (100 mg, 238.4 umol, 1 eq) in DCM (2 mL) was added TFA (1.5 g, 13.5 mmol, 1 mL, 56.7 eq). The reaction mixture was stirred at 25° C. for 12 hr. The reaction mixture was diluted with methanol (2 mL) and filtered. The residue was purified by prep-HPLC (TFA condition; column: Phenomenex luna C18 100*40 mm*5 um; mobile phase: [water(0.1% TFA)-ACN]; B %: 1%-35%, 8 min) to give 2-((5,6-dimethyl-6H-pyrido[4,3-b]carbazol-9-yl)oxy)-N-methylethanamine.

MS (ESI): m/z=320.1 [M+H]⁺

Other compounds disclosed herein can be synthesized similarly as described in the above examples.

Inhibition assay of compounds. The IC₅₀ values for inhibition of RNA translation for Compounds 1-84, 202-257, and 262-321 were measured. Data are presented in Tables D, E, F, and G below. These experiments were completed by co-transfecting HEK293T cells with (G₄C₂)₆₆-No A TG-GFP (SEQ ID NO: 11) and a plasmid encoding containing mCherry (ATG-mCherry). The transfected cells were then treated with varying amounts of compound and the percent inhibition of RAN translation was comparing the GFP signal in treated cells to cells treated with vehicle. Percent inhibition was plotted as a function of compound concentration and the resulting curve was fit to a four-parameter logistic curve fit (Sigma Plot).

The BLI K_{d} data in Table G below was obtained using the below procedure. Biotin-labeled RNA was incubated with test compounds in a streptavidin-labeled biosensor from ForteBio. Affinity of the test compounds for the target RNA was measured in a ForteBio Octet RED384 BLI system.

TABLE D

| | TR-FRET Kᵢ | RANT IC₂₅ |
|---|---|---|
| ID No. | (μM) | (μM) |
| 1 | >200 | >5 |
| 2 | >200 | — |
| 3 | 8.3 | 0.1 |
| 4a | 4.5 | 0.9 |
| 4b | >200 | — |
| 5 | 20 | 0.5 |
| 6 | 32 | >5 |
| 7 | 12 | >5 |
| 8 | 11 | 0.36 |
| 9 | 10 | 0.27 |
| 10 | 7.3 | 3.2 |
| 11 | 16 | 0.4 |
| 12 | 18 | 0.3 |
| 13 | >200 | — |
| 14 | >200 | — |
| 15 | >200 | — |
| 16 | >200 | — |
| 17 | >200 | — |
| 18 | >200 | — |
| 19 | >200 | — |
| 20 | >200 | — |
| 21 | >200 | — |
| 22 | >200 | — |
| 23 | 9.4 | >5 |
| 24 | >200 | — |
| 25 | 39 | 0.27 |
| 26 | 5.9 | 0.57 |
| 27 | 9.1 | 1.0 |
| 28 | 8.1 | 0.64 |
| 29 | 17 | 0.74 |
| 30 | 2.5 | 2.2 |
| 31 | 4.5 | 2.8 |
| 32 | 9.5 | 1.4 |
| 33 | 5.2 | 3.7 |
| 34 | 6.1 | 1.2 |
| 35 | 38 | >5 |
| 36 | 3.9 | >5 |
| 37 | 0.84 | 2.3 |
| 38 | 0.76 | 0.74 |
| 39 | 0.80 | 1.3 |
| 40 | >200 | — |
| 41 | 0.6 | 0.67 |
| 42 | 9.2 | >5 |
| 43 | 27 | — |
| 44a | 10 | — |
| 44b | >200 | — |
| 45 | 21 | — |
| 46 | 4.6 | — |
| 47 | >200 | — |
| 48 | >200 | — |
| 49 | >200 | — |
| 50 | 33 | 0.27 |
| 51 | 6.0 | >5 |
| 52 | 5.6 | 3.1 |
| 53 | >200 | — |
| 54 | 13 | 1.3 |
| 55 | 1.5 | 0.92 |
| 56 | 1.0 | 0.67 |
| 57 | 5.2 | 4.0 |
| 58 | 1.0 | — |
| 59 | 1.3 | — |
| 60 | 3.8 | >5 |
| 61 | 0.90 | 1.0 |
| 62 | >200 | >5 |
| 63 | 1.8 | 4.8 |
| 64 | >200 | >5 |
| 65 | 4.2 | — |
| 66 | >200 | — |
| 67 | >200 | — |
| 68 | 4.0 | 0.72 |
| 69 | 12 | 1.6 |
| 70 | 36 | 3.6 |
| 71 | 107 | >5 |
| 72 | 15 | >5 |
| 73 | 13 | 0.84 |
| 74 | 2.5 | >5 |
| 75 | 29 | 1.4 |
| 76 | 4.2 | 2.2 |
| 77 | 1.2 | 1.7 |
| 78 | 3.6 | 1.6 |
| 79 | 12 | 1.1 |
| 80a | 30 | >5 |
| 80b | 9.2 | 3.1 |
| 81 | >200 | — |
| 82 | 20 | >5 |
| 83 | 2.3 | 1.1 |
| 84 | 13 | >5 |

TABLE E

| ID No. | IC₅₀ (μM) |
|---|---|
| 202 | 12 |
| 203 | >12.5 |
| 204 | 12.5 |
| 205 | 12.5 |
| 206 | >12.5 |
| 207 | 6 |
| 208 | 2.2 |
| 209 | 3.1 |
| 210 | 10.3 |
| 211 | 3.1 |
| 212 | 3.1 |

TABLE F

| | TR-FRET Kᵢ | RANT IC₂₅ |
|---|---|---|
| ID No. | (μM) | (μM) |
| 213 | 1.2 | 1.7 |
| 214 | 3.6 | 1.0 |
| 215 | 12 | >5 |
| 216 | 6.9 | 1.7 |
| 217 | 8.5 | >5 |
| 218 | 0.80 | 0.84 |
| 219 | 1.2 | >5 |
| 220 | 2.3 | >5 |
| 221 | 0.7 | 0.66 |
| 222 | 6.1 | >5 |
| 223 | 3.1 | 3.8 |
| 224 | 8.6 | >5 |
| 225 | >200 | — |
| 226 | 13 | — |
| 227 | 1.4 | >5 |
| 228 | 1.4 | 1.3 |
| 229 | 4.2 | 2.2 |
| 230 | 1.2 | 4.3 |
| 231 | >200 | >5 |
| 232 | 2.0 | >5 |
| 233 | 7.5 | — |
| 234 | 0.8 | 0.65 |
| 235 | >200 | >5 |
| 236 | 14 | >5 |
| 237 | 3.6 | 2.0 |
| 238 | 20 | — |
| 239 | 24 | — |
| 240 | 20 | — |
| 241 | 1.8 | — |
| 242 | 0.50 | 1.5 |
| 243 | 0.60 | 1.2 |
| 244 | 0.40 | 2.4 |
| 245 | 0.80 | 1.7 |
| 246 | 1.6 | >5 |
| 247 | >200 | >5 |
| 248 | >200 | >5 |
| 249 | 22 | >5 |
| 250 | 11 | >5 |
| 251 | 19 | — |
| 252 | >200 | >5 |
| 253 | 14 | >5 |
| 254 | 1.8 | >5 |
| 255 | 8.8 | 1.25 |
| 256 | 42 | >5 |
| 257 | 5.7 | 2.7 |

TABLE G

| | BLI K_{d} | RANT IC₅₀ |
|---|---|---|
| ID No | (μM) | (μM) |
| 262 | 5.2 | 6.6 |
| 263 | 0.61 | >100 |
| 264 | 0.18 | >100 |
| 265 | 0.72 | >100 |
| 266 | 0.69 | 7.8 |
| 267 | 8.0 | >21 |
| 268 | 8.7 | 2.9 |
| 269 | 1.4 | >100 |
| 270 | 0.51 | 0.36 |
| 271 | 0.13 | >100 |
| 272 | 7.7 | 2.0 |
| 273 | 0.14 | 0.39 |
| 274 | 0.20 | 11 |
| 275 | 0.062 | 20 |
| 276 | 0.041 | 12 |
| 277 | 0.020 | 1.5 |
| 278 | 0.083 | 21 |
| 279 | 0.25 | 2.4 |
| 280 | 0.88 | 35 |
| 281 | 0.46 | 17 |
| 282 | 0.14 | 0.67 |
| 283 | 0.090 | 2.4 |
| 284 | 0.35 | 1.8 |
| 285 | 0.12 | 0.90 |
| 286 | 2.6 | 10 |
| 287 | 1.5 | 1.4 |
| 288 | 9.6 | 2.0 |
| 289 | 5.0 | 2.4 |
| 290 | >20 | 8.3 |
| 291 | 3.8 | 1.8 |
| 292 | 5.5 | 13 |
| 293 | 3.5 | ND |
| 294 | 0.30 | 13 |
| 295 | 0.35 | 41 |
| 296 | 5.3 | 3.0 |
| 297 | 2.7 | 2.9 |
| 298 | 6.6 | 1.4 |
| 299 | 0.24 | 3.8 |
| 300 | 0.35 | 8.6 |
| 301 | ND | 13 |
| 302 | 8.2 | 9.1 |
| 303 | 0.18 | >100 |
| 304 | 1.4 | 5.9 |
| 305 | 23 | 18 |
| 306 | 1.2 | 14 |
| 307 | ND | >82 |
| 308 | 0.56 | >100 |
| 309 | 1.4 | 41 |
| 310 | 0.97 | 38 |
| 311 | 2.3 | 20 |
| 312 | 1.3 | 7.4 |
| 313 | 4.7 | >100 |
| 314 | 3.0 | >60 |
| 315 | 11 | >100 |
| 316 | 3.4 | 10 |
| 317 | 5.0 | 5.0 |
| 318 | 6.0 | 16 |
| 319 | 7.8 | 12 |
| 320 | 100 | 1.9 |
| 321 | 0.71 | 3.5 |

### REFERENCES

Almeida, S., Gascon, E., Tran, H., Chou, H. J., Gendron, T. F., Degroot, S., Tapper, A. R., Sellier, C., Charlet-Berguerand, N., Karydas, A., et al. (2013). Modeling key pathological features of frontotemporal dementia with C9ORF72 repeat expansion in iPSC-derived human neurons. Acta Neuropathol. 126, 385-399.
Ambrus, A., Chen, D., Dai, J., Bialis, T., Jones, R. A., and Yang, D. (2006). Human telomeric sequence forms a hybrid-type intramolecular G-quadruplex structure with mixed parallel/antiparallel strands in potassium solution. Nucleic Acids Res. 34, 2723-2735.
Ash, P. E., Bieniek, K. F., Gendron, T. F., Caulfield, T., Lin, W. L., Dejesus-Hernandez, M., van Blitterswijk, M. M., Jansen-West, K., Paul, J. W., 3rd, Rademakers, R., et al. (2013). Unconventional translation of C9ORF72 GGGGCC expansion generates insoluble polypeptides specific to c9FTD/ALS. Neuron 77, 639-646.
Biffi, G., Di Antonio, M., Tannahill, D., and Balasubramanian, S. (2014). Visualization and selective chemical targeting of RNA G-quadruplex structures in the cytoplasm of human cells. Nat. Chem. 6, 75-80.
Bugaut, A., and Balasubramanian, S. (2012). 5-EUTR RNA G-quadruplexes: translation regulation and targeting. Nucleic Acids Res. 40, 4727-4741.
Burger, A. M., Dai, F., Schultes, C. M., Reszka, A. P., Moore, M. J., Double, J. A., and Neidle, S. (2005). The G-quadruplex-interactive molecule BRACO-19 inhibits tumor growth, consistent with telomere targeting and interference with telomerase function. Cancer Res. 65, 1489-1496.
Cammas, A., and Millevoi, S. (2017). RNA G-quadruplexes: emerging mechanisms in disease. Nucleic Acids Res. 45, 1584-1595.
Chen, C. Z., Sobczak, K., Hoskins, J., Southall, N., Marugan, J. J., Zheng, W., Thornton, C. A., and Austin, C. P. (2012). Two high-throughput screening assays for aberrant RNA-protein interactions in myotonic dystrophy type 1. Anal. Bioanal. Chem. 402, 1889-1898.
Chew, J., Gendron, T. F., Prudencio, M., Sasaguri, H., Zhang, Y. J., Castanedes-Casey, M., Lee, C. W., Jansen-West, K., Kurti, A., Murray, M. E., et al. (2015). Neurodegeneration. C9ORF72 repeat expansions in mice cause TDP-43 pathology, neuronal loss, and behavioral deficits. Science 348, 1151-1154.
Chou, M. Y., Rooke, N., Turck, C. W., and Black, D. L. (1999). hnRNP H is a component of a splicing enhancer complex that activates a c-src alternative exon in neuronal cells. Mol. Cell. Biol. 19, 69-77.
Collie, G. W., Campbell, N. H., and Neidle, S. (2015). Loop flexibility in human telomeric quadruplex small-molecule complexes. Nucleic Acids Res. 43, 4785-4799.
Conlon, E. G., Lu, L., Sharma, A., Yamazaki, T., Tang, T., Shneider, N. A., and Manley, J. L. (2016). The C9ORF72 GGGGCC expansion forms RNA G-quadruplex inclusions and sequesters hnRNP H to disrupt splicing in ALS brains. Elife 5, pii: e17820.
DeJesus-Hernandez, M., Mackenzie, I. R., Boeve, B. F., Boxer, A. L., Baker, M., Rutherford, N.J., Nicholson, A. M., Finch, N. A., Flynn, H., Adamson, J., et al. (2011). Expanded GGGGCC hexanucleotide repeat in noncoding region of C9ORF72 causes chromosome 9p-linked FTD and ALS. Neuron 72, 245-256.
Disney, M. D., Liu, B., Yang, W., Sellier, C., Tran, T., Charlet-Berguerand, N., and Childs-Disney, J. L. (2012). A small molecule that targets r(CGG)exp and improves defects in fragile X-associated tremor ataxia syndrome. ACS Chem. Biol. 7, 1711-1718.
Dominguez, C., Fisette, J. F., Chabot, B., and Allain, F. H. (2010). Structural basis of G-tract recognition and encaging by hnRNP F quasi-RRMs. Nat. Struct. Mol. Biol. 17, 853-861.
Donnelly, C. J., Zhang, P. W., Pham, J. T., Haeusler, A. R., Mistry, N. A., Vidensky, S., Daley, E. L., Poth, E. M., Hoover, B., Fines, D. M., et al. (2013). RNA toxicity from the ALS/FTD C9ORF72 expansion is mitigated by antisense intervention. Neuron 80, 415-428.
Endoh, T., Kawasaki, Y., and Sugimoto, N. (2013). Translational halt during elongation caused by G-quadruplex formed by mRNA. Methods 64, 73-78.
Fay, M. M., Anderson, P. J., and Ivanov, P. (2017). ALS/FTD-Associated C9ORF72 Repeat RNA Promotes Phase Transitions In Vitro and in Cells. Cell Rep. 21, 3573-3584.
Franceschin, M., Rossetti, L., DAmbrosio, A., Schirripa, S., Bianco, A., Ortaggi, G., Savino, M., Schultes, C., and Neidle, S. (2006). Natural and synthetic G-quadruplex interactive berberine derivatives. Bioorg. Med. Chem. Lett. 16, 1707-1711.
Gendron, T. F., Bieniek, K. F., Zhang, Y. J., Jansen-West, K., Ash, P. E., Caulfield, T., Daughrity, L., Dunmore, J. H., Castanedes-Casey, M., Chew, J., et al. (2013). Antisense transcripts of the expanded C9ORF72 hexanucleotide repeat form nuclear RNA foci and undergo repeat-associated non-ATG translation in c9FTD/ALS. Acta Neuropathol. 126, 829-844.
Gendron, T. F., Chew, J., Stankowski, J. N., Hayes, L. R., Zhang, Y. J., Prudencio, M., Carlomagno, Y., Daughrity, L. M., Jansen-West, K., Perkerson, E. A., et al. (2017). Poly(GP) proteins are a useful pharmacodynamic marker for C9ORF72-associated amyotrophic lateral sclerosis. Sci Transl Med 9, pii: eaai7866.
Gray, R. D., and Chaires, J. B. (2008). Kinetics and mechanism of K+- and Na+-induced folding of models of human telomeric DNA into G-quadruplex structures. Nucleic Acids Res. 36, 4191-4203.
Gray, R. D., Trent, J. O., and Chaires, J. B. (2014). Folding and unfolding pathways of the human telomeric G-quadruplex. J. Mol. Biol. 426, 1629-1650.
Guo, J. U., and Bartel, D. P. (2016). RNA G-quadruplexes are globally unfolded in eukaryotic cells and depleted in bacteria. Science 353, 1382.
Haeusler, A. R., Donnelly, C. J., Periz, G., Simko, E. A., Shaw, P. G., Kim, M. S., Maragakis, N.J., Troncoso, J. C., Pandey, A., Sattler, R., et al. (2014). C9orf72 nucleotide repeat structures initiate molecular cascades of disease. Nature 507, 195-200.
Hershman, S. G., Chen, Q., Lee, J. Y., Kozak, M. L., Yue, P., Wang, L. S., and Johnson, F. B. (2008). Genomic distribution and functional analyses of potential G-quadruplex-forming sequences in Saccharomyces cerevisiae. Nucleic Acids Res. 36, 144-156.
Huang, F. C., Chang, C. C., Lou, P. J., Kuo, I. C., Chien, C. W., Chen, C. T., Shieh, F. Y., Chang, T. C., and Lin, J. J. (2008). G-quadruplex stabilizer 3,6-bis(1-methyl-4-vinylpyridinium)carbazole diiodide induces accelerated senescence and inhibits tumorigenic properties in cancer cells. Mol. Cancer Res. 6, 955-964.
Jiang, J., Zhu, Q., Gendron, T. F., Saberi, S., McAlonis-Downes, M., Seelman, A., Stauffer, J. E., Jafar-Nejad, P., Drenner, K., Schulte, D., et al. (2016). Gain of Toxicity from ALS/FTD-Linked Repeat Expansions in C9ORF72 Is Alleviated by Antisense Oligonucleotides Targeting GGGGCC-Containing RNAs. Neuron 90, 535-550.
Kovanda, A., Zalar, M., Sket, P., Plavec, J., and Rogelj, B. (2015). Anti-sense DNA d(GGCCCC)n expansions in C9ORF72 form i-motifs and protonated hairpins. Sci. Rep. 5, 17944.
Kuo, M. H., Wang, Z. F., Tseng, T. Y., Li, M. H., Hsu, S. T., Lin, J. J., and Chang, T. C. (2015). Conformational transition of a hairpin structure to G-quadruplex within the WNT1 gene promoter. J. Am. Chem. Soc. 137, 210-218.
Lane, A. N., Chaires, J. B., Gray, R. D., and Trent, J. O. (2008). Stability and kinetics of G-quadruplex structures. Nucleic Acids Res. 36, 5482-5515.
Lee, Y. B., Chen, H. J., Peres, J. N., Gomez-Deza, J., Attig, J., Stalekar, M., Troakes, C., Nishimura, A. L., Scotter, E. L., Vance, C., et al. (2013). Hexanucleotide repeats in ALS/FTD form length-dependent RNA foci, sequester RNA binding proteins, and are neurotoxic. Cell Rep. 5, 1178-1186.
Lim, K. W., Amrane, S., Bouaziz, S., Xu, W., Mu, Y., Patel, D. J., Luu, K. N., and Phan, A. T. (2009). Structure of the human telomere in K+ solution: a stable basket-type G-quadruplex with only two G-tetrad layers. J. Am. Chem. Soc. 131, 4301-4309.
Lin, C., Wu, G., Wang, K., Onel, B., Sakai, S., Shao, Y., and Yang, D. (2018). Molecular recognition of the hybrid-2 human telomeric G-quadruplex by epiberberine: insights into conversion of telomeric G-quadruplex structures. Angew. Chem. Int. Ed. Engl. in press, https://doi.org/10.1002/anie.201804667.
Liu, W., Ikeda, Y., Hishikawa, N., Yamashita, T., Deguchi, K., and Abe, K. (2014). Characteristic RNA foci of the abnormal hexanucleotide GGCCUG repeat expansion in spinocerebellar ataxia type 36 (Asidan). Eur. J. Neurol. 21, 1377-1386.
Luu, K. N., Phan, A. T., Kuryavyi, V., Lacroix, L., and Patel, D. J. (2006). Structure of the human telomere in K+ solution: an intramolecular (3+1) G-quadruplex scaffold. J. Am. Chem. Soc. 128, 9963-9970.
Marky, L. A., and Breslauer, K. J. (1987). Calculating thermodynamic data for transitions of any molecularity from equilibrium melting curves. Biopolymers 26, 1601-1620.
Mashimo, T., Sannohe, Y., Yagi, H., and Sugiyama, H. (2008). Folding pathways of hybrid-1 and hybrid-2 G-quadruplex structures. Nucleic Acids Symp. Ser. 409-410.
Mashimo, T., and Sugiyama, H. (2007). Folding pathways of human telomeric hybrid G-quadruplex structure. Nucleic Acids Symp. Ser. 239-240.
Matsuzono, K., Imamura, K., Murakami, N., Tsukita, K., Yamamoto, T., Izumi, Y., Kaji, R., Ohta, Y., Yamashita, T., Abe, K., et al. (2017). Antisense Oligonucleotides Reduce RNA Foci in Spinocerebellar Ataxia 36 Patient iPSCs. Mol. Ther. Nucleic Acids 8, 211-219.
Mizielinska, S., Gronke, S., Niccoli, T., Ridler, C. E., Clayton, E. L., Devoy, A., Moens, T., Norona, F. E., Woollacott, I. O., Pietrzyk, J., et al. (2014). C9orf72 repeat expansions cause neurodegeneration in Drosophila through arginine-rich proteins. Science 345, 1192-1194.
Mizielinska, S., and Isaacs, A. M. (2014). C9orf72 amyotrophic lateral sclerosis and frontotemporal dementia: gain or loss of function? Cur. Opin. Neurol. 27, 515-523.
Moye, A. L., Porter, K. C., Cohen, S. B., Phan, T., Zyner, K. G., Sasaki, N., Lovrecz, G. O., Beck, J. L., and Bryan, T. M. (2015). Telomeric G-quadruplexes are a substrate and site of localization for human telomerase. Nat. Commun. 6, 7643.
Murat, P., Zhong, J., Lekieffre, L., Cowieson, N. P., Clancy, J. L., Preiss, T., Balasubramanian, S., Khanna, R., and Tellam, J. (2014). G-quadruplexes regulate Epstein-Barr virus-encoded nuclear antigen 1 mRNA translation. Nat. Chem. Biol. 10, 358-364.
ORourke, J. G., Bogdanik, L., Muhammad, A. K., Gendron, T. F., Kim, K. J., Austin, A., Cady, J., Liu, E. Y., Zarrow, J., Grant, S., et al. (2015). C9orf72 BAC transgenic mice display typical pathologic features of ALS/FTD. Neuron 88, 892-901.
Obayashi, M., Stevanin, G., Synofzik, M., Monin, M. L., Duyckaerts, C., Sato, N., Streichenberger, N., Vighetto, A., Desestret, V., Tesson, C., et al. (2015). Spinocerebellar ataxia type 36 exists in diverse populations and can be caused by a short hexanucleotide GGCCTG repeat expansion. J. Neurol. Neurosurg. Psychiatry 86, 986-995.
Parkesh, R., Childs-Disney, J. L., Nakamori, M., Kumar, A., Wang, E., Wang, T., Hoskins, J., Tran, T., Housman, D., Thornton, C. A., et al. (2012). Design of a bioactive small molecule that targets the myotonic dystrophy type 1 RNA via an RNA motif-ligand database and chemical similarity searching. J. Am. Chem. Soc. 134, 4731-4742.
Phan, A. T., Kuryavyi, V., Luu, K. N., and Patel, D. J. (2007). Structure of two intramolecular G-quadruplexes formed by natural human telomere sequences in K⁺ solution. Nucleic Acids Res. 35, 6517-6525.
Prudencio, M., Belzil, V. V., Batra, R., Ross, C. A., Gendron, T. F., Pregent, L. J., Murray, M. E., Overstreet, K. K., Piazza-Johnston, A. E., Desaro, P., et al. (2015). Distinct brain transcriptome profiles in C9orf72-associated and sporadic ALS. Nat. Neurosci. 18, 1175-1182.
Renton, A. E., Majounie, E., Waite, A., Simon-Sanchez, J., Rollinson, S., Gibbs, J. R., Schymick, J. C., Laaksovirta, H., van Swieten, J. C., Myllykangas, L., et al. (2011). A hexanucleotide repeat expansion in C9ORF72 is the cause of chromosome 9p21-linked ALS-FTD. Neuron 72, 257-268.
Rode, A. B., Endoh, T., and Sugimoto, N. (2016). tRNA shifts the G-quadruplex-hairpin conformational equilibrium in RNA towards the hairpin conformer. Angew. Chem. Int. Ed. Engl. 55, 14315-14319.
Rodriguez, R., Miller, K. M., Forment, J. V., Bradshaw, C. R., Nikan, M., Britton, S., Oelschlaegel, T., Xhemalce, B., Balasubramanian, S., and Jackson, S. P. (2012). Small-molecule-induced DNA damage identifies alternative DNA structures in human genes. Nat. Chem. Biol. 8, 301-310.
Rzuczek, S. G., Pilch, D. S., Liu, A., Liu, L., LaVoie, E. J., and Rice, J. E. (2010). Macrocyclic pyridyl polyoxazoles: selective RNA and DNA G-quadruplex ligands as antitumor agents. J. Med. Chem. 53, 3632-3644.
Sabharwal, N. C., Savikhin, V., Turek-Herman, J. R., Nicoludis, J. M., Szalai, V. A., and Yatsunyk, L. A. (2014). N-methylmesoporphyrin IX fluorescence as a reporter of strand orientation in guanine quadruplexes. FEBS J. 281, 1726-1737.
Siddiqui-Jain, A., Grand, C. L., Bearss, D. J., and Hurley, L. H. (2002). Direct evidence for a G-quadruplex in a promoter region and its targeting with a small molecule to repress c-MYC transcription. Proc. Natl. Acad. Sci. U.S.A 99, 11593-11598.
Simone, R., Balendra, R., Moens, T. G., Preza, E., Wilson, K. M., Heslegrave, A., Woodling, N. S., Niccoli, T., Gilbert-Jaramillo, J., Abdelkarim, S., et al. (2018). G-quadruplex-binding small molecules ameliorate C9orf72 FTD/ALS pathology in vitro and in vivo. EMBO Mol. Med. 10, 22-31.
Simone, R., Fratta, P., Neidle, S., Parkinson, G. N., and Isaacs, A. M. (2015). G-quadruplexes: Emerging roles in neurodegenerative diseases and the non-coding transcriptome. FEBS Lett. 589, 1653-1668.
Su, Z., Zhang, Y., Gendron, T. F., Bauer, P. O., Chew, J., Yang, W. Y., Fostvedt, E., Jansen-West, K., Belzil, V. V., Desaro, P., et al. (2014). Discovery of a biomarker and lead small molecules to target r(GGGGCC)-associated defects in c9FTD/ALS. Neuron 83, 1043-1050.
Tabet, R., Schaeffer, L., Freyermuth, F., Jambeau, M., Workman, M., Lee, C. Z., Lin, C. C., Jiang, J., Jansen-West, K., Abou-Hamdan, H., et al. (2018). CUG initiation and frameshifting enable production of dipeptide repeat proteins from ALS/FTD C9ORF72 transcripts. Nat. Commun. 9, 152.
Taylor, J. P. (2014). Neurodegenerative diseases: G-quadruplex poses quadruple threat. Nature 507, 175-177.
Taylor, J. P., Brown, R. H., Jr., and Cleveland, D. W. (2016). Decoding ALS: from genes to mechanism. Nature 539, 197-206.
Tran, T., Childs-Disney, J. L., Liu, B., Guan, L., Rzuczek, S., and Disney, M. D. (2014). Targeting the r(CGG) repeats that cause FXTAS with modularly assembled small molecules and oligonucleotides. ACS Chem. Biol. 9, 904-912.
Wang, J. M., Huang, F. C., Kuo, M. H., Wang, Z. F., Tseng, T. Y., Chang, L. C., Yen, S. J., Chang, T. C., and Lin, J. J. (2014). Inhibition of cancer cell migration and invasion through suppressing the Wnt1-mediating signal pathway by G-quadruplex structure stabilizers. J. Biol. Chem. 289, 14612-14623.
Xu, H., Di Antonio, M., McKinney, S., Mathew, V., Ho, B., ONeil, N. J., Santos, N. D., Silvester, J., Wei, V., Garcia, J., et al. (2017). CX-5461 is a DNA G-quadruplex stabilizer with selective lethality in BRCA1/2 deficient tumours. Nat. Commun. 8, 14432.
Yang, W. Y., He, F., Strack, R. L., Oh, S. Y., Frazer, M., Jaffrey, S. R., Todd, P. K., and Disney, M. D. (2016). Small molecule recognition and tools to study modulation of r(CGG)^{exp} in fragile X-associated tremor ataxia syndrome. ACS Chem. Biol. 11, 2456-2465.
Yang, W. Y., Wilson, H. D., Velagapudi, S. P., and Disney, M. D. (2015). Inhibition of non-ATG translational events in cells via covalent small molecules targeting RNA. J. Am. Chem. Soc. 137, 5336-5345.
Zamiri, B., Reddy, K., Macgregor, R. B., Jr., and Pearson, C. E. (2014). TMPyP4 porphyrin distorts RNA G-quadruplex structures of the disease-associated r(GGGGCC)n repeat of the C9orf72 gene and blocks interaction of RNA-binding proteins. J. Biol. Chem. 289, 4653-4659.
Zhang, A. Y., and Balasubramanian, S. (2012). The kinetics and folding pathways of intramolecular G-quadruplex nucleic acids. J. Am. Chem. Soc. 134, 19297-19308.
Zhang, J. H., Chung, T. D., and Oldenburg, K. R. (1999). A simple statistical parameter for use in evaluation and validation of high throughput screening assays. J. Biomol. Screen. 4, 67-73.
Zu, T., Liu, Y., Banez-Coronel, M., Reid, T., Pletnikova, O., Lewis, J., Miller, T. M., Harms, M. B., Falchook, A. E., Subramony, S. H., et al. (2013). RAN proteins and RNA foci from antisense transcripts in C9ORF72 ALS and frontotemporal dementia. Proc. Natl. Acad. Sci. U.S.A. 110, E4968-4977.

We claim:

1. A compound, or pharmaceutically acceptable salt thereof, having a structure of Formula (I): 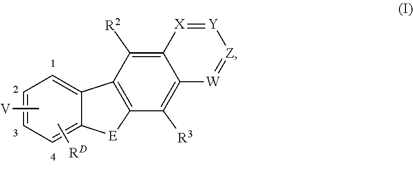
wherein
one or two of W, X, Y, and Z is N and the others are CR⁴, or each of W, X, Y, and Z is CR⁴;
E is NR⁶, O, or S;
V is H, halo, OR¹, C(O)NR^{N}R¹, C(O)OR¹, or NR^{A}R^{B};
R¹ is H, C₁₋₆alkyl, C₁₋₆haloalkyl, C₁₋₆cyanoalkyl, C₁₋₆hydroxyalkyl, C₁₋₆alkylene-C₁₋₆ alkoxy, C₂₋₆alkenyl, C₂₋₆alkynyl, C₀₋₆alkylene-C(O)C₁₋₆alkyl, C₀₋₆alkylene-CO₂R^{N}, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-aryl, C₀₋₆alkylene-(5-10 membered heteroaryl having 1-3 ring heteroatoms selected from N, O and S), C₁₋₆alkylene-C(NH)NR^{A}R^{B}, C₁₋₆alkylene-NR^{A}R^{B}, C₁₋₆alkylene-C(O)NR^{A}R^{B}, C(O)—C₃₋₄carbocyclyl, or C(O)-(3-14 membered heterocyclyl having 1-3 ring heteroatoms selected from N, O and S), and the carbocyclyl, aryl, heterocyclyl, or heteroaryl is optionally substituted with 1 or 2 R^{C} groups;
each of R², R³, and R⁴ is independently H, halo, CN, OH, C₁₋₆alkyl, C₁₋₆haloalkyl, C₁₋₆alkoxy, C₂₋₆alkenyl, C₂₋₆alkynyl, C₀₋₆alkylene-aryl, NR^{A}R^{B}, SO₀₋₂C₁₋₆alkyl, SO₀₋₂aryl, C(O)C₁₋₆alkyl, C₀₋₆alkylene-C₃₋₁₄carbocyclyl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), C(O)C₃₋₁₄carbocyclyl, CO₂H, C(O)NR^{N}—C₁₋₆alkylene-NR^{A}R^{B}, or C₁₋₆alkylene-NR^{A}R^{B}, and the carbocyclyl, heterocyclyl, aryl, or heteroaryl is optionally substituted with 1 or 2 R^{C} groups;
R⁶ is H, C₁₋₆alkyl, C₁₋₆haloalkyl, C₁₋₈alkylene-NR^{A}R^{B}, C₁₋₆alkylene-C₃₋₁₄carbocyclyl, C₁₋₆alkylene-(3-14 membered heterocyclyl having 1-3 ring heteroatoms selected from N, O and S), C₁₋₆alkylene-aryl, C₁₋₆hydroxyalkylene-aryl, or C₁₋₆alkylene-(5-10 membered heteroaryl having 1-3 ring heteroatoms selected from N, O and S), and carbocyclyl, heterocyclyl, aryl or heteroaryl is optionally substituted with 1 or 2 R^{C};
R^{A} and R^{B} are independently H, C₁₋₆alkyl, C₁₋₆haloalkyl, C₁₋₆hydroxyalkyl, C₂₋₆alkenyl, C₂₋₆alkynyl, C₂₋₈alkylene-C₁₋₆alkoxy, SO₀₋₂C₁₋₆alkyl, SO₀₋₂aryl, C(O)C₁₋₈alkyl, C₂₋₈alkylene-N(R^{N})R^{N}, C₁₋₆alkylene-C(O)NR^{N}R^{N}, C(O)C₃₋₁₄carbocyclyl, C₀₋₆alkylene-C₃₋₁₄carbocyclyl, C₀₋₆alkylene-aryl, C₀₋₆alkylene-(3-14 membered heterocyclyl having 1-3 ring heteroatoms selected from N, O and S), C₀₋₆alkylene-(5-10 membered heteroaryl having 1-3 ring heteroatoms selected from N, O and S), and the carbocyclyl, heterocyclyl, or heteroaryl is optionally substituted with 1 or 2 R^{C} groups; or
R^{A} and R^{B} together with the nitrogen to which they are attached can form a 4-8 membered heterocyclyl comprising 0-2 additional ring heteroatoms selected from N, O, and S, and the heterocyclyl is optionally substituted with 1 or 2 R^{C} groups and optionally has a spiro cyclopropyl substituent;
each R^{N} is independently H or C₁₋₄alkyl;
each R^{C} is independently halo, OH, oxo, NR^{N}R^{N}, C₁₋₆alkyl, C₁₋₆alkoxy, C(O)C₁₋₆alkyl, CO₂C₁₋₆alkyl, or C₀₋₆alkylene-(3-14 membered heterocyclyl having 1-3 ring heteroatoms selected from N, O and S); and
R^{D} is H, C₁₋₆alkyl, C₁₋₆haloalkyl, or halo;
with the proviso that when R² and R³ are each Me, R^{D} is H, V is OR¹, Y is N, and each of W, X, and Z is CR⁴, then
(i) X is CH and OR¹ is not OH or OMe; or
(ii) E is not NH or NMe; or
(iii) OR¹ is not at the 2-position.

2. The compound or salt of claim 1, wherein V is at the 1-position.

3. The compound or salt of claim 1, wherein V is at the 2-position.

4. The compound or salt of claim 1, wherein V is at the 3-position.

5. The compound or salt of claim 1, wherein V is at the 4-position.

6. The compound or salt of any one of claims 2 to 5, wherein V is OR¹.

7. The compound or salt of any one of claims 2 to 5, wherein V is C(O)NR^{N}R¹ or C(O)R¹.

8. The compound or salt of claim 6 or 7, wherein R¹ is C₁₋₆alkyl, C₀₋₆alkylene-CO₂R^{N}, C₁₋₆alkylene-C(NH)NR^{A}R^{B}, C₁₋₆alkyleneNR^{A}R^{B}, C₁₋₆alkylene-C(O)NR^{A}R^{B}, or C₁₋₆alkylene(3-14 membered heterocyclyl having 1-3 ring heteroatoms selected from N, O and S).

9. The compound or salt of claim 8, wherein R¹ is C₁₋₈alkyleneNR^{A}R^{B}.

10. The compound or salt of claim 9, wherein R¹ is C₁₋₆alkylene-piperazinyl, C₁₋₆alkylene-(N-methylpiperazinyl), C₁₋₆alkylene-morpholinyl, C₁₋₆alkylene-N(Me)₂, C₁₋₆alkylene-piperazinyl, C₁₋₆alkylene-N(Et)₂, C₁₋₆alkylene-pyrrolidinyl, C₁₋₆alkylene-dihydroimidazolyl, or C₁₋₆alkylene-piperidinyl.

11. The compound or salt of claim 8, wherein R¹ is C₁₋₈alkylene-C(NH)NR^{A}R^{B}.

12. The compound or salt of claim 11, wherein R¹ is C₁₋₆alkylene-C(NH)NH₂.

13. The compound or salt of claim 8, wherein R¹ is C₁₋₆alkylene(3-14 membered heterocyclyl having 1-3 ring heteroatoms selected from N, O and S).

14. The compound or salt of claim 13, wherein R¹ is C₁₋₆alkylene-(4,5-dihydro-2-imidazolyl).

15. The compound or salt of claim 8, wherein R¹ is C₁₋₈alkylene-C(O)NR^{A}R^{B}.

16. The compound or salt of claim 15, wherein R¹ is C₁₋₆alkylene-C(O)NH₂, C₁₋₆ alkylene-C(O)NMe₂, C₁₋₆alkylene-C(O)NHMe, C₁₋₆alkylene-C(O)-piperazinyl, C₁₋₆alkylene-C(O)-(methylpiperazinyl), C₁₋₆alkylene-C(O)NHC₁₋₆alkylene-morpholinyl, C₁₋₆alkylene-C(O)NHC₁₋₆alkylene-NH₂, C₁₋₆alkylene-C(O)NHC₁₋₆alkylene-NHMe, or C₁₋₆alkylene-C(O)NHC₁₋₆alkylene-NMe₂.

17. The compound or salt of any one of claims 2 to 5, wherein V is NR^{A}R^{B}.

18. The compound or salt of claim 17, wherein V is piperazinyl optionally substituted with methyl or optionally comprising a spiro cyclopropyl substituent.

19. The compound or salt of any one of claims 1 to 18, wherein E is O.

20. The compound or salt of any one of claims 1 to 18, wherein E is S.

21. The compound or salt of any one of claims 1 to 18, wherein E is NR⁶.

22. The compound or salt of claim 21, wherein R⁶ is C₁₋₆alkyl or C₁₋₆haloalkyl.

23. The compound or salt of claim 21, wherein R⁶ is H or Me.

24. The compound or salt of claim 21, wherein R⁶ is C₁₋₆alkylene-NR^{A}R^{B}.

25. The compound or salt of claim 24, wherein R⁶ is C₁₋₆alkylene-piperazinyl, C₁₋₆alkylene-(methylpiperazinyl), C₁₋₆alkylene-morpholinyl, C₁₋₆alkylene-N(Me)₂, C₁₋₆alkylene-piperazinyl, C₁₋₆alkylene-N(Et)₂, C₁₋₆alkylene-pyrrolidinyl, or C₁₋₆alkylene-piperidinyl.

26. The compound or salt of claim 21, wherein R⁶ is C₁₋₆alkylene-C₃₋₁₄carbocyclyl, C₁₋₆alkylene-(3-14 membered heterocyclyl having 1-3 ring heteroatoms selected from N, O and S), C₁₋₆alkylene-aryl, C₁₋₆hydroxyalkylene-aryl, or C₁₋₆alkylene-(5-10 membered heteroaryl having 1-3 ring heteroatoms selected from N, O and S).

27. The compound or salt of claim 26, wherein R⁶ is benzyl, C₁₋₆hydroxyalkylenephenyl, C₁₋₆alkylene-pyridinyl, C₁₋₆alkylene-tetrahydrothiophenedioxide, C₁₋₆ alkylene-imidazoyl, C₁₋₆alkylene-(methylimidazolyl), or C₁₋₆alkylene-(4,5-dihydro-2-imidazolyl).

28. The compound or salt of any one of claims 1 to 27, wherein R² is H.

29. The compound or salt of claim 28, wherein R³ is Me.

30. The compound or salt of any one of claims 1 to 27, wherein R² is Me.

31. The compound or salt of claim 30, wherein R³ is Me.

32. The compound or salt of any one of claims 1 to 27, wherein R² is OH.

33. The compound or salt of any one of claims 1 to 28, 30, and 32, wherein R³ is H.

34. The compound or salt of any one of claims 1 to 28, 30, and 32, wherein R³ is Me.

35. The compound or salt of any one of claims 1 to 28, 30, and 32, wherein R³ is F or CF₃.

36. The compound or salt of any one of claims 1 to 35, wherein X is N and W, Y, and Z are each CR⁴.

37. The compound or salt of claim 36, wherein each R⁴ is H.

38. The compound or salt of any one of claims 1 to 35, wherein Y is N and W, X, and Z are each CR⁴.

39. The compound or salt of claim 38, wherein each R⁴ is H.

40. The compound or salt of any one of claims 1 to 35, wherein Z is N and W, X, and Y are each CR⁴.

41. The compound or salt of claim 40, wherein each R⁴ is H.

42. The compound or salt of any one of claims 1 to 35, wherein W is N and X, Y, and Z are each CR⁴.

43. The compound or salt of claim 42, wherein each R⁴ is H.

44. The compound or salt of any one of claims 1 to 35, wherein each of X, Y, Z, and W is CR⁴.

45. The compound or salt of claim 44, wherein each R⁴ is H.

46. The compound or salt of any one of claims 1 to 54, wherein each R⁴ is independently H, CO₂H, C(O)NR^{N}—C₁-C₆-alkylene-NR^{A}R^{B}, or C₁₋₆alkylene-NR^{A}R^{B}.

47. The compound or salt of claim 46, wherein one R⁴ is CO₂H, C(O)NR^{N}—C₁-C₆-alkylene-NR^{A}R^{B}, or C₁₋₆alkylene-NR^{A}R^{B}, and all other R⁴ are H.

48. The compound or salt of claim 46 or 47, wherein at least one R⁴ is C(O)NHC₁₋₆ alkyleneNMe₂ or CO₂H.

49. The compound or salt of any one of claims 1 to 48, wherein R^{D} is H.

50. The compound or salt of any one of claims 1 to 48, wherein R^{D} is F or Cl.

51. A compound, or pharmaceutically acceptable salt thereof, as listed in Table A.

52. A compound, or pharmaceutically acceptable salt thereof, as listed in Table B.

53. A compound, or pharmaceutically acceptable salt thereof, as listed in Table C.

54. A compound, or pharmaceutically acceptable salt thereof, having a structure of Formula (Xa): 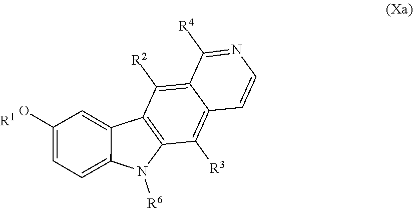
wherein
R¹ is H, C₁₋₆alkyl, C₁₋₆alkylene-C(O)C₁₋₆alkyl, C₁₋₈alkylene-NR^{A}R^{B}, C₁₋₆alkylene-(3- to 14-membered heterocyclyl), or C(O)-(3- to 14-membered heterocyclyl), wherein 1-4 heterocyclyl ring members are independently selected from N, O, and S; and the heterocyclyl is optionally substituted with 1 or 2 R^{C} groups;
R^{A} and R^{B} are independently selected from the group consisting of H, C₁₋₆alkyl, C₂₋₆alkenyl, C₂₋₆alkynyl, S(O)₀₋₂—C₁₋₆alkyl, S(O)₀₋₂-aryl, C(O)C₂₋₈alkyl, C(O)C₃₋₁₄carbocyclyl, C₃₋₁₄ carbocyclyl, C₂₋₈alkylene-C₃₋₁₄carbocyclyl, aryl, 3- to 14-membered heterocyclyl, C₁₋₆alkylene-(3- to 14-membered heterocyclyl), and 5- to 10-membered heteroaryl, and wherein 1-4 heteroaryl or 1-4 heterocyclyl ring atoms are independently selected from N, O, and S, and the carbocyclyl, aryl, heteroaryl, or heterocyclyl is optionally substituted with 1 or 2 R^{C} groups;
each R^{C} is independently halo, OH, C₁₋₆alkoxy, C(O)C₁₋₆alkyl, C(O)OC₁₋₆alkyl, C₁₋₆alkyl, 3- to 14-membered heterocyclyl, and wherein 1-4 heterocyclyl ring members are independently selected from N, O, and S;
R⁴ is H, C₁₋₆alkyl, NR^{A}R^{B}, C(O)OC₁₋₆alkyl, C(O)NR^{A}—C₁₋₆alkylene-NR^{A}R^{B}, or C(O)NR^{A}—C₁₋₆alkylene-OR^{B};
R² and R³ are each independently H or CH₃; and
R⁶ is H or CH₃.

55. A pharmaceutical composition comprising the compound or salt of any one of claims 1 to 54 and a pharmaceutically acceptable excipient.

56. A method of treating a subject suffering from a disease or condition characterized by an expanded G₄C₂ repeat RNA (r(G₄C₂)^{exp}) comprising administering a therapeutically effective amount of the compound or salt of any one of claims 1 to 54.

57. The method of claim 56, wherein the disease or condition is amyotrophic lateral sclerosis (ALS) or frontotemporal dementia (FTD).

58. The method of claim 56, wherein the method comprises inhibiting a RAN translation which produces toxic dipeptide repeat proteins.

59. The method of claim 58, where the method comprises inhibiting nuclear foci.

60. The method of claim 58 or 59, wherein the method comprises rescue of nuclear pore dysfunction.

61. The method of any one of claims 58 to 60, wherein the method comprises rescue of protein localization.


SEQUENCE LISTING (22 sequences)

SEQ ID NO: 1 - RNA, 24 nt, Artificial Sequence
  Synthetic oligonucleotide
ggggccggggccggggccggggcc

SEQ ID NO: 2 - RNA, 48 nt, Artificial Sequence
  Synthetic oligonucleotide
ggggccggggccggggccggggccggggccggggccggggccggggcc

SEQ ID NO: 3 - RNA, 24 nt, Artificial Sequence
  Synthetic oligonucleotide
ggccccggccccggccccggcccc

SEQ ID NO: 4 - RNA, 48 nt, Artificial Sequence
  Synthetic oligonucleotide
ggccccggccccggccccggccccggccccggccccggccccggcccc

SEQ ID NO: 5 - DNA, 48 bp, Artificial Sequence
  Synthetic oligonucleotide
ggggccggggccggggccggggccggggccggggccggggccggggcc

SEQ ID NO: 6 - RNA, 24 nt, Artificial Sequence
  Synthetic oligonucleotide
cggcggcggcggcggcggcggcgg

SEQ ID NO: 7 - RNA, 36 nt, Artificial Sequence
  Synthetic oligonucleotide
cggcggcggcggcggcggcggcggcggcggcggcgg

SEQ ID NO: 8 - RNA, 20 nt, Artificial Sequence
  Synthetic oligonucleotide
ggccggccgaaaggccggcc

SEQ ID NO: 9 - RNA, 36 nt, Artificial Sequence
  Synthetic oligonucleotide
ggccggccggccggccgaaaggccggccggccggcc

SEQ ID NO: 10 - RNA, 20 nt, Artificial Sequence
  Synthetic oligonucleotide
gcgcgcgcgaaagcgcgcgc

SEQ ID NO: 11 - DNA, 396 bp, Artificial Sequence
  Synthetic oligonucleotide
ggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcc

SEQ ID NO: 12 - RNA, 396 nt, Artificial Sequence
  Synthetic oligonucleotide
ggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcc

SEQ ID NO: 13 - DNA, 372 bp, Artificial Sequence
  Synthetic oligonucleotide
tgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcctgggcc

SEQ ID NO: 14 - DNA, 434 bp, Artificial Sequence
  Synthetic oligonucleotide
tggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcctggggcc

SEQ ID NO: 15 - RNA, 36 nt, Artificial Sequence
  Synthetic oligonucleotide
ggccggccggccggccaaaaggccggccggccggcc

SEQ ID NO: 16 - RNA, 48 nt, Artificial Sequence
  Synthetic oligonucleotide
ugggccugggccugggccugggccugggccugggccugggccugggcc

SEQ ID NO: 17 - RNA, 33 nt, Artificial Sequence
  Synthetic oligonucleotide
uaggguuaggguaggguuaggguaggguuaggg

SEQ ID NO: 18 - RNA, 48 nt, Artificial Sequence; identical to SEQ ID NO: 16
  Synthetic oligonucleotide

SEQ ID NO: 19 - RNA, 33 nt, Artificial Sequence; identical to SEQ ID NO: 17
  Synthetic oligonucleotide

SEQ ID NO: 20 - RNA, 23 nt, Artificial Sequence
  Synthetic oligonucleotide
uaggguuaggguuaggguuaggg

SEQ ID NO: 21 - RNA, 372 nt, Artificial Sequence
  Synthetic oligonucleotide
ugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggccugggcc

SEQ ID NO: 22 - RNA, 12 nt, Artificial Sequence
  Synthetic oligonucleotide
ggggccggggcc